(19) United States
(12) Patent Application Publication
(10) Pub. No.: US 2023/0278995 A1
(43) Pub. Date: Sep. 7, 2023

(54) 1,3,4-OXADIAZOLE DERIVATIVE COMPOUNDS AS HISTONE DEACETYLASE 6 INHIBITOR, AND THE PHARMACEUTICAL COMPOSITION COMPRISING THE SAME

(71) Applicant: CHONG KUN DANG PHARMACEUTICAL CORP., Seoul (KR)

(72) Inventors: Chang Kon Lee, Yongin-si, Gyeonggi-do (KR); Moo Sung Ko, Yongin-si, Gyeonggi-do (KR); Seok Hyoun Yun, Yongin-si, Gyeonggi-do (KR); Hyunjin Michael Kim, Yongin-si, Gyeonggi-do (KR)

(21) Appl. No.: 17/995,947

(22) PCT Filed: Apr. 12, 2021
(86) PCT No.: PCT/KR2021/004544

§ 371 Date: Oct. 11, 2022

(30) Foreign Application Priority Data
Apr. 13, 2020 (KR) 10-2020-0044730

(51) Int. Cl. C07D 413/12; C07D 413/14

(52) U.S. Cl. CPC C07D 413/12; C07D 413/14

(57) ABSTRACT

The present invention relates to a novel compound having a histone deacetylase 6 (HDAC6) inhibitory activity, an optical isomer thereof or a pharmaceutically acceptable salt thereof, the use thereof for preparing a therapeutic medicament, a pharmaceutical composition containing the same, and a treatment method using the composition, and a preparation method thereof. The novel compound, the optical isomer thereof, or the pharmaceutically acceptable salt thereof according to the present invention has the HDAC6 inhibitory activity, which is effective in the prevention or treatment of HDAC6-mediated diseases including cancer, inflammatory diseases, autoimmune diseases, neurological or neurodegenerative diseases.

### TECHNICAL FIELD

The present invention relates to a 1,3,4-oxadiazole derivative compound having a histone deacetylase 6 (HDAC6) inhibitory activity, an optical isomer thereof, a pharmaceutically acceptable salt thereof; the use for preparing a therapeutic medicament; a treatment method using the same; a pharmaceutical composition containing the same; and a preparation method thereof.

### BACKGROUND ART

Post-translational modifications such as acetylation in cells are very important regulatory modules at the center of biological processes and are strictly controlled by a number of enzymes. Histones are core proteins that make up the chromatin, acting as spools around which DNA winds to help condensation of DNA. In addition, the balance between acetylation and deacetylation of histones plays a very important role in gene expression.

Histone deacetylases (HDACs) are enzymes that remove the acetyl group of the histone protein lysine residues constituting the chromatin, which are known to be associated with gene silencing and to induce cell cycle arrest, angiogenesis inhibition, immune regulation, cell death, and the like (Hassig et al., Curr. Opin. Chem. Biol. 1997, 1, 300-308). Further, it has been reported that inhibition of HDAC enzyme function induces cancer cell death by reducing the activity of cancer cell survivalrelated factors and activating cancer cell death-related factors in vivo (Warrell et al, J. Natl. Cancer Inst. 1998, 90, 1621-1625).

In humans, 18 HDACs are known and are classified into 4 groups depending on their homology with yeast HDACs. Here, 11 HDACs using zinc as a cofactor can be divided into three groups of Class I (HDACs 1, 2, 3, and 8), Class II (IIa: HDACs 4, 5, 7, and 9; IIb: HDACs 6 and 10) and Class IV (HDAC11). Further, 7 HDACs of Class III (SIRT 1-7) employ NAD⁺ as a cofactor instead of zinc (Bolden et al., Nat. Rev. Drug. Discov. 2006, 5(9), 769-784).

Various HDAC inhibitors are in the preclinical or clinical development stage. However, until now, only non-selective HDAC inhibitors are known as anticancer agents, wherein vorinostat (SAHA) and romidepsin (FK228) have been approved as treatments for cutaneous T-cell lymphoma, and panobinostat (LBH-589) has been approved as a treatment for multiple myeloma. However, non-selective HDACs inhibitors are generally known to cause side effects such as fatigue and nausea, and the like, at high doses (Piekarz et al., Pharmaceuticals 2010, 3, 2751-2767). These side effects are reported to be caused by inhibition of Class I HDACs, and due to these side effects, non-selective HDACs inhibitors have been limited in drug development in fields other than anticancer agents (Witt et al., Cancer Letters 277 (2009) 8.21).

Meanwhile, it has been reported that selective Class II HDAC inhibition may not show the toxicity seen in Class I HDAC inhibition, and if a selective Class II HDAC inhibitor is developed, side effects such as toxicity caused by the non-selective HDAC inhibition may be solved, and thus the selective HDAC inhibitor has the potential to be developed as effective therapeutic agent for various diseases (Matthias et al., Mol. Cell. Biol. 2008, 28, 1688-1701).

HDAC6, one of the Class IIb HDACs, is mainly present in the cytoplasma and is known to be involved in deacetylation of a number of non-histone substrates (HSP90, cortactin, and the like) including tubulin proteins (Yao et al., Mol. Cell 2005, 18, 601-607). The HDAC6 has two catalytic domains, and the C-terminal of zinc-finger domain may bind to ubiquitinated proteins. Since the HDAC6 has a large number of non-histone proteins as substrates, it is known to play an important role in various diseases such as cancer, inflammatory diseases, autoimmune diseases, neurological diseases, and neurodegenerative disorders, and the like (Santo et al., Blood 2012 119: 2579-258; Vishwakarma et al., International Immunopharmacology 2013, 16, 72-78; Hu et al., J. Neurol. Sci. 2011, 304, 1-8).

A common structural feature of various HDAC inhibitors is that they consist of a cap group, a linker group, and a zinc-binding group (ZBG), as shown in the structure of vorinostat below. Many researchers have studied the inhibitory activity and selectivity for enzymes through structural modifications of the cap group and linker group. Among the groups, the zinc-binding group is known to play a more important role in the enzyme inhibitory activity and selectivity (Wiest et al., J. Org. Chem. 2013 78: 5051-5065; Methot et al., Bioorg. Med. Chem. Lett. 2008, 18, 973-978).

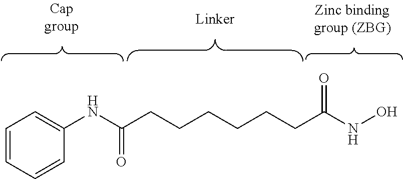 [SMILES: O=C(CCCCCCC(=O)Nc1ccccc1)NO]

Most of the zinc-binding groups are hydroxamic acid or benzamide, and among them, hydroxamic acid derivatives exhibit a strong HDAC inhibitory effect, but have problems such as low bioavailability and severe off-target activity. Since benzamide contains aniline, there is a problem that toxic metabolites may be caused in vivo (Woster et al., Med. Chem. Commun. 2015, online publication).

Therefore, for the treatment of cancer, inflammatory diseases, autoimmune diseases, neurological diseases, and neurodegenerative disorders, and the like, there is a need to develop a selective HDAC6 inhibitor having a zinc-binding group with improved bioavailability without side effects, unlike non-selective inhibitors with side effects.

### DISCLOSURE OF INVENTION

### Technical Problem

An object of the present invention is to provide a 1,3,4-oxadiazole derivative compound having a selective histone deacetylase 6 (HDAC6) inhibitory activity, an optical isomer thereof, or a pharmaceutically acceptable salt thereof.

Another object of the present invention is to provide a pharmaceutical composition including a 1,3,4-oxadiazole derivative compound having a selective HDAC6 inhibitory activity, an optical isomer thereof, or a pharmaceutically acceptable salt thereof.

Still another object of the present invention is to provide a preparation method thereof.

Still another object of the present invention is to provide a pharmaceutical composition including the compounds for preventing or treating histone deacetylase 6 (HDAC6)-mediated diseases including infectious diseases; neoplasm; endocrine, nutritional and metabolic diseases; mental and behavioral disorders; neurological diseases; diseases of eyes and adnexa; circulatory diseases; respiratory diseases; digestive diseases; skin and subcutaneous tissue diseases; musculoskeletal and connective tissue diseases; or congenital malformations, alterations, and chromosomal abnormalities.

Still another object of the present invention is to provide the use of the compounds for preparing a medicament for preventing or treating HDAC6-mediated diseases.

Still another object of the present invention is to provide a method for preventing or treating HDAC6-mediated diseases including administering a therapeutically effective amount of the composition including the compounds as described above.

### Solution to Problem

The present inventors found a 1,3,4-oxadiazole derivative compound having a histone deacetylase 6 (HDAC6) inhibitory activity to inhibit or treat HDAC6-mediated diseases, and completed the present invention.

1,3,4-Oxadiazole Derivative Compound

In one general aspect, the present invention provides a 1,3,4-oxadiazole derivative compound represented by Chemical Formula I below, an optical isomer thereof, or a pharmaceutically acceptable salt thereof:

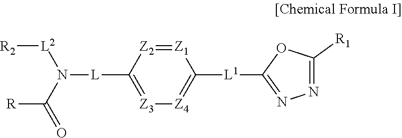

in the Chemical Formula I above,
Z₁ to Z₄ are each independently N, CH or CX, wherein Z₁ to Z₄ may not be three or more Ns at the same time;
L, L¹ or L² is each independently —(C₀-C₂alkylene)-;
R₁ is —CH₂X or —CX₃;
R₂ is aryl or heteroaryl, wherein at least one —H of the aryl or heteroaryl may each independently be substituted with —X, —OH, —(C₁-C₄alkyl) or —O(C₁-C₄alkyl);
R is

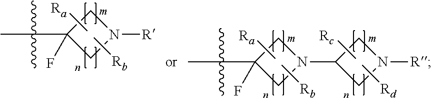

Rₐ to R_{d} are each independently —H or —(C₁-C₄alkyl);
R′ and R″ are each independently —H, —(C₁-C₄alkyl), —(C₃-C₇cycloalkyl), —(C₂-C₆ heterocycloalkyl), —(C₁-C₄alkyl)-(C₃-C₇cycloalkyl), —(C₁-C₄alkyl)-(C₂-C₆ heterocycloalkyl), —C(═O)—(C₁-C₄alkyl), —C(═O)—(C₃-C₇Cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄ alkyl) may be substituted with —X, —OH, —N(CH₃)₂ or —O(C₁-C₄alkyl), and at least one —H of —(C₃-C₇cycloalkyl), —(C₂-C₆heterocycloalkyl), —(C₁-C₄alkyl)-(C₃-C₇ cycloalkyl), —(C₁-C₄alkyl)-(C₂-C₆heterocycloalkyl) or —C(═O)—(C₃-C₇cycloalkyl) ring may be substituted with —X, —OH or —(C₁-C₄alkyl);
m or n is each independently 1, 2 or 3; and
X is F, Cl, Br or I.

Further, according to an embodiment of the present invention, in the Chemical Formula I above,
Z₁ to Z₄ are each independently N, CH or CX, wherein Z₁ to Z₄ may not be two or more Ns at the same time;
L, L₁ or L² is each independently —(C₀-C₂alkylene)-;
R₁ is —CH₂X or —CX₃;
R₂ is aryl, wherein at least one —H of the aryl may each independently be substituted with —X, —OH, —(C₁-C₄alkyl) or —O(C₁-C₄alkyl);
R is

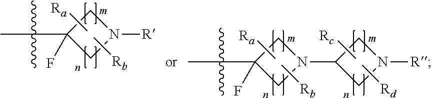

Rₐ to R_{d} are each independently —H or —(C₁-C₄alkyl);
R′ and R″ are each independently —H, —(C₁-C₄alkyl), —(C₃-C₇cycloalkyl), —(C₂-C₆ heterocycloalkyl), —(C₁-C₄alkyl)-(C₃-C₇cycloalkyl), —(C₁-C₄alkyl)-(C₂-C₆ heterocycloalkyl), —C(═O)—(C₁-C₄alkyl), —C(═O)—(C₃-C₇Cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄ alkyl) may be substituted with —X, —OH, —N(CH₃)₂ or —O(C₁-C₄alkyl), and at least one —H of —(C₃-C₇cycloalkyl), —(C₂-C₆heterocycloalkyl), —(C₁-C₄alkyl)-(C₃-C₇ cycloalkyl), —(C₁-C₄alkyl)-(C₂-C₆heterocycloalkyl) or —C(═O)—(C₃-C₇cycloalkyl) ring may be substituted with —X, —OH or —(C₁-C₄alkyl);
m or n is each independently 1, 2 or 3; and
X is F, Cl or Br.

Further, according to an embodiment of the present invention, in the Chemical Formula I above,
Z₁ to Z₄ are each independently N, CH or CX, wherein Z₁ to Z₄ may not be two or more Ns at the same time;
L is —(C₁alkylene)-;
L₁ or L² is each independently —(C₀alkylene)-;
R₁ is —CH₂X or —CX₃;
R₂ is aryl, wherein at least one —H of the aryl may each independently be substituted with —X;
R is

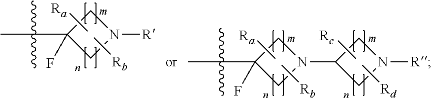

Rₐ to R_{d} are each independently —H;
R′ and R″ are each independently —H, —(C₁-C₄alkyl), —(C₃-C₇cycloalkyl), —(C₂-C₆ heterocycloalkyl), —(C₁-C₄alkyl)-(C₃-C₇cycloalkyl), —(C₁-C₄alkyl)-(C₂-C₆ heterocycloalkyl), —C(═O)—(C₁-C₄alkyl), —C(═O)—(C₃-C₇Cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄ alkyl) may be substituted with —X, —OH, —N(CH₃)₂ or —O(C₁-C₄alkyl), and at least one —H of —(C₃-C₇cycloalkyl) ring may be substituted with —X;
m or n is each independently 1 or 2; and
X is F or C₁.

Further, according to an embodiment of the present invention, in the Chemical Formula I above,
Z₁ to Z₄ are each independently N, CH or CF, wherein Z₁ to Z₄ may not be two or more Ns at the same time;
L is —(C₁alkylene)-;
L₁ or L² is each independently —(C₀alkylene)-;
R₁ is —CF₂H or —CF₃;
R₂ is aryl, wherein at least one —H of the aryl may each independently be substituted with —F;
R is

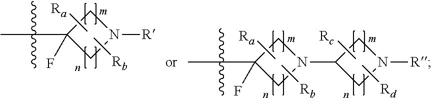

Rₐ to R_{d} are each independently —H;
R′ is —H, —(C₁-C₄alkyl), —(C₃-C₇cycloalkyl), —(C₂-C₆heterocycloalkyl), —(C₁-C₄ alkyl)-(C₃-C₇cycloalkyl), —(C₁-C₄alkyl)-(C₂-C₆heterocycloalkyl), —C(═O)—(C₁-C₄ alkyl), —C(═O)—(C₃-C₇cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄alkyl) may be substituted with —X, —OH, —N(CH₃)₂ or —O(C₁-C₄alkyl), and at least one —H of —(C₃-C₇ cycloalkyl) ring may be substituted with —X;
R″ is —(C₁-C₄alkyl), —(C₃-C₇cycloalkyl) or —(C₂-C₆heterocycloalkyl);
m or n is each independently 1 or 2; and
X is F or Cl.

In addition, according to an embodiment of the present invention, specific compounds represented by Chemical Formula I of the present invention are shown in Table 1 below:

TABLE 1

| Ex. | Comp. | Structure |
|---|---|---|
| 1 | 2865 | 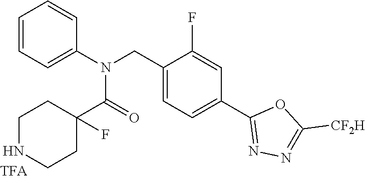 [SMILES: O=C(N(Cc1ccc(-c2nnc(C(F)F)o2)cc1F)c1ccccc1)C1(F)CCNCC1] |
| 2 | 2866 | 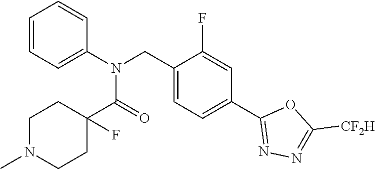 [SMILES: CN1CCC(F)(C(=O)N(Cc2ccc(-c3nnc(C(F)F)o3)cc2F)c2ccccc2)CC1] |
| 3 | 2867 | 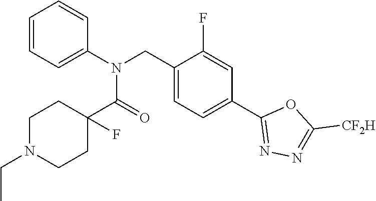 [SMILES: CCN1CCC(F)(C(=O)N(Cc2ccc(-c3nnc(C(F)F)o3)cc2F)c2ccccc2)CC1] |
| 4 | 2868 | 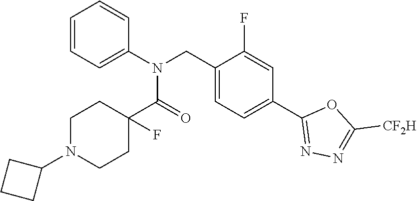 [SMILES: O=C(N(Cc1ccc(-c2nnc(C(F)F)o2)cc1F)c1ccccc1)C1(F)CCN(C2CCC2)CC1] |
| 5 | 2869 | 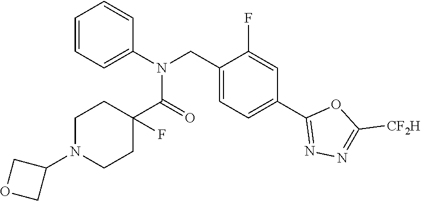 [SMILES: O=C(N(Cc1ccc(-c2nnc(C(F)F)o2)cc1F)c1ccccc1)C1(F)CCN(C2COC2)CC1] |
| 6 | 2951 | 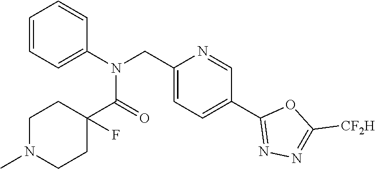 [SMILES: CN1CCC(F)(C(=O)N(Cc2ccc(-c3nnc(C(F)F)o3)cn2)c2ccccc2)CC1] |
| 7 | 2952 | 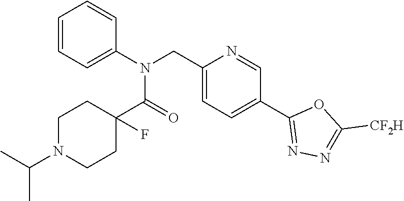 [SMILES: CC(C)N1CCC(F)(C(=O)N(Cc2ccc(-c3nnc(C(F)F)o3)cn2)c2ccccc2)CC1] |
| 8 | 2953 | 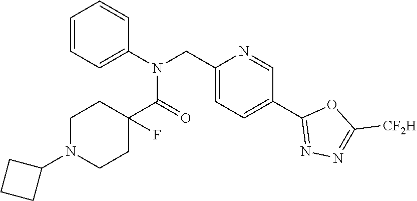 [SMILES: O=C(N(Cc1ccc(-c2nnc(C(F)F)o2)cn1)c1ccccc1)C1(F)CCN(C2CCC2)CC1] |
| 9 | 2954 | 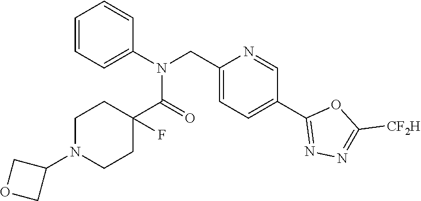 [SMILES: O=C(N(Cc1ccc(-c2nnc(C(F)F)o2)cn1)c1ccccc1)C1(F)CCN(C2COC2)CC1] |
| 10 | 2969 | 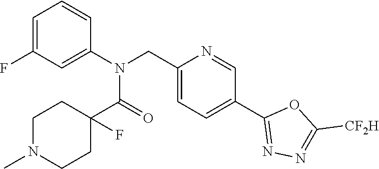 [SMILES: CN1CCC(F)(C(=O)N(Cc2ccc(-c3nnc(C(F)F)o3)cn2)c2cccc(F)c2)CC1] |
| 11 | 2970 | 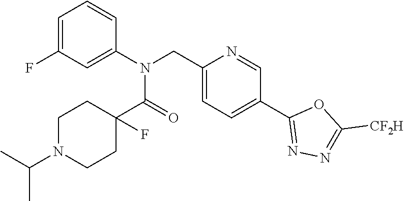 [SMILES: CC(C)N1CCC(F)(C(=O)N(Cc2ccc(-c3nnc(C(F)F)o3)cn2)c2cccc(F)c2)CC1] |
| 12 | 2971 | 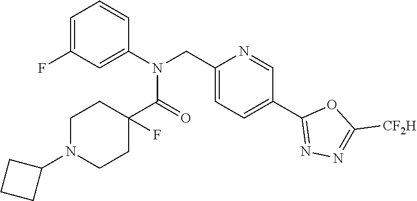 [SMILES: O=C(N(Cc1ccc(-c2nnc(C(F)F)o2)cn1)c1cccc(F)c1)C1(F)CCN(C2CCC2)CC1] |
| 13 | 2972 | 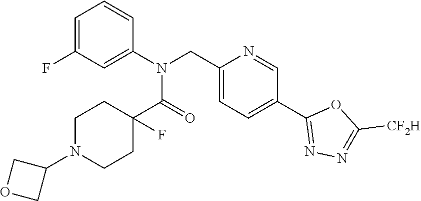 [SMILES: O=C(N(Cc1ccc(-c2nnc(C(F)F)o2)cn1)c1cccc(F)c1)C1(F)CCN(C2COC2)CC1] |
| 14 | 2973 | 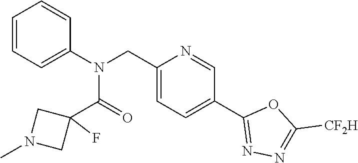 [SMILES: CN1CC(F)(C(=O)N(Cc2ccc(-c3nnc(C(F)F)o3)cn2)c2ccccc2)C1] |
| 15 | 2974 | 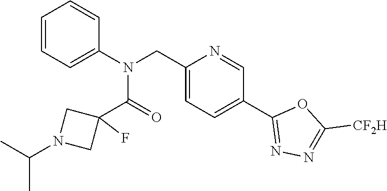 [SMILES: CC(C)N1CC(F)(C(=O)N(Cc2ccc(-c3nnc(C(F)F)o3)cn2)c2ccccc2)C1] |
| 16 | 2975 | 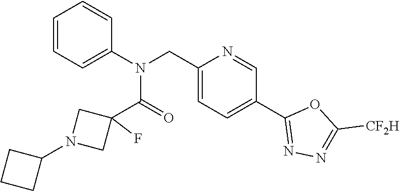 [SMILES: O=C(N(Cc1ccc(-c2nnc(C(F)F)o2)cn1)c1ccccc1)C1(F)CN(C2CCC2)C1] |
| 17 | 2976 | 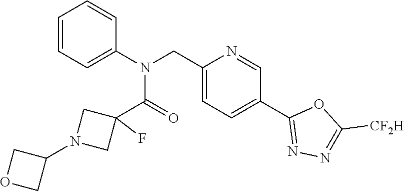 [SMILES: O=C(N(Cc1ccc(-c2nnc(C(F)F)o2)cn1)c1ccccc1)C1(F)CN(C2COC2)C1] |
| 18 | 2995 | 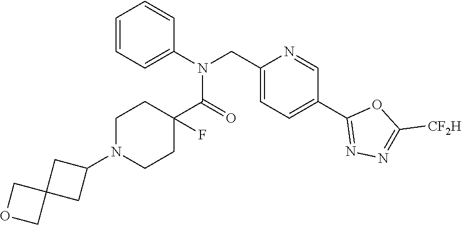 [SMILES: O=C(N(Cc1ccc(-c2nnc(C(F)F)o2)cn1)c1ccccc1)C1(F)CCN(C2CC3(COC3)C2)CC1] |
| 19 | 2996 | 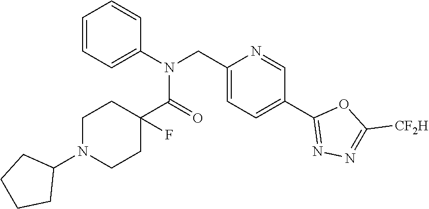 [SMILES: O=C(N(Cc1ccc(-c2nnc(C(F)F)o2)cn1)c1ccccc1)C1(F)CCN(C2CCCC2)CC1] |
| 20 | 2997 | 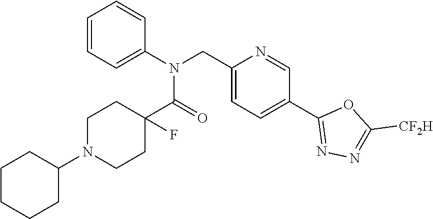 [SMILES: O=C(N(Cc1ccc(-c2nnc(C(F)F)o2)cn1)c1ccccc1)C1(F)CCN(C2CCCCC2)CC1] |
| 21 | 2998 | 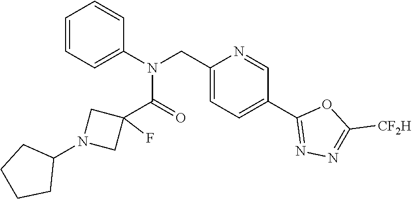 [SMILES: O=C(N(Cc1ccc(-c2nnc(C(F)F)o2)cn1)c1ccccc1)C1(F)CN(C2CCCC2)C1] |
| 22 | 2999 | 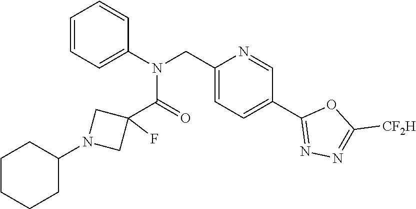 |
| 23 | 3000 | 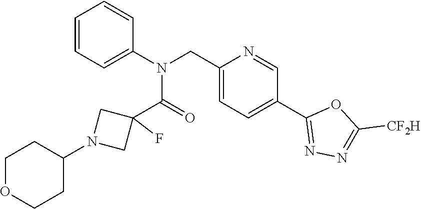 |
| 24 | 3001 | 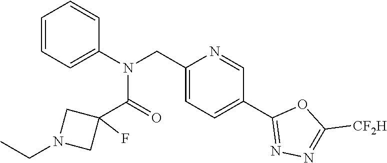 |
| 25 | 3002 | 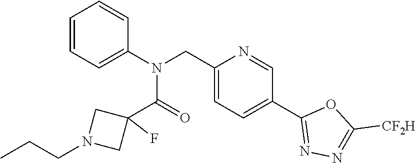 [SMILES: CCCN1CC(F)(C(=O)N(Cc2ccc(-c3nnc(C(F)F)o3)cn2)c2ccccc2)C1] |
| 26 | 3003 | 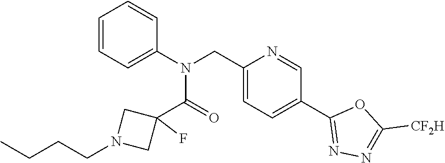 |
| 27 | 3004 | 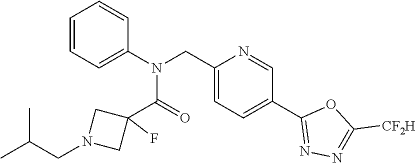 [SMILES: CC(C)CN1CC(F)(C(=O)N(Cc2ccc(-c3nnc(C(F)F)o3)cn2)c2ccccc2)C1] |
| 28 | 3005 | 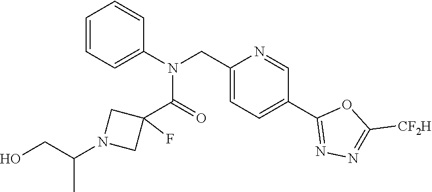 |
| 29 | 3006 | 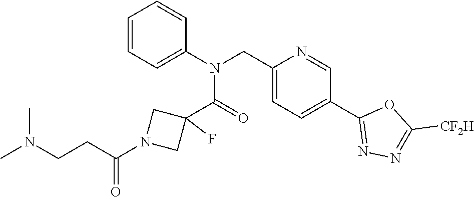 [SMILES: CN(C)CCC(=O)N1CC(F)(C(=O)N(Cc2ccc(-c3nnc(C(F)F)o3)cn2)c2ccccc2)C1] |
| 30 | 3007 | 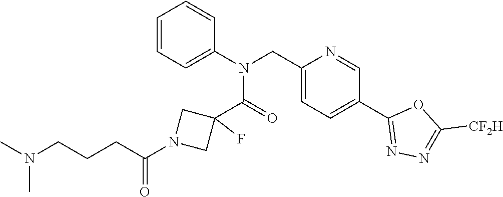 |
| 31 | 3047 | 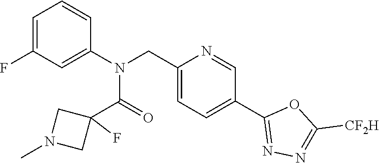 [SMILES: CN1CC(F)(C(=O)N(Cc2ccc(-c3nnc(C(F)F)o3)cn2)c2cccc(F)c2)C1] |
| 32 | 3048 | 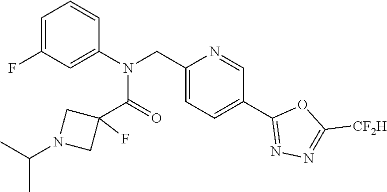 |
| 33 | 3049 | 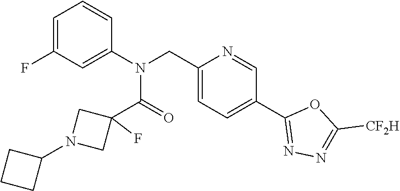 |
| 34 | 3050 | 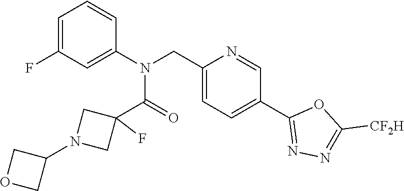 [SMILES: O=C(N(Cc1ccc(-c2nnc(C(F)F)o2)cn1)c1cccc(F)c1)C1(F)CN(C2COC2)C1] |
| 35 | 3051 | 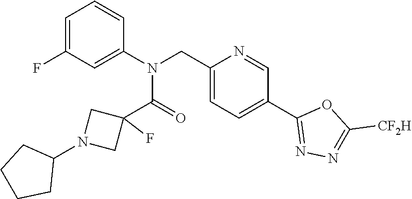 |
| 36 | 3052 | 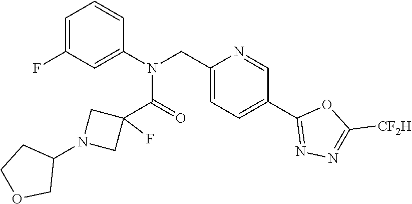 [SMILES: O=C(N(Cc1ccc(-c2nnc(C(F)F)o2)cn1)c1cccc(F)c1)C1(F)CN(C2CCOC2)C1] |
| 37 | 3053 | 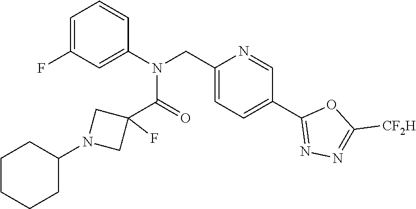 |
| 38 | 3054 | 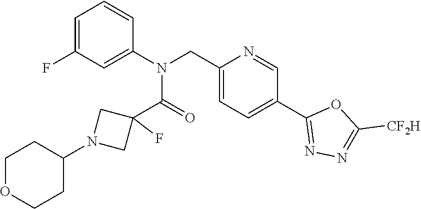 [SMILES: O=C(N(Cc1ccc(-c2nnc(C(F)F)o2)cn1)c1cccc(F)c1)C1(F)CN(C2CCOCC2)C1] |
| 39 | 3055 | 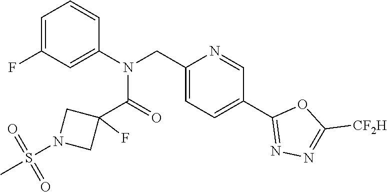 [SMILES: CS(=O)(=O)N1CC(F)(C(=O)N(Cc2ccc(-c3nnc(C(F)F)o3)cn2)c2cccc(F)c2)C1] |
| 40 | 3090 | 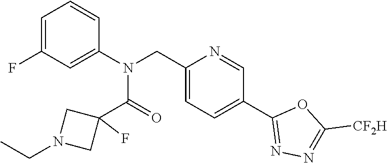 [SMILES: CCN1CC(F)(C(=O)N(Cc2ccc(-c3nnc(C(F)F)o3)cn2)c2cccc(F)c2)C1] |
| 41 | 3091 | 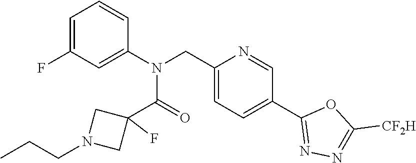 [SMILES: CCCN1CC(F)(C(=O)N(Cc2ccc(-c3nnc(C(F)F)o3)cn2)c2cccc(F)c2)C1] |
| 42 | 3092 | 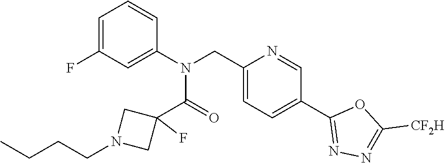 |
| 43 | 3093 | 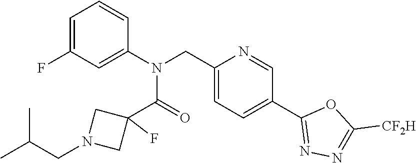 [SMILES: CC(C)CN1CC(F)(C(=O)N(Cc2ccc(-c3nnc(C(F)F)o3)cn2)c2cccc(F)c2)C1] |
| 44 | 3094 | 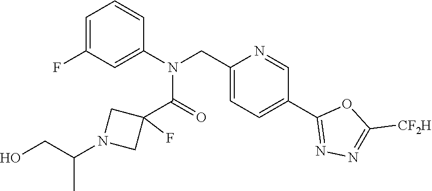 |
| 45 | 3095 | 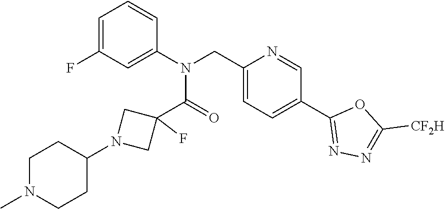 [SMILES: CN1CCC(N2CC(F)(C(=O)N(Cc3ccc(-c4nnc(C(F)F)o4)cn3)c3cccc(F)c3)C2)CC1] |
| 46 | 3096 | 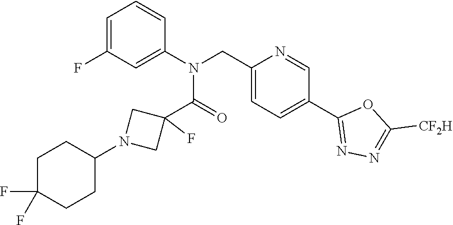 |
| 47 | 3097 | 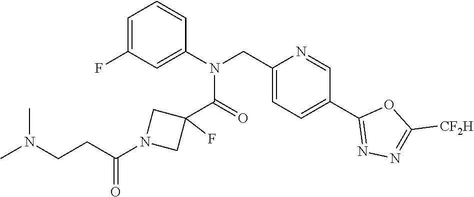 [SMILES: CN(C)CCC(=O)N1CC(F)(C(=O)N(Cc2ccc(-c3nnc(C(F)F)o3)cn2)c2cccc(F)c2)C1] |
| 48 | 3098 | 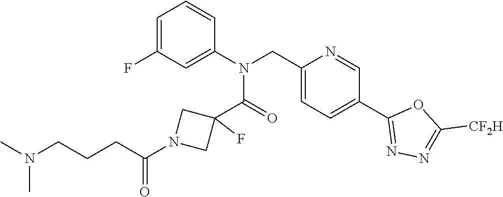 |
| 49 | 3105 | 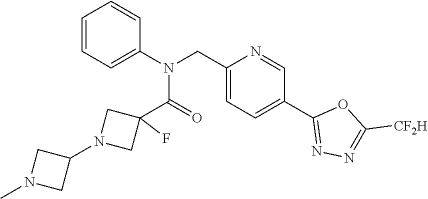 [SMILES: CN1CC(N2CC(F)(C(=O)N(Cc3ccc(-c4nnc(C(F)F)o4)cn3)c3ccccc3)C2)C1] |
| 50 | 3106 | 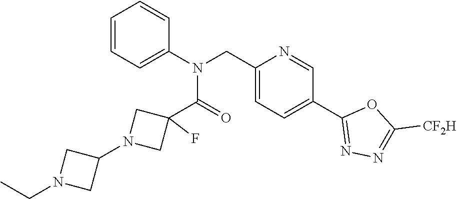 [SMILES: CCN1CC(N2CC(F)(C(=O)N(Cc3ccc(-c4nnc(C(F)F)o4)cn3)c3ccccc3)C2)C1] |
| 51 | 3107 | 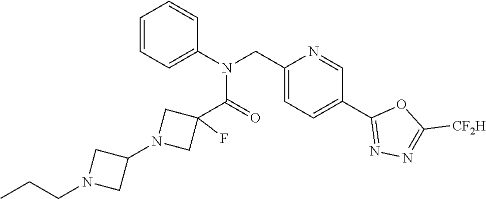 [SMILES: CCCN1CC(N2CC(F)(C(=O)N(Cc3ccc(-c4nnc(C(F)F)o4)cn3)c3ccccc3)C2)C1] |
| 52 | 3108 | 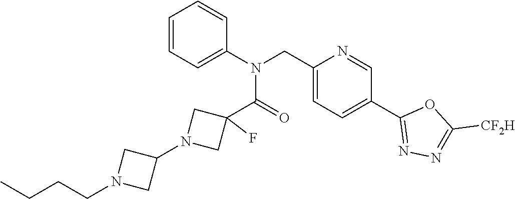 [SMILES: CCCCN1CC(N2CC(F)(C(=O)N(Cc3ccc(-c4nnc(C(F)F)o4)cn3)c3ccccc3)C2)C1] |
| 53 | 3109 | 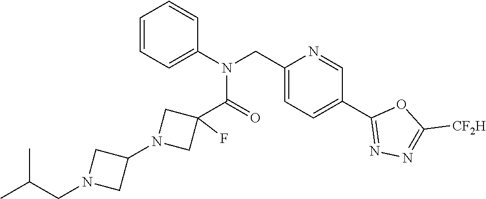 [SMILES: CC(C)CN1CC(N2CC(F)(C(=O)N(Cc3ccc(-c4nnc(C(F)F)o4)cn3)c3ccccc3)C2)C1] |
| 54 | 3110 | 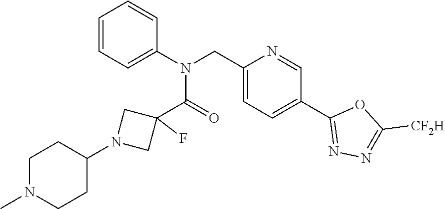 [SMILES: CN1CCC(N2CC(F)(C(=O)N(Cc3ccc(-c4nnc(C(F)F)o4)cn3)c3ccccc3)C2)CC1] |
| 55 | 3111 | 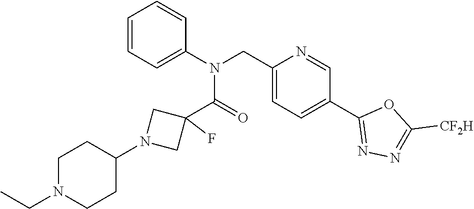 [SMILES: CCN1CCC(N2CC(F)(C(=O)N(Cc3ccc(-c4nnc(C(F)F)o4)cn3)c3ccccc3)C2)CC1] |
| 56 | 3112 | 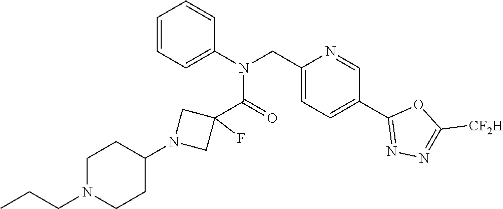 [SMILES: CCCN1CCC(N2CC(F)(C(=O)N(Cc3ccc(-c4nnc(C(F)F)o4)cn3)c3ccccc3)C2)CC1] |
| 57 | 3113 | 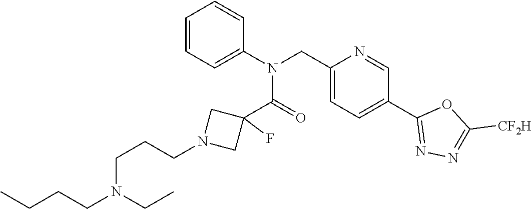 [SMILES: CCCCN(CC)CCCN1CC(F)(C(=O)N(Cc2ccc(-c3nnc(C(F)F)o3)cn2)c2ccccc2)C1] |
| 58 | 3114 | 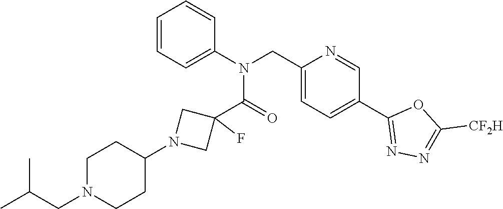 [SMILES: CC(C)CN1CCC(N2CC(F)(C(=O)N(Cc3ccc(-c4nnc(C(F)F)o4)cn3)c3ccccc3)C2)CC1] |
| 59 | 3115 | 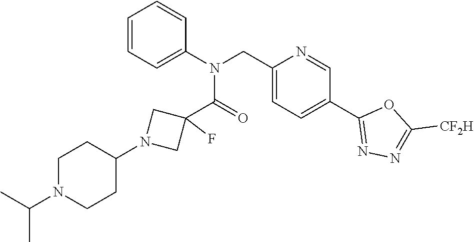 [SMILES: CC(C)N1CCC(N2CC(F)(C(=O)N(Cc3ccc(-c4nnc(C(F)F)o4)cn3)c3ccccc3)C2)CC1] |
| 60 | 3152 | 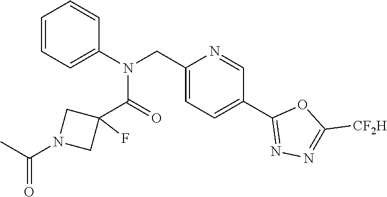 [SMILES: CC(=O)N1CC(F)(C(=O)N(Cc2ccc(-c3nnc(C(F)F)o3)cn2)c2ccccc2)C1] |
| 61 | 3153 | 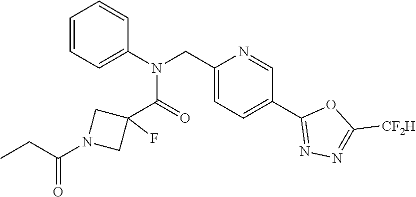 [SMILES: CCC(=O)N1CC(F)(C(=O)N(Cc2ccc(-c3nnc(C(F)F)o3)cn2)c2ccccc2)C1] |
| 62 | 3154 | 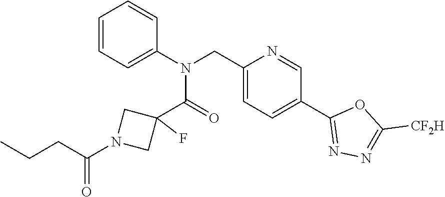 [SMILES: CCCC(=O)N1CC(F)(C(=O)N(Cc2ccc(-c3nnc(C(F)F)o3)cn2)c2ccccc2)C1] |
| 63 | 3155 | 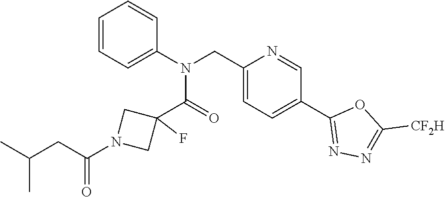 [SMILES: CC(C)CC(=O)N1CC(F)(C(=O)N(Cc2ccc(-c3nnc(C(F)F)o3)cn2)c2ccccc2)C1] |
| 64 | 3156 | 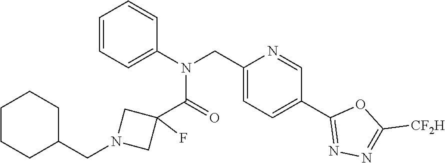 [SMILES: O=C(N(Cc1ccc(-c2nnc(C(F)F)o2)cn1)c1ccccc1)C1(F)CN(CC2CCCCC2)C1] |
| 65 | 3157 | 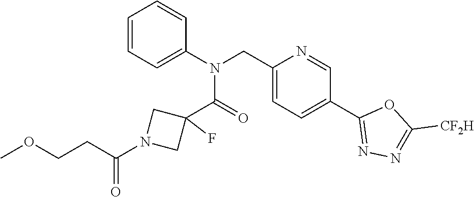 [SMILES: COCCC(=O)N1CC(F)(C(=O)N(Cc2ccc(-c3nnc(C(F)F)o3)cn2)c2ccccc2)C1] |
| 66 | 3158 | 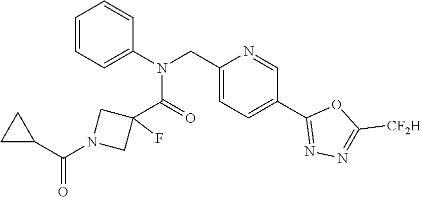 [SMILES: O=C(C1CC1)N1CC(F)(C(=O)N(Cc2ccc(-c3nnc(C(F)F)o3)cn2)c2ccccc2)C1] |
| 67 | 3159 | 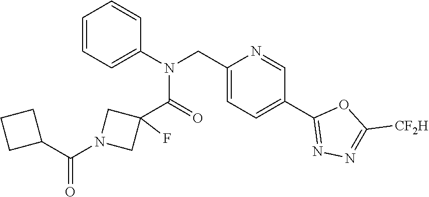 [SMILES: O=C(C1CCC1)N1CC(F)(C(=O)N(Cc2ccc(-c3nnc(C(F)F)o3)cn2)c2ccccc2)C1] |
| 68 | 3160 | 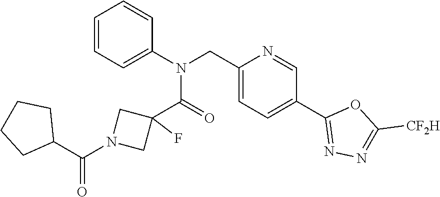 [SMILES: O=C(C1CCCC1)N1CC(F)(C(=O)N(Cc2ccc(-c3nnc(C(F)F)o3)cn2)c2ccccc2)C1] |
| 69 | 3161 | 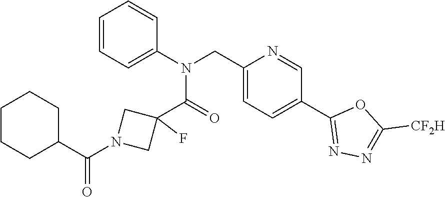 [SMILES: O=C(C1CCCCC1)N1CC(F)(C(=O)N(Cc2ccc(-c3nnc(C(F)F)o3)cn2)c2ccccc2)C1] |
| 70 | 3162 | 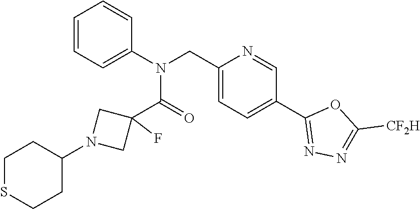 [SMILES: O=C(N(Cc1ccc(-c2nnc(C(F)F)o2)cn1)c1ccccc1)C1(F)CN(C2CCSCC2)C1] |
| 71 | 3163 | 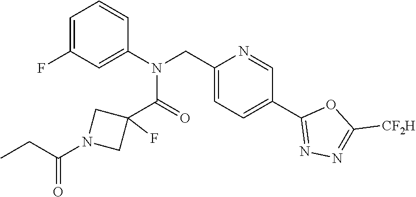 [SMILES: CCC(=O)N1CC(F)(C(=O)N(Cc2ccc(-c3nnc(C(F)F)o3)cn2)c2cccc(F)c2)C1] |
| 72 | 3164 | 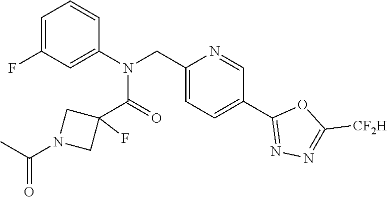 [SMILES: CC(=O)N1CC(F)(C(=O)N(Cc2ccc(-c3nnc(C(F)F)o3)cn2)c2cccc(F)c2)C1] |
| 73 | 3165 | 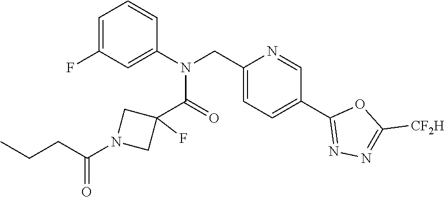 [SMILES: CCCC(=O)N1CC(F)(C(=O)N(Cc2ccc(-c3nnc(C(F)F)o3)cn2)c2cccc(F)c2)C1] |
| 74 | 3166 | 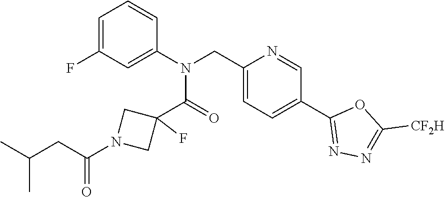 [SMILES: CC(C)CC(=O)N1CC(F)(C(=O)N(Cc2ccc(-c3nnc(C(F)F)o3)cn2)c2cccc(F)c2)C1] |
| 75 | 3167 | 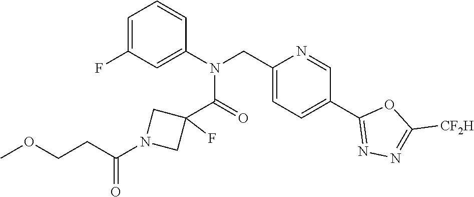 [SMILES: COCCC(=O)N1CC(F)(C(=O)N(Cc2ccc(-c3nnc(C(F)F)o3)cn2)c2cccc(F)c2)C1] |
| 76 | 3168 | 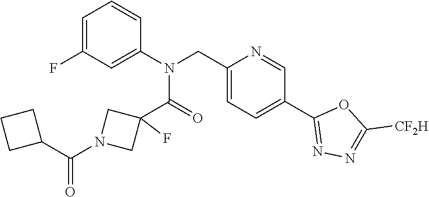 [SMILES: O=C(C1CCC1)N1CC(F)(C(=O)N(Cc2ccc(-c3nnc(C(F)F)o3)cn2)c2cccc(F)c2)C1] |
| 77 | 3169 | 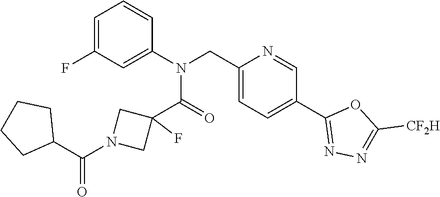 [SMILES: O=C(C1CCCC1)N1CC(F)(C(=O)N(Cc2ccc(-c3nnc(C(F)F)o3)cn2)c2cccc(F)c2)C1] |
| 78 | 3170 | 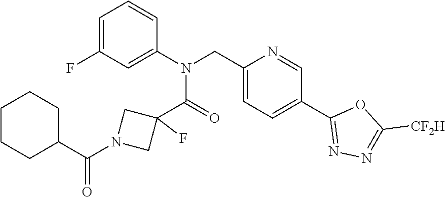 [SMILES: O=C(C1CCCCC1)N1CC(F)(C(=O)N(Cc2ccc(-c3nnc(C(F)F)o3)cn2)c2cccc(F)c2)C1] |
| 79 | 3171 | 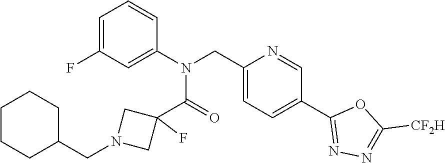 [SMILES: O=C(N(Cc1ccc(-c2nnc(C(F)F)o2)cn1)c1cccc(F)c1)C1(F)CN(CC2CCCCC2)C1] |
| 80 | 3172 | 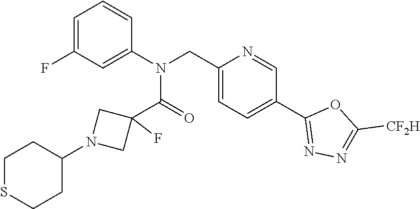 [SMILES: O=C(N(Cc1ccc(-c2nnc(C(F)F)o2)cn1)c1cccc(F)c1)C1(F)CN(C2CCSCC2)C1] |
| 81 | 3216 | 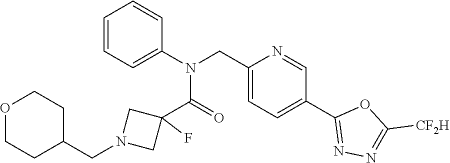 [SMILES: O=C(N(Cc1ccc(-c2nnc(C(F)F)o2)cn1)c1ccccc1)C1(F)CN(CC2CCOCC2)C1] |
| 82 | 3217 | 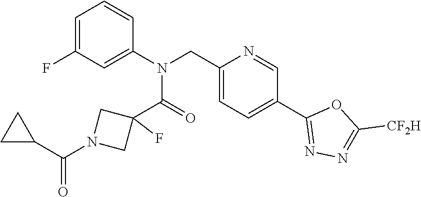 [SMILES: O=C(C1CC1)N1CC(F)(C(=O)N(Cc2ccc(-c3nnc(C(F)F)o3)cn2)c2cccc(F)c2)C1] |
| 83 | 3218 | 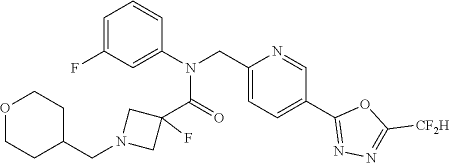 [SMILES: O=C(N(Cc1ccc(-c2nnc(C(F)F)o2)cn1)c1cccc(F)c1)C1(F)CN(CC2CCOCC2)C1] |
| 84 | 3219 | 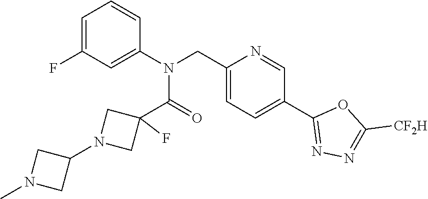 [SMILES: CN1CC(N2CC(F)(C(=O)N(Cc3ccc(-c4nnc(C(F)F)o4)cn3)c3cccc(F)c3)C2)C1] |
| 85 | 3220 | 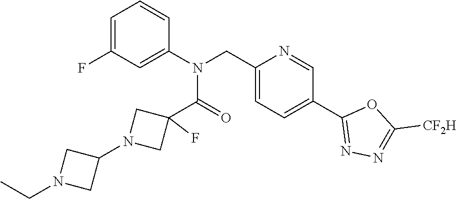 [SMILES: CCN1CC(N2CC(F)(C(=O)N(Cc3ccc(-c4nnc(C(F)F)o4)cn3)c3cccc(F)c3)C2)C1] |
| 86 | 3221 | 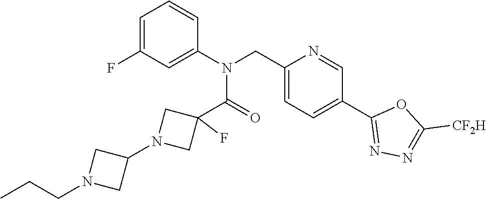 [SMILES: CCCN1CC(N2CC(F)(C(=O)N(Cc3ccc(-c4nnc(C(F)F)o4)cn3)c3cccc(F)c3)C2)C1] |
| 87 | 3222 | 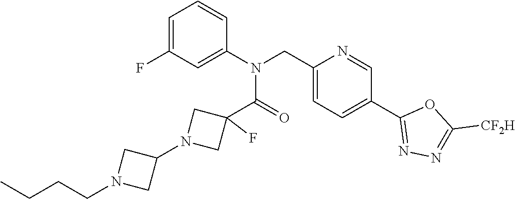 [SMILES: CCCCN1CC(N2CC(F)(C(=O)N(Cc3ccc(-c4nnc(C(F)F)o4)cn3)c3cccc(F)c3)C2)C1] |
| 88 | 3223 | 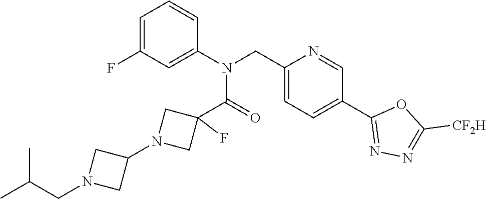 [SMILES: CC(C)CN1CC(N2CC(F)(C(=O)N(Cc3ccc(-c4nnc(C(F)F)o4)cn3)c3cccc(F)c3)C2)C1] |
| 89 | 3224 | 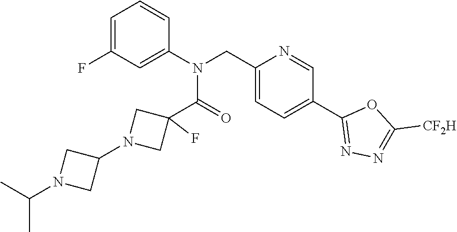 [SMILES: CC(C)N1CC(N2CC(F)(C(=O)N(Cc3ccc(-c4nnc(C(F)F)o4)cn3)c3cccc(F)c3)C2)C1] |
| 90 | 3389 | 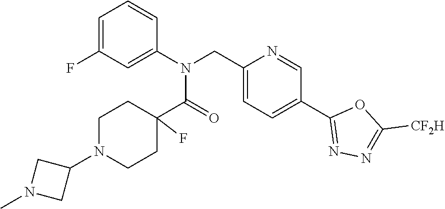 [SMILES: CN1CC(N2CCC(F)(C(=O)N(Cc3ccc(-c4nnc(C(F)F)o4)cn3)c3cccc(F)c3)CC2)C1] |
| 91 | 3390 | 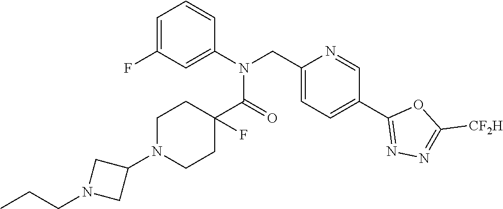 [SMILES: CCCN1CC(N2CCC(F)(C(=O)N(Cc3ccc(-c4nnc(C(F)F)o4)cn3)c3cccc(F)c3)CC2)C1] |
| 92 | 3391 | 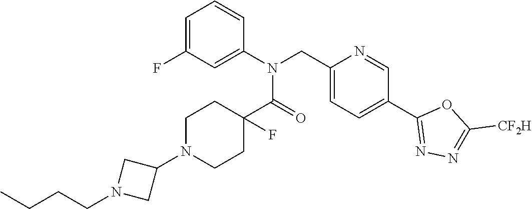 [SMILES: CCCCN1CC(N2CCC(F)(C(=O)N(Cc3ccc(-c4nnc(C(F)F)o4)cn3)c3cccc(F)c3)CC2)C1] |
| 93 | 3392 | 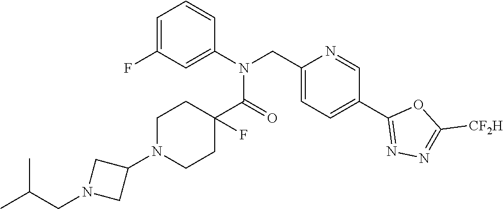 [SMILES: CC(C)CN1CC(N2CCC(F)(C(=O)N(Cc3ccc(-c4nnc(C(F)F)o4)cn3)c3cccc(F)c3)CC2)C1] |
| 94 | 3393 | 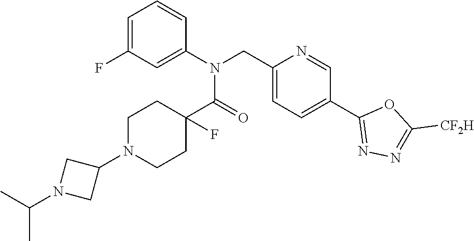 [SMILES: CC(C)N1CC(N2CCC(F)(C(=O)N(Cc3ccc(-c4nnc(C(F)F)o4)cn3)c3cccc(F)c3)CC2)C1] |
| 95 | 3394 | 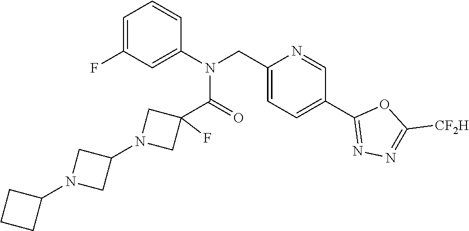 [SMILES: O=C(N(Cc1ccc(-c2nnc(C(F)F)o2)cn1)c1cccc(F)c1)C1(F)CN(C2CN(C3CCC3)C2)C1] |
| 96 | 3395 | 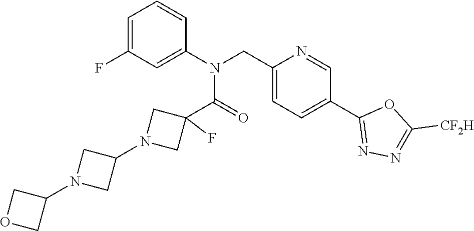 [SMILES: O=C(N(Cc1ccc(-c2nnc(C(F)F)o2)cn1)c1cccc(F)c1)C1(F)CN(C2CN(C3COC3)C2)C1] |
| 97 | 3396 | 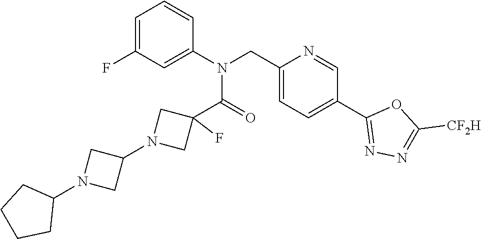 [SMILES: O=C(N(Cc1ccc(-c2nnc(C(F)F)o2)cn1)c1cccc(F)c1)C1(F)CN(C2CN(C3CCCC3)C2)C1] |
| 98 | 3397 | 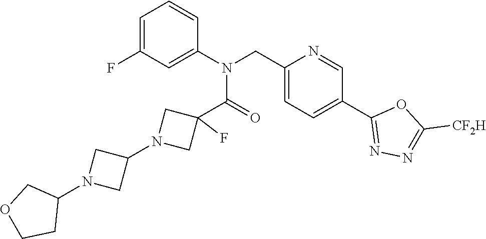 [SMILES: O=C(N(Cc1ccc(-c2nnc(C(F)F)o2)cn1)c1cccc(F)c1)C1(F)CN(C2CN(C3CCOC3)C2)C1] |
| 99 | 3398 | 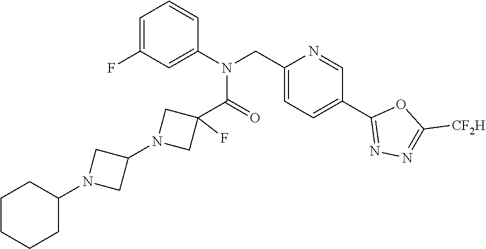 [SMILES: O=C(N(Cc1ccc(-c2nnc(C(F)F)o2)cn1)c1cccc(F)c1)C1(F)CN(C2CN(C3CCCCC3)C2)C1] |
| 100 | 3399 | 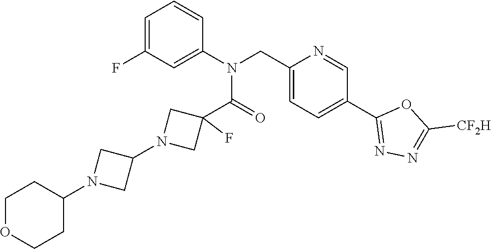 [SMILES: O=C(N(Cc1ccc(-c2nnc(C(F)F)o2)cn1)c1cccc(F)c1)C1(F)CN(C2CN(C3CCOCC3)C2)C1] |
| 101 | 3400 | 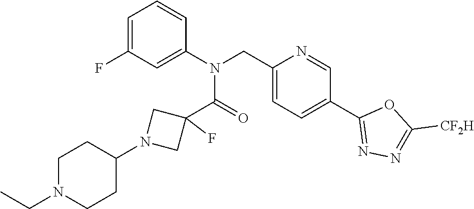 [SMILES: CCN1CCC(N2CC(F)(C(=O)N(Cc3ccc(-c4nnc(C(F)F)o4)cn3)c3cccc(F)c3)C2)CC1] |
| 102 | 3401 | 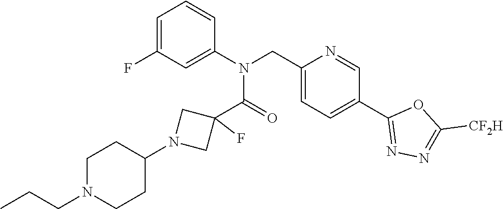 [SMILES: CCCN1CCC(N2CC(F)(C(=O)N(Cc3ccc(-c4nnc(C(F)F)o4)cn3)c3cccc(F)c3)C2)CC1] |
| 103 | 3402 | 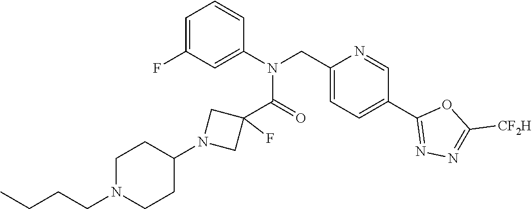 [SMILES: CCCCN1CCC(N2CC(F)(C(=O)N(Cc3ccc(-c4nnc(C(F)F)o4)cn3)c3cccc(F)c3)C2)CC1] |
| 104 | 3403 | 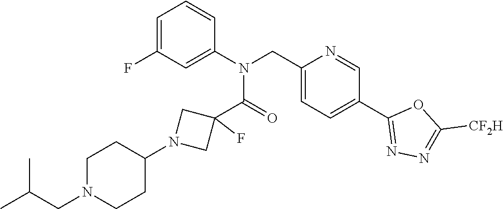 [SMILES: CC(C)CN1CCC(N2CC(F)(C(=O)N(Cc3ccc(-c4nnc(C(F)F)o4)cn3)c3cccc(F)c3)C2)CC1] |
| 105 | 3404 | 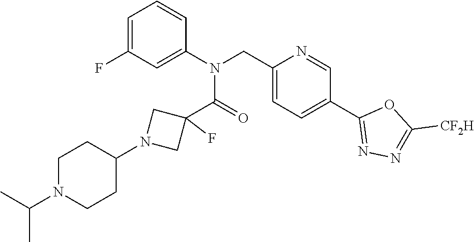 [SMILES: CC(C)N1CCC(N2CC(F)(C(=O)N(Cc3ccc(-c4nnc(C(F)F)o4)cn3)c3cccc(F)c3)C2)CC1] |
| 106 | 3405 | 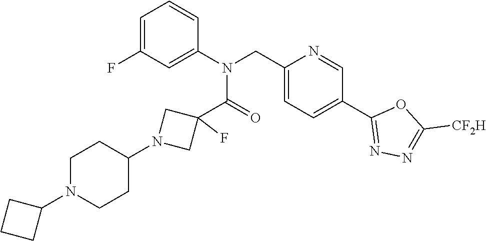 [SMILES: O=C(N(Cc1ccc(-c2nnc(C(F)F)o2)cn1)c1cccc(F)c1)C1(F)CN(C2CCN(C3CCC3)CC2)C1] |
| 107 | 3406 | 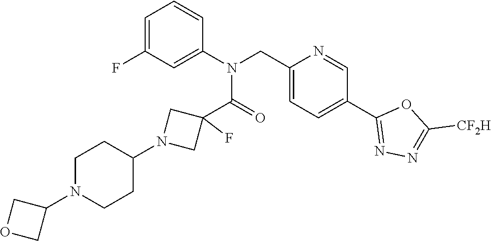 [SMILES: O=C(N(Cc1ccc(-c2nnc(C(F)F)o2)cn1)c1cccc(F)c1)C1(F)CN(C2CCN(C3COC3)CC2)C1] |
| 108 | 3407 | 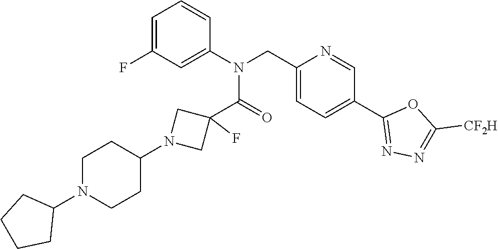 [SMILES: O=C(N(Cc1ccc(-c2nnc(C(F)F)o2)cn1)c1cccc(F)c1)C1(F)CN(C2CCN(C3CCCC3)CC2)C1] |
| 109 | 3408 | 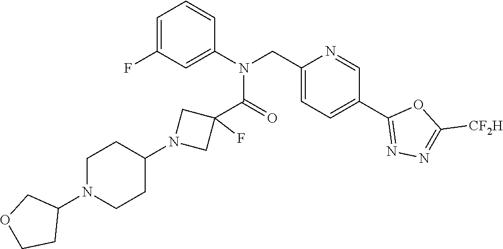 [SMILES: O=C(N(Cc1ccc(-c2nnc(C(F)F)o2)cn1)c1cccc(F)c1)C1(F)CN(C2CCN(C3CCOC3)CC2)C1] |
| 110 | 3409 | 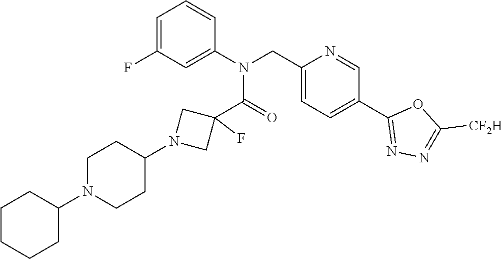 [SMILES: O=C(N(Cc1ccc(-c2nnc(C(F)F)o2)cn1)c1cccc(F)c1)C1(F)CN(C2CCN(C3CCCCC3)CC2)C1] |
| 111 | 3410 | 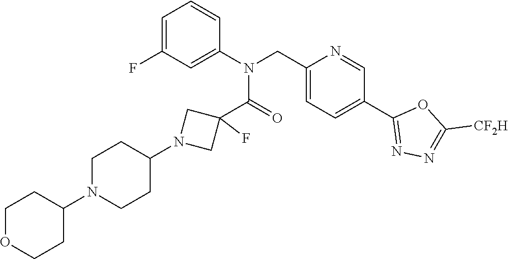 [SMILES: O=C(N(Cc1ccc(-c2nnc(C(F)F)o2)cn1)c1cccc(F)c1)C1(F)CN(C2CCN(C3CCOCC3)CC2)C1] |
| 112 | 3429 | 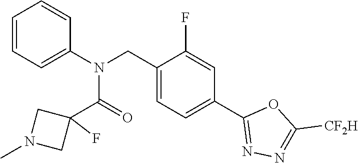 [SMILES: CN1CC(F)(C(=O)N(Cc2ccc(-c3nnc(C(F)F)o3)cc2F)c2ccccc2)C1] |
| 113 | 3430 | 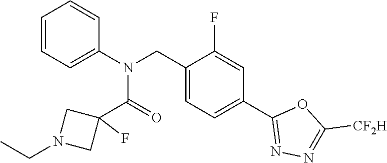 [SMILES: CCN1CC(F)(C(=O)N(Cc2ccc(-c3nnc(C(F)F)o3)cc2F)c2ccccc2)C1] |
| 114 | 3431 | 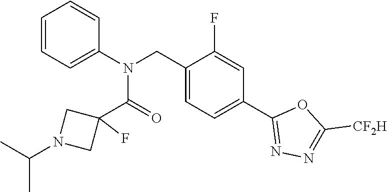 [SMILES: CC(C)N1CC(F)(C(=O)N(Cc2ccc(-c3nnc(C(F)F)o3)cc2F)c2ccccc2)C1] |
| 115 | 3432 | 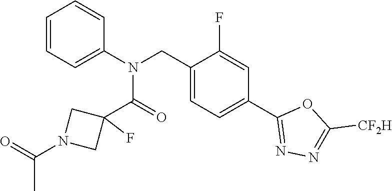 [SMILES: CC(=O)N1CC(F)(C(=O)N(Cc2ccc(-c3nnc(C(F)F)o3)cc2F)c2ccccc2)C1] |
| 116 | 3433 | 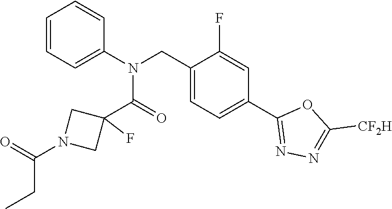 [SMILES: CCC(=O)N1CC(F)(C(=O)N(Cc2ccc(-c3nnc(C(F)F)o3)cc2F)c2ccccc2)C1] |
| 117 | 3434 | 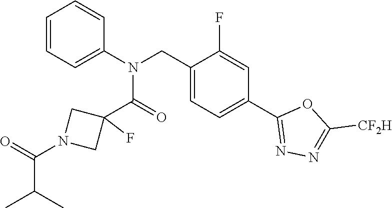 [SMILES: CC(C)C(=O)N1CC(F)(C(=O)N(Cc2ccc(-c3nnc(C(F)F)o3)cc2F)c2ccccc2)C1] |
| 118 | 3435 | 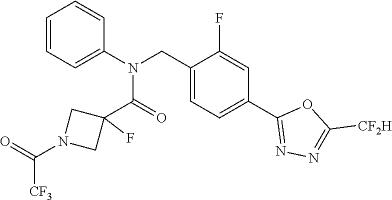 [SMILES: O=C(N1CC(F)(C(=O)N(Cc2ccc(-c3nnc(C(F)F)o3)cc2F)c2ccccc2)C1)C(F)(F)F] |
| 119 | 3436 | 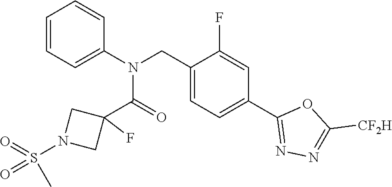 [SMILES: CS(=O)(=O)N1CC(F)(C(=O)N(Cc2ccc(-c3nnc(C(F)F)o3)cc2F)c2ccccc2)C1] |
| 120 | 3437 | 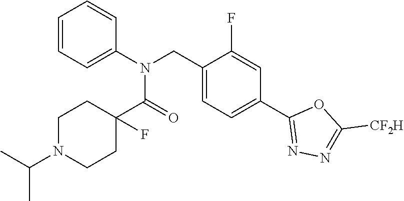 [SMILES: CC(C)N1CCC(F)(C(=O)N(Cc2ccc(-c3nnc(C(F)F)o3)cc2F)c2ccccc2)CC1] |
| 121 | 3438 | 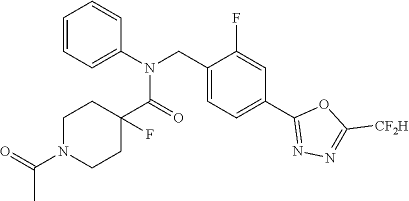 [SMILES: CC(=O)N1CCC(F)(C(=O)N(Cc2ccc(-c3nnc(C(F)F)o3)cc2F)c2ccccc2)CC1] |
| 122 | 3439 | 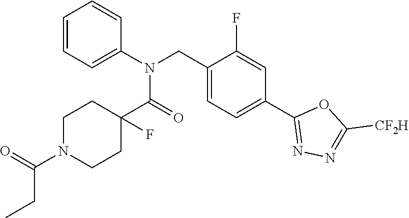 [SMILES: CCC(=O)N1CCC(F)(C(=O)N(Cc2ccc(-c3nnc(C(F)F)o3)cc2F)c2ccccc2)CC1] |
| 123 | 3440 | 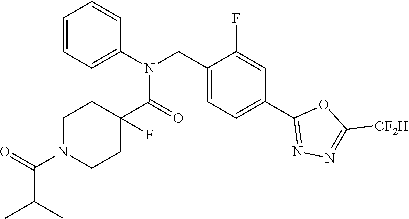 [SMILES: CC(C)C(=O)N1CCC(F)(C(=O)N(Cc2ccc(-c3nnc(C(F)F)o3)cc2F)c2ccccc2)CC1] |
| 124 | 3441 | 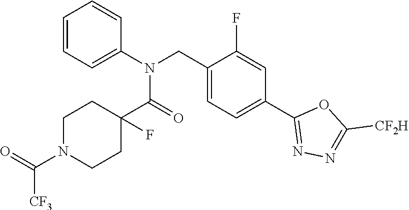 [SMILES: O=C(N1CCC(F)(C(=O)N(Cc2ccc(-c3nnc(C(F)F)o3)cc2F)c2ccccc2)CC1)C(F)(F)F] |
| 125 | 3442 | 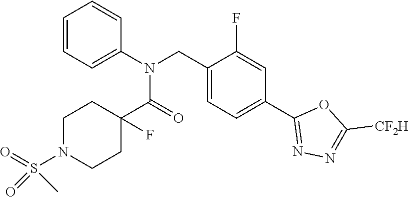 [SMILES: CS(=O)(=O)N1CCC(F)(C(=O)N(Cc2ccc(-c3nnc(C(F)F)o3)cc2F)c2ccccc2)CC1] |
| 126 | 3443 | 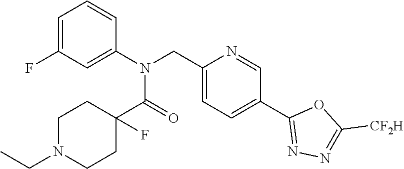 [SMILES: CCN1CCC(F)(C(=O)N(Cc2ccc(-c3nnc(C(F)F)o3)cn2)c2cccc(F)c2)CC1] |
| 127 | 6890 | 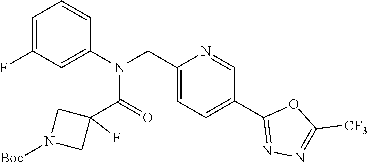 [SMILES: CC(C)(C)OC(=O)N1CC(F)(C(=O)N(Cc2ccc(-c3nnc(C(F)(F)F)o3)cn2)c2cccc(F)c2)C1] |
| 128 | 6891 | 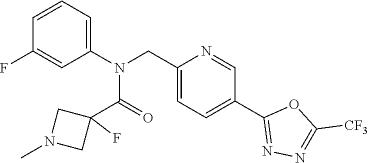 [SMILES: CN1CC(F)(C(=O)N(Cc2ccc(-c3nnc(C(F)(F)F)o3)cn2)c2cccc(F)c2)C1] |

In the present invention, the pharmaceutically acceptable salt refers to a salt commonly used in the pharmaceutical industry, for example, may include inorganic ionic salts prepared from calcium, potassium, sodium, and magnesium, and the like, inorganic acid salts prepared from hydrochloric acid, nitric acid, phosphoric acid, bromic acid, iodic acid, perchloric acid, and sulfuric acid, and the like; organic acid salts prepared from acetic acid, trifluoroacetic acid, citric acid, maleic acid, succinic acid, oxalic acid, benzoic acid, tartaric acid, fumaric acid, manderic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like; sulfonic acid salts prepared from methanesulfonic acid, ethanesulfonic acid, benzenesulfonic acid, p-toluenesulfonic acid, and naphthalenesulfonic acid, and the like; amino acid salts prepared from glycine, arginine, lysine, and the like; and amine salts prepared with trimethylamine, triethylamine, ammonia, pyridine, picoline, and the like, but types of salts referred to in the present invention are not limited by these salts listed above.

The compound represented by Chemical Formula I of the present invention may contain one or more asymmetric carbons, thereby being able to exist as a racemate, a racemic mixture, a single enantiomer, a diastereomeric mixture, and each diastereomer. These isomers may be separated using conventional techniques, for example, by partitioning, such as by column chromatography, HPLC, or the like, the compound represented by Chemical Formula I. Alternatively, stereoisomers of each of the compounds represented by Chemical Formula I may be stereospecifically synthesized using optically pure starting materials and/or reagents with known arrangement.

Method for Preparing 1,3,4-Oxadiazole Derivative Compound

The present invention provides a method for preparing a 1,3,4-oxadiazole derivative compound represented by Chemical Formula I, an optical isomer thereof, or a pharmaceutically acceptable salt thereof.

A preferred method for preparing the 1,3,4-oxadiazole derivative compound represented by Chemical Formula I, the optical isomer thereof, or the pharmaceutically acceptable salt thereof according to the present invention is the same as Reaction Schemes 1 and 2 below, which also includes preparation methods modified to a level obvious to those skilled in the art.

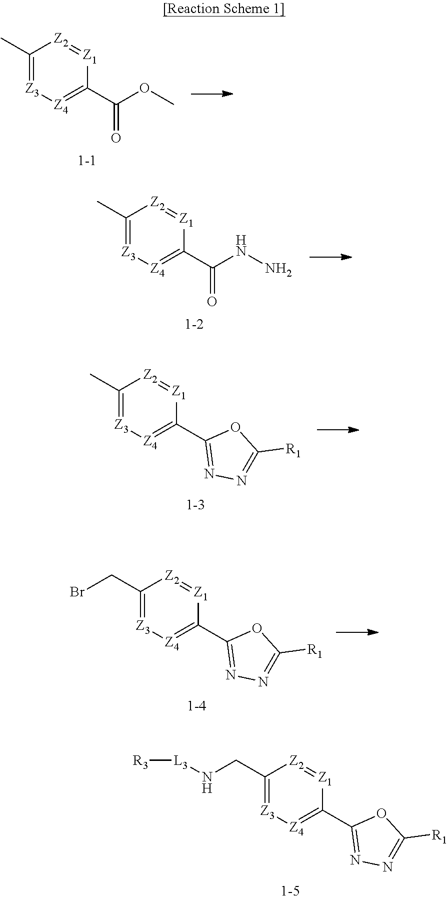

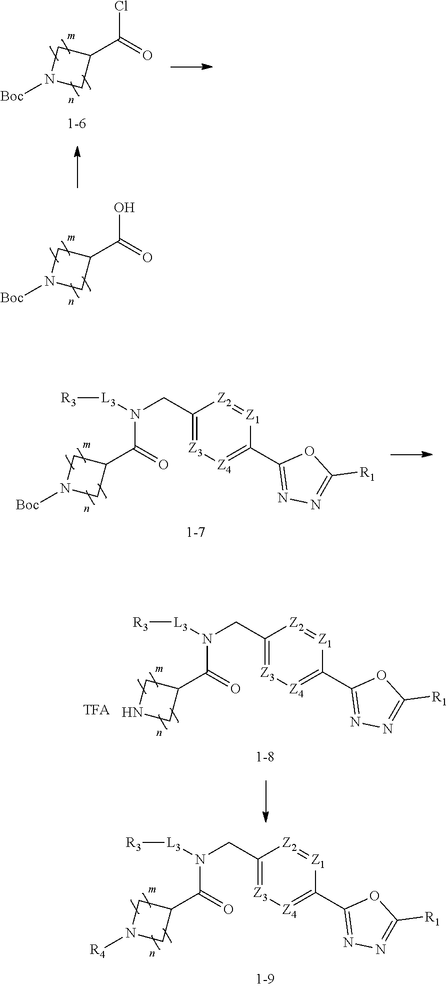

[Reaction Scheme 1] shows a method for synthesizing a compound having an alpha fluoroamide structure. First, Compound 1-1 is reacted with hydrazine to synthesize Hydrazide Compound 1-2. A cyclization reaction with difluoro acetic anhydride or trifluoro acetic anhydride is performed to synthesize Compound 1-3, followed by the bromination reaction to synthesize Compound 1-4. By reacting with aniline into which a substituent is introduced, Compound 1-5 is synthesized. Compound 1-6 is synthesized by reacting oxalyl chloride with carboxylic acid into with fluorine is introduced at the alpha position, and then is reacted with Compound 1-5 to synthesize Compound 1-7. Compound 1-8 from which the protecting group is removed under an acid condition is synthesized, and Title Compound 1-9 is synthesized by introducing various functional groups.

Compounds prepared by the above Reaction Scheme are 2865, 2866, 2867, 2868, 2869, 2951, 2952, 2953, 2954, 2969, 2970, 2971, 2972, 2973, 2974, 2975, 2976, 2995, 2996, 2997, 2998, 2999, 3000, 3001, 3002, 3003, 3004, 3005, 3006, 3007, 3047, 3048, 3049, 3050, 3051, 3052, 3053, 3054, 3055, 3090, 3091, 3092, 3093, 3094, 3095, 3096, 3097, 3098, 3152, 3153, 3154, 3155, 3156, 3157, 3158, 3159, 3160, 3161, 3162, 3163, 3164, 3165, 3166, 3167, 3168, 3169, 3170, 3171, 3172, 3216, 3217 3218, 3429, 3430, 3431, 3432, 3433, 3434, 3435, 3436, 3437, 3438, 3439, 3440, 3441, 3442, 3443, 6890, and 6891.

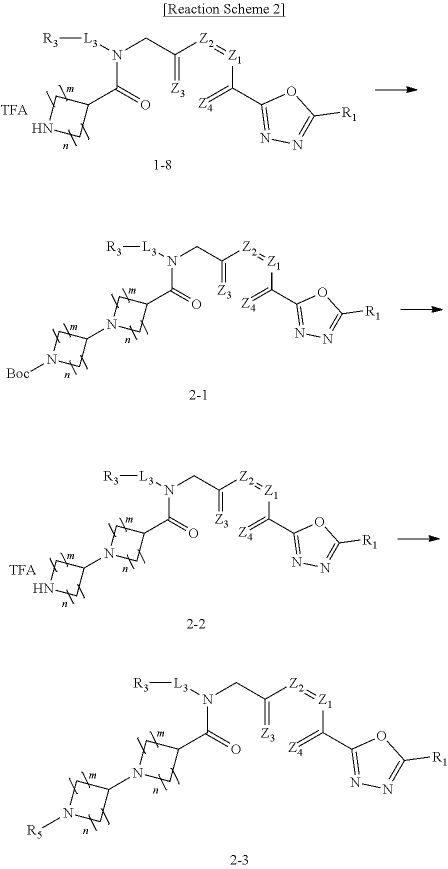

[Reaction Scheme 2] also shows a method for synthesizing a compound having an alpha fluoroamide structure. First, Compound 1-8 synthesized in Reaction Scheme 1 is subjected to a reductive amination reaction to synthesize Compound 2-1. Compound 2-2 from which the protecting group is removed under an acid condition is synthesized, and Title Compound 2-3 is synthesized by introducing various functional groups.

Compounds prepared by the above Reaction Scheme are 3105, 3106, 3107, 3108, 3109, 3110, 3111, 3112, 3113, 3114, 3115, 3219, 3220, 3221, 3222, 3223, 3224, 3389, 3390, 3391, 3392, 3393, 3394, 3395, 3396, 3397, 3398, 3399, 3400, 3401, 3402, 3403, 3404, 3405, 3406, 3407, 3408, 3409, and 3410.

Composition Including 1,3,4-Oxadiazole Derivative Compound, Use Thereof, and Treatment Method Using the Same

The present invention provides a pharmaceutical composition for preventing or treating histone deacetylase 6-mediated diseases containing the compound represented by Chemical Formula I below, the optical isomer thereof, or the pharmaceutically acceptable salt thereof as an active ingredient:

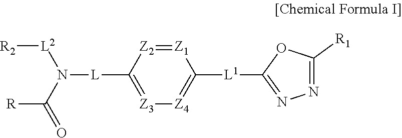

The Chemical Formula I is the same as defined above.

The pharmaceutical composition of the present invention exhibits a remarkable effect in the prevention or treatment of histone deacetylase 6-mediated diseases by selectively inhibiting a histone deacetylase 6.

The histone deacetylase 6-mediated diseases include infectious diseases such as prion disease; neoplasm such as benign tumors (e.g. myelodysplastic syndrome) or malignant tumors (e.g. multiple myeloma, lymphoma, leukemia, lung cancer, colorectal cancer, colon cancer, prostate cancer, urinary tract epithelial cell carcinoma, breast cancer, melanoma, skin cancer, liver cancer, brain cancer, stomach cancer, ovarian cancer, pancreatic cancer, head and neck cancer, oral cancer or glioma); endocrine, nutritional and metabolic diseases such as Wilson's disease, amyloidosis or diabetes; mental and behavioral disorders such as depression or Rett syndrome; neurological diseases such as central nervous system atrophy (e.g. Huntington's disease, spinal muscular atrophy (SMA), spinal cerebellar ataxia (SCA)), neurodegenerative diseases (e.g. Alzheimer's disease), movement disorders (e.g. Parkinson's disease), neuropathy (e.g. hereditary neuropathy (Charcot-Marie-Tooth disease), sporadic neuropathy, inflammatory neuropathy, drug-induced neuropathy), motor neuropathy (e.g. amyotrophic lateral sclerosis (ALS)), or central nervous system demyelination (e.g. multiple sclerosis (MS)); diseases of eyes and adnexa such as uveitis; circulatory diseases such as atrial fibrillation, stroke, and the like; respiratory diseases such as asthma; digestive diseases such as alcoholic liver disease, inflammatory bowel disease, Crohn's disease, ulcerative bowel disease, and the like; skin and subcutaneous tissue diseases such as psoriasis; musculoskeletal and connective tissue diseases such as rheumatoid arthritis, osteoarthritis, systemic lupus erythematosus (SLE), and the like; or congenital malformations, alterations, and chromosomal abnormalities such as autosomal dominant polycystic kidney disease, and also include symptoms or diseases related to abnormal functions of histone deacetylase.

The pharmaceutically acceptable salt is the same as described above in the pharmaceutically acceptable salt of the compound represented by Chemical Formula I of the present invention.

The pharmaceutical composition of the present invention may further include one or more pharmaceutically acceptable carriers for administration, in addition to the compound represented by Chemical Formula I, the optical isomer thereof, or the pharmaceutically acceptable salt thereof. The pharmaceutically acceptable carrier may be used by mixing saline, sterile water, Ringer's solution, buffered saline, dextrose solution, maltodextrin solution, glycerol, ethanol and one or more of these ingredients, and if necessary, other conventional additives such as antioxidants, buffers, bacteriostatic agents, and the like, may be added. Further, injectable formulations such as aqueous solutions, suspensions, emulsions, and the like, pills, capsules, granules or tablets may be formulated by further adding diluents, dispersants, surfactants, binders and lubricants. Accordingly, the composition of the present invention may be a patch, liquid, pill, capsule, granule, tablet, suppository, or the like. These formulations may be prepared by a conventional method used for formulation in the art or by a method disclosed in Remington's Pharmaceutical Science (latest edition), Mack Publishing Company, Easton Pa., and formulated into various formulations depending on respective diseases or ingredients.

The composition of the present invention may be administered orally or parenterally (for example, intravenously, subcutaneously, intraperitoneally or topically) depending on the desired method, and the dosage range varies depending on the patient's weight, age, sex, health condition, diet, administration time, administration method, excretion rate, and severity of disease, and the like. The daily dose of the compound represented by Chemical Formula I of the present invention may be about 1 to 1000 mg/kg, preferably 5 to 100 mg/kg, and may be administered once a day or divided into several times a day.

The pharmaceutical composition of the present invention may further include one or more active ingredients exhibiting the same or similar medicinal effects in addition to the compound represented by Chemical Formula I above, the optical isomer thereof, or the pharmaceutically acceptable salt thereof.

The present invention provides a method for preventing or treating histone deacetylase 6-mediated diseases including administering a therapeutically effective amount of the compound represented by Chemical Formula I, the optical isomer thereof, or the pharmaceutically acceptable salt thereof.

The term “therapeutically effective amount” used in the present invention refers to an amount of the compound represented by Chemical Formula I that is effective for preventing or treating the histone deacetylase 6-mediated diseases.

In addition, the present invention provides a method for selectively inhibiting HDAC6 by administering the compound represented by Chemical Formula I, the optical isomer thereof, or the pharmaceutically acceptable salt thereof to a mammal including humans.

The method for preventing or treating the histone deacetylase 6-mediated diseases of the present invention also includes administering the compound represented by Chemical Formula I to treat the disease itself before the onset of the symptom, but also to inhibit or avoid the symptom thereof. In the management of the disease, prophylactic or therapeutic dose of a specific active ingredient will vary depending on the nature and severity of the disease or condition, and the route to which the active ingredient is administered. The dose and frequency of dose will vary depending on the age, weight and response of the individual patients. A suitable dosage regimen may be readily selected by a person having ordinary knowledge in the art considering these factors for granted. In addition, the method for preventing or treating histone deacetylase 6-mediated diseases of the present invention may further include administrating a therapeutically effective amount of an additional active agent useful for the treatment of the disease together with the compound represented by Chemical Formula I, wherein the additional active agent may exhibit synergistic or auxiliary effects together with the compound represented by Chemical Formula I.

The present invention also aims to provide the use of the compound represented by Chemical Formula I above, the optical isomer thereof, or the pharmaceutically acceptable salt thereof for preparing a medicament for treating histone deacetylase 6-mediated diseases. The compound represented by Chemical Formula I above for preparing the medicament may be mixed with acceptable adjuvants, diluents, carriers, and the like, and may be prepared as a complex formulation with other active agents to have a synergistic effect of active ingredients.

Matters mentioned in the uses, compositions and treatment methods of the present invention are applied equally as long as they are inconsistent with each other.

### Advantageous Effects of Invention

The compound represented by Chemical Formula I above of the present invention, the optical isomer thereof, or the pharmaceutically acceptable salt thereof, is able to selectively inhibit histone deacetylase 6 (HDAC6), thereby having remarkably excellent preventive or therapeutic effects on HDAC6-mediated diseases.

### BEST MODE FOR CARRYING OUT THE INVENTION

Hereinafter, the present invention will be described in more detail through Examples and Experimental Examples. However, these Examples and the like are only examples of the present invention, and the scope of the present invention is not limited thereto.

### Preparation of 1,3,4-Oxadiazole Derivative Compound

A specific method for preparing the compound represented by Chemical Formula I is as follows.

### Example 1: Synthesis of Compound 2865, N-(4-(5-(difluoromethyl)-1,3,4-oxadiazol-2-yl)-2-fluorobenzyl)-4-fluoro-N-phenylpiperidine-4-carboxamide 2,2,2-trifluoroacetate

### [Step 1] Synthesis of N4-(5-(difluoromethyl)-1,3,4-oxadiazol-2-yl)-fluorobenzyl)aniline

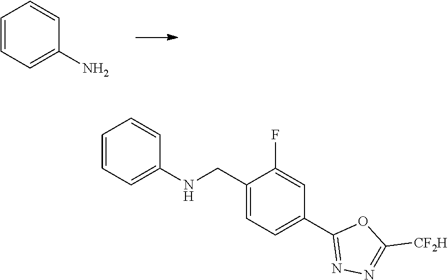 [SMILES: Nc1ccccc1 ; Fc1cc(-c2nnc(C(F)F)o2)ccc1CNc1ccccc1]

Aniline (0.980 mL, 10.738 mmol), 2-(4-(bromomethyl)-3-fluorophenyl)-5-(difluoromethyl)-1,3,4-oxadiazole (4.286 g, 13.959 mmol), potassium carbonate (2.968 g, 21.475 mmol), and potassium iodide (0.178 g, 1.074 mmol) were dissolved in N,N-dimethylformamide (25 mL) at room temperature. The resulting solution was stirred at the same temperature for 16 hours. Water was poured into the concentrate obtained by removing the solvent from the reaction mixture under reduced pressure, followed by extraction with dichloromethane. The obtained product was filtered through a plastic filter to remove a solid residue and an aqueous layer, and then concentrated under reduced pressure. The concentrate was purified by column chromatography (SiO₂, 80 g cartridge; ethyl acetate/hexane=5% to 50%) and concentrated to obtain the title compound (1.900 g, 55.4%) as a colorless oil.

### [Step 2] Synthesis of tert-butyl 4-(chlorocarbonyl)-4-fluoropiperidine-1-carboxylate

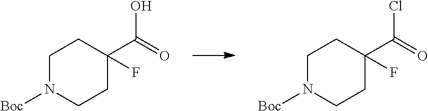 [SMILES: CC(C)(C)OC(=O)N1CCC(F)(C(=O)O)CC1 ; CC(C)(C)OC(=O)N1CCC(F)(C(=O)Cl)CC1]

1-(Tert-butoxycarbonyl)-4-fluoropiperidine-4-carboxylic acid (1,000 g, 4.044 mmol) was dissolved in dichloromethane (25 mL), and oxalyl chloride (0.417 mL, 4.853 mmol) and N,N-dimethylformamide (0.031 mL, 0.404 mmol) were added at 0° C. and stirred at room temperature for 2 hours. After removing the solvent from the reaction mixture under reduced pressure, the title compound (1.070 g, 99.6%) was obtained as a colorless oil.

### [Step 3] Synthesis of tert-butyl 4-((4-(5-(difluoromethyl)-1,3,4-oxadiazol-2-yl)-2-fluorobenzyl)(phenyl)carbamoyl)-4-fluoropiperidine-1-carboxylate

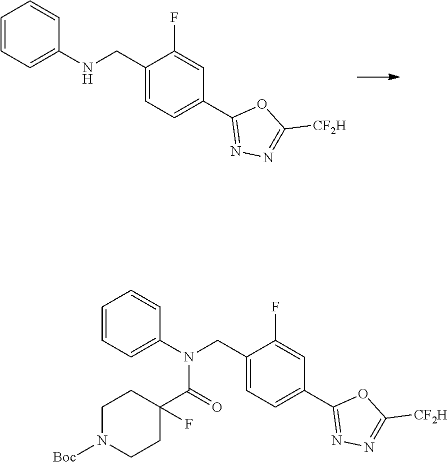 [SMILES: Fc1cc(-c2nnc(C(F)F)o2)ccc1CNc1ccccc1 ; CC(C)(C)OC(=O)N1CCC(F)(C(=O)N(Cc2ccc(-c3nnc(C(F)F)o3)cc2F)c2ccccc2)CC1]

To a solution in which N-(4-(5-(difluoromethyl)-1,3,4-oxadiazol-2-yl)-2-fluorobenzyl)aniline (0.900 g, 2.819 mmol) prepared in step 1 and triethylamine (1.179 mL, 8.456 mmol) were dissolved in dichloromethane (35 mL) at room temperature, tert-butyl 4-(chlorocarbonyl)-4-fluoropiperidine-1-carboxylate (0.974 g, 3.664 mmol) prepared in step 2 was added and stirred at the same temperature for 16 hours. A saturated aqueous ammonium chloride solution was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 40 g cartridge; ethyl acetate/hexane=5% to 35%) and concentrated to obtain the title compound (0.570 g, 36.9%) as a foamy solid.

### [Step 4] Synthesis of Compound 2865

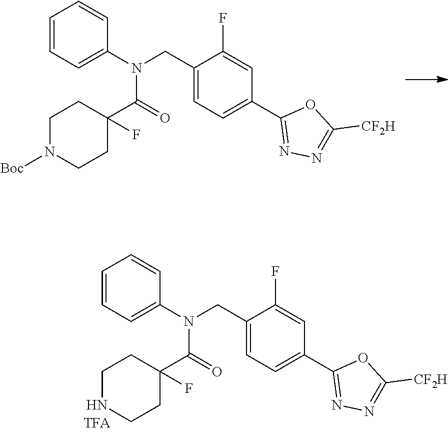 [SMILES: CC(C)(C)OC(=O)N1CCC(F)(C(=O)N(Cc2ccc(-c3nnc(C(F)F)o3)cc2F)c2ccccc2)CC1 ; O=C(N(Cc1ccc(-c2nnc(C(F)F)o2)cc1F)c1ccccc1)C1(F)CCNCC1]

Tert-butyl 4-((4-(5-(difluoromethyl)-1,3,4-oxadiazol-2-yl)-2-fluorobenzyl)(phenyl)carbamoyl)-4-fluoropiperidine-1-carboxylate (0.350 g, 0.638 mmol) prepared in step 3 was dissolved in dichloromethane (20 mL), and trifluoroacetic acid (0.977 mL, 12.761 mmol) was added at 0° C. and stirred at room temperature for 16 hours. After removing the solvent from the reaction mixture under reduced pressure, the title compound (0.355 g, 98.9%) was obtained as a foamy solid.

¹H NMR (400 MHz, MeOD) δ 7.91 (m, 1H), 7.76 (m, 1H), 7.60 (m, 1H), 7.36-7.71 (m, 6H), 5.08 (s, 2H), 3.11 (m, 2H), 2.84 (m, 2H), 2.44-2.27 (m, 2H), 2.04 (m, 2H);

LRMS (ES) m/z 449.4 (M++1).

### Example 2: Synthesis of Compound 2866, N-(4-(5-(difluoromethyl)-1,3,4-oxadiazol-2-yl)-2-fluorobenzyl)-4-fluoro-1-methyl-N-phenylpiperidine-4-carboxamide

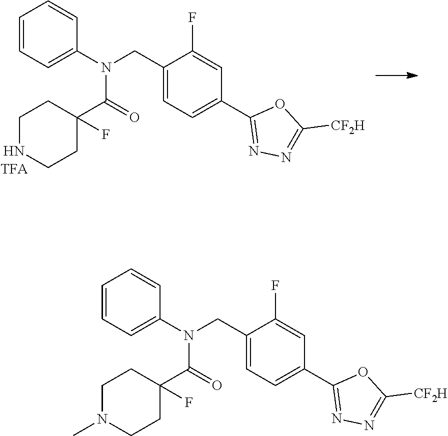 [SMILES: O=C(N(Cc1ccc(-c2nnc(C(F)F)o2)cc1F)c1ccccc1)C1(F)CCNCC1 ; CN1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.070 g, 0.124 mmol) prepared in step 4 of Example 1, paraformaldehyde (0.007 g, 0.249 mmol), and acetic acid (0.007 mL, 0.124 mmol) were dissolved in dichloromethane (4 mL), and the resulting solution was stirred at room temperature for 1 hour. Sodium triacetoxyborohydride (0.079 g, 0.373 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.025 g, 43.4%) as a foamy solid.

¹H NMR (400 MHz, CDCl₃) δ 7.89 (m, 1H), 7.74 (m, 1H), 7.58 (m, 1H), 7.33 (m, 3H), 7.06-6.80 (m, 3H), 5.03 (s, 2H), 2.96 (m, 2H), 2.56-2.34 (m, 7H), 1.99 (m, 2H);

LRMS (ES) m/z 463.6 (M++1).

### Example 3: Synthesis of Compound 2867, N-(4-(5-(difluoromethyl)-1,3,4-oxadiazol-2-yl)-2-fluorobenzyl)-1-ethyl-4-fluoro-N-phenylpiperidine-4-carboxamide

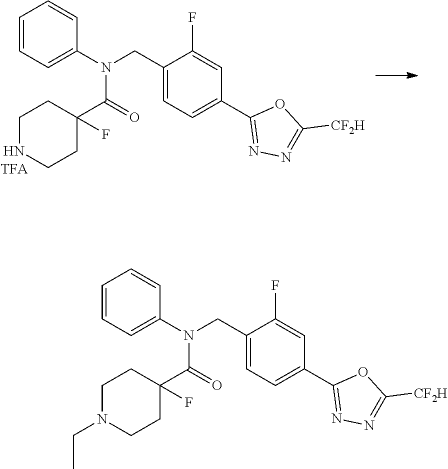 [SMILES: O=C(N(Cc1ccc(-c2nnc(C(F)F)o2)cc1F)c1ccccc1)C1(F)CCNCC1 ; CCN1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.070 g, 0.124 mmol) prepared in step 4 of Example 1, acetaldehyde (0.011 g, 0.249 mmol), and acetic acid (0.007 mL, 0.124 mmol) were dissolved in dichloromethane (4 mL), and the resulting solution was stirred at room temperature for 1 hour. Sodium triacetoxyborohydride (0.079 g, 0.373 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.024 g, 40.5%) as a foamy solid.

¹H NMR (400 MHz, CDCl₃) δ 7.89 (m, 1H), 7.74 (m, 1H), 7.71 (m, 1H), 7.57 (m, 3H), 7.06-6.80 (m, 3H), 5.03 (s, 2H), 3.04 (m, 2H), 2.64-2.35 (m, 6H), 2.00 (m, 2H), 1.15 (m, 3H);

LRMS (ES) m/z 477.6 (M++1).

### Example 4: Synthesis of Compound 2868, 1-cyclobutyl-N-(4-(5-(difluoromethyl)-1,3,4-oxadiazol-2-yl)-2-fluorobenzyl)-4-fluoro-N-phenylpiperidine-4-carboxamide

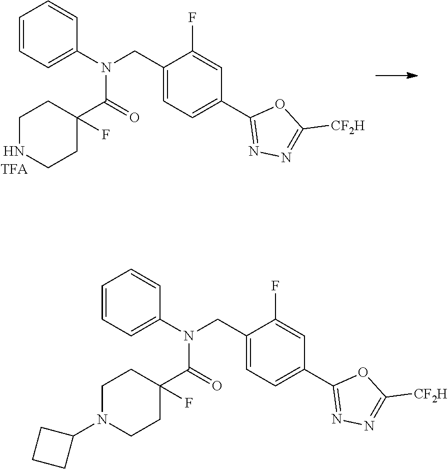 [SMILES: O=C(N(Cc1ccc(-c2nnc(C(F)F)o2)cc1F)c1ccccc1)C1(F)CCNCC1 ; O=C(N(Cc1ccc(-c2nnc(C(F)F)o2)cc1F)c1ccccc1)C1(F)CCN(C2CCC2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.070 g, 0.124 mmol) prepared in step 4 of Example 1, cyclobutanone (0.019 mL, 0.249 mmol), and acetic acid (0.007 mL, 0.124 mmol) were dissolved in dichloromethane (4 mL), and the resulting solution was stirred at room temperature for 1 hour. Sodium triacetoxyborohydride (0.079 g, 0.373 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.022 g, 35.2%) as a foamy solid.

¹H NMR (400 MHz, CDCl₃) δ 7.89 (m, 1H), 7.74 (m, 1H), 7.71 (m, 1H), 7.32 (m, 3H), 7.06-6.60 (m, 3H), 5.03 (s, 2H), 2.75 (m, 3H), 2.45-2.31 (m, 2H), 2.02-1.90 (m, 8H), 1.73-1.63 (m, 2H);

LRMS (ES) m/z 503.4 (M++1).

### Example 5: Synthesis of Compound 2869, N-(4-(5-(difluoromethyl)-1,3,4-oxadiazol-2-yl)-2-fluorobenzyl)-4-fluoro-1-(oxetan-3-yl)-N-phenylpiperidine-4-carboxamide

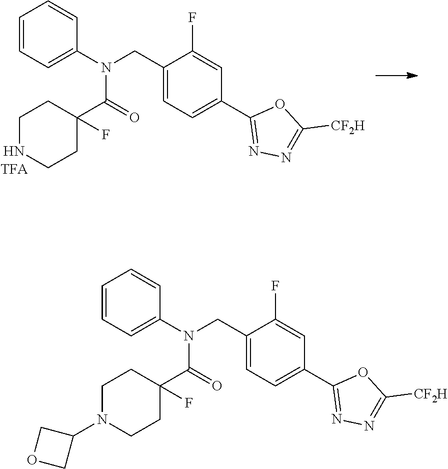 [SMILES: O=C(N(Cc1ccc(-c2nnc(C(F)F)o2)cc1F)c1ccccc1)C1(F)CCNCC1 ; O=C(N(Cc1ccc(-c2nnc(C(F)F)o2)cc1F)c1ccccc1)C1(F)CCN(C2COC2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.070 g, 0.124 mmol) prepared in step 4 of Example 1, oxetan-3-one (0.016 mL, 0.249 mmol), and acetic acid (0.007 mL, 0.124 mmol) were dissolved in dichloromethane (4 mL), and the resulting solution was stirred at room temperature for 1 hour. Sodium triacetoxyborohydride (0.079 g, 0.373 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.022 g, 35.0%) as a foamy solid.

¹H NMR (400 MHz, CDCl₃) δ 7.89 (m, 1H), 7.74 (m, 1H), 7.59 (m, 1H), 7.33 (m, 3H), 7.06-6.80 (m, 3H), 5.04 (s, 2H), 4.61 (m, 4H), 3.44 (m, 1H), 2.58 (m, 2H), 2.47-2.31 (m, 2H), 2.02-1.91 (m, 4H);

LRMS (ES) m/z 505.4 (M++1).

### Example 6: Synthesis of Compound 2951, N-((5-(5-(difluoromethyl)-1,3,4-oxadiazol-2-yl)pyridin-2-yl)methyl)-4-fluoro-1-meth yl-N-phenylpiperidine-4-carboxamide

### [Step 1] Synthesis of 6-methylnicotinohydrazide

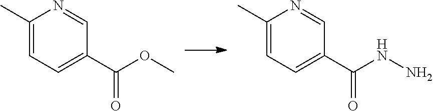

A solution that methyl 6-methylnicotinate (25.000 g, 165.377 mmol) and hydrazine monohydrate (40.188 mL, 826.884 mmol) were dissolved in ethanol (220 mL) at room temperature was heated to reflux for 16 hours, and then the temperature was lowered to room temperature to terminate the reaction. The solvent was removed from the reaction mixture under reduced pressure. The precipitated solid was filtered, washed with hexane, and dried to obtain the title compound (25.000 g, 100.0%) as a white solid.

### [Step 2] Synthesis of 2-(difluoromethyl)-5-(6-methylpyridin-3-yl)-1,3,4-oxadiazole

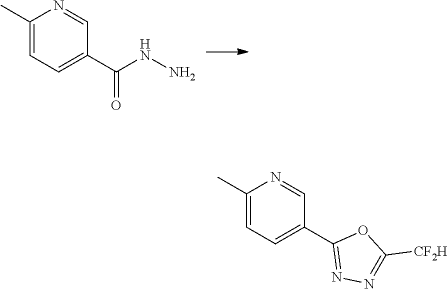

6-Methylnicotinohydrazide (15.000 g, 99.226 mmol) prepared in step 1 and imidazole (20.265 g, 297.678 mmol) were dissolved in dichloromethane (250 mL). 2,2-Difluoroacetic anhydride (37.008 mL, 297.678 mmol) was added at 0° C. and heated to reflux for 16 hours, and then the temperature was lowered to room temperature to terminate the reaction. Water was poured into the reaction mixture, followed by extraction with dichloromethane. The organic layer was washed with a saturated aqueous sodium chloride solution, and dried over anhydrous magnesium sulfate. The obtained product was filtered and concentrated under reduced pressure to obtain the title compound (20.900 g, 99.7%) as a red solid.

### [Step 3] Synthesis of 2-(6-(bromomethyl)pyridin-3-yl)-5-(difluoromethyl)-1,3,4-oxadiazole

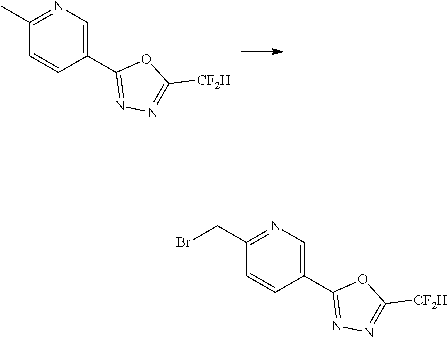 [SMILES: Cc1ccc(-c2nnc(C(F)F)o2)cn1 ; FC(F)c1nnc(-c2ccc(CBr)nc2)o1]

2-(Difluoromethyl)-5-(6-methylpyridin-3-yl)-1,3,4-oxadiazole (20.900 g, 98.972 mmol) prepared in step 2 was dissolved in 1,2-dichloroethane (200 mL). Azobisisobutyronitrile (AIBN, 0.813 g, 4.949 mmol) and 1-bromopyrrolidine-2,5-one (NBS, 22.900 g, 128.664 mmol) were added at room temperature and heated to reflux for 16 hours, and then the temperature was lowered to room temperature to terminate the reaction. Water was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified by column chromatography (SiO₂, 80 g cartridge; ethyl acetate/hexane=5% to 50%) and concentrated to obtain the title compound (5.400 g, 18.8%) as a red solid.

### [Step 4] Synthesis of N-((5-(5-(difluoromethyl)-1,3,4-oxadiazol-2-yl)pyridin-2-yl)methyl)aniline

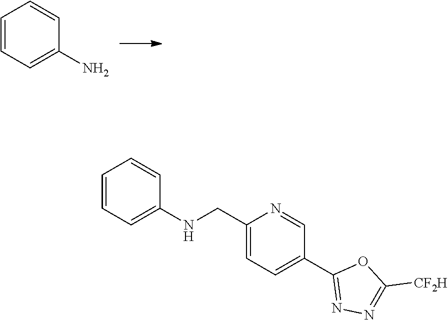 [SMILES: Nc1ccccc1 ; FC(F)c1nnc(-c2ccc(CNc3ccccc3)nc2)o1]

Aniline (0.490 mL, 5.369 mmol), 2-(6-(bromomethyl)pyridin-3-yl)-5-(difluoromethyl)-1,3,4-oxadiazole (1.635 g, 5.637 mmol) prepared in step 3, potassium carbonate (1.484 g, 10.738 mmol), and potassium iodide (0.089 g, 0.537 mmol) were dissolved in N,N-dimethylformamide (15 mL) at room temperature. The resulting solution was stirred at the same temperature for 16 hours. Water was poured into the concentrate obtained by removing the solvent from the reaction mixture under reduced pressure, followed by extraction with dichloromethane. The obtained product was filtered through a plastic filter to remove a solid residue and an aqueous layer, and then concentrated under reduced pressure. The concentrate was purified by column chromatography (SiO₂, 80 g cartridge; ethyl acetate/hexane=5% to 50%) and concentrated to obtain the title compound (1.300 g, 80.1%) as a yellow solid.

### [Step 5] Synthesis of tert-butyl 4-(((5-(5-(difluoromethyl)-1,3,4-oxadiazol-2-yl)pyridin-2-yl)methyl)(phenyl)carbamoyl)-4-fluoropiperidine-1-carboxylate

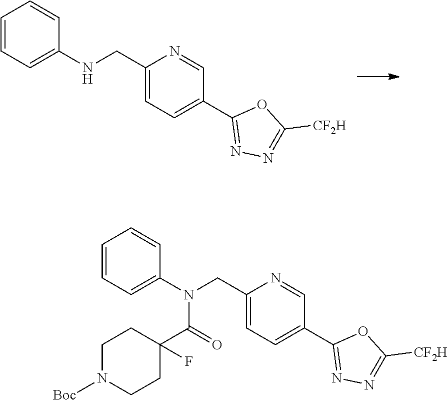 [SMILES: FC(F)c1nnc(-c2ccc(CNc3ccccc3)nc2)o1 ; CC(C)(C)OC(=O)N1CCC(F)(C(=O)N(Cc2ccc(-c3nnc(C(F)F)o3)cn2)c2ccccc2)CC1]

To a solution in which N-((5-(5-(difluoromethyl)-1,3,4-oxadiazol-2-yl)pyridin-2-yl)methyl)aniline (0.700 g, 2.316 mmol) prepared in step 4 and triethylamine (0.968 mL, 6.947 mmol) were dissolved in dichloromethane (35 mL) at room temperature, tert-butyl 4-(chlorocarbonyl)-4-fluoropiperidine-1-carboxylate (0.861 g, 3.242 mmol) prepared in step 2 of Example 1 was added and stirred at the same temperature for 16 hours. A saturated aqueous ammonium chloride solution was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 40 g cartridge; ethyl acetate/hexane=5% to 35%) and concentrated to obtain the title compound (0.400 g, 32.5%) as a foamy solid.

### [Step 6] Synthesis of N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate

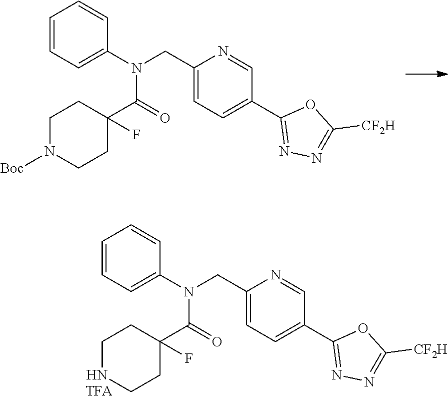 [SMILES: CC(C)(C)OC(=O)N1CCC(F)(C(=O)N(Cc2ccc(-c3nnc(C(F)F)o3)cn2)c2ccccc2)CC1 ; O=C(N(Cc1ccc(-c2nnc(C(F)F)o2)cn1)c1ccccc1)C1(F)CCNCC1]

Tert-butyl 4-(((5-(5-(difluoromethyl)-1,3,4-oxadiazol-2-yl)pyridin-2-yl)methyl)(phenyl)carbamoy 1)-4-fluoropiperidine-1-carboxylate (0.300 g, 0.564 mmol) prepared in step 5 was dissolved in dichloromethane (15 mL), and trifluoroacetic acid (0.432 mL, 5.644 mmol) was added at 0° C. and stirred at room temperature for 16 hours. After removing the solvent from the reaction mixture under reduced pressure, the title compound (0.305 g, 99.1%) was obtained as a foamy solid.

### [Step 7] Synthesis of Compound 2951

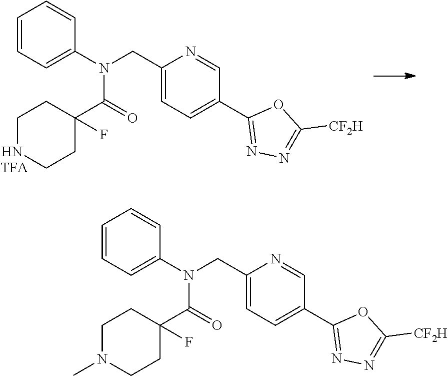 [SMILES: O=C(N(Cc1ccc(-c2nnc(C(F)F)o2)cn1)c1ccccc1)C1(F)CCNCC1 ; CN1CCC(F)(C(=O)N(Cc2ccc(-c3nnc(C(F)F)o3)cn2)c2ccccc2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.075 g, 0.138 mmol) prepared in step 6, paraformaldehyde (0.008 g, 0.275 mmol), and acetic acid (0.008 mL, 0.138 mmol) were dissolved in dichloromethane (4 mL), and the resulting solution was stirred at room temperature for 1 hour. Sodium triacetoxyborohydride (0.087 g, 0.413 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.023 g, 37.6%) as a foamy solid.

¹H NMR (400 MHz, CDCl₃) δ 9.23 (m, 1H), 8.33 (m, 1H), 7.46 (m, 1H), 7.33 (m, 3H), 7.23 (m, 2H), 6.94 (m, 1H), 5.04 (s, 2H), 3.30 (m, 2H), 2.76 (m, 2H), 2.63 (m, 5H), 2.12 (m, 2H);

LRMS (ES) m/z 446.4 (M++1).

### Example 7: Synthesis of Compound 2952, N-((5-(5-(difluoromethyl)-1,3,4-oxadiazol-2-yl)pyridin-2-yl)methyl)-4-fluoro-1-isopropyl-N-phenylpiperidine-4-carboxamide

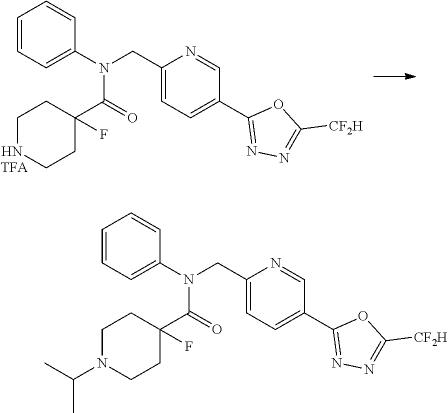 [SMILES: O=C(N(Cc1ccc(-c2nnc(C(F)F)o2)cn1)c1ccccc1)C1(F)CCNCC1 ; CC(C)N1CCC(F)(C(=O)N(Cc2ccc(-c3nnc(C(F)F)o3)cn2)c2ccccc2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.075 g, 0.138 mmol) prepared in step 6 of Example 6, acetone (0.020 mL, 0.275 mmol), and acetic acid (0.008 mL, 0.138 mmol) were dissolved in dichloromethane (4 mL), and the resulting solution was stirred at room temperature for 1 hour. Sodium triacetoxyborohydride (0.087 g, 0.413 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.018 g, 27.6%) as a foamy solid.

¹H NMR (400 MHz, CDCl₃) δ 9.24 (m, 1H), 8.36 (m, 1H), 7.47 (m, 1H), 7.34 (m, 3H), 7.23 (m, 2H), 6.95 (m, 1H), 5.05 (s, 2H), 3.44 (m, 3H), 2.90-2.86 (m, 4H), 2.18 (m, 2H), 1.28 (m, 6H);

LRMS (ES) m/z 474.4 (M++1).

### Example 8: Synthesis of Compound 2953, 1-cyclobutyl-N-((5-(5-(difluoromethyl)-1,3,4-oxadiazol-2-yl)pyridin-2-yl)methyl)-4-fluoro-N-phenylpiperidine-4-carboxamide

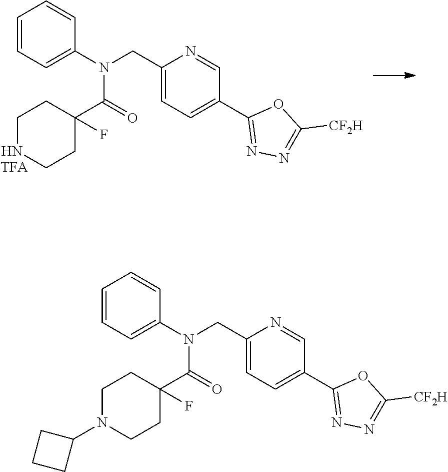 [SMILES: O=C(N(Cc1ccc(-c2nnc(C(F)F)o2)cn1)c1ccccc1)C1(F)CCNCC1 ; O=C(N(Cc1ccc(-c2nnc(C(F)F)o2)cn1)c1ccccc1)C1(F)CCN(C2CCC2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.075 g, 0.138 mmol) prepared in step 6 of Example 6, cyclobutanone (0.021 mL, 0.275 mmol), and acetic acid (0.008 mL, 0.138 mmol) were dissolved in dichloromethane (4 mL), and the resulting solution was stirred at room temperature for 1 hour. Sodium triacetoxyborohydride (0.087 g, 0.413 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.022 g, 33.0%) as a foamy solid.

¹H NMR (400 MHz, CDCl₃) δ 9.24 (m, 1H), 8.35 (m, 1H), 7.37 (m, 1H), 7.33 (m, 3H), 7.23 (m, 2H), 6.95 (m, 1H), 5.06 (s, 2H), 3.18-3.08 (m, 3H), 2.63-2.52 (m, 4H), 2.33 (m, 2H), 2.08 (m, 4H), 1.84-1.68 (m, 2H);

LRMS (ES) m/z 486.4 (M++1).

### Example 9: Synthesis of Compound 2954, N-((5-(5-(difluoromethyl)-1,3,4-oxadiazol-2-yl)pyridin-2-yl)methyl)-4-fluoro-1-(oxetan-3-yl)-N-phenylpiperidine-4-carboxamide

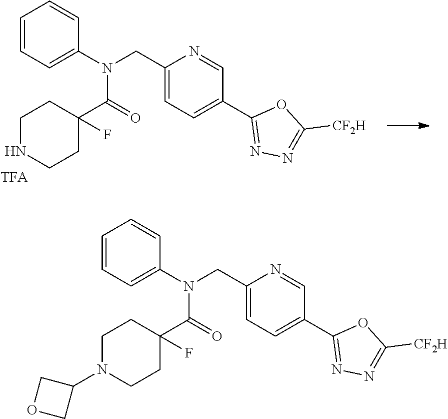 [SMILES: O=C(N(Cc1ccc(-c2nnc(C(F)F)o2)cn1)c1ccccc1)C1(F)CCNCC1 ; O=C(N(Cc1ccc(-c2nnc(C(F)F)o2)cn1)c1ccccc1)C1(F)CCN(C2COC2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.075 g, 0.138 mmol) prepared in step 6 of Example 6, oxetan-3-one (0.018 mL, 0.275 mmol), and acetic acid (0.008 mL, 0.138 mmol) were dissolved in dichloromethane (4 mL), and the resulting solution was stirred at room temperature for 1 hour. Sodium triacetoxyborohydride (0.087 g, 0.413 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO 2, 4 g cartridge; methanol/dichloromethane=0% to 10%) and concentrated to obtain the title compound (0.024 g, 35.8%) as a foamy solid.

¹H NMR (400 MHz, CDCl₃) δ 9.24 (m, 1H), 8.35 (m, 1H), 7.35 (m, 1H), 7.32 (m, 3H), 7.22 (m, 2H), 6.95 (m, 1H), 5.08 (s, 2H), 4.60 (m, 4H), 3.45 (m, 1H), 2.58 (m, 2H), 2.43-2.33 (m, 2H), 1.97 (m, 4H);

LRMS (ES) m/z 488.5 (M++1).

### Example 10: Synthesis of Compound 2969, N-((5-(5-(difluoromethyl)-1,3,4-oxadiazol-2-yl)pyridin-2-yl)methyl)-4-fluoro-N-(3-fluorophenyl)-1-methylpiperidine-4-carboxamide

### [Step 1] Synthesis of N-((5-(5-(difluoromethyl)-1,3,4-oxadiazol-2-yl)pyridin-2-yl)methyl)-3-fluoroaniline

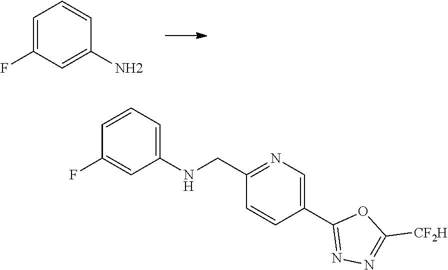

To a solution in which 3-fluoroaniline (1.000 g, 8.999 mmol) and potassium carbonate (1.866 g, 13.499 mmol) were dissolved in N,N-dimethylformamide (40 mL) at room temperature, 2-(6-(bromomethyl)pyridin-3-yl)-5-(difluoromethyl)-1,3,4-oxadiazole (2.480 g, 8.549 mmol) prepared in step 3 of Example 6 and potassium iodide (0.747 g, 4.500 mmol) were added and stirred at the same temperature for 18 hours. Water was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified by column chromatography (SiO 2, 40 g cartridge; ethyl acetate/hexane=0% to 60%) and concentrated to obtain the title compound (2.340 g, 81.2%) as a yellow solid.

### [Step 2] Synthesis of tert-butyl 4-(((5-(5-(difluoromethyl)-1,3,4-oxadiazol-2-yl)pyridin-2-yl)methyl)(3-fluorophenyl)carbamoyl)-4-fluoropiperidine-1-carboxylate

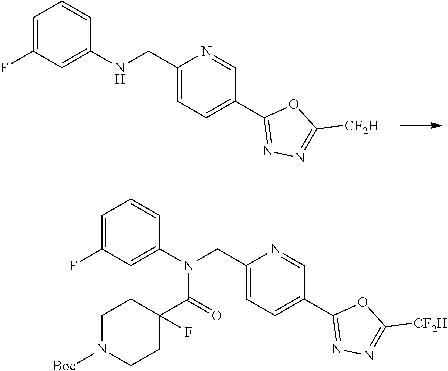 [SMILES: Fc1cccc(NCc2ccc(-c3nnc(C(F)F)o3)cn2)c1 ; CC(C)(C)OC(=O)N1CCC(F)(C(=O)N(Cc2ccc(-c3nnc(C(F)F)o3)cn2)c2cccc(F)c2)CC1]

To a solution in which N-((5-(5-(difluoromethyl)-1,3,4-oxadiazol-2-yl)pyridin-2-yl)methyl)-3-fluoroaniline (0.490 g, 1.530 mmol) prepared in step 1 and triethylamine (0.640 mL, 4.590 mmol) were dissolved in dichloromethane (20 mL) at room temperature, tert-butyl 4-(chlorocarbonyl)-4-fluoropiperidine-1-carboxylate (0.528 g, 1.989 mmol) prepared in step 2 of Example 1 was added and stirred at the same temperature for 16 hours. A saturated aqueous ammonium chloride solution was poured into the reaction mixture, followed by extraction with dichloromethane. The organic layer was washed with a saturated aqueous water solution, dried over anhydrous magnesium sulfate, filtered, and concentrated under reduced pressure. The concentrate was purified by column chromatography (SiO 2, 12 g cartridge; ethyl acetate/hexane=0% to 30%) and concentrated to obtain the title compound (0.430 g, 51.1%) as a yellow solid.

### [Step 3] Synthesis of N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate

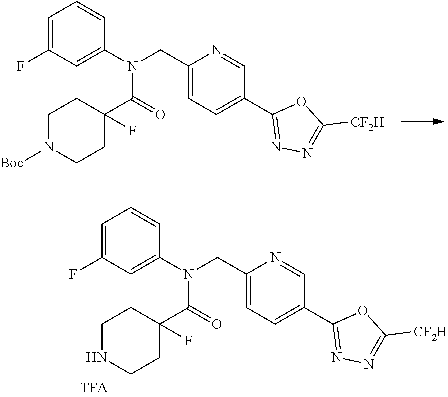 [SMILES: CC(C)(C)OC(=O)N1CCC(F)(C(=O)N(Cc2ccc(-c3nnc(C(F)F)o3)cn2)c2cccc(F)c2)CC1 ; O=C(N(Cc1ccc(-c2nnc(C(F)F)o2)cn1)c1cccc(F)c1)C1(F)CCNCC1]

Tert-butyl 4-(((5-(5-(difluoromethyl)-1,3,4-oxadiazol-2-yl)pyridin-2-yl)methyl)(3-fluorophenyl)carbamoyl)-4-fluoropiperidine-1-carboxylate (0.430 g, 0.782 mmol) prepared in step 2, and trifluoroacetic acid (1.198 mL, 15.650 mmol) were dissolved in dichloromethane (30 mL) at room temperature, and the resulting solution was stirred at the same temperature for 18 hours. After removing the solvent from the reaction mixture under reduced pressure, the title compound (0.350 g, 99.5%) was obtained as a brown liquid.

### [Step 4] Synthesis of Compound 2969

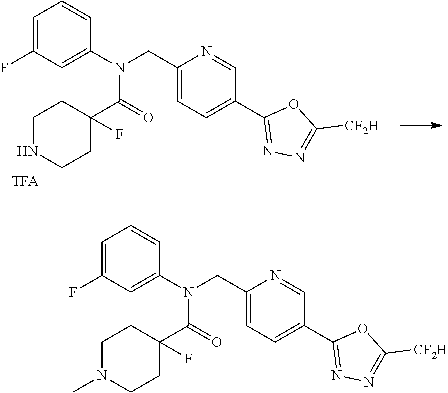 [SMILES: O=C(N(Cc1ccc(-c2nnc(C(F)F)o2)cn1)c1cccc(F)c1)C1(F)CCNCC1 ; CN1CCC(F)(C(=O)N(Cc2ccc(-c3nnc(C(F)F)o3)cn2)c2cccc(F)c2)CC1]

N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate (0.088 g, 0.196 mmol) prepared in step 3, formaldehyde (0.012 g, 0.392 mmol), acetic acid (0.011 mL, 0.196 mmol), and sodium triacetoxyborohydride (0.125 g, 0.587 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.011 g, 12.1%) as a white solid.

¹H NMR (400 MHz, CDCl₃) δ 9.32-9.19 (m, 1H), 8.38 (dd, J=8.2, 2.2 Hz, 1H), 7.53 (d, J=8.2 Hz, 1H), 7.32 (ddd, J=13.5, 6.8, 4.2 Hz, 1H), 7.09-6.81 (m, 4H), 5.06 (s, 2H), 2.72 (d, J=11.2 Hz, 2H), 2.50-2.31 (m, 2H), 2.28 (s, 3H), 2.16 (t, J=11.6 Hz, 2H), 2.02-1.89 (m, 2H);

LRMS (ES) m/z 464.6 (M++1).

### Example 11: Synthesis of Compound 2970, N-((5-(5-(difluoromethyl)-1,3,4-oxadiazol-2-yl)pyridin-2-yl)methyl)-4-fluoro-N-(3-fluorophenyl)-1-isopropylpiperidine-4-carboxamide

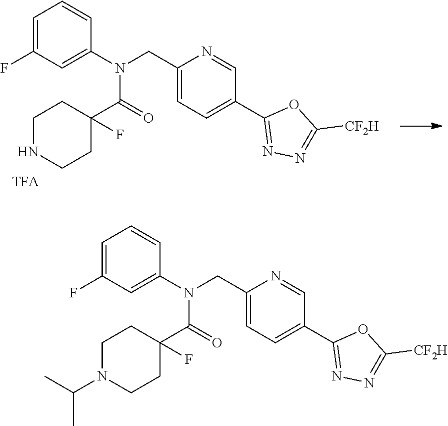 [SMILES: O=C(N(Cc1ccc(-c2nnc(C(F)F)o2)cn1)c1cccc(F)c1)C1(F)CCNCC1 ; CC(C)N1CCC(F)(C(=O)N(Cc2ccc(-c3nnc(C(F)F)o3)cn2)c2cccc(F)c2)CC1]

N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate (0.088 g, 0.196 mmol) prepared in step 3 of Example 10, propan-2-one (0.023 g, 0.392 mmol), acetic acid (0.011 mL, 0.196 mmol), and sodium triacetoxyborohydride (0.125 g, 0.587 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.024 g, 24.9%) as a white solid.

¹H NMR (400 MHz, CDCl₃) δ 9.27 (d, J=1.6 Hz, 1H), 8.39 (dd, J=8.2, 2.2 Hz, 1H), 7.54 (d, J=8.3 Hz, 1H), 7.36-7.29 (m, 1H), 7.00 (ddd, J=73.7, 45.8, 33.6 Hz, 4H), 5.07 (s, 2H), 2.74 (s, 2H), 2.45-2.24 (m, 4H), 1.98 (d, J=11.1 Hz, 3H), 1.04 (d, J=6.5 Hz, 6H);

LRMS (ES) m/z 492.5 (M++1).

### Example 12: Synthesis of Compound 2971, 1-cyclobutyl-N-((5-(5-(difluoromethyl)-1,3,4-oxadiazol-2-yl)pyridin-2-yl)methyl)-4-fluoro-N-(3-fluorophenyl)piperidine-4-carboxamide

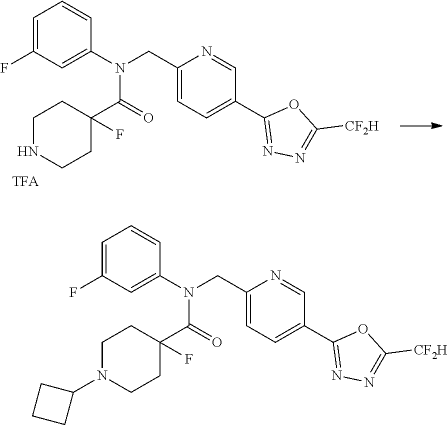 [SMILES: O=C(N(Cc1ccc(-c2nnc(C(F)F)o2)cn1)c1cccc(F)c1)C1(F)CCNCC1 ; O=C(N(Cc1ccc(-c2nnc(C(F)F)o2)cn1)c1cccc(F)c1)C1(F)CCN(C2CCC2)CC1]

N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate (0.088 g, 0.196 mmol) prepared in step 3 of Example 10, cyclobutanone (0.027 g, 0.392 mmol), acetic acid (0.011 mL, 0.196 mmol), and sodium triacetoxyborohydride (0.125 g, 0.587 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.035 g, 35.5%) as a white solid.

¹H NMR (400 MHz, CDCl₃) δ 9.26 (d, J=1.6 Hz, 1H), 8.39 (dd, J=8.2, 2.2 Hz, 1H), 7.53 (d, J=8.2 Hz, 1H), 7.35-7.29 (m, 1H), 7.10-6.80 (m, 4H), 5.07 (s, 2H), 2.70 (t, J=11.7 Hz, 3H), 2.45-2.22 (m, 2H), 2.07-1.83 (m, 7H), 1.75-1.59 (m, 3H);

LRMS (ES) m/z 504.4 (M++1).

### Example 13: Synthesis of Compound 2972, N-((5-(5-(difluoromethyl)-1,3,4-oxadiazol-2-yl)pyridin-2-yl)methyl)-4-fluoro-N-(3-fluorophenyl)-1-(oxetan-3-yl)piperidine-4-carboxamide

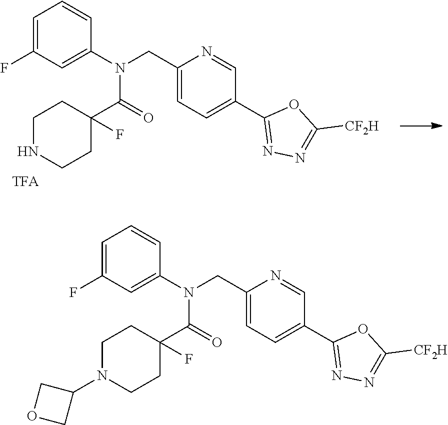 [SMILES: O=C(N(Cc1ccc(-c2nnc(C(F)F)o2)cn1)c1cccc(F)c1)C1(F)CCNCC1 ; O=C(N(Cc1ccc(-c2nnc(C(F)F)o2)cn1)c1cccc(F)c1)C1(F)CCN(C2COC2)CC1]

N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate (0.088 g, 0.196 mmol) prepared in step 3 of Example 10, oxetan-3-one (0.028 g, 0.392 mmol), acetic acid (0.011 mL, 0.196 mmol), and sodium triacetoxyborohydride (0.125 g, 0.587 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.045 g, 45.5%) as a white solid.

¹H NMR (400 MHz, CDCl₃) δ 9.28 (d, J=1.5 Hz, 1H), 8.39 (dd, J=8.2, 2.2 Hz, 1H), 7.53 (d, J=8.3 Hz, 1H), 7.36-7.29 (m, 1H), 7.10-6.81 (m, 4H), 5.07 (s, 2H), 4.62 (dt, J=15.9, 6.4 Hz, 4H), 3.47 (p, J=6.6 Hz, 1H), 2.59 (d, J=8.6 Hz, 2H), 2.49-2.27 (m, 2H), 2.00 (dt, J=24.8, 12.4 Hz, 4H);

LRMS (ES) m/z 506.4 (M++1).

### Example 14: Synthesis of Compound 2973, N-((5-(5-(difluoromethyl)-1,3,4-oxadiazol-2-yl)pyridin-2-yl)methyl)-3-fluoro-1-meth yl-N-phenylazetidine-3-carboxamide

### [Step 1] Synthesis of tert-butyl 3-(chlorocarbonyl)-3-fluoroazetidine-1-carboxylate

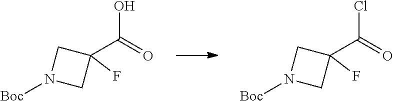 [SMILES: CC(C)(C)OC(=O)N1CC(F)(C(=O)O)C1 ; CC(C)(C)OC(=O)N1CC(F)(C(=O)Cl)C1]

1-(Tert-butoxycarbonyl)-3-fluoroazetidine-3-carboxylic acid (0.500 g, 2.281 mmol) was dissolved in dichloromethane (20 mL). Oxalyl chloride (2.00 M solution in DCM, 1.483 mL, 2.965 mmol) and N,N-dimethylformamide (0.018 mL, 0.228 mmol) were added at 0° C., and stirred at room temperature for 2 hours. After removing the solvent from the reaction mixture under reduced pressure, the title compound (0.540 g, 99.6%) was obtained as a beige solid.

### [Step 2] Synthesis of tert-butyl 3-(((5-(5-(difluoromethyl)-1,3,4-oxadiazol-2-yl)pyridin-2-yl)methyl)(phenyl)carbamoyl)-3-fluoroazetidine-1-carboxylate

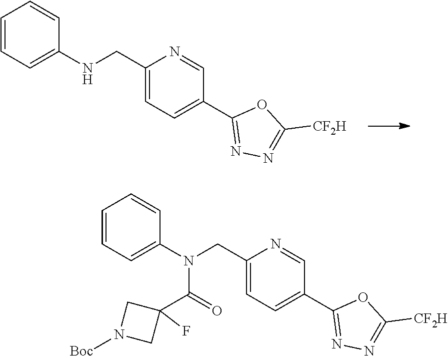 [SMILES: FC(F)c1nnc(-c2ccc(CNc3ccccc3)nc2)o1 ; CC(C)(C)OC(=O)N1CC(F)(C(=O)N(Cc2ccc(-c3nnc(C(F)F)o3)cn2)c2ccccc2)C1]

To a solution in which N-((5-(5-(difluoromethyl)-1,3,4-oxadiazol-2-yl)pyridin-2-yl)methyl)aniline (0.500 g, 1.654 mmol) prepared in step 4 of Example 6 and triethylamine (0.692 mL, 4.962 mmol) were dissolved in dichloromethane (35 mL) at room temperature, tert-butyl 3-(chlorocarbonyl)-3-fluoroazetidine-1-carboxylate (0.511 g, 2.150 mmol) prepared in step 1 was added and stirred at the same temperature for 16 hours. A saturated aqueous ammonium chloride solution was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO 2, 40 g cartridge; ethyl acetate/hexane=5% to 50%) and concentrated to obtain the title compound (0.610 g, 73.2%) as a foamy solid.

### [Step 3] Synthesis of N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate

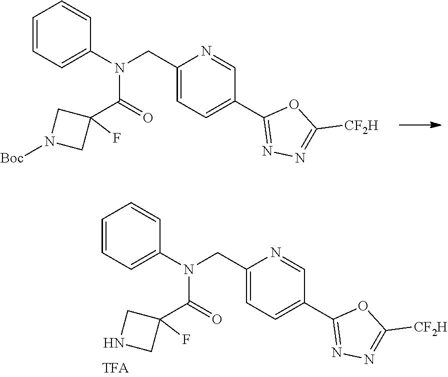 [SMILES: CC(C)(C)OC(=O)N1CC(F)(C(=O)N(Cc2ccc(-c3nnc(C(F)F)o3)cn2)c2ccccc2)C1 ; O=C(N(Cc1ccc(-c2nnc(C(F)F)o2)cn1)c1ccccc1)C1(F)CNC1]

Tert-butyl 3-(((5-(5-(difluoromethyl)-1,3,4-oxadiazol-2-yl)pyridin-2-yl)methyl)(phenyl)carbamoyl)-3-fluoroazetidine-1-carboxylate (0.200 g, 0.397 mmol) prepared in step 2 was dissolved in dichloromethane (12 mL). Trifluoroacetic acid (0.913 mL, 11.917 mmol) was added at 0° C., and stirred at room temperature for 16 hours. After removing the solvent from the reaction mixture under reduced pressure, the title compound (0.200 g, 97.3%) was obtained as a foamy solid.

### [Step 4] Synthesis of Compound 2973

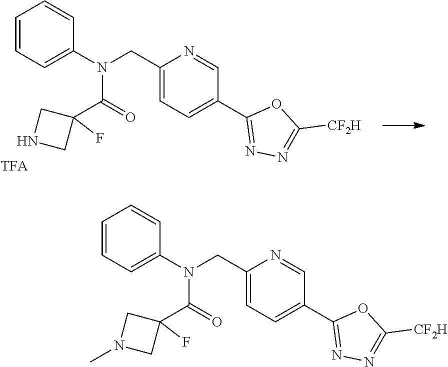 [SMILES: O=C(N(Cc1ccc(-c2nnc(C(F)F)o2)cn1)c1ccccc1)C1(F)CNC1 ; CN1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.050 g, 0.097 mmol) prepared in step 3, paraformaldehyde (0.006 g, 0.193 mmol), and acetic acid (0.006 mL, 0.097 mmol) were dissolved in dichloromethane (4 mL), and the resulting solution was stirred at room temperature for 1 hour. Sodium triacetoxyborohydride (0.061 g, 0.290 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO 2, 4 g cartridge; methanol/dichloromethane=0% to 10%) and concentrated to obtain the title compound (0.021 g, 52.1%) as a foamy solid.

¹H NMR (400 MHz, CDCl₃) δ 9.25 (m, 1H), 8.39 (m, 1H), 7.59 (m, 1H), 7.35 (m, 3H), 7.25 (m, 2H), 6.95 (m, 1H), 5.12 (s, 2H), 3.60 (m, 2H), 3.18 (m, 2H), 2.34 (s, 3H);

LRMS (ES) m/z 418.5 (M++1).

### Example 15: Synthesis of Compound 2974, N-((5-(5-(difluoromethyl)-1,3,4-oxadiazol-2-yl)pyridin-2-yl)methyl)-3-fluoro-1-isopropyl-N-phenylazetidine-3-carboxamide

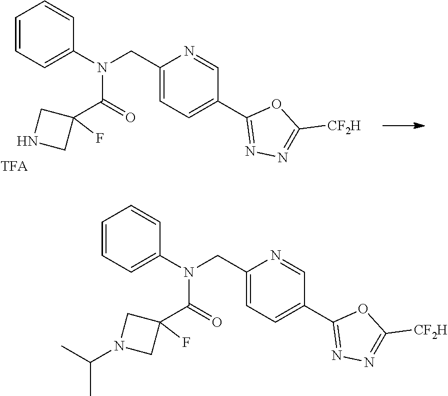 [SMILES: O=C(N(Cc1ccc(-c2nnc(C(F)F)o2)cn1)c1ccccc1)C1(F)CNC1 ; CC(C)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.050 g, 0.097 mmol) prepared in step 3 of Example 14, acetone (0.014 mL, 0.193 mmol), and acetic acid (0.006 mL, 0.097 mmol) were dissolved in dichloromethane (4 mL), and the resulting solution was stirred at room temperature for 1 hour. Sodium triacetoxyborohydride (0.061 g, 0.290 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO 2, 4 g cartridge; methanol/dichloromethane=0% to 10%) and concentrated to obtain the title compound (0.022 g, 51.1%) as a foamy solid.

¹H NMR (400 MHz, CDCl₃) δ 9.25 (m, 1H), 8.38 (m, 1H), 7.58 (m, 1H), 7.35 (m, 3H), 7.25 (m, 2H), 6.95 (m, 1H), 5.12 (s, 2H), 3.53 (m, 2H), 3.11 (m, 2H), 2.30 (m, 1H), 0.90 (m, 6H);

LRMS (ES) m/z 446.6 (M++1).

### Example 16: Synthesis of Compound 2975, 1-cyclobutyl-N-((5-(5-(difluoromethyl)-1,3,4-oxadiazol-2-yl)pyridin-2-yl)methyl)-3-fluoro-N-phenylazetidine-3-carboxamide

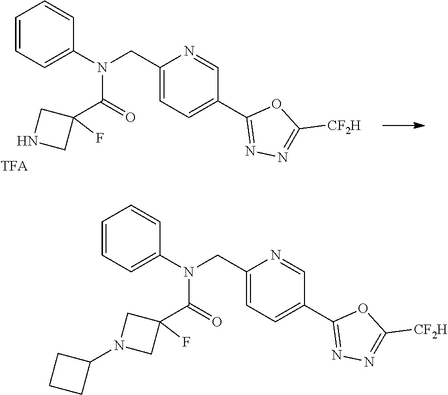 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(C2CCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.050 g, 0.097 mmol) prepared in step 3 of Example 14, cyclobutanone (0.014 mL, 0.193 mmol), and acetic acid (0.006 mL, 0.097 mmol) were dissolved in dichloromethane (4 mL), and the resulting solution was stirred at room temperature for 1 hour. Sodium triacetoxyborohydride (0.061 g, 0.290 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.025 g, 56.6%) as a white solid.

¹H NMR (400 MHz, CDCl₃) δ 9.24 (m, 1H), 8.38 (m, 1H), 7.57 (m, 1H), 7.35 (m, 3H), 7.25 (m, 2H), 6.95 (m, 1H), 5.12 (s, 2H), 3.53 (m, 2H), 3.12 (m, 1H), 3.06 (m, 2H), 1.91 (m, 2H), 1.66 (m, 4H);

LRMS (ES) m/z 458.5 (M++1).

### Example 17: Synthesis of Compound 2976, N-((5-(5-(difluoromethyl)-1,3,4-oxadiazol-2-yl)pyridin-2-yl)methyl)-3-fluoro-1-(oxetan-3-yl)-N-phenylazetidine-3-carboxamide

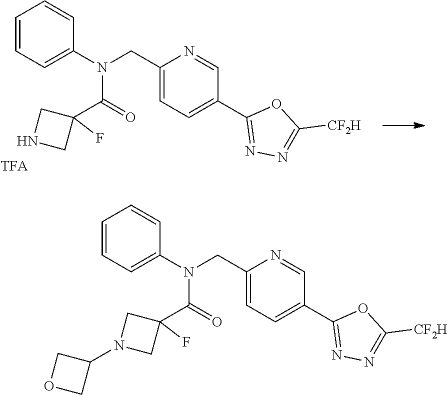 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(C2COC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.050 g, 0.097 mmol) prepared in step 3 of Example 14, oxetan-3-one (0.012 mL, 0.193 mmol), and acetic acid (0.006 mL, 0.097 mmol) were dissolved in dichloromethane (4 mL), and the resulting solution was stirred at room temperature for 1 hour. Sodium triacetoxyborohydride (0.061 g, 0.290 mmol) was added and further stirred at the same temperature for 16 hours. Water was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; methanol/dichloromethane=0% to 10%) and concentrated to obtain the title compound (0.024 g, 54.1%) as a white solid.

¹H NMR (400 MHz, CDCl₃) δ 9.26 (m, 1H), 8.38 (m, 1H), 7.57 (m, 1H), 7.35 (m, 3H), 7.25 (m, 2H), 6.95 (m, 1H), 5.13 (s, 2H), 4.67 (m, 2H), 4.47 (m, 2H), 3.80 (m, 3H), 3.25 (m, 2H);

LRMS (ES) m/z 460.6 (M++1).

### Example 18: Synthesis of Compound 2995, N-((5-(5-(difluoromethyl)-1,3,4-oxadiazol-2-yl)pyridin-2-yl)methyl)-4-fluoro-N-phenyl-1-(2-oxaspiro[3.3]heptan-6-yl)piperidine-4-carboxamide

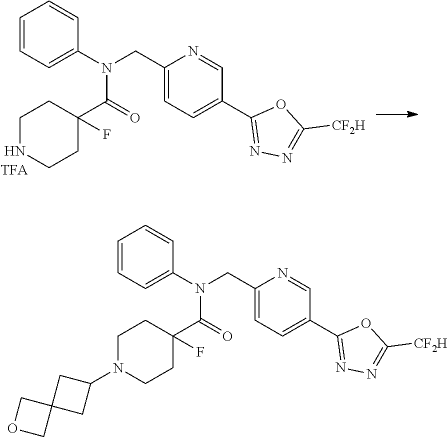 [SMILES: O=C(N(Cc1ccc(-c2nnc(C(F)F)o2)cn1)c1ccccc1)C1(F)CCNCC1 ; O=C(N(Cc1ccc(-c2nnc(C(F)F)o2)cn1)c1ccccc1)C1(F)CCN(C2CC3(COC3)C2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.096 g, 0.223 mmol) prepared in step 6 of Example 6, 2-oxaspiro[3.3]heptan-6-one (0.050 g, 0.445 mmol), acetic acid (0.013 mL, 0.223 mmol), and sodium triacetoxyborohydride (0.141 g, 0.668 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.032 g, 27.3%) as a white solid.

¹H NMR (400 MHz, CDCl₃) δ 9.25 (d, J=2.2 Hz, 1H), 8.37 (dd, J=8.2, 2.3 Hz, 1H), 7.54 (d, J=8.2 Hz, 1H), 7.38-7.29 (m, 3H), 7.21 (dd, J=7.9, 1.6 Hz, 2H), 6.95 (t, J=51.7 Hz, 1H), 5.09 (s, 2H), 4.70 (s, 2H), 4.59 (s, 2H), 2.66 (d, J=11.7 Hz, 2H), 2.48 (dd, J=15.2, 7.8 Hz, 1H), 2.41-2.31 (m, 3H), 2.26 (dd, J=13.7, 4.7 Hz, 1H), 1.98 (ddd, J=40.7, 19.6, 8.9 Hz, 6H);

LRMS (ES) m/z 529.4 (M++1).

### Example 19: Synthesis of Compound 2996, 1-cyclopentyl-N-((5-(5-(difluoromethyl)-1,3,4-oxadiazol-2-yl)pyridin-2-yl)methyl)-4-fluoro-N-phenylpiperidine-4-carboxamide

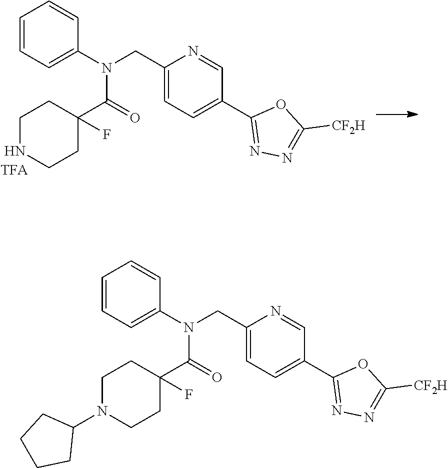 [SMILES: O=C(N(Cc1ccc(-c2nnc(C(F)F)o2)cn1)c1ccccc1)C1(F)CCNCC1 ; O=C(N(Cc1ccc(-c2nnc(C(F)F)o2)cn1)c1ccccc1)C1(F)CCN(C2CCCC2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.096 g, 0.223 mmol) prepared in step 6 of Example 6, cyclopentanone (0.037 g, 0.445 mmol), acetic acid (0.013 mL, 0.223 mmol), and sodium triacetoxyborohydride (0.141 g, 0.668 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.037 g, 33.3%) as a white solid.

¹H NMR (400 MHz, CDCl₃) δ 9.25 (d, J=2.2 Hz, 1H), 8.38 (dd, J=8.2, 2.2 Hz, 1H), 7.55 (d, J=8.3 Hz, 1H), 7.40-7.30 (m, 3H), 7.22 (dd, J=7.7, 1.7 Hz, 2H), 6.95 (t, J=51.7 Hz, 1H), 5.09 (s, 2H), 3.01 (d, J=11.4 Hz, 2H), 2.67 (d, J=7.2 Hz, 1H), 2.59-2.38 (m, 2H), 2.38-2.24 (m, 2H), 1.98 (d, J=11.7 Hz, 2H), 1.82 (d, J=21.6 Hz, 2H), 1.71 (s, 2H), 1.54 (s, 4H);

LRMS (ES) m/z 501.4 (M++1).

### Example 20: Synthesis of Compound 2997, 1-cyclohexyl-N-((5-(5-(difluoromethyl)-1,3,4-oxadiazol-2-yl)pyridin-2-yl)methyl)-4-fluoro-N-phenylpiperidine-4-carboxamide

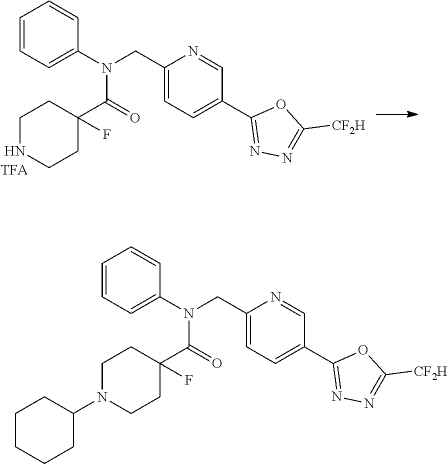 [SMILES: O=C(N(Cc1ccc(-c2nnc(C(F)F)o2)cn1)c1ccccc1)C1(F)CCNCC1 ; O=C(N(Cc1ccc(-c2nnc(C(F)F)o2)cn1)c1ccccc1)C1(F)CCN(C2CCCCC2)CC1]

N-((5-(5-(difluoromethyl)-1,3,4-oxadiazol-2-yl)pyridin-2-yl)methyl)-4-fluoro-N-phenylpiperidine-4-carboxamide 2,2,2-trifluoroacetate (0.096 g, 0.223 mmol) prepared in step 6 of Example 6, cyclohexanone (0.044 g, 0.445 mmol), acetic acid (0.013 mL, 0.223 mmol), and sodium triacetoxyborohydride (0.141 g, 0.668 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.044 g, 38.5%) as a white solid.

¹H NMR (400 MHz, CDCl₃) δ 9.25 (d, J=2.2 Hz, 1H), 8.38 (dd, J=8.2, 2.3 Hz, 1H), 7.56 (d, J=8.1 Hz, 1H), 7.39-7.30 (m, 3H), 7.24-7.19 (m, 2H), 6.97 (dd, J=65.0, 38.4 Hz, 1H), 5.09 (s, 2H), 2.78 (s, 2H), 2.39 (d, J=43.3 Hz, 5H), 1.97 (s, 2H), 1.78 (s, 5H), 1.21 (s, 5H);

LRMS (ES) m/z 515.5 (M++1).

### Example 21: Synthesis of Compound 2998, 1-cyclopentyl-N-((5-(5-(difluoromethyl)-1,3,4-oxadiazol-2-yl)pyridin-2-yl)methyl)-3-fluoro-N-phenylazetidine-3-carboxamide

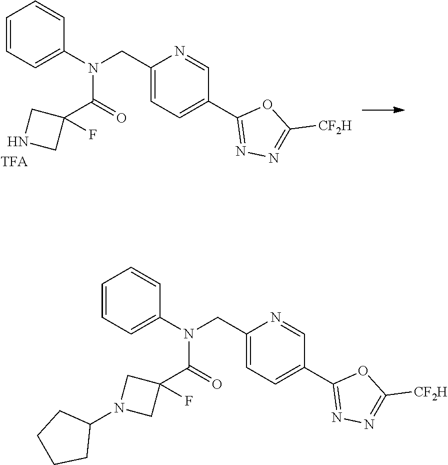 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(C2CCCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.090 g, 0.223 mmol) prepared in step 3 of Example 14, cyclopentanone (0.038 g, 0.446 mmol), acetic acid (0.013 mL, 0.223 mmol), and sodium triacetoxyborohydride (0.142 g, 0.669 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.051 g, 48.5%) as a white solid.

¹H NMR (400 MHz, CDCl₃) δ 9.29-9.20 (m, 1H), 8.38 (dd, J=8.2, 2.2 Hz, 1H), 7.59 (d, J=8.4 Hz, 1H), 7.41-7.31 (m, 3H), 7.27-7.22 (m, 2H), 6.95 (t, J=51.7 Hz, 1H), 5.12 (s, 2H), 3.51 (dd, J=23.8, 10.3 Hz, 2H), 3.11 (dd, J=21.8, 10.4 Hz, 2H), 2.69 (d, J=5.2 Hz, 1H), 1.68-1.60 (m, 2H), 1.60-1.43 (m, 4H), 1.28 (d, J=6.1 Hz, 2H);

LRMS (ES) m/z 473.4 (M++1).

### Example 22: Synthesis of Compound 2999, 1-cyclohexyl-N-((5-(5-(difluoromethyl)-1,3,4-oxadiazol-2-yl)pyridin-2-yl)methyl)-3-fluoro-N-phenylazetidine-3-carboxamide

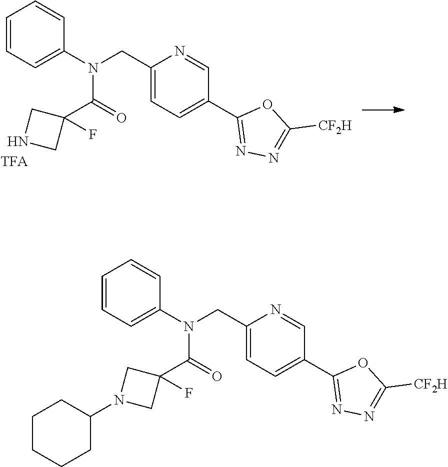 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(C2CCCCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.090 g, 0.223 mmol) prepared in step 3 of Example 14, cyclohexanone (0.044 g, 0.445 mmol), acetic acid (0.013 mL, 0.223 mmol), and sodium triacetoxyborohydride (0.142 g, 0.669 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.050 g, 46.2%) as a white solid.

¹H NMR (400 MHz, CDCl₃) δ 9.25 (d, J=1.6 Hz, 1H), 8.39 (dd, J=8.2, 2.2 Hz, 1H), 7.60 (d, J=8.0 Hz, 1H), 7.40-7.32 (m, 3H), 7.25 (d, J=8.1 Hz, 2H), 6.95 (t, J=51.7 Hz, 1H), 5.13 (s, 2H), 3.53 (dd, J=23.2, 9.8 Hz, 2H), 3.11 (dd, J=21.4, 9.4 Hz, 2H), 2.01-1.85 (m, 2H), 1.72 (d, J=28.0 Hz, 2H), 1.38-1.24 (m, 2H), 1.24-1.10 (m, 3H), 0.97 (d, J=11.8 Hz, 2H);

LRMS (ES) m/z 487.5 (M++1).

### Example 23: Synthesis of Compound 3000, N-((5-(5-(difluoromethyl)-1,3,4-oxadiazol-2-yl)pyridin-2-yl)methyl)-3-fluoro-N-phenyl-1-(tetrahydro-2H-pyran-4-yl)azetidine-3-carboxamide

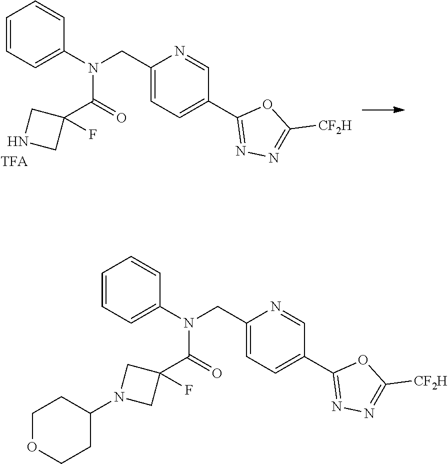 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(C2CCOCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.090 g, 0.223 mmol) prepared in step 3 of Example 14, tetrahydro-4H-pyran-4-one (0.045 g, 0.446 mmol), acetic acid (0.013 mL, 0.223 mmol), and sodium triacetoxyborohydride (0.142 g, 0.669 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.032 g, 29.4%) as a white solid.

¹H NMR (400 MHz, CDCl₃) δ 9.28-9.23 (m, 1H), 8.39 (dd, J=8.2, 2.2 Hz, 1H), 7.59 (d, J=8.3 Hz, 1H), 7.41-7.32 (m, 3H), 7.26 (d, J=8.0 Hz, 2H), 6.95 (t, J=51.6 Hz, 1H), 5.13 (s, 2H), 3.93 (dt, J=11.4, 3.6 Hz, 2H), 3.57 (dd, J=23.2, 10.0 Hz, 2H), 3.35 (td, J=11.2, 1.9 Hz, 2H), 3.13 (dd, J=21.6, 10.1 Hz, 2H), 2.24 (s, 1H), 1.57 (d, J=13.2 Hz, 2H), 1.33 (td, J=14.5, 4.7 Hz, 2H);

LRMS (ES) m/z 488.5 (M++1).

### Example 24: Synthesis of Compound 3001, N-((5-(5-(difluoromethyl)-1,3,4-oxadiazol-2-yl)pyridin-2-yl)methyl)-1-ethyl-3-fluoro-N-phenylazetidine-3-carboxamide

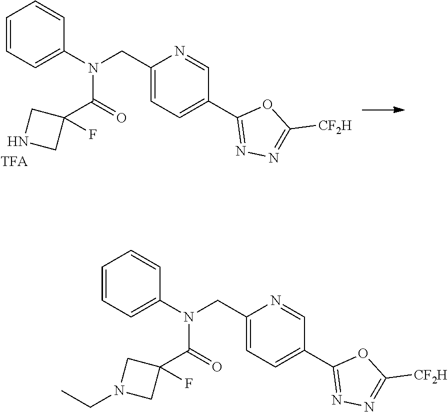 [SMILES: O=C(N(Cc1ccc(-c2nnc(C(F)F)o2)cn1)c1ccccc1)C1(F)CNC1 ; CCN1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, acetaldehyde (0.010 g, 0.238 mmol), acetic acid (0.007 mL, 0.119 mmol), and sodium triacetoxyborohydride (0.076 g, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.042 g, 81.8%) as a yellow solid.

¹H NMR (400 MHz, CDCl₃) δ 9.26 (d, J=1.6 Hz, 1H), 8.39 (dd, J=8.2, 2.2 Hz, 1H), 7.61 (d, J=8.2 Hz, 1H), 7.40-7.34 (m, 3H), 7.26 (d, J=8.1 Hz, 2H), 6.96 (t, J=51.7 Hz, 1H), 5.13 (s, 2H), 3.75 (dd, J=22.9, 10.9 Hz, 2H), 3.24 (dd, J=21.6, 10.5 Hz, 2H), 2.59 (q, J=7.2 Hz, 2H), 0.98 (t, J=7.2 Hz, 3H);

LRMS (ES) m/z 433.4 (M++1).

### Example 25: Synthesis of Compound 3002, N-((5-(5-(difluoromethyl)-1,3,4-oxadiazol-2-yl)pyridin-2-yl)methyl)-3-fluoro-N-phenyl-1-propylazetidine-3-carboxamide

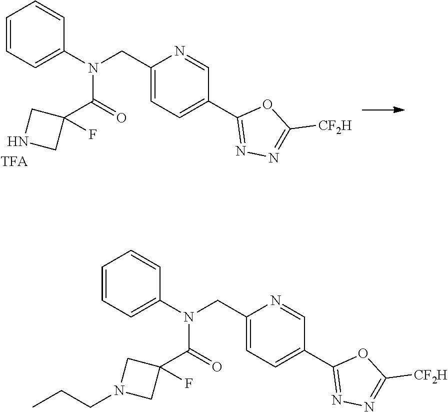 [SMILES: O=C(N(Cc1ccc(-c2nnc(C(F)F)o2)cn1)c1ccccc1)C1(F)CNC1 ; CCCN1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, propioaldehyde (0.014 g, 0.238 mmol), acetic acid (0.007 mL, 0.119 mmol), and sodium triacetoxyborohydride (0.076 g, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.029 g, 54.7%) as a yellow solid.

¹H NMR (400 MHz, CDCl₃) δ 9.25 (d, J=1.5 Hz, 1H), 8.39 (dd, J=8.2, 2.2 Hz, 1H), 7.59 (d, J=8.4 Hz, 1H), 7.41-7.30 (m, 3H), 7.27-7.20 (m, 2H), 6.95 (t, J=51.7 Hz, 1H), 5.13 (s, 2H), 3.56 (dd, J=22.8, 10.1 Hz, 2H), 3.14 (dd, J=21.6, 9.3 Hz, 2H), 2.40 (t, J=7.4 Hz, 2H), 1.32 (dt, J=19.6, 9.8 Hz, 2H), 0.87 (t, J=7.4 Hz, 3H);

LRMS (ES) m/z 447.5 (M++1).

### Example 26: Synthesis of Compound 3003, 1-butyl-N-((5-(5-(difluoromethyl)-1,3,4-oxadiazol-2-yl)pyridin-2-yl)methyl)-3-fluoro-N-phenylazetidine-3-carboxamide

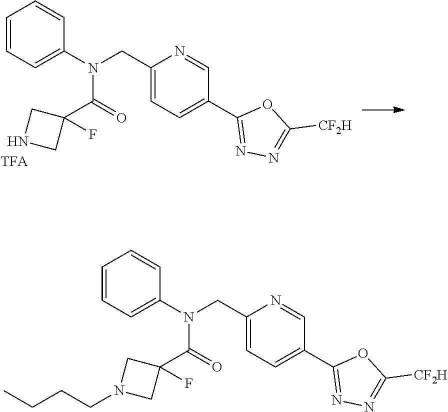 [SMILES: O=C(N(Cc1ccc(-c2nnc(C(F)F)o2)cn1)c1ccccc1)C1(F)CNC1 ; CCCCN1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, butyraldehyde (0.017 g, 0.238 mmol), acetic acid (0.007 mL, 0.119 mmol), and sodium triacetoxyborohydride (0.076 g, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.038 g, 69.5%) as a yellow solid.

¹H NMR (400 MHz, CDCl₃) δ 9.25 (d, J=1.5 Hz, 1H), 8.39 (dd, J=8.2, 2.2 Hz, 1H), 7.59 (d, J=8.1 Hz, 1H), 7.41-7.32 (m, 3H), 7.27-7.21 (m, 2H), 6.95 (t, J=51.7 Hz, 1H), 5.13 (s, 2H), 3.55 (dd, J=22.8, 9.4 Hz, 2H), 3.14 (dd, J=21.5, 10.3 Hz, 2H), 2.42 (s, 2H), 1.34-1.24 (m, 4H), 0.88 (t, J=7.1 Hz, 3H);

LRMS (ES) m/z 461.5 (M++1).

### Example 27: Synthesis of Compound 3004, N-((5-(5-(difluoromethyl)-1,3,4-oxadiazol-2-yl)pyridin-2-yl)methyl)-3-fluoro-1-isobutyl-N-phenylazetidine-3-carboxamide

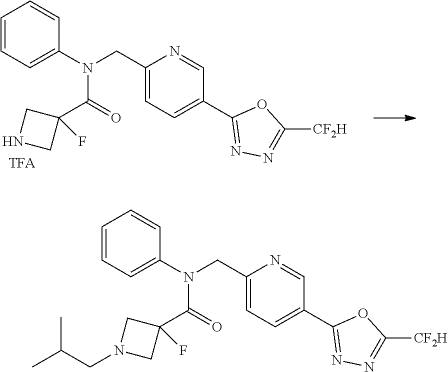 [SMILES: O=C(N(Cc1ccc(-c2nnc(C(F)F)o2)cn1)c1ccccc1)C1(F)CNC1 ; CC(C)CN1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, isobutyraldehyde (0.017 g, 0.238 mmol), acetic acid (0.007 mL, 0.119 mmol), and sodium triacetoxyborohydride (0.076 g, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.040 g, 73.2%) as a yellow solid.

¹H NMR (400 MHz, CDCl₃) δ 9.26 (d, J=1.5 Hz, 1H), 8.39 (dd, J=8.2, 2.2 Hz, 1H), 7.59 (d, J=8.2 Hz, 1H), 7.41-7.33 (m, 3H), 7.27-7.20 (m, 2H), 6.95 (t, J=51.7 Hz, 1H), 5.13 (s, 2H), 3.62-3.46 (m, 2H), 3.15 (dd, J=21.8, 9.7 Hz, 2H), 2.25 (d, J=7.1 Hz, 2H), 1.54 (dt, J=13.3, 6.8 Hz, 1H), 0.85 (d, J=6.7 Hz, 6H);

LRMS (ES) m/z 460.4 (M++1).

### Example 28: Synthesis of Compound 3005, N-((5-(5-(difluoromethyl)-1,3,4-oxadiazol-2-yl)pyridin-2-yl)methyl)-3-fluoro-1-(1-hydroxypropan-2-yl)-N-phenylazetidine-3-carboxamide

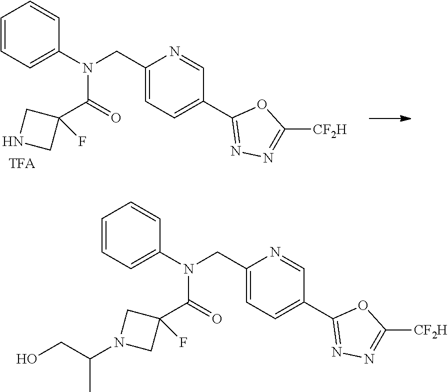 [SMILES: O=C(N(Cc1ccc(-c2nnc(C(F)F)o2)cn1)c1ccccc1)C1(F)CNC1 ; CC(CO)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, 1-hydroxypropan-2-one (0.018 g, 0.238 mmol), acetic acid (0.007 mL, 0.119 mmol), and sodium triacetoxyborohydride (0.076 g, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.023 g, 41.9%) as a yellow solid.

¹H NMR (400 MHz, CDCl₃) δ 9.27 (d, J=1.6 Hz, 1H), 8.39 (dd, J=8.2, 2.2 Hz, 1H), 7.57 (d, J=8.2 Hz, 1H), 7.38 (dd, J=8.6, 3.2 Hz, 3H), 7.25 (d, J=7.7 Hz, 2H), 6.96 (t, J=51.7 Hz, 1H), 5.12 (s, 2H), 3.94-3.75 (m, 2H), 3.62-3.51 (m, 1H), 3.47-3.26 (m, 3H), 2.60 (s, 1H), 0.99 (d, J=6.5 Hz, 3H);

LRMS (ES) m/z 463.5 (M++1).

### Example 29: Synthesis of Compound 3006, N-((5-(5-(difluoromethyl)-1,3,4-oxadiazol-2-yl)pyridin-2-yl)methyl)-1-(3-(dimethylamino)propanoyl)-3-fluoro-N-phenylazetidine-3-carboxamide

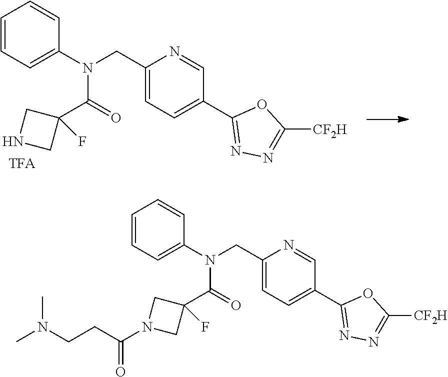 [SMILES: O=C(N(Cc1ccc(-c2nnc(C(F)F)o2)cn1)c1ccccc1)C1(F)CNC1 ; CN(C)CCC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, 3-(dimethylamino)propanoyl chloride (0.021 g, 0.155 mmol), and triethylamine (0.050 mL, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.029 g, 48.5%) as a yellow solid.

¹H NMR (400 MHz, CDCl₃) δ 9.29 (d, J=1.8 Hz, 1H), 8.40 (dd, J=8.2, 2.2 Hz, 1H), 7.54 (d, J=8.1 Hz, 1H), 7.43-7.35 (m, 3H), 7.32-7.24 (m, 2H), 6.96 (t, J=51.7 Hz, 1H), 5.13 (s, 2H), 4.76 (dd, J=21.7, 10.2 Hz, 1H), 4.36 (dd, J=22.7, 12.1 Hz, 1H), 4.04 (dd, J=22.6, 10.5 Hz, 1H), 3.69 (dd, J=22.9, 11.7 Hz, 1H), 2.65 (t, J=7.2 Hz, 2H), 2.29 (d, J=10.1 Hz, 8H);

LRMS (ES) m/z 504.4 (M++1).

### Example 30: Synthesis of Compound 3007, N-((5-(5-(difluoromethyl)-1,3,4-oxadiazol-2-yl)pyridin-2-yl)methyl)-1-(4-(dimethylamino)butanoyl)-3-fluoro-N-phenylazetidine-3-carboxamide

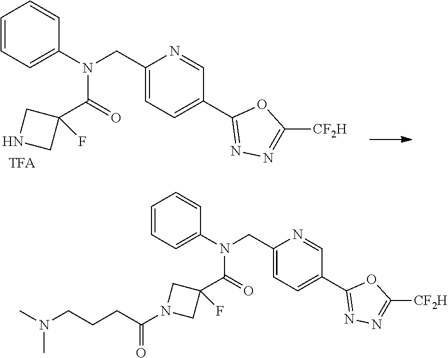 [SMILES: O=C(N(Cc1ccc(-c2nnc(C(F)F)o2)cn1)c1ccccc1)C1(F)CNC1 ; CN(C)CCCC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.048 g, 0.119 mmol) prepared in step 3 of Example 14, 4-(dimethylamino)butanoyl chloride (0.023 g, 0.155 mmol), and triethylamine (0.050 mL, 0.357 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.033 g, 53.7%) as a yellow solid.

¹H NMR (400 MHz, CDCl₃) δ 9.28 (d, J=1.8 Hz, 1H), 8.39 (dd, J=8.2, 2.2 Hz, 1H), 7.54 (d, J=8.2 Hz, 1H), 7.44-7.36 (m, 3H), 7.28 (d, J=5.9 Hz, 2H), 6.96 (t, J=51.7 Hz, 1H), 5.13 (s, 2H), 4.73 (dd, J=22.1, 11.0 Hz, 1H), 4.38 (dd, J=23.1, 11.7 Hz, 1H), 4.02 (dd, J=22.7, 10.4 Hz, 1H), 3.70 (dd, J=22.8, 12.3 Hz, 1H), 2.48 (d, J=6.7 Hz, 2H), 2.36 (s, 6H), 2.16 (t, J=7.1 Hz, 2H), 1.90-1.79 (m, 2H);

LRMS (ES) m/z 517.4 (M++1).

### Example 31: Synthesis of Compound 3047, N-((5-(5-(difluoromethyl)-1,3,4-oxadiazol-2-yl)pyridin-2-yl)methyl)-3-fluoro-N-(3-fluorophenyl)-1-methylazetidine-3-carboxamide

### [Step 1] Synthesis of 3-(((5-(5-(difluoromethyl)-1,3,4-oxadiazol-2-yl)pyridin-2-yl)methyl)(3-fluorophenyl)carbamoyl)-3-fluoroazetidine-1-carboxylate

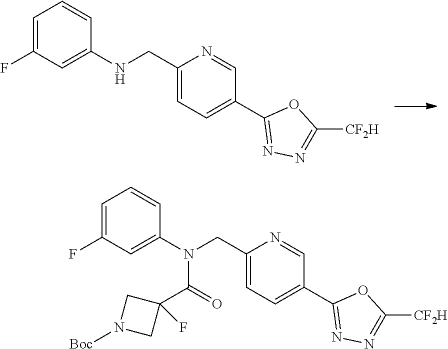 [SMILES: Fc1cccc(NCc2ccc(-c3nnc(C(F)F)o3)cn2)c1 ; CC(C)(C)OC(=O)N1CC(F)(C(=O)N(Cc2ccc(-c3nnc(C(F)F)o3)cn2)c2cccc(F)c2)C1]

To a solution in which N-((5-(5-(difluoromethyl)-1,3,4-oxadiazol-2-yl)pyridin-2-yl)methyl)-3-fluoroaniline (1.100 g, 3.434 mmol) prepared in step 1 of Example 10 and triethylamine (1.436 mL, 10.303 mmol) were dissolved in dichloromethane (20 mL) at room temperature, tertbutyl 3-(chlorocarbonyl)-3-fluoroazetidine-1-carboxylate (1.061 g, 4.465 mmol) prepared in step 1 of Example 14 was added and stirred at the same temperature for 16 hours. A saturated aqueous ammonium chloride solution was poured into the reaction mixture, followed by extraction with dichloromethane. The organic layer was washed with water, dried over anhydrous magnesium sulfate, filtered, and concentrated under reduced pressure. The concentrate was purified by column chromatography (SiO 2, 12 g cartridge; ethyl acetate/hexane=0% to 30%) and concentrated to obtain the title compound (1.210 g, 67.6%) as a yellow solid.

### [Step 2] Synthesis of N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate

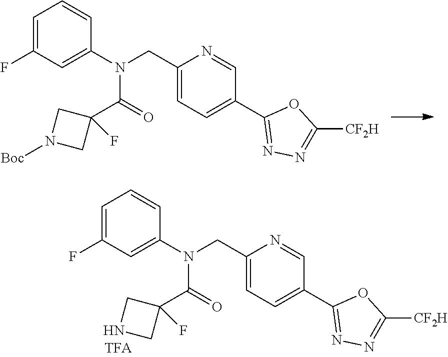 [SMILES: CC(C)(C)OC(=O)N1CC(F)(C(=O)N(Cc2ccc(-c3nnc(C(F)F)o3)cn2)c2cccc(F)c2)C1 ; O=C(N(Cc1ccc(-c2nnc(C(F)F)o2)cn1)c1cccc(F)c1)C1(F)CNC1]

Tert-butyl 3-(((5-(5-(difluoromethyl)-1,3,4-oxadiazol-2-yl)pyridin-2-yl)methyl)(3-fluorophenyl)carbamoyl)-3-fluoroazetidine-1-carboxylate (1.080 g, 2.145 mmol) prepared in step 1, and trifluoroacetic acid (3.285 mL, 42.901 mmol) were dissolved in dichloromethane (30 mL) at room temperature, and the resulting solution was stirred at the same temperature for 18 hours. After removing the solvent from the reaction mixture under reduced pressure, the title compound (0.865 g, 100.0%) was obtained as a brown gel.

### [Step 3] Synthesis of Compound 3047

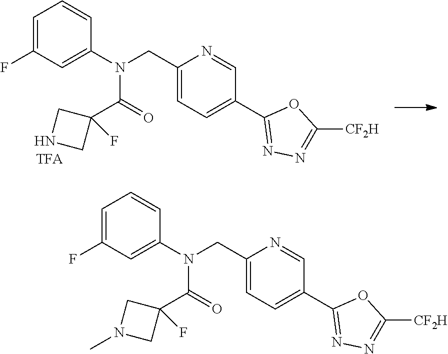 [SMILES: O=C(N(Cc1ccc(-c2nnc(C(F)F)o2)cn1)c1cccc(F)c1)C1(F)CNC1 ; CN1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2, formaldehyde (0.007 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.032 g, 61.9%) as a white solid.

¹H NMR (400 MHz, CDCl₃) δ 9.28 (d, J=1.5 Hz, 1H), 8.40 (dd, J=8.2, 2.3 Hz, 1H), 7.57 (d, J=8.2 Hz, 1H), 7.39-7.31 (m, 1H), 7.01 (dt, J=83.3, 28.8 Hz, 4H), 5.10 (s, 2H), 3.67 (s, 2H), 3.21 (s, 2H), 2.36 (s, 3H);

LRMS (ES) m/z 437.5 (M++1).

### Example 32: Synthesis of Compound 3048, N-((5-(5-(difluoromethyl)-1,3,4-oxadiazol-2-yl)pyridin-2-yl)methyl)-3-fluoro-N-(3-fluorophenyl)-1-isopropylazetidine-3-carboxamide

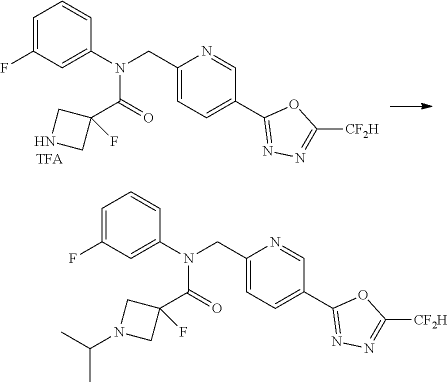 [SMILES: O=C(N(Cc1ccc(-c2nnc(C(F)F)o2)cn1)c1cccc(F)c1)C1(F)CNC1 ; CC(C)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, propan-2-one (0.014 g, 0.237 mmol), acetic acid (0.011 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.026 g, 47.3%) as a white solid.

¹H NMR (400 MHz, CDCl₃) δ 9.27 (d, J=1.6 Hz, 1H), 8.40 (dd, J=8.2, 2.3 Hz, 1H), 7.58 (d, J=7.8 Hz, 1H), 7.39-7.31 (m, 1H), 7.11-6.81 (m, 4H), 5.10 (s, 2H), 3.61 (s, 2H), 3.16 (s, 2H), 2.34 (d, J=6.0 Hz, 1H), 0.92 (d, J=6.2 Hz, 6H);

LRMS (ES) m/z 465.4 (M++1).

### Example 33: Synthesis of Compound 3049, 1-cyclobutyl-N-((5-(5-(difluoromethyl)-1,3,4-oxadiazol-2-yl)pyridin-2-yl)methyl)-3-fluoro-N-(3-fluorophenyl)azetidine-3-carboxamide

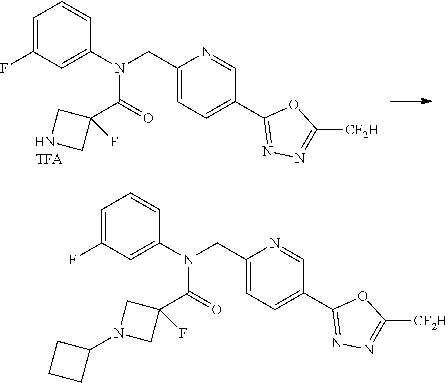 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclobutanone (0.017 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.019 g, 33.7%) as a white solid.

¹H NMR (400 MHz, CDCl₃) δ 9.27 (d, J=2.2 Hz, 1H), 8.40 (dd, J=8.2, 2.3 Hz, 1H), 7.58 (d, J=8.1 Hz, 1H), 7.39-7.31 (m, 1H), 7.01 (dt, J=86.0, 28.3 Hz, 4H), 5.10 (s, 2H), 3.66 (s, 2H), 3.15 (d, J=7.2 Hz, 3H), 2.00-1.92 (m, 2H), 1.78 (dd, J=18.8, 9.9 Hz, 2H), 1.61 (s, 2H);

LRMS (ES) m/z 477.4 (M++1).

### Example 34: Synthesis of Compound 3050, N-((5-(5-(difluoromethyl)-1,3,4-oxadiazol-2-yl)pyridin-2-yl)meth 1)-3-fluoro-N-(3-fluorophenyl)-1-(oxetan-3-yl)azetidine-3-carboxamide

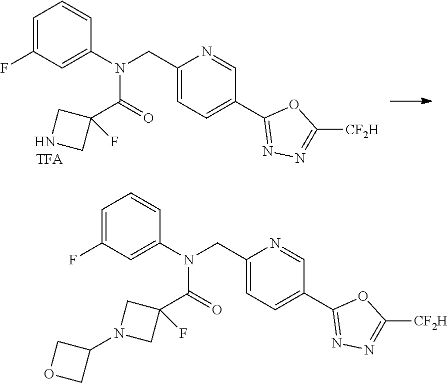 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2COC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, oxetan-3-one (0.017 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.033 g, 58.3%) as a white solid.

¹H NMR (400 MHz, CDCl₃) δ 9.29 (d, J=1.5 Hz, 1H), 8.40 (dd, J=8.2, 2.2 Hz, 1H), 7.56 (d, J=8.1 Hz, 1H), 7.40-7.32 (m, 1H), 7.13-6.82 (m, 4H), 5.11 (s, 2H), 4.69 (t, J=6.9 Hz, 2H), 4.54-4.47 (m, 2H), 3.91-3.74 (m, 3H), 3.34 (d, J=22.0 Hz, 2H);

LRMS (ES) m/z 478.3 (M++1).

### Example 35: Synthesis of Compound 3051, 1-cyclopentyl-N-((5-(5-(difluoromethyl)-1,3,4-oxadiazol-2-yl)pyridin-2-yl)methyl)-3-fluoro-N-(3-fluorophenyl)azetidine-3-carboxamide

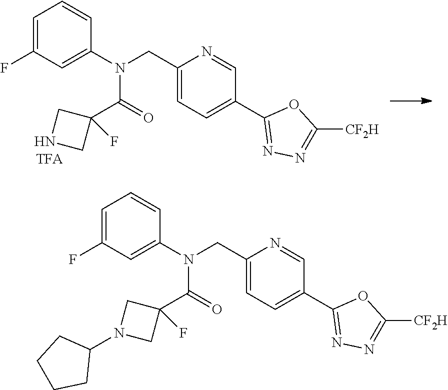 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclopentanone (0.020 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.036 g, 62.0%) as a white solid.

¹H NMR (400 MHz, CDCl₃) δ 9.27 (d, J=1.5 Hz, 1H), 8.40 (dd, J=8.2, 2.2 Hz, 1H), 7.57 (d, J=8.1 Hz, 1H), 7.38-7.30 (m, 1H), 7.12-6.80 (m, 4H), 5.10 (s, 2H), 3.56 (d, J=23.0 Hz, 2H), 3.18 (d, J=13.0 Hz, 2H), 2.73 (s, 1H), 1.71-1.64 (m, 2H), 1.60-1.44 (m, 4H), 1.29 (d, J=7.4 Hz, 2H);

LRMS (ES) m/z 491.5 (M++1).

### Example 36: Synthesis of Compound 3052, N-((5-(5-(difluoromethyl)-1,3,4-oxadiazol-2-yl)pyridin-2-yl)methyl)-3-fluoro-N-(3-fluorophenyl)-1-(tetrahydrofuran-3-yl)azetidine-3-carboxamide

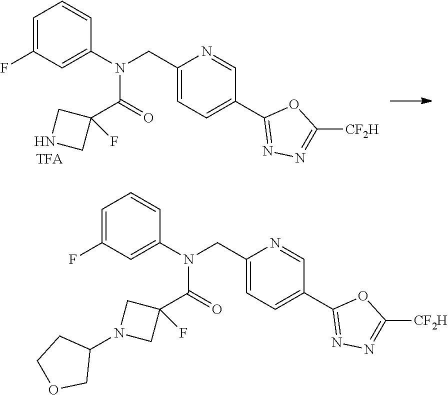 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCOC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, dihydrofuran-3(2H)-one (0.020 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.024 g, 41.2%) as a white solid.

¹H NMR (400 MHz, CDCl₃) δ 9.27 (d, J=1.5 Hz, 1H), 8.40 (dd, J=8.2, 2.2 Hz, 1H), 7.57 (d, J=8.1 Hz, 1H), 7.38-7.30 (m, 1H), 7.12-6.80 (m, 4H), 5.10 (s, 2H), 3.56 (d, J=23.0 Hz, 2H), 3.18 (d, J=13.0 Hz, 2H), 2.73 (s, 1H), 1.71-1.64 (m, 2H), 1.60-1.44 (m, 4H), 1.29 (d, J=7.4 Hz, 2H);

LRMS (ES) m/z 492.3 (M++1).

### Example 37: Synthesis of Compound 3053, 1-cyclohexyl-N-((5-(5-(difluoromethyl)-1,3,4-oxadiazol-2-yl)pyridin-2-yl)methyl)-3-fluoro-N-(3-fluorophenyl)azetidine-3-carboxamide

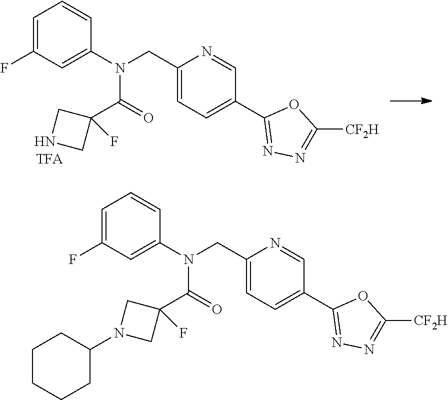 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCCCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclohexanone (0.023 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.030 g, 50.2%) as a white solid.

¹H NMR (400 MHz, CDCl₃) δ 9.29-9.24 (m, 1H), 8.39 (dd, J=8.2, 2.2 Hz, 1H), 7.57 (d, J=8.5 Hz, 1H), 7.39-7.31 (m, 1H), 7.10-6.81 (m, 4H), 5.10 (s, 2H), 3.69-3.52 (m, 3H), 3.18 (d, J=12.1 Hz, 2H), 2.00 (s, 1H), 1.95-1.85 (m, 2H), 1.81-1.69 (m, 3H), 1.36-1.25 (m, 2H), 1.24-1.14 (m, 2H);

LRMS (ES) m/z 505.4 (M++1).

### Example 38: Synthesis of Compound 3054, N-((5-(5-(difluoromethyl)-1,3,4-oxadiazol-2-yl)pyridin-2-yl)methyl)-3-fluoro-N-(3-fluorophenyl)-1-(tetrahydro-2H-pyran-4-yl)azetidine-3-carboxamide

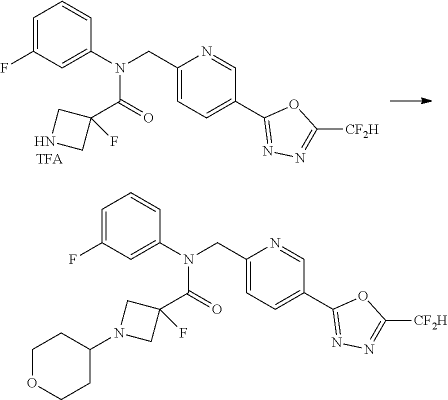 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCOCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, tetrahydro-4H-pyran-4-one (0.024 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.027 g, 45.0%) as a white solid.

¹H NMR (400 MHz, CDCl₃) δ 9.28 (d, J=1.5 Hz, 1H), 8.40 (dd, J=8.2, 2.2 Hz, 1H), 7.57 (d, J=8.3 Hz, 1H), 7.39-7.31 (m, 1H), 7.12-6.81 (m, 4H), 5.10 (s, 2H), 3.94 (dt, J=7.2, 3.8 Hz, 2H), 3.63 (d, J=11.9 Hz, 2H), 3.36 (td, J=11.2, 2.2 Hz, 2H), 3.21 (d, J=22.0 Hz, 2H), 2.28 (s, 1H), 1.58 (s, 2H), 1.41-1.29 (m, 2H);

LRMS (ES) m/z 507.4 (M++1).

### Example 39: Synthesis of Compound 3055, N-((5-(5-(difluoromethyl)-1,3,4-oxadiazol-2-yl)pyridin-2-yl)methyl)-3-fluoro-N-(3-fluorophenyl)-1-(methylsulfonyl)azetidine-3-carboxamide

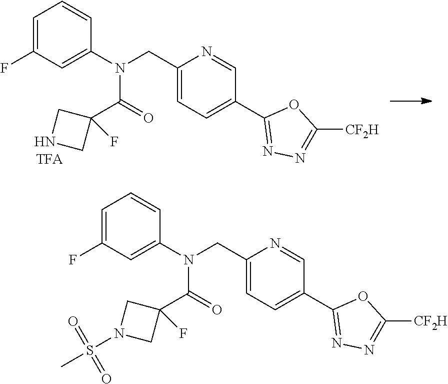 [SMILES: O=C(N(Cc1ccc(-c2nnc(C(F)F)o2)cn1)c1cccc(F)c1)C1(F)CNC1 ; CS(=O)(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, triethylamine (0.033 ml, 0.237 mmol), and methanesulfonyl chloride (0.009 mL, 0.119 mmol) were dissolved in dichloromethane (5 mL), and the resulting solution was stirred at 0° C. for 1 hour and further stirred at room temperature for 2 hours. A saturated aqueous sodium bicarbonate solution was poured into the concentrate obtained by removing the solvent from the reaction mixture under reduced pressure, followed by extraction with dichloromethane. The obtained product was filtered through a plastic filter to remove a solid residue and an aqueous solution layer, and then concentrated under reduced pressure. The concentrate was purified by chromatography (SiO 2 plate, 20×20×1 mm; dichloromethane/methanol=0% to 10%) and concentrated to obtain the title compound (0.041 g, 69.2%) as a white solid.

¹H NMR (400 MHz, CDCl₃) δ 9.31 (d, J=2.1 Hz, 1H), 8.42 (dd, J=8.2, 2.2 Hz, 1H), 7.54 (d, J=8.2 Hz, 1H), 7.39 (dd, J=14.5, 7.4 Hz, 1H), 7.16-6.82 (m, 4H), 5.11 (s, 2H), 4.46 (dd, J=22.1, 10.5 Hz, 2H), 3.82 (dd, J=22.1, 10.3 Hz, 2H), 2.91 (s, 3H);

LRMS (ES) m/z 500.4 (M++1).

### Example 40: Synthesis of Compound 3090, N-((5-(5-(difluoromethyl)-1,3,4-oxadiazol-2-yl)pyridin-2-yl)methyl)-1-ethyl-3-fluoro-N-(3-fluorophenyl)azetidine-3-carboxamide

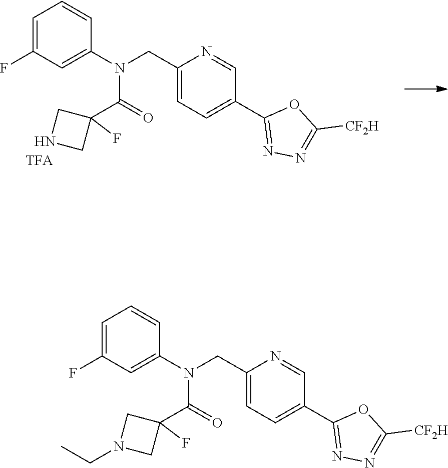 [SMILES: O=C(N(Cc1ccc(-c2nnc(C(F)F)o2)cn1)c1cccc(F)c1)C1(F)CNC1 ; CCN1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, acetaldehyde (0.010 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.026 g, 48.8%) as a yellow gel.

¹H NMR (400 MHz, CDCl₃) δ 9.18 (d, J=1.8 Hz, 1H), 8.30 (dd, J=8.2, 2.2 Hz, 1H), 7.48 (d, J=8.0 Hz, 1H), 7.30-7.21 (m, 1H), 6.93 (ddd, J=86.8, 42.3, 27.5 Hz, 4H), 5.01 (s, 2H), 3.53 (dd, J=21.1, 9.8 Hz, 2H), 3.10 (dd, J=22.0, 9.2 Hz, 2H), 2.40 (q, J=7.1 Hz, 2H), 0.91-0.82 (m, 3H);

LRMS (ES) m/z 450.5 (M++1).

### Example 41: Synthesis of Compound 3091, N-((5-(5-(difluoromethyl)-1,3,4-oxadiazol-2-yl)pyridin-2-yl)methyl)-3-fluoro-N-(3-fluorophenyl)-1-propylazetidine-3-carboxamide

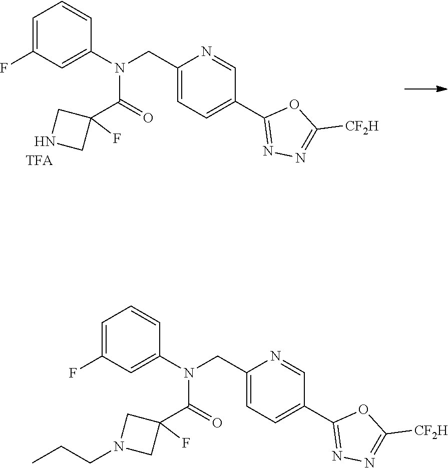 [SMILES: O=C(N(Cc1ccc(-c2nnc(C(F)F)o2)cn1)c1cccc(F)c1)C1(F)CNC1 ; CCCN1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, propioaldehyde (0.014 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.034 g, 61.8%) as a yellow gel.

¹H NMR (400 MHz, CDCl₃) δ 9.18 (d, J=1.7 Hz, 1H), 8.30 (dd, J=8.2, 2.2 Hz, 1H), 7.48 (d, J=8.2 Hz, 1H), 7.29-7.21 (m, 1H), 7.02-6.68 (m, 4H), 5.01 (s, 2H), 3.52 (dd, J=22.8, 9.0 Hz, 2H), 3.11 (dd, J=21.4, 8.9 Hz, 2H), 2.33 (t, J=7.4 Hz, 2H), 1.26 (dq, J=14.9, 7.4 Hz, 2H), 0.79 (t, J=7.4 Hz, 3H);

LRMS (ES) m/z 464.3 (M++1).

### Example 42: Synthesis of Compound 3092, 1-butyl-N-((5-(5-(difluoromethyl)-1,3,4-oxadiazol-2-yl)pyridin-2-yl)methyl)-3-fluoro-N-(3-fluorophenyl)azetidine-3-carboxamide

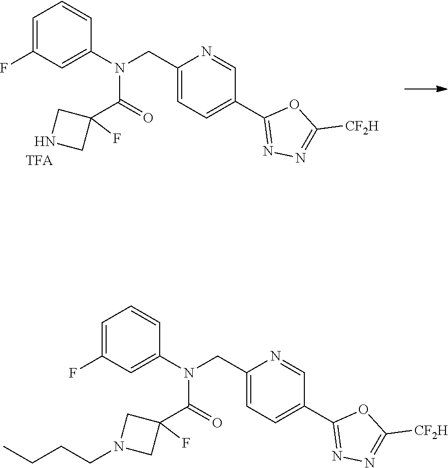 [SMILES: O=C(N(Cc1ccc(-c2nnc(C(F)F)o2)cn1)c1cccc(F)c1)C1(F)CNC1 ; CCCCN1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, butyraldehyde (0.017 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.050 g, 88.3%) as a yellow gel.

¹H NMR (400 MHz, CDCl₃) δ 9.27 (d, J=1.7 Hz, 1H), 8.40 (dd, J=8.2, 2.2 Hz, 1H), 7.56 (t, J=7.9 Hz, 1H), 7.40-7.30 (m, 1H), 7.12-6.79 (m, 4H), 5.10 (s, 2H), 3.61 (dd, J=22.3, 7.1 Hz, 2H), 3.27-3.10 (m, 2H), 2.44 (s, 2H), 1.34-1.24 (m, 4H), 0.89 (t, J=7.1 Hz, 3H);

LRMS (ES) m/z 478.3 (M++1).

### Example 43: Synthesis of Compound 3093, N-((5-(5-(difluoromethyl)-1,3,4-oxadiazol-2-yl)pyridin-2-yl)methyl)-3-fluoro-N-(3-fluorophenyl)-1-isobutylazetidine-3-carboxamide

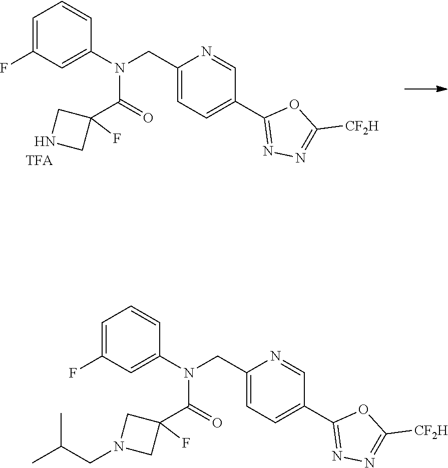 [SMILES: O=C(N(Cc1ccc(-c2nnc(C(F)F)o2)cn1)c1cccc(F)c1)C1(F)CNC1 ; CC(C)CN1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, isobutyraldehyde (0.017 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.041 g, 72.4%) as a yellow gel.

¹H NMR (400 MHz, CDCl₃) δ 9.28 (d, J=1.6 Hz, 1H), 8.40 (dd, J=8.2, 2.2 Hz, 1H), 7.56 (t, J=7.3 Hz, 1H), 7.39-7.30 (m, 1H), 7.11-6.79 (m, 4H), 5.10 (s, 2H), 3.56 (dd, J=13.2, 8.8 Hz, 2H), 3.21 (dd, J=22.0, 9.3 Hz, 2H), 2.26 (d, J=7.0 Hz, 2H), 1.55 (dt, J=13.6, 6.8 Hz, 1H), 0.87 (d, J=6.7 Hz, 6H);

LRMS (ES) m/z 479.4 (M++1).

### Example 44: Synthesis of Compound 3094, N-((5-(5-(difluoromethyl)-1,3,4-oxadiazol-2-yl)pyridin-2-yl)methyl)-3-fluoro-N-(3-fluorophenyl)-1-(1-hydroxypropan-2-yl)azetidine-3-carboxamide

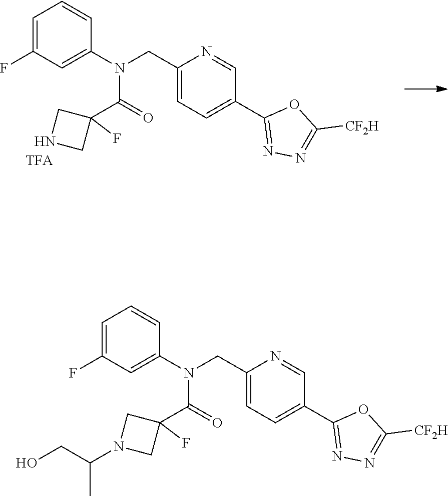 [SMILES: O=C(N(Cc1ccc(-c2nnc(C(F)F)o2)cn1)c1cccc(F)c1)C1(F)CNC1 ; CC(CO)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, 1-hydroxypropan-2-one (0.018 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.041 g, 72.1%) as a yellow gel.

¹H NMR (400 MHz, CDCl₃) δ 9.29 (d, J=1.7 Hz, 1H), 8.40 (dd, J=8.2, 2.2 Hz, 1H), 7.55 (d, J=8.5 Hz, 1H), 7.40-7.31 (m, 1H), 7.13-6.78 (m, 4H), 5.08 (d, J=13.5 Hz, 2H), 3.83 (t, J=30.9 Hz, 2H), 3.61-3.49 (m, 1H), 3.44-3.27 (m, 3H), 2.56 (s, 1H), 0.99 (d, J=6.5 Hz, 3H);

LRMS (ES) m/z 480.5 (M++1).

### Example 45: Synthesis of Compound 3095, N-((5-(5-(difluoromethyl)-1,3,4-oxadiazol-2-yl)pyridin-2-yl)methyl)-3-fluoro-N-(3-fluorophenyl)-1-(1-methylpiperidin-4-yl)azetidine-3-carboxamide

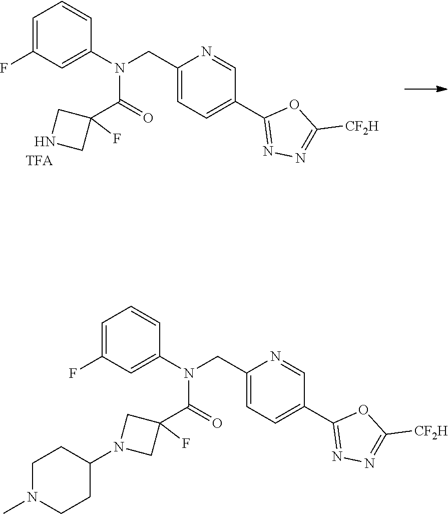 [SMILES: O=C(N(Cc1ccc(-c2nnc(C(F)F)o2)cn1)c1cccc(F)c1)C1(F)CNC1 ; CN1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, 1-methylpiperidin-4-one (0.027 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.037 g, 60.1%) as a yellow gel.

¹H NMR (400 MHz, CDCl₃) δ 9.29 (d, J=1.7 Hz, 1H), 8.40 (dd, J=8.2, 2.2 Hz, 1H), 7.54 (d, J=8.1 Hz, 1H), 7.40-7.31 (m, 1H), 7.01 (dt, J 76.0, 29.3 Hz, 4H), 5.10 (s, 2H), 3.63 (dd, J=21.3, 7.9 Hz, 2H), 3.19 (dd, J=21.3, 9.0 Hz, 2H), 3.00 (t, J=8.6 Hz, 2H), 2.76 (dd, J=19.4, 13.1 Hz, 2H), 2.53 (d, J=10.0 Hz, 3H), 2.36 (s, 1H), 1.90 (s, 2H), 1.57 (s, 2H);

LRMS (ES) m/z 519.4 (M++1).

### Example 46: Synthesis of Compound 3096, 1-(4,4-difluorocyclohexyl)-N-((5-(5-(difluoromethyl)-1,3,4-oxadiazol-2-yl)pyridin-2-yl)methyl)-3-fluoro-N-(3-fluorophenyl)azetidine-3-carboxamide

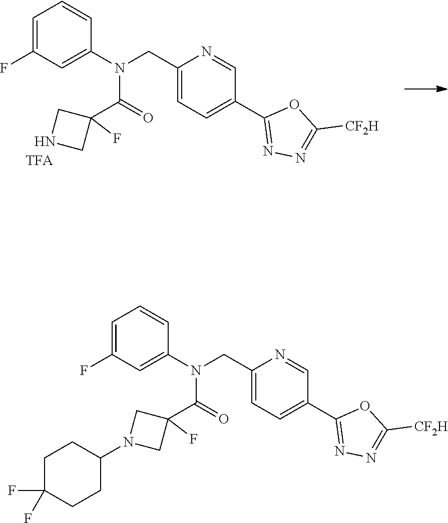 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCC(F)(F)CC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, 4,4-difluorocyclohexan-1-one (0.032 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.034 g, 53.1%) as a yellow gel.

¹H NMR (400 MHz, CDCl₃) δ 9.28 (d, J=1.7 Hz, 1H), 8.40 (dd, J=8.2, 2.2 Hz, 1H), 7.56 (d, J=8.3 Hz, 1H), 7.41-7.31 (m, 1H), 7.14-6.75 (m, 4H), 5.10 (s, 2H), 3.62 (dd, J=22.1, 8.6 Hz, 2H), 3.19 (dd, J=21.0, 9.0 Hz, 2H), 2.24 (s, 1H), 2.12-1.97 (m, 2H), 1.79-1.58 (m, 4H), 1.47 (dd, J=21.3, 13.0 Hz, 2H);

LRMS (ES) m/z 541.6 (M++1).

### Example 47: Synthesis of Compound 3097, N-((5-(5-(difluoromethyl)-1,3,4-oxadiazol-2-yl)pyridin-2-yl)methyl)-1-(3-(dimethylamino)propanoyl)-3-fluoro-N-(3-fluorophenyl)azetidine-3-carboxamide

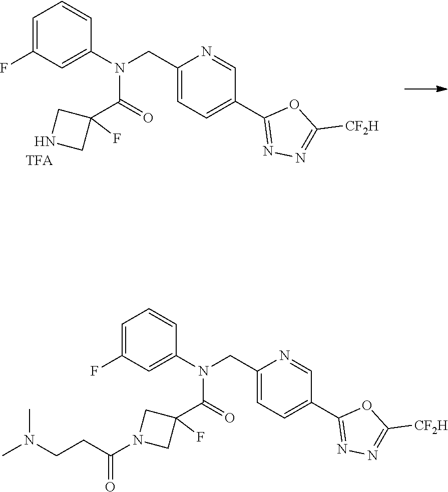 [SMILES: O=C(N(Cc1ccc(-c2nnc(C(F)F)o2)cn1)c1cccc(F)c1)C1(F)CNC1 ; CN(C)CCC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 3 of Example 31, 3-(dimethylamino)propanoyl chloride (0.021 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.035 g, 56.7%) as a yellow gel.

¹H NMR (400 MHz, CDCl₃) δ 9.21 (d, J=1.8 Hz, 1H), 8.32 (dd, J=8.2, 2.2 Hz, 1H), 7.45 (d, J=8.1 Hz, 1H), 7.29 (dd, J=14.8, 7.9 Hz, 1H), 7.05-6.69 (m, 4H), 5.01 (s, 2H), 4.70 (dd, J=21.2, 10.8 Hz, 1H), 4.46 (dd, J=21.4, 12.0 Hz, 1H), 3.99 (dd, J=21.8, 10.2 Hz, 1H), 3.78 (dd, J=21.7, 11.7 Hz, 1H), 3.07 (q, J=7.3 Hz, 2H), 2.70 (s, 6H), 1.26 (t, J=7.3 Hz, 2H);

LRMS (ES) m/z 521.5 (M++1).

### Example 48: Synthesis of Compound 3098, N-((5-(5-(difluoromethyl)-1,3,4-oxadiazol-2-yl)pyridin-2-yl)methyl)-1-(4-(dimethylamino)butanoyl)-3-fluoro-N-(3-fluorophenyl)azetidine-3-carboxamide

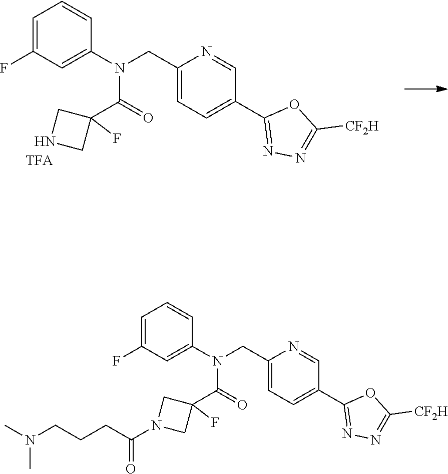 [SMILES: O=C(N(Cc1ccc(-c2nnc(C(F)F)o2)cn1)c1cccc(F)c1)C1(F)CNC1 ; CN(C)CCCC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, 4-(dimethylamino)butanoyl chloride (0.023 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.029 g, 45.7%) as a yellow gel.

¹H NMR (400 MHz, CDCl₃) δ 9.30 (d, J=1.8 Hz, 1H), 8.41 (dd, J=8.2, 2.2 Hz, 1H), 7.53 (d, J=8.2 Hz, 1H), 7.38 (dd, J=14.4, 8.0 Hz, 1H), 7.03 (dt, J=75.6, 30.1 Hz, 4H), 5.10 (d, J=1.9 Hz, 2H), 4.72 (dd, J=21.3, 10.5 Hz, 1H), 4.48 (dd, J=21.6, 11.6 Hz, 1H), 4.03 (dd, J=21.8, 9.8 Hz, 1H), 3.82 (dd, J=22.8, 11.9 Hz, 1H), 2.78 (s, 6H), 1.35 (t, J=7.3 Hz, 6H);

LRMS (ES) m/z 535.3 (M++1).

### Example 49: Synthesis of Compound 3105, N-((5-(5-(difluoromethyl)-1,3,4-oxadiazol-2-yl)pyridin-2-yl)methyl)-3-fluoro-1′-methyl-N-phenyl-[1,3′-biazetidine]-3-carboxamide

### [Step 1] Synthesis of tert-butyl 3-(((5-(5-(difluoromethyl)-1,3,4-oxadiazol-2-yl)pyridin-2-yl)methyl)(phenyl)carbamoyl)-3-fluoro-[1,3′-biazetidine]-1′-carboxylate

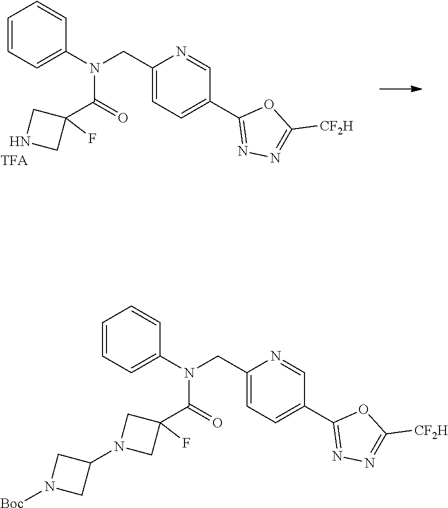 [SMILES: O=C(N(Cc1ccc(-c2nnc(C(F)F)o2)cn1)c1ccccc1)C1(F)CNC1 ; CC(C)(C)OC(=O)N1CC(N2CC(F)(C(=O)N(Cc3ccc(-c4nnc(C(F)F)o4)cn3)c3ccccc3)C2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.250 g, 0.620 mmol) prepared in step 3 of Example 14, tert-butyl 3-oxoazetidine-1-carboxylate (0.212 g, 1.240 mmol), acetic acid (0.035 mL, 0.620 mmol), and sodium triacetoxyborohydride (0.394 g, 1.859 mmol) were dissolved in dichloromethane (20 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by column chromatography (SiO₂, 12 g cartridge; dichloromethane/methanol=0% to 10%) and concentrated to obtain the title compound (0.300 g, 86.7%) as a yellow gel.

### [Step 2] Synthesis of N-((5-(5-(difluoromethyl)-1,3,4-oxadiazol-2-yl)pyridin-2-yl)methyl)-3-fluoro-N-phenyl-[1,3′-biazetidine]-3-carboxamide 2,2,2-trifluoroacetate

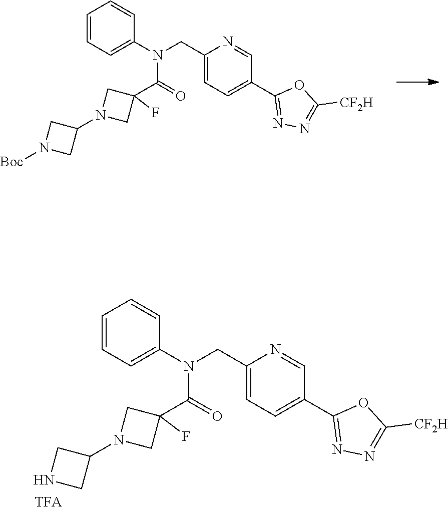 [SMILES: CC(C)(C)OC(=O)N1CC(N2CC(F)(C(=O)N(Cc3ccc(-c4nnc(C(F)F)o4)cn3)c3ccccc3)C2)C1 ; O=C(N(Cc1ccc(-c2nnc(C(F)F)o2)cn1)c1ccccc1)C1(F)CN(C2CNC2)C1]

Tert-butyl 3-(((5-(5-(difluoromethyl)-1,3,4-oxadiazol-2-yl)pyridin-2-yl)methyl)(phenyl)carbamoyl)-3-fluoro-[1,3′-biazetidine]-1′-carboxylate (0.262 g, 0.469 mmol) prepared in step 1, and trifluoroacetic acid (0.718 mL, 9.381 mmol) were dissolved in dichloromethane (10 mL) at room temperature, and the resulting solution was stirred at the same temperature for 18 hours. After removing the solvent from the reaction mixture under reduced pressure, the title compound (0.215 g, 100.0%) was obtained as a yellow gel.

### [Step 3] Synthesis of Compound 3105

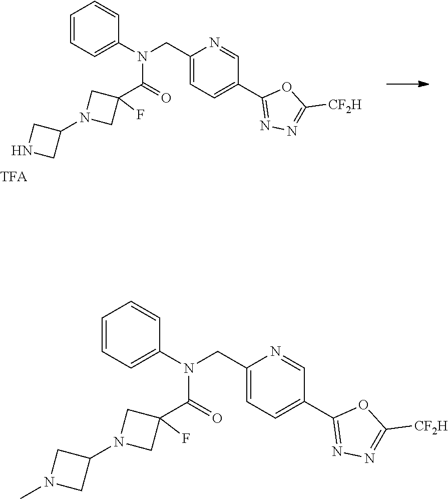 [SMILES: O=C(N(Cc1ccc(-c2nnc(C(F)F)o2)cn1)c1ccccc1)C1(F)CN(C2CNC2)C1 ; CN1CC(N2CC(F)(C(=O)N(Cc3ccc(-c4nnc(C(F)F)o4)cn3)c3ccccc3)C2)C1]

N-((5-(5-(difluoromethyl)-1,3,4-oxadiazol-2-yl)pyridin-2-yl)methyl)-3-fluoro-N-phenyl-[1,3′-biazetidine]-3-carboxamide 2,2,2-trifluoroacetate (0.040 g, 0.087 mmol) prepared in step 2, formaldehyde (0.005 g, 0.175 mmol), acetic acid (0.005 mL, 0.087 mmol), and sodium triacetoxyborohydride (0.055 g, 0.262 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.014 g, 34.0%) as a yellow gel.

¹H NMR (400 MHz, CDCl₃) δ 9.27 (d, J=1.6 Hz, 1H), 8.39 (dd, J=8.1, 2.2 Hz, 1H), 7.57 (d, J=8.0 Hz, 1H), 7.40-7.34 (m, 3H), 7.26 (d, J=7.9 Hz, 2H), 6.97 (dd, J=64.5, 38.9 Hz, 1H), 5.12 (s, 2H), 3.86 (s, 2H), 3.69 (dd, J=21.1, 9.0 Hz, 2H), 3.55-3.47 (m, 1H), 3.31-3.12 (m, 4H), 2.60 (s, 3H);

LRMS (ES) m/z 473.5 (M++1).

### Example 50: Synthesis of Compound 3106, N-((5-(5-(difluoromethyl)-1,3,4-oxadiazol-2-yl)pyridin-2-yl)methyl)-1′-ethyl-3-fluoro-N-phenyl-[1,3′-biazetidine]-3-carboxamide

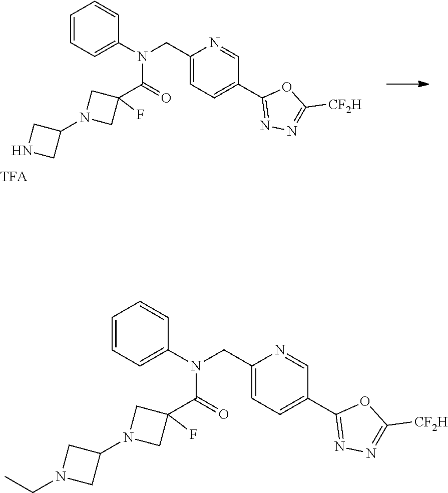 [SMILES: O=C(N(Cc1ccc(-c2nnc(C(F)F)o2)cn1)c1ccccc1)C1(F)CN(C2CNC2)C1 ; CCN1CC(N2CC(F)(C(=O)N(Cc3ccc(-c4nnc(C(F)F)o4)cn3)c3ccccc3)C2)C1]

N-((5-(5-(difluoromethyl)-1,3,4-oxadiazol-2-yl)pyridin-2-yl)methyl)-3-fluoro-N-phenyl-[1,3′-biazetidine]-3-carboxamide 2,2,2-trifluoroacetate (0.040 g, 0.087 mmol) prepared in step 2 of Example 49, acetaldehyde (0.008 g, 0.175 mmol), acetic acid (0.007 mL, 0.087 mmol), and sodium triacetoxyborohydride (0.055 g, 0.262 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.022 g, 51.8%) as a yellow gel.

¹H NMR (400 MHz, CDCl₃) δ 9.26 (d, J=2.1 Hz, 1H), 8.39 (dd, J=8.2, 2.2 Hz, 1H), 7.58 (d, J=8.3 Hz, 1H), 7.38 (dt, J=10.6, 3.6 Hz, 3H), 7.25 (d, J=8.0 Hz, 2H), 6.97 (dd, J=64.9, 38.4 Hz, 1H), 5.12 (s, 2H), 3.75-3.60 (m, 2H), 3.43 (dt, J=29.9, 6.6 Hz, 3H), 3.17 (dd, J=21.7, 10.1 Hz, 2H), 2.96 (d, J=6.9 Hz, 2H), 2.56 (q, J=7.2 Hz, 2H), 1.00 (t, J=7.2 Hz, 3H);

LRMS (ES) m/z 488.5 (M++1).

### Example 51: Synthesis of Compound 3107, N-((5-(5-(difluoromethyl)-1,3,4-oxadiazol-2-yl)pyridin-2-yl)methyl)-3-fluoro-N-phenyl-1′-propyl-[1,3′-biazetidine]-3-carboxamide

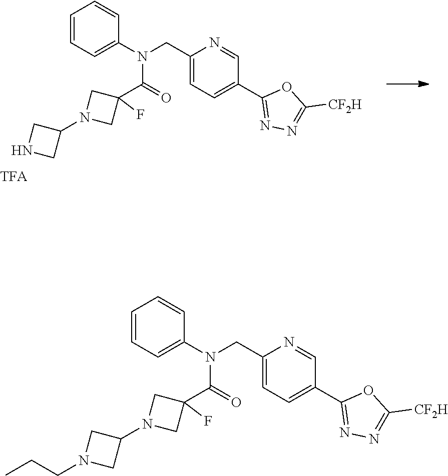 [SMILES: O=C(N(Cc1ccc(-c2nnc(C(F)F)o2)cn1)c1ccccc1)C1(F)CN(C2CNC2)C1 ; CCCN1CC(N2CC(F)(C(=O)N(Cc3ccc(-c4nnc(C(F)F)o4)cn3)c3ccccc3)C2)C1]

N-((5-(5-(difluoromethyl)-1,3,4-oxadiazol-2-yl)pyridin-2-yl)methyl)-3-fluoro-N-phenyl-[1,3′-biazetidine]-3-carboxamide 2,2,2-trifluoroacetate (0.040 g, 0.087 mmol) prepared in step 2 of Example 49, propioaldehyde (0.010 g, 0.175 mmol), acetic acid (0.005 mL, 0.087 mmol), and sodium triacetoxyborohydride (0.055 g, 0.262 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.018 g, 41.2%) as a yellow gel.

¹H NMR (400 MHz, CDCl₃) δ 9.27 (d, J=1.7 Hz, 1H), 8.40 (dd, J=8.2, 2.2 Hz, 1H), 7.57 (d, J=8.3 Hz, 1H), 7.38 (d, J=7.1 Hz, 3H), 7.25 (d, J=5.4 Hz, 2H), 6.96 (t, J=51.7 Hz, 1H), 5.12 (s, 2H), 3.97 (s, 2H), 3.68 (dd, J=23.3, 12.6 Hz, 3H), 3.34-3.11 (m, 4H), 2.80 (s, 2H), 1.54 (dd, J=15.2, 7.8 Hz, 2H), 0.94 (t, J=7.4 Hz, 3H);

LRMS (ES) m/z 501.3 (M++1).

### Example 52: Synthesis of Compound 3108, 1′-butyl-N-((5-(5-(difluoromethyl)-1,3,4-oxadiazol-2-yl)pyridin-2-yl)methyl)-3-fluoro-N-phenyl-[1,3′-biazetidine]-3-carboxamide

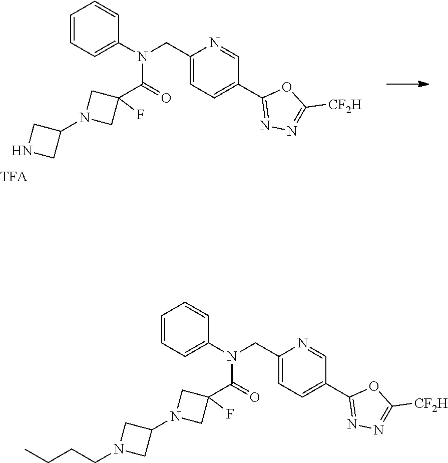 [SMILES: O=C(N(Cc1ccc(-c2nnc(C(F)F)o2)cn1)c1ccccc1)C1(F)CN(C2CNC2)C1 ; CCCCN1CC(N2CC(F)(C(=O)N(Cc3ccc(-c4nnc(C(F)F)o4)cn3)c3ccccc3)C2)C1]

N-((5-(5-(difluoromethyl)-1,3,4-oxadiazol-2-yl)pyridin-2-yl)methyl)-3-fluoro-N-phenyl-[1,3′-biazetidine]-3-carboxamide 2,2,2-trifluoroacetate (0.040 g, 0.087 mmol) prepared in step 2 of Example 49, butyraldehyde (0.013 g, 0.175 mmol), acetic acid (0.005 mL, 0.087 mmol), and sodium triacetoxyborohydride (0.055 g, 0.262 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.018 g, 40.1%) as a yellow gel.

¹H NMR (400 MHz, CDCl₃) δ 9.26 (d, J=1.6 Hz, 1H), 8.39 (dd, J=8.2, 2.2 Hz, 1H), 7.58 (d, J=8.3 Hz, 1H), 7.37 (d, J=7.3 Hz, 3H), 7.25 (d, J=8.1 Hz, 2H), 6.96 (t, J=51.7 Hz, 1H), 5.12 (s, 2H), 3.66 (d, J=21.7 Hz, 3H), 3.46 (s, 1H), 3.17 (dd, J=21.7, 9.9 Hz, 2H), 3.04 (s, 2H), 2.59 (s, 2H), 1.43-1.24 (m, 5H), 0.91 (t, J=7.2 Hz, 3H);

LRMS (ES) m/z 516.5 (M++1).

### Example 53: Synthesis of Compound 3109, N-((5-(5-(difluoromethyl)-1,3,4-oxadiazol-2-yl)pyridin-2-yl)methyl)-3-fluoro-1′-isobutyl-N-phenyl-[1,3′-biazetidine]-3-carboxamide

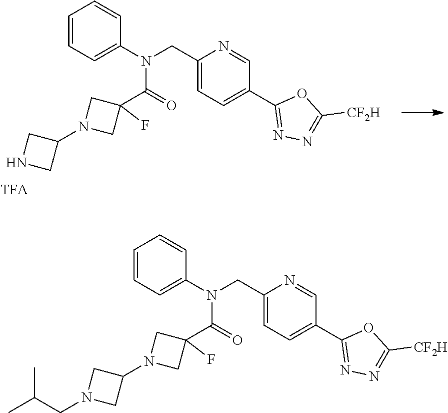 [SMILES: O=C(N(Cc1ccc(-c2nnc(C(F)F)o2)cn1)c1ccccc1)C1(F)CN(C2CNC2)C1 ; CC(C)CN1CC(N2CC(F)(C(=O)N(Cc3ccc(-c4nnc(C(F)F)o4)cn3)c3ccccc3)C2)C1]

N-((5-(5-(difluoromethyl)-1,3,4-oxadiazol-2-yl)pyridin-2-yl)methyl)-3-fluoro-N-phenyl-[1,3′-biazetidine]-3-carboxamide 2,2,2-trifluoroacetate (0.040 g, 0.087 mmol) prepared in step 2 of Example 49, isobutyraldehyde (0.013 g, 0.175 mmol), acetic acid (0.005 mL, 0.087 mmol), and sodium triacetoxyborohydride (0.055 g, 0.262 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.016 g, 35.6%) as a yellow gel.

¹H NMR (400 MHz, CDCl₃) δ 9.26 (d, J=2.1 Hz, 1H), 8.39 (dd, J=8.2, 2.2 Hz, 1H), 7.59 (d, J=8.2 Hz, 1H), 7.36 (t, J=5.4 Hz, 3H), 7.25 (d, J=8.2 Hz, 2H), 6.97 (dd, J=64.8, 38.6 Hz, 1H), 5.13 (s, 2H), 3.67 (dd, J=22.5, 9.8 Hz, 2H), 3.37 (s, 3H), 3.17 (dd, J=22.4, 10.4 Hz, 2H), 2.89 (s, 2H), 2.26 (d, J=6.6 Hz, 2H), 1.67-1.49 (m, 1H), 0.88 (d, J=6.7 Hz, 6H);

LRMS (ES) m/z 516.5 (M++1).

### Example 54: Synthesis of Compound 3110, N-((5-(5-(difluoromethyl)-1,3,4-oxadiazol-2-yl)pyridin-2-yl)methyl)-3-fluoro-1-(1-methylpiperidin-4-yl)-N-phenylazetidine-3-carboxamide

### [Step 1] Synthesis of tert-butyl 4-(3-(((5-(5-(difluoromethyl)-1,3,4-oxadiazol-2-yl)pyridin-2-yl)methyl)(phenyl)carbamoyl)-3-fluoroazetidine-1-ylpiperidine-1-carboxylate

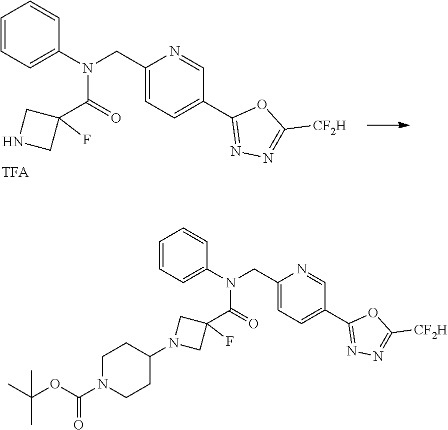 [SMILES: O=C(N(Cc1ccc(-c2nnc(C(F)F)o2)cn1)c1ccccc1)C1(F)CNC1 ; CC(C)(C)OC(=O)N1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (1,000 g, 2.479 mmol) prepared in step 3 of Example 14, tert-butyl 4-oxopiperidine-1-carboxylate (0.988 g, 4.958 mmol), acetic acid (0.142 mL, 2.479 mmol), and sodium triacetoxyborohydride (1.576 g, 7.437 mmol) were dissolved in dichloromethane (50 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The organic layer was washed with a saturated aqueous water solution, dried over anhydrous magnesium sulfate, filtered, and concentrated under reduced pressure. The concentrate was purified by column chromatography (SiO₂, 40 g cartridge; methanol/dichloromethane=0% to 10%) and concentrated to obtain the title compound (1.100 g, 75.6%) as white solid.

### [Step 2] Synthesis of N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate

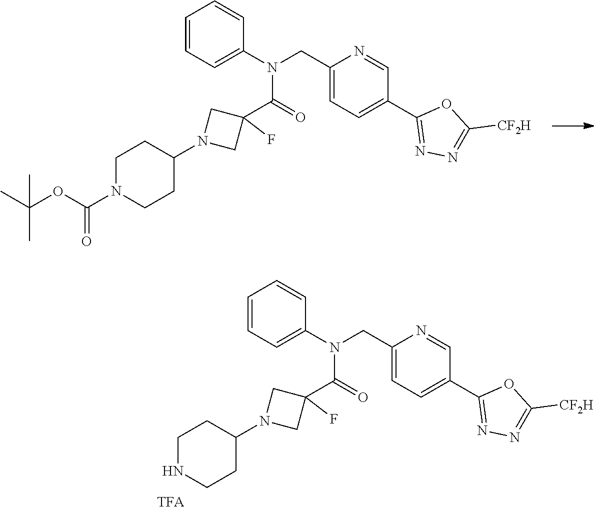 [SMILES: CC(C)(C)OC(=O)N1CCC(N2CC(F)(C(=O)N(Cc3ccc(-c4nnc(C(F)F)o4)cn3)c3ccccc3)C2)CC1 ; O=C(N(Cc1ccc(-c2nnc(C(F)F)o2)cn1)c1ccccc1)C1(F)CN(C2CCNCC2)C1]

Tert-butyl 4-(3-(((5-(5-(difluoromethyl)-1,3,4-oxadiazol-2-yl)pyridin-2-yl)methyl)(phenyl)carbamoyl)-3-fluoroazetidine-1-yl)piperidine-1-carboxylate (1.000 g, 1.705 mmol) prepared in step 1, and trifluoroacetic acid (0.718 mL, 8.523 mmol) were dissolved in dichloromethane (10 mL) at room temperature, and the resulting solution was stirred at the same temperature for 3 hours. After removing the solvent from the reaction mixture under reduced pressure, the title compound (0.800 g, 96.5%) was obtained as a yellow gel.

### [Step 3] Synthesis of Compound 3110

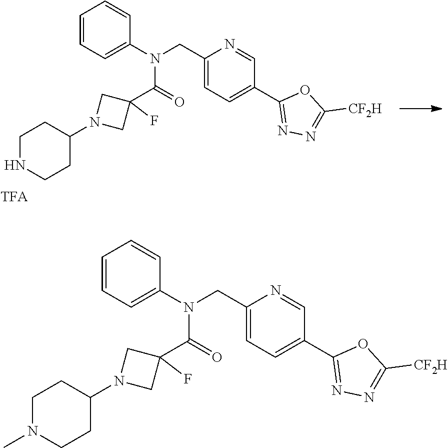 [SMILES: O=C(N(Cc1ccc(-c2nnc(C(F)F)o2)cn1)c1ccccc1)C1(F)CN(C2CCNCC2)C1 ; CN1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate (0.040 g, 0.082 mmol) prepared in step 2, formaldehyde (0.005 g, 0.164 mmol), acetic acid (0.005 mL, 0.082 mmol), and sodium triacetoxyborohydride (0.052 g, 0.247 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 48.6%) as a yellow gel.

¹H NMR (400 MHz, CDCl₃) δ 9.26 (d, J=2.1 Hz, 1H), 8.39 (dd, J=8.2, 2.2 Hz, 1H), 7.58 (d, J=8.3 Hz, 1H), 7.37 (d, J=7.2 Hz, 3H), 7.26 (d, J=8.0 Hz, 2H), 6.95 (t, J=51.7 Hz, 1H), 5.13 (s, 2H), 3.55 (dd, J=23.1, 9.9 Hz, 2H), 3.12 (dd, J=21.5, 10.2 Hz, 2H), 2.83 (s, 2H), 2.35 (s, 3H), 2.11 (s, 3H), 1.71 (s, 2H), 1.41 (s, 2H);

LRMS (ES) m/z 501.4 (M++1).

### Example 55: Synthesis of Compound 3111, N-((5-(5-(difluoromethyl)-1,3,4-oxadiazol-2-yl)pyridin-2-yl)methyl)-1-(1-ethylpiperidin-4-yl)-3-fluoro-N-phenylazetidine-3-carboxamide

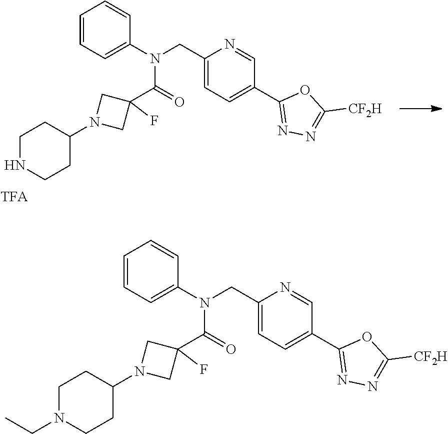 [SMILES: O=C(N(Cc1ccc(-c2nnc(C(F)F)o2)cn1)c1ccccc1)C1(F)CN(C2CCNCC2)C1 ; CCN1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate (0.040 g, 0.082 mmol) prepared in step 2 of Example 54, acetaldehyde (0.007 g, 0.164 mmol), acetic acid (0.007 mL, 0.082 mmol), and sodium triacetoxyborohydride (0.052 g, 0.247 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.017 g, 40.2%) as a yellow gel.

¹H NMR (400 MHz, CDCl₃) δ 9.27 (d, J=2.0 Hz, 1H), 8.39 (dd, J=8.3, 2.2 Hz, 1H), 7.57 (d, J=7.8 Hz, 1H), 7.38 (d, J=7.4 Hz, 3H), 7.25 (s, 2H), 6.96 (t, J=51.6 Hz, 1H), 5.13 (s, 2H), 3.55 (dd, J=23.4, 9.9 Hz, 2H), 3.12 (dd, J=21.4, 9.5 Hz, 2H), 2.93 (s, 2H), 1.72 (s, 7H), 1.47 (s, 2H), 1.20 (s, 3H);

LRMS (ES) m/z 515.5 (M++1).

### Example 56: Synthesis of Compound 3112, N-((5-(5-(difluoromethyl)-1,3,4-oxadiazol-2-yl)pyridin-2-yl)methyl)-3-fluoro-N-phenyl-1-(1-propylpiperidin-4-yl)azetidine-3-carboxamide

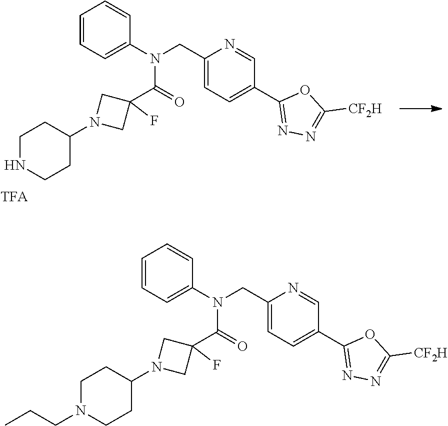 [SMILES: O=C(N(Cc1ccc(-c2nnc(C(F)F)o2)cn1)c1ccccc1)C1(F)CN(C2CCNCC2)C1 ; CCCN1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate (0.040 g, 0.082 mmol) prepared in step 2 of Example 54, propioaldehyde (0.010 g, 0.164 mmol), acetic acid (0.005 mL, 0.082 mmol), and sodium triacetoxyborohydride (0.052 g, 0.247 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.011 g, 25.3%) as a yellow gel.

¹H NMR (400 MHz, CDCl₃) δ 9.26 (d, J=1.6 Hz, 1H), 8.39 (dd, J=8.2, 2.2 Hz, 1H), 7.58 (d, J=8.2 Hz, 1H), 7.37 (d, J=7.2 Hz, 3H), 7.26 (d, J=7.9 Hz, 2H), 6.96 (t, J=51.6 Hz, 1H), 5.13 (s, 2H), 3.55 (dd, J=23.2, 9.9 Hz, 2H), 3.12 (dd, J=21.4, 10.2 Hz, 2H), 2.90 (s, 2H), 1.68 (s, 9H), 1.42 (s, 2H), 0.92 (t, J=7.4 Hz, 3H);

LRMS (ES) m/z 529.6 (M++1).

### Example 57: Synthesis of Compound 3113, 1-(1-butylpiperidin-4-yl)-N-((5-(5-(difluoromethyl)-1,3,4-oxadiazol-2-yl)pyridin-2-yl)methyl)-3-fluoro-N-phenylazetidine-3-carboxamide

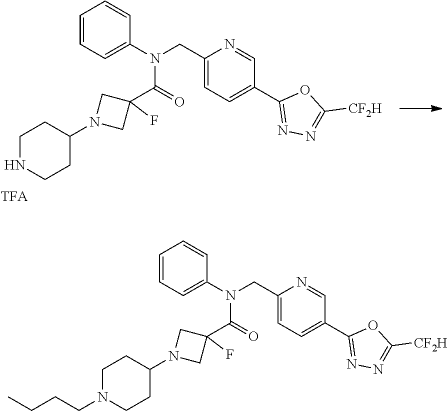 [SMILES: O=C(N(Cc1ccc(-c2nnc(C(F)F)o2)cn1)c1ccccc1)C1(F)CN(C2CCNCC2)C1 ; CCCCN1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate (0.040 g, 0.082 mmol) prepared in step 2 of Example 54, butyraldehyde (0.012 g, 0.164 mmol), acetic acid (0.005 mL, 0.082 mmol), and sodium triacetoxyborohydride (0.052 g, 0.247 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.022 g, 49.3%) as a yellow gel.

¹H NMR (400 MHz, CDCl₃) δ 9.27 (d, J=1.8 Hz, 1H), 8.39 (dd, J=8.1, 2.2 Hz, 1H), 7.57 (d, J=8.2 Hz, 1H), 7.37 (d, J=7.1 Hz, 3H), 7.26 (d, J=8.1 Hz, 2H), 6.97 (dd, J=64.6, 38.7 Hz, 1H), 5.13 (s, 2H), 3.54 (d, J=22.8 Hz, 2H), 3.12 (d, J=12.0 Hz, 2H), 2.89 (s, 2H), 1.66 (s, 10H), 1.41-1.29 (m, 3H), 0.93 (t, J=7.3 Hz, 3H);

LRMS (ES) m/z 543.6 (M++1).

### Example 58: Synthesis of Compound 3114, N-((5-(5-(difluoromethyl)-1,3,4-oxadiazol-2-yl)pyridin-2-yl)methyl)-3-fluoro-1-(1-iso butylpiperidin-4-yl)-N-phenylazetidine-3-carboxamide

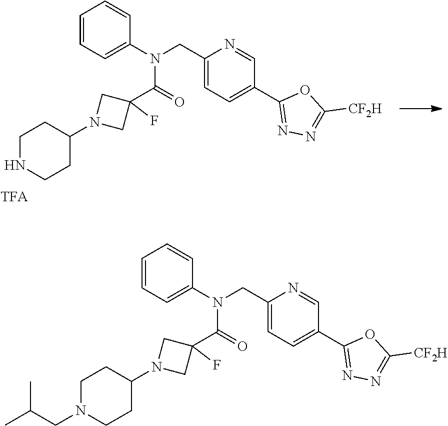 [SMILES: O=C(N(Cc1ccc(-c2nnc(C(F)F)o2)cn1)c1ccccc1)C1(F)CN(C2CCNCC2)C1 ; CC(C)CN1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate (0.040 g, 0.082 mmol) prepared in step 2 of Example 54, isobutyraldehyde (0.012 g, 0.164 mmol), acetic acid (0.005 mL, 0.082 mmol), and sodium triacetoxyborohydride (0.052 g, 0.247 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.017 g, 38.1%) as a yellow gel.

¹H NMR (400 MHz, CDCl₃) δ 9.26 (d, J=1.6 Hz, 1H), 8.39 (dd, J=8.2, 2.2 Hz, 1H), 7.59 (d, J=8.3 Hz, 1H), 7.40-7.34 (m, 3H), 7.26 (d, J=7.9 Hz, 2H), 6.95 (t, J=51.6 Hz, 1H), 5.13 (s, 2H), 3.54 (dd, J=23.1, 9.9 Hz, 2H), 3.12 (dd, J=21.2, 10.2 Hz, 2H), 2.76 (s, 2H), 2.06 (s, 2H), 1.91 (s, 2H), 1.77 (s, 2H), 1.59 (s, 2H), 1.28 (s, 2H), 0.89 (d, J=6.1 Hz, 6H);

LRMS (ES) m/z 544.5 (M++1).

### Example 59: Synthesis of Compound 3115, N-((5-(5-(difluoromethyl)-1,3,4-oxadiazol-2-yl)pyridin-2-yl)methyl)-3-fluoro-1-(1-iso propylpiperidin-4-yl)-N-phenylazetidine-3-carboxamide

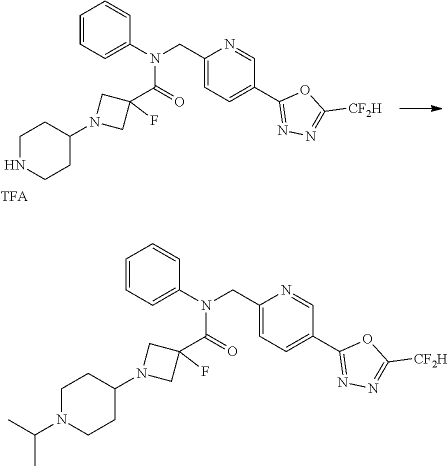 [SMILES: O=C(N(Cc1ccc(-c2nnc(C(F)F)o2)cn1)c1ccccc1)C1(F)CN(C2CCNCC2)C1 ; CC(C)N1CCC(N2CC(F)(C(=O)N(Cc3ccc(-c4nnc(C(F)F)o4)cn3)c3ccccc3)C2)CC1]

N-((5-(5-(difluoromethyl)-1,3,4-oxadiazol-2-yl)pyridin-2-yl)methyl)-3-fluoro-N-phenyl-1-(piperidin-4-yl)azetidine-3-carboxamide 2,2,2-trifluoroacetate (0.040 g, 0.082 mmol) prepared in step 2 of Example 54, propan-2-one (0.010 g, 0.164 mmol), acetic acid (0.005 mL, 0.082 mmol), and sodium triacetoxyborohydride (0.052 g, 0.247 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.016 g, 36.8%) as a yellow gel.

¹H NMR (400 MHz, CDCl₃) δ 9.28 (d, J=2.2 Hz, 1H), 8.39 (dd, J=8.2, 2.2 Hz, 1H), 7.55 (d, J=7.9 Hz, 1H), 7.39 (d, J=7.1 Hz, 3H), 7.26 (s, 2H), 6.96 (t, J=51.7 Hz, 1H), 5.12 (s, 2H), 3.55 (dd, J=23.0, 9.5 Hz, 2H), 3.41 (s, 1H), 3.12 (dd, J=21.0, 10.1 Hz, 3H), 2.12 (s, 2H), 1.85 (s, 4H), 1.64 (s, 2H), 1.30 (d, J=6.7 Hz, 6H);

LRMS (ES) m/z 529.3 (M++1).

### Example 60: Synthesis of Compound 3152, 1-acetyl-N-((5-(5-(difluoromethyl)-1,3,4-oxadiazol-2-yl)pyridin-2-yl)methyl)-3-fluoro-N-phenylazetidine-3-carboxamide

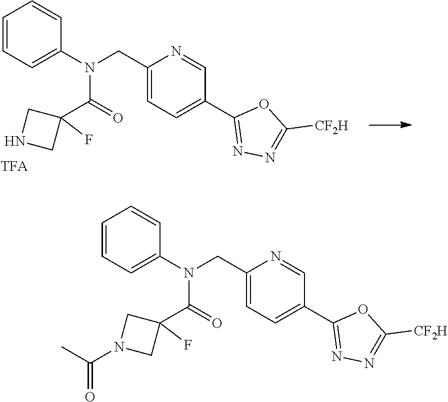 [SMILES: O=C(N(Cc1ccc(-c2nnc(C(F)F)o2)cn1)c1ccccc1)C1(F)CNC1 ; CC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, acetyl chloride (0.014 m, 0.193 mmol), and triethylamine (0.062 mL, 0.446 mmol) were dissolved in dichloromethane (5 mL) at room temperature, and the resulting solution was stirred at the same temperature for 18 hours. Water was poured into the reaction mixture, followed by extraction with dichloromethane. The organic layer was washed with a saturated aqueous sodium chloride solution, dried over anhydrous sodium sulfate, filtered, and concentrated under reduced pressure. The concentrate was purified by column chromatography (SiO₂, 12 g cartridge; ethyl acetate/hexane=0% to 30%) and concentrated to obtain the title compound (0.015 g, 22.6%) as a yellow gel.

¹H NMR (400 MHz, CDCl₃) δ 9.20 (d, J=1.8 Hz, 1H), 8.31 (dd, J=8.2, 2.2 Hz, 1H), 7.45 (d, J=8.2 Hz, 1H), 7.37-7.23 (m, 3H), 7.20 (d, J=4.3 Hz, 2H), 6.88 (dd, J=64.1, 39.2 Hz, 1H), 5.04 (s, 2H), 4.67 (dd, J=21.4, 10.4 Hz, 1H), 4.25 (dd, J=22.5, 11.7 Hz, 1H), 3.94 (dd, J=22.7, 9.6 Hz, 1H), 3.58 (dd, J=23.1, 11.8 Hz, 1H), 1.79 (s, 3H);

LRMS (ES) m/z 446.5 (M++1).

### Example 61: Synthesis of Compound 3153, N-((5-(5-(difluoromethyl)-1,3,4-oxadiazol-2-yl)pyridin-2-yl)methyl)-3-fluoro-N-phenyl-1-propionylazetidine-3-carboxamide

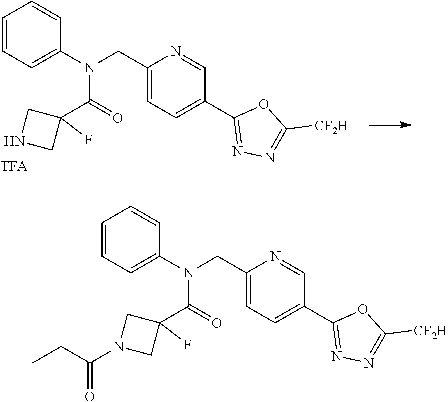 [SMILES: O=C(N(Cc1ccc(-c2nnc(C(F)F)o2)cn1)c1ccccc1)C1(F)CNC1 ; CCC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, propionyl chloride (0.018 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.024 g, 35.1%) as a yellow gel.

¹H NMR (400 MHz, CDCl₃) δ 9.29 (d, J=1.7 Hz, 1H), 8.40 (dd, J=8.2, 2.2 Hz, 1H), 7.54 (d, J=8.2 Hz, 1H), 7.48-7.34 (m, 3H), 7.28 (d, J=4.5 Hz, 2H), 6.97 (dd, J=64.2, 39.1 Hz, 1H), 5.13 (s, 2H), 4.74 (dd, J=21.6, 10.4 Hz, 1H), 4.35 (dd, J=22.6, 11.8 Hz, 1H), 4.02 (dd, J=22.8, 9.9 Hz, 1H), 3.68 (dd, J=23.1, 11.7 Hz, 1H), 2.11 (q, J=7.5 Hz, 2H), 1.12 (t, J=7.5 Hz, 3H);

LRMS (ES) m/z 460.2 (M++1).

### Example 62: Synthesis of Compound 3154, 1-butyryl-N-((5-(5-(difluoromethyl)-1,3,4-oxadiazol-2-yl)pyridin-2-yl)methyl)-3-fluoro-N-phenylazetidine-3-carboxamide

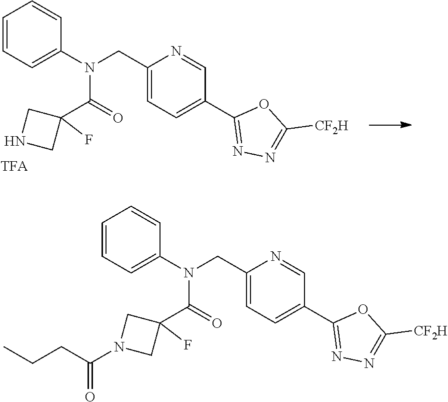 [SMILES: O=C(N(Cc1ccc(-c2nnc(C(F)F)o2)cn1)c1ccccc1)C1(F)CNC1 ; CCCC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, butyryl chloride (0.021 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.021 g, 29.8%) as a yellow gel.

¹H NMR (400 MHz, CDCl₃) δ 9.20 (d, J=1.8 Hz, 1H), 8.31 (dd, J=8.2, 2.2 Hz, 1H), 7.45 (d, J=8.2 Hz, 1H), 7.36-7.26 (m, 3H), 7.19 (d, J=4.5 Hz, 2H), 6.88 (dd, J=64.1, 39.2 Hz, 1H), 5.04 (s, 2H), 4.66 (dd, J=21.7, 10.4 Hz, 1H), 4.25 (dd, J=22.4, 12.2 Hz, 1H), 3.93 (dd, J=22.6, 10.0 Hz, 1H), 3.58 (dd, J=23.5, 12.0 Hz, 1H), 1.97 (t, J=7.5 Hz, 2H), 1.54 (dq, J=14.8, 7.5 Hz, 2H), 0.85 (t, J=7.4 Hz, 3H);

LRMS (ES) m/z 475.5 (M++1).

### Example 63: Synthesis of Compound 3155, N-((5-(5-(difluoromethyl)-1,3,4-oxadiazol-2-yl)pyridin-2-yl)methyl)-3-fluoro-1-(3-methylbutanoyl)-N-phenylazetidine-3-carboxamide

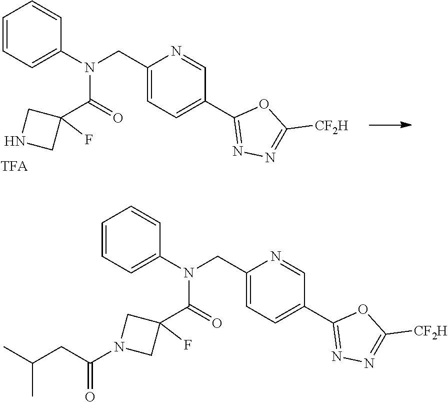 [SMILES: O=C(N(Cc1ccc(-c2nnc(C(F)F)o2)cn1)c1ccccc1)C1(F)CNC1 ; CC(C)CC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, 3-methylbutanoyl chloride (0.023 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.019 g, 26.2%) as a yellow gel.

¹H NMR (400 MHz, CDCl₃) δ 9.29 (d, J=1.8 Hz, 1H), 8.40 (dd, J=8.2, 2.2 Hz, 1H), 7.54 (d, J=8.2 Hz, 1H), 7.45-7.35 (m, 3H), 7.28 (d, J=5.0 Hz, 2H), 6.97 (dd, J=64.2, 39.1 Hz, 1H), 5.13 (s, 2H), 4.76 (dd, J=21.6, 10.4 Hz, 1H), 4.34 (dd, J=22.8, 12.3 Hz, 1H), 4.03 (dd, J=22.8, 10.1 Hz, 1H), 3.67 (dd, J=23.2, 12.4 Hz, 1H), 2.11 (td, J=13.5, 6.7 Hz, 1H), 1.96 (d, J=7.1 Hz, 2H), 0.95 (t, J=6.5 Hz, 6H);

LRMS (ES) m/z 488.3 (M++1).

### Example 64: Synthesis of Compound 3156, 1-(cyclohexylmethyl)-N-((5-(5-(difluoromethyl)-1,3,4-oxadiazol-2-yl)pyridin-2-yl)methyl)-3-fluoro-N-phenylazetidine-3-carboxamide

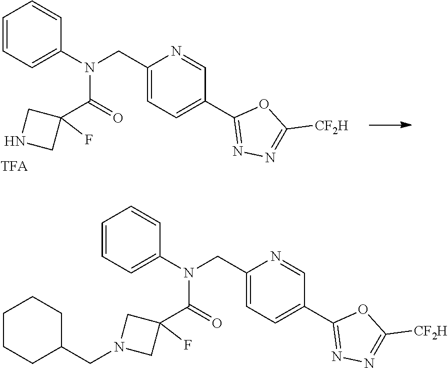 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(CC2CCCCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, cyclohexanecarbaldehyde (0.033 g, 0.297 mmol), acetic acid (0.009 mL, 0.149 mmol), and sodium triacetoxyborohydride (0.095 g, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.024 g, 32.3%) as a yellow gel.

¹H NMR (400 MHz, CDCl₃) δ 9.16 (d, J=1.7 Hz, 1H), 8.29 (dd, J=8.2, 2.2 Hz, 1H), 7.50 (d, J=8.2 Hz, 1H), 7.31-7.23 (m, 3H), 7.16 (dd, J=8.0, 1.4 Hz, 2H), 6.88 (dd, J=65.1, 38.3 Hz, 1H), 5.03 (s, 2H), 3.45 (dd, J=22.6, 8.4 Hz, 2H), 3.06 (dd, J=20.8, 9.5 Hz, 2H), 2.19 (d, J=6.6 Hz, 2H), 1.77-1.64 (m, 2H), 1.60 (s, 2H), 1.27-0.95 (m, 5H), 0.76 (dd, J=21.4, 11.4 Hz, 2H);

LRMS (ES) m/z 501.4 (M++1).

### Example 65: Synthesis of Compound 3157, N-((5-(5-(difluoromethyl)-1,3,4-oxadiazol-2-yl)pyridin-2-yl)methyl)-3-fluoro-1-(3-methoxypropanoyl)-N-phenylazetidine-3-carboxamide

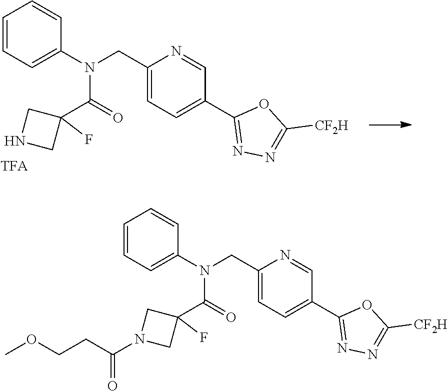 [SMILES: O=C(N(Cc1ccc(-c2nnc(C(F)F)o2)cn1)c1ccccc1)C1(F)CNC1 ; COCCC(=O)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, 3-methoxypropanoyl chloride (0.024 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.031 g, 42.6%) as a yellow gel.

¹H NMR (400 MHz, CDCl₃) δ 9.19 (d, J=1.8 Hz, 1H), 8.31 (dd, J=8.2, 2.2 Hz, 1H), 7.45 (d, J=8.2 Hz, 1H), 7.40-7.26 (m, 3H), 7.19 (d, J=4.9 Hz, 2H), 6.88 (dd, J=64.4, 38.9 Hz, 1H), 5.04 (s, 2H), 4.67 (dd, J=21.5, 10.2 Hz, 1H), 4.27 (dd, J=22.5, 12.3 Hz, 1H), 3.96 (dd, J=22.7, 10.1 Hz, 1H), 3.71-3.58 (m, 1H), 3.58-3.50 (m, 2H), 3.28-3.23 (m, 3H), 2.24 (t, J=6.2 Hz, 2H);

LRMS (ES) m/z 490.2 (M++1).

### Example 66: Synthesis of Compound 3158, 1-(cyclopropanecarbonyl)-N-((5-(5-(difluoromethyl)-1,3,4-oxadiazol-2-yl)pyridin-2-yl)methyl)-3-fluoro-N-phenylazetidine-3-carboxamide

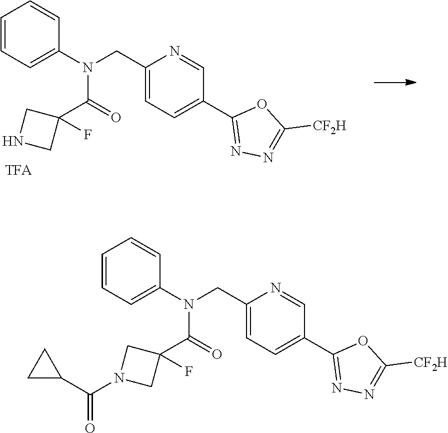 [SMILES: O=C(N(Cc1ccc(-c2nnc(C(F)F)o2)cn1)c1ccccc1)C1(F)CNC1 ; O=C(C1CC1)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, cyclopropanecarbonyl chloride (0.020 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.019 g, 27.1%) as a yellow gel.

¹H NMR (400 MHz, CDCl₃) δ 9.29 (d, J=1.8 Hz, 1H), 8.40 (dd, J=8.2, 2.2 Hz, 1H), 7.55 (d, J=8.1 Hz, 1H), 7.46-7.33 (m, 3H), 7.33-7.20 (m, 2H), 6.97 (dd, J=64.1, 39.2 Hz, 1H), 5.14 (s, 2H), 4.88 (dd, J=21.1, 10.1 Hz, 1H), 4.35 (dd, J=22.9, 11.2 Hz, 1H), 4.15 (dd, J=22.1, 9.9 Hz, 1H), 3.68 (dd, J=23.5, 11.9 Hz, 1H), 3.46 (d, J=27.5 Hz, 1H), 1.71 (ddd, J=12.7, 8.0, 4.7 Hz, 1H), 1.36 (ddd, J=12.5, 8.1, 4.6 Hz, 1H), 1.16 (d, J=12.6 Hz, 1H), 0.83-0.77 (m, 1H);

LRMS (ES) m/z 472.2 (M++1).

### Example 67: Synthesis of Compound 3159, 1-(cyclobutanecarbonyl)-N-((5-(5-(difluoromethyl)-1,3,4-oxadiazol-2-yl)pyridin-2-yl)methyl)-3-fluoro-N-phenylazetidine-3-carboxamide

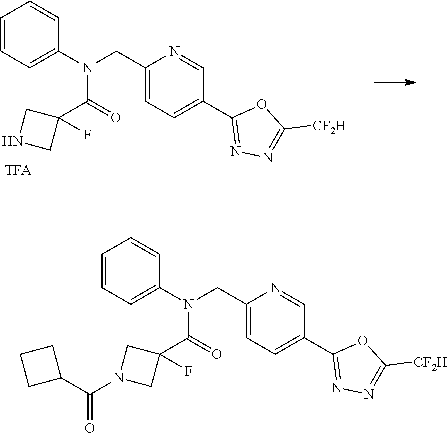 [SMILES: O=C(N(Cc1ccc(-c2nnc(C(F)F)o2)cn1)c1ccccc1)C1(F)CNC1 ; O=C(C1CCC1)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, cyclobutanecarbonyl chloride (0.023 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 27.7%) as a yellow gel.

¹H NMR (400 MHz, CDCl₃) δ 9.19 (d, J=1.8 Hz, 1H), 8.30 (dd, J=8.2, 2.2 Hz, 1H), 7.44 (d, J=8.2 Hz, 1H), 7.37-7.25 (m, 3H), 7.18 (d, J=8.3 Hz, 2H), 6.88 (dd, J=64.3, 39.0 Hz, 1H), 5.03 (s, 2H), 4.58 (dd, J=21.8, 10.3 Hz, 1H), 4.24 (dd, J=22.7, 11.7 Hz, 1H), 3.86 (dd, J=22.7, 10.5 Hz, 1H), 3.57 (dd, J=23.3, 11.6 Hz, 1H), 2.92 (p, J=8.5 Hz, 1H), 2.31-2.12 (m, 2H), 2.06-1.96 (m, 2H), 1.96-1.74 (m, 2H);

LRMS (ES) m/z 486.3 (M++1).

### Example 68: Synthesis of Compound 3160, 1-(cyclopentanecarbonyl)-N-((5-(5-(difluoromethyl)-1,3,4-oxadiazol-2-yl)pyridin-2-yl)methyl)-3-fluoro-N-phenylazetidine-3-carboxamide

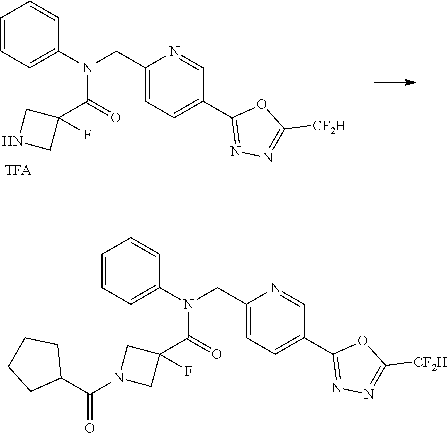 [SMILES: O=C(N(Cc1ccc(-c2nnc(C(F)F)o2)cn1)c1ccccc1)C1(F)CNC1 ; O=C(C1CCCC1)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, cyclopentanecarbonyl chloride (0.026 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.011 g, 14.8%) as a yellow gel.

¹H NMR (400 MHz, CDCl₃) δ 9.29 (d, J=1.8 Hz, 1H), 8.40 (dd, J=8.2, 2.2 Hz, 1H), 7.55 (d, J=8.2 Hz, 1H), 7.40 (dd, J=8.5, 3.1 Hz, 3H), 7.28 (s, 2H), 6.97 (dd, J=64.1, 39.2 Hz, 1H), 5.14 (s, 2H), 4.78 (dd, J=21.7, 10.1 Hz, 1H), 4.33 (dd, J=22.7, 11.9 Hz, 1H), 4.05 (dd, J=22.9, 10.1 Hz, 1H), 3.67 (dd, J=23.3, 11.4 Hz, 1H), 2.53 (d, J=7.8 Hz, 1H), 1.74 (s, 8H);

LRMS (ES) m/z 500.2 (M++1).

### Example 69: Synthesis of Compound 3161, 1-(cyclohexanecarbonyl)-N-((5-(5-(difluoromethyl)-1,3,4-oxadiazol-2-yl)pyridin-2-yl)methyl)-3-fluoro-N-phenylazetidine-3-carboxamide

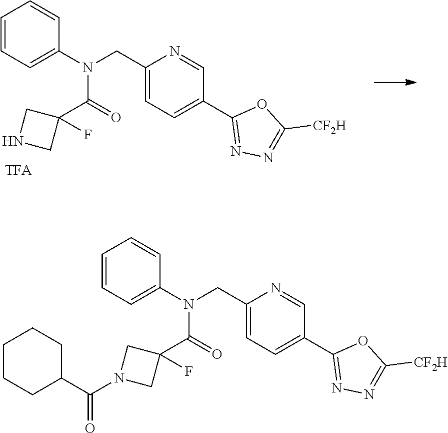 [SMILES: O=C(N(Cc1ccc(-c2nnc(C(F)F)o2)cn1)c1ccccc1)C1(F)CNC1 ; O=C(C1CCCCC1)N1CC(F)(C(=O)N(Cc2ccc(-c3nnc(C(F)F)o3)cn2)c2ccccc2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, cyclohexanecarbonyl chloride (0.028 g, 0.193 mmol), and triethylamine (0.062 mL, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.032 g, 41.9%) as a yellow gel.

¹H NMR (400 MHz, CDCl₃) δ 9.28 (d, J=1.8 Hz, 1H), 8.40 (dd, J=8.2, 2.2 Hz, 1H), 7.54 (d, J=8.2 Hz, 1H), 7.46-7.36 (m, 3H), 7.28 (d, J=6.9 Hz, 2H), 6.97 (dd, J=64.2, 39.1 Hz, 1H), 5.13 (s, 2H), 4.80 (dd, J=21.2, 10.3 Hz, 1H), 4.32 (dd, J=22.4, 11.9 Hz, 1H), 4.07 (dd, J=22.6, 10.3 Hz, 1H), 3.65 (dd, J=23.2, 12.2 Hz, 1H), 2.10 (ddd, J=11.6, 7.5, 3.2 Hz, 1H), 1.86-1.68 (m, 4H), 1.44 (dt, J=33.2, 16.7 Hz, 2H), 1.26 (d, J=15.1 Hz, 4H);

LRMS (ES) m/z 514.3 (M++1).

### Example 70: Synthesis of Compound 3162, N-((5-(5-(difluoromethyl)-1,3,4-oxadiazol-2-yl)pyridin-2-yl)methyl)-3-fluoro-N-phenyl-1-(tetrahydro-2H-thiopyran-4-yl)azetidine-3-carboxamide

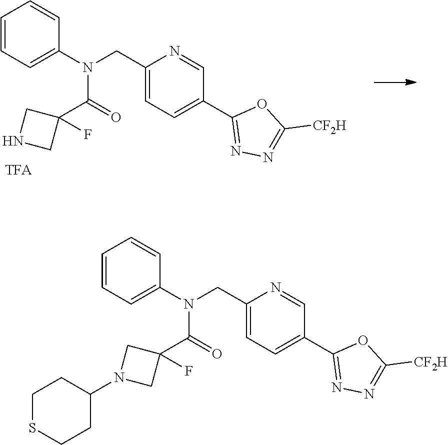 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(C2CCSCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, tetrahydro-4H-thiopyran-4-one (0.035 g, 0.297 mmol), acetic acid (0.009 mL, 0.149 mmol), and sodium triacetoxyborohydride (0.095 g, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.019 g, 25.4%) as a yellow gel.

¹H NMR (400 MHz, CDCl₃) δ 9.17 (d, J=1.8 Hz, 1H), 8.30 (dd, J=8.2, 2.2 Hz, 1H), 7.49 (d, J=8.2 Hz, 1H), 7.29 (dt, J=10.5, 3.6 Hz, 3H), 7.22-7.10 (m, 2H), 6.86 (t, J=51.7 Hz, 1H), 5.03 (s, 2H), 3.47 (dd, J=22.9, 8.7 Hz, 2H), 3.04 (dd, J=21.3, 9.1 Hz, 2H), 2.59 (t, J=11.8 Hz, 2H), 2.49-2.37 (m, 2H), 1.98 (s, 1H), 1.81 (d, J=13.0 Hz, 2H), 1.48-1.30 (m, 2H);

LRMS (ES) m/z 505.3 (M++1).

### Example 71: Synthesis of Compound 3163, N-((5-(5-(difluoromethyl)-1,3,4-oxadiazol-2-yl)pyridin-2-yl)methyl)-3-fluoro-N-(3-fluorophenyl)-1-propionylazetidine-3-carboxamide

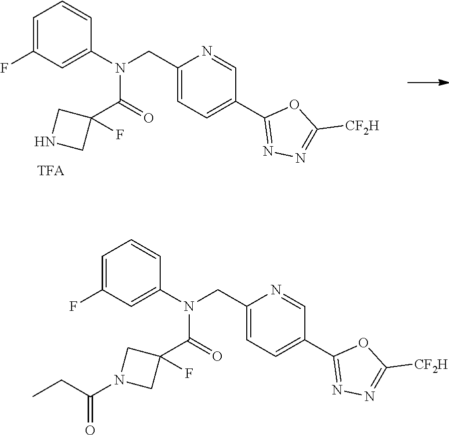 [SMILES: O=C(N(Cc1ccc(-c2nnc(C(F)F)o2)cn1)c1cccc(F)c1)C1(F)CNC1 ; CCC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, propionyl chloride (0.014 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.023 g, 40.6%) as a yellow gel.

¹H NMR (400 MHz, CDCl₃) δ 9.21 (d, J=1.8 Hz, 1H), 8.32 (dd, J=8.2, 2.2 Hz, 1H), 7.44 (d, J=8.2 Hz, 1H), 7.29 (dd, J=14.8, 8.2 Hz, 1H), 6.93 (dt, J=75.5, 30.1 Hz, 4H), 5.01 (s, 2H), 4.67 (dd, J=21.3, 9.9 Hz, 1H), 4.30 (dd, J=21.9, 11.3 Hz, 1H), 3.97 (dd, J=22.3, 9.6 Hz, 1H), 3.68 (dd, J=23.6, 12.3 Hz, 1H), 2.03 (q, J=7.5 Hz, 2H), 1.03 (t, J=7.5 Hz, 3H);

LRMS (ES) m/z 478.6 (M++1).

### Example 72: Synthesis of Compound 3164, 1-acetyl-N-((5-(5-(difluoromethyl)-1,3,4-oxadiazol-2-yl)pyridin-2-yl)methyl)-3-fluoro-N-(3-fluorophenyl)azetidine-3-carboxamide

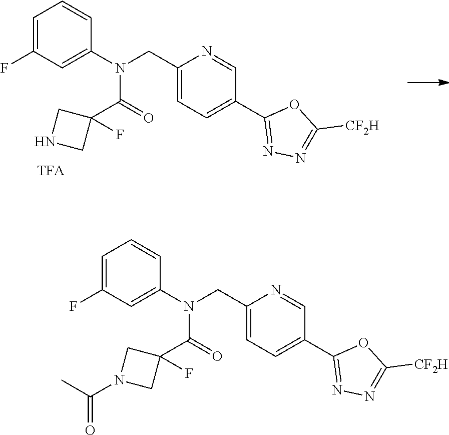 [SMILES: O=C(N(Cc1ccc(-c2nnc(C(F)F)o2)cn1)c1cccc(F)c1)C1(F)CNC1 ; CC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, acetyl chloride (0.011 m, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.022 g, 40.0%) as a yellow gel.

¹H NMR (400 MHz, CDCl₃) δ 9.31 (d, J=1.8 Hz, 1H), 8.41 (dd, J=8.2, 2.2 Hz, 1H), 7.53 (d, J=8.3 Hz, 1H), 7.38 (dd, J=14.6, 8.3 Hz, 1H), 7.19-6.74 (m, 4H), 5.10 (s, 2H), 4.78 (dd, J=21.3, 9.6 Hz, 1H), 4.39 (dd, J=22.1, 11.3 Hz, 1H), 4.08 (dd, J=22.0, 10.1 Hz, 1H), 3.76 (dd, J=22.7, 12.1 Hz, 1H), 1.90 (s, 3H);

LRMS (ES) m/z 464.2 (M++1).

### Example 73: Synthesis of Compound 3165, 1-butyryl-N-((5-(5-(difluoromethyl)-1,3,4-oxadiazol-2-yl)pyridin-2-yl)methyl)-3-fluoro-N-(3-fluorophenyl)azetidine-3-carboxamide

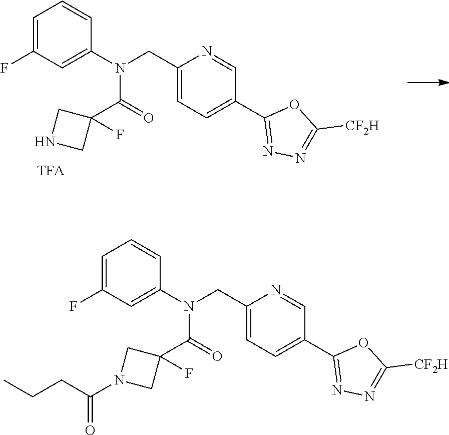 [SMILES: O=C(N(Cc1ccc(-c2nnc(C(F)F)o2)cn1)c1cccc(F)c1)C1(F)CNC1 ; CCCC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, butyryl chloride (0.016 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.021 g, 36.0%) as a yellow gel.

¹H NMR (400 MHz, CDCl₃) δ 9.21 (d, J=1.8 Hz, 1H), 8.32 (dd, J=8.2, 2.2 Hz, 1H), 7.44 (d, J=8.2 Hz, 1H), 7.29 (dd, J=14.7, 8.1 Hz, 1H), 6.93 (dt, J=75.8, 30.1 Hz, 4H), 5.01 (s, 2H), 4.68 (dd, J=21.0, 10.0 Hz, 1H), 4.29 (dd, J=22.1, 11.7 Hz, 1H), 3.98 (dd, J=22.3, 10.0 Hz, 1H), 3.67 (dd, J=22.8, 11.7 Hz, 1H), 1.98 (t, J=7.4 Hz, 2H), 1.55 (dq, J=14.7, 7.4 Hz, 2H), 0.86 (t, J=7.4 Hz, 3H);

LRMS (ES) m/z 493.5 (M++1).

### Example 74: Synthesis of Compound 3166, N-((5-(5-(difluoromethyl)-1,3,4-oxadiazol-2-yl)pyridin-2-yl)methyl)-3-fluoro-N-(3-fluorophenyl)-1-(3-methylbutanoyl)azetidine-3-carboxamide

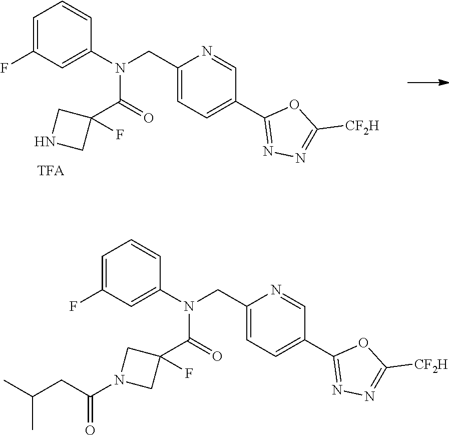 [SMILES: O=C(N(Cc1ccc(-c2nnc(C(F)F)o2)cn1)c1cccc(F)c1)C1(F)CNC1 ; CC(C)CC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, 3-methylbutanoyl chloride (0.019 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.025 g, 41.7%) as a yellow gel.

¹H NMR (400 MHz, CDCl₃) δ 9.21 (d, J=1.8 Hz, 1H), 8.32 (dd, J=8.2, 2.2 Hz, 1H), 7.44 (d, J=8.1 Hz, 1H), 7.29 (dd, J=14.9, 7.8 Hz, 1H), 6.93 (dt, J=75.7, 30.1 Hz, 4H), 5.01 (s, 2H), 4.68 (dd, J=21.2, 10.3 Hz, 1H), 4.29 (dd, J=22.0, 11.5 Hz, 1H), 3.98 (dd, J=22.3, 10.4 Hz, 1H), 3.67 (dd, J=23.0, 11.6 Hz, 1H), 2.10-1.97 (m, 1H), 1.88 (d, J=7.1 Hz, 2H), 0.89-0.84 (m, 6H);

LRMS (ES) m/z 506.2 (M++1).

### Example 75: Synthesis of Compound 3167, N-((5-(5-(difluoromethyl)-1,3,4-oxadiazol-2-yl)pyridin-2-yl)methyl)-3-fluoro-N-(3-fluorophenyl)-1-(3-methoxypropanoyl)azetidine-3-carboxamide

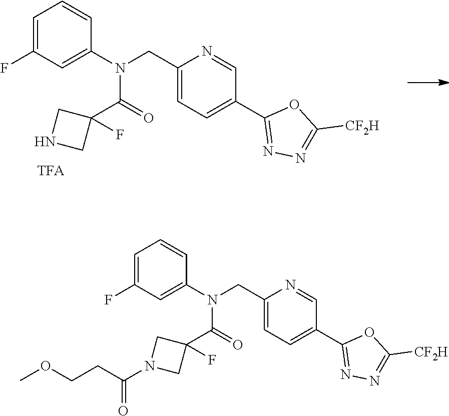 [SMILES: O=C(N(Cc1ccc(-c2nnc(C(F)F)o2)cn1)c1cccc(F)c1)C1(F)CNC1 ; COCCC(=O)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, 3-methoxypropanoyl chloride (0.019 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.025 g, 41.5%) as a yellow gel.

¹H NMR (400 MHz, CDCl₃) δ 9.21 (d, J=1.7 Hz, 1H), 8.32 (dd, J=8.2, 2.2 Hz, 1H), 7.44 (d, J=8.1 Hz, 1H), 7.28 (dd, J=14.6, 8.3 Hz, 1H), 7.09-6.68 (m, 4H), 5.01 (s, 2H), 4.69 (dd, J=21.9, 10.1 Hz, 1H), 4.31 (dd, J=22.3, 11.9 Hz, 1H), 4.01 (dd, J=22.6, 10.6 Hz, 1H), 3.81-3.64 (m, 1H), 3.57 (q, J=6.2 Hz, 2H), 3.25 (s, 3H), 2.26 (t, J=6.2 Hz, 2H);

LRMS (ES) m/z 508.2 (M++1).

### Example 76: Synthesis of Compound 3168, 1-(cyclobutanecarbonyl)-N-((5-(5-(difluoromethyl)-1,3,4-oxadiazol-2-yl)pyridin-2-yl)methyl)-3-fluoro-N-(3-fluorophenyl)azetidine-3-carboxamide

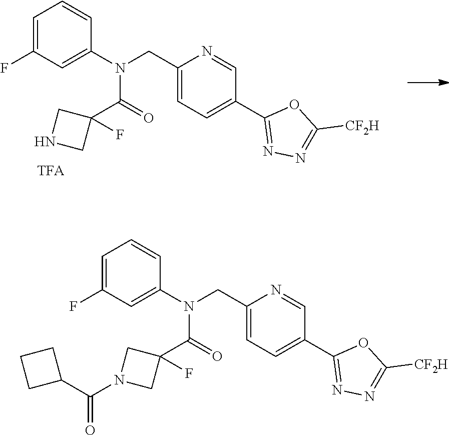 [SMILES: O=C(N(Cc1ccc(-c2nnc(C(F)F)o2)cn1)c1cccc(F)c1)C1(F)CNC1 ; O=C(C1CCC1)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclobutanecarbonyl chloride (0.018 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.025 g, 41.8%) as a yellow gel.

¹H NMR (400 MHz, CDCl₃) δ 9.21 (d, J=1.9 Hz, 1H), 8.31 (dd, J=8.2, 2.2 Hz, 1H), 7.43 (d, J=8.2 Hz, 1H), 7.28 (dd, J=14.5, 8.0 Hz, 1H), 6.95 (ddd, J=99.3, 44.9, 30.2 Hz, 4H), 5.00 (s, 2H), 4.60 (dd, J=21.5, 10.1 Hz, 1H), 4.28 (dd, J=22.2, 11.7 Hz, 1H), 3.90 (dd, J=22.4, 10.5 Hz, 1H), 3.66 (dd, J=23.3, 11.9 Hz, 1H), 2.93 (p, J=8.6 Hz, 1H), 2.22 (tt, J=17.6, 8.7 Hz, 2H), 2.00 (dt, J=25.6, 12.9 Hz, 2H), 1.93-1.76 (m, 2H);

LRMS (ES) m/z 504.2 (M++1).

### Example 77: Synthesis of Compound 3169, 1-(cyclopentanecarbonyl)-N-((5-(5-(difluoromethyl)-1,3,4-oxadiazol-2-yl)pyridin-2-yl)methyl)-3-fluoro-N-(3-fluorophenyl)azetidine-3-carboxamide

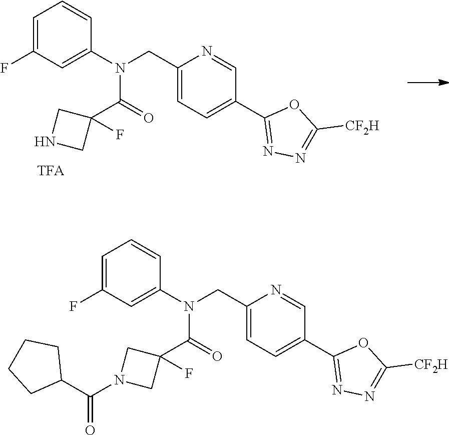 [SMILES: O=C(N(Cc1ccc(-c2nnc(C(F)F)o2)cn1)c1cccc(F)c1)C1(F)CNC1 ; O=C(C1CCCC1)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclopentanecarbonyl chloride (0.020 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.033 g, 53.7%) as a yellow gel.

¹H NMR (400 MHz, CDCl₃) δ 9.21 (d, J=1.8 Hz, 1H), 8.32 (dd, J=8.2, 2.2 Hz, 1H), 7.44 (d, J=8.1 Hz, 1H), 7.29 (dd, J=14.7, 8.0 Hz, 1H), 6.93 (dt, J=75.6, 30.1 Hz, 4H), 5.01 (s, 2H), 4.70 (dd, J=21.3, 10.3 Hz, 1H), 4.28 (dd, J=22.2, 11.7 Hz, 1H), 4.00 (dd, J=22.4, 9.8 Hz, 1H), 3.66 (dd, J=23.0, 11.9 Hz, 1H), 2.45 (d, J=7.5 Hz, 1H), 1.66 (s, 6H), 1.47 (d, J=5.1 Hz, 2H);

LRMS (ES) m/z 518.2 (M++1).

### Example 78: Synthesis of Compound 3170, 1-(cyclohexanecarbonyl)-N-((5-(5-(difluoromethyl)-1,3,4-oxadiazol-2-yl)pyridin-2-yl)methyl)-3-fluoro-N-(3-fluorophenyl)azetidine-3-carboxamide

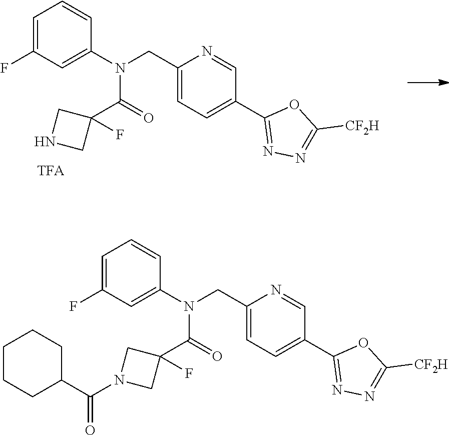 [SMILES: O=C(N(Cc1ccc(-c2nnc(C(F)F)o2)cn1)c1cccc(F)c1)C1(F)CNC1 ; O=C(C1CCCCC1)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclohexanecarbonyl chloride (0.023 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.031 g, 49.2%) as a yellow gel.

¹H NMR (400 MHz, CDCl₃) δ 9.21 (d, J=1.8 Hz, 1H), 8.32 (dd, J=8.2, 2.2 Hz, 1H), 7.44 (d, J=8.4 Hz, 1H), 7.29 (dd, J=14.7, 8.1 Hz, 1H), 7.08-6.69 (m, 4H), 5.01 (s, 2H), 4.72 (dd, J=21.2, 9.5 Hz, 1H), 4.27 (dd, J=21.4, 11.8 Hz, 1H), 4.02 (dd, J=22.4, 9.1 Hz, 1H), 3.65 (dd, J=22.4, 11.7 Hz, 1H), 2.03 (t, J=11.7 Hz, 1H), 1.70 (s, 3H), 1.44-1.31 (m, 3H), 1.23-1.09 (m, 4H);

LRMS (ES) m/z 532.3 (M++1).

### Example 79: Synthesis of Compound 3171, 1-(cyclohexylmethyl)-N-((5-(5-(difluoromethyl)-1,3,4-oxadiazol-2-yl)pyridin-2-yl)methyl)-3-fluoro-N-(3-fluorophenyl)azetidine-3-carboxamide

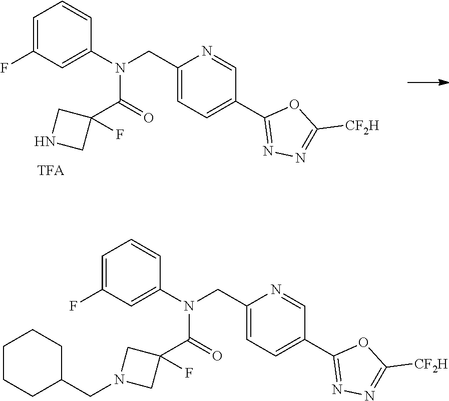 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(CC2CCCCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclohexanecarbaldehyde (0.027 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.028 g, 45.6%) as a yellow gel.

¹H NMR (400 MHz, CDCl₃) δ 9.18 (d, J=1.7 Hz, 1H), 8.30 (dd, J=8.2, 2.2 Hz, 1H), 7.48 (d, J=8.2 Hz, 1H), 7.25 (td, J=8.2, 6.4 Hz, 1H), 6.91 (dt, J=84.0, 29.1 Hz, 4H), 5.00 (s, 2H), 3.52 (dd, J=25.3, 13.7 Hz, 2H), 3.12 (d, J=12.5 Hz, 2H), 2.19 (t, J=13.2 Hz, 2H), 1.77-1.52 (m, 9H), 0.77 (dd, J=22.1, 11.5 Hz, 2H);

LRMS (ES) m/z 519.3 (M++1).

### Example 80: Synthesis of Compound 3172, N-((5-(5-(difluoromethyl)-1,3,4-oxadiazol-2-yl)pyridin-2-yl)methyl)-3-fluoro-N-(3-fluorophenyl)-1-(tetrahydro-2H-thiopyran-4-yl)azetidine-3-carboxamide

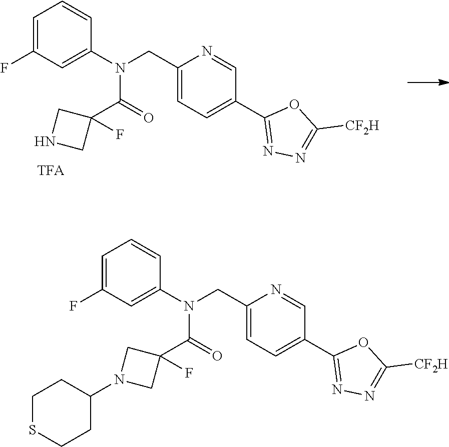 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(C2CCSCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, tetrahydro-4H-thiopyran-4-one (0.028 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.018 g, 29.1%) as a yellow gel.

¹H NMR (400 MHz, CDCl₃) δ 9.19 (d, J=1.7 Hz, 1H), 8.30 (dd, J=8.2, 2.2 Hz, 1H), 7.47 (d, J=8.1 Hz, 1H), 7.33-7.21 (m, 1H), 7.04-6.70 (m, 4H), 5.01 (s, 2H), 3.64-3.39 (m, 2H), 3.22-3.02 (m, 2H), 2.61 (d, J=13.9 Hz, 2H), 2.52-2.35 (m, 2H), 1.99 (d, J=10.9 Hz, 1H), 1.83 (d, J=13.0 Hz, 2H), 1.39 (dd, J=20.5, 10.2 Hz, 2H);

LRMS (ES) m/z 523.2 (M++1).

### Example 81: Synthesis of Compound 3216, N-((5-(5-(difluoromethyl)-1,3,4-oxadiazol-2-yl)pyridin-2-yl)methyl)-3-fluoro-N-phenyl-1-(tetrahydro-2H-pyran-4-yl)methyl)azetidine-3-carboxamide

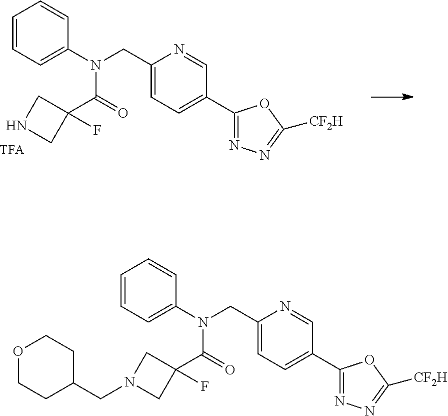 [SMILES: O=C(N(Cc1ccc(-c2nnc(C(F)F)o2)cn1)c1ccccc1)C1(F)CNC1 ; O=C(N(Cc1ccc(-c2nnc(C(F)F)o2)cn1)c1ccccc1)C1(F)CN(CC2CCOCC2)C1]

N-((5-(5-(difluoromethyl)-1,3,4-oxadiazol-2-yl)pyridin-2-yl)methyl)-3-fluoro-N-phenylazetidine-3-carboxamide 2,2,2-trifluoroacetate (0.060 g, 0.149 mmol) prepared in step 3 of Example 14, tetrahydro-2H-pyran-4-carbaldehyde (0.034 g, 0.297 mmol), acetic acid (0.009 mL, 0.149 mmol), and sodium triacetoxyborohydride (0.095 g, 0.446 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.022 g, 29.5%) as a yellow gel.

¹H NMR (400 MHz, CDCl₃) δ 9.26 (d, J=1.7 Hz, 1H), 8.39 (dd, J=8.2, 2.2 Hz, 1H), 7.57 (t, J=9.6 Hz, 1H), 7.37 (d, J=7.1 Hz, 3H), 7.25 (d, J=7.9 Hz, 2H), 6.97 (dd, J=64.8, 38.5 Hz, 1H), 5.12 (s, 2H), 3.99-3.89 (m, 2H), 3.65-3.44 (m, 2H), 3.34 (dd, J=11.6, 10.2 Hz, 2H), 3.17 (d, J=13.5 Hz, 2H), 2.34 (s, 2H), 1.58 (d, J=13.1 Hz, 2H), 1.32-1.16 (m, 3H);

LRMS (ES) m/z 503.3 (M++1).

### Example 82: Synthesis of Compound 3217, 1-(cyclopropanecarbonyl)-N-((5-(5-(difluoromethyl)-1,3,4-oxadiazol-2-yl)pyridin-2-yl)methyl)-3-fluoro-N-(3-fluorophenyl)azetidine-3-carboxamide

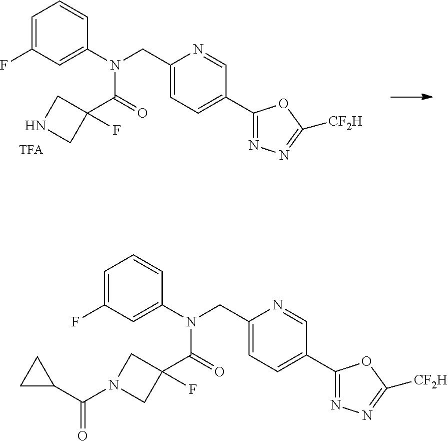 [SMILES: O=C(N(Cc1ccc(-c2nnc(C(F)F)o2)cn1)c1cccc(F)c1)C1(F)CNC1 ; O=C(C1CC1)N1CC(F)(C(=O)N(Cc2ccc(-c3nnc(C(F)F)o3)cn2)c2cccc(F)c2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, cyclopropanecarbonyl chloride (0.016 g, 0.154 mmol), and triethylamine (0.050 mL, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.014 g, 24.1%) as a yellow gel.

¹H NMR (400 MHz, CDCl₃) δ 9.31 (d, J=1.8 Hz, 1H), 8.41 (dd, J=8.2, 2.2 Hz, 1H), 7.54 (d, J=8.2 Hz, 1H), 7.38 (dd, J=14.6, 8.3 Hz, 1H), 7.15-6.82 (m, 4H), 5.11 (s, 2H), 4.91 (s, 1H), 4.40 (s, 1H), 4.20 (d, J=15.3 Hz, 1H), 3.76 (s, 1H), 3.47 (s, 1H), 1.02-0.96 (m, 2H), 0.83-0.75 (m, 2H);

LRMS (ES) m/z 490.5 (M++1).

### Example 83: Synthesis of Compound 3218, N-((5-(5-(difluoromethyl)-1,3,4-oxadiazol-2-yl)pyridin-2-yl)methyl)-3-fluoro-N-(3-fluorophenyl)-1-(tetrahydro-2H-pyran-4-yl)methyl)azetidine-3-carboxamide

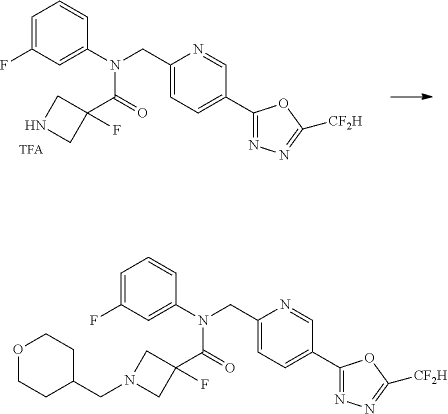 [SMILES: O=C(N(Cc1ccc(-c2nnc(C(F)F)o2)cn1)c1cccc(F)c1)C1(F)CNC1 ; O=C(N(Cc1ccc(-c2nnc(C(F)F)o2)cn1)c1cccc(F)c1)C1(F)CN(CC2CCOCC2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.050 g, 0.119 mmol) prepared in step 2 of Example 31, tetrahydro-2H-pyran-4-carbaldehyde (0.027 g, 0.237 mmol), acetic acid (0.007 mL, 0.119 mmol), and sodium triacetoxyborohydride (0.075 g, 0.356 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.022 g, 35.7%) as a yellow gel.

¹H NMR (400 MHz, CDCl₃) δ 9.28 (d, J=1.8 Hz, 1H), 8.40 (dd, J=8.2, 2.2 Hz, 1H), 7.56 (d, J=8.0 Hz, 1H), 7.40-7.31 (m, 1H), 7.02 (ddd, J=86.4, 42.5, 28.2 Hz, 4H), 5.10 (s, 2H), 3.99-3.88 (m, 2H), 3.65 (s, 2H), 3.56-3.46 (m, 1H), 3.42-3.30 (m, 2H), 3.21 (s, 2H), 2.37 (s, 2H), 1.37-1.16 (m, 4H);

LRMS (ES) m/z 521.4 (M++1).

### Example 84: Synthesis of Compound 3219, N-((5-(5-(difluoromethyl)-1,3,4-oxadiazol-2-yl)pyridin-2-yl)methyl)-3-fluoro-N-(3-fluorophenyl)-1′-methyl-[1,3′-biazetidine]-3-carboxamide

### [Step 1] Synthesis of tert-butyl 3-(((5-(5-(difluoromethyl)-1,3,4-oxadiazol-2-yl)pyridin-2-yl)methyl)(3-fluorophenyl)carbamoyl)-3-fluoro-[1,3′-biazetidine]-1′-carboxylate

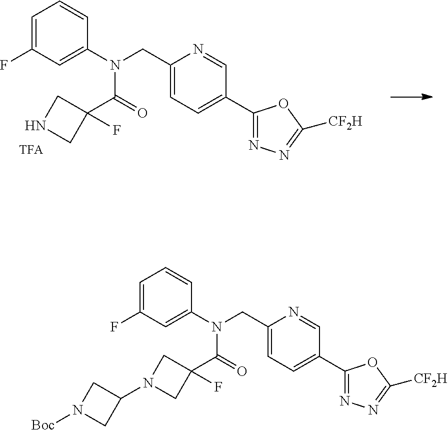 [SMILES: O=C(N(Cc1ccc(-c2nnc(C(F)F)o2)cn1)c1cccc(F)c1)C1(F)CNC1 ; CC(C)(C)OC(=O)N1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.250 g, 0.593 mmol) prepared in step 2 of Example 31, tert-butyl 3-oxoazetidine-1-carboxylate (0.203 g, 1.187 mmol), acetic acid (0.034 mL, 0.593 mmol), and sodium triacetoxyborohydride (0.377 g, 1.780 mmol) were dissolved in dichloromethane (20 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by column chromatography (SiO₂, 12 g cartridge; dichloromethane/methanol=0% to 10%) and concentrated to obtain the title compound (0.300 g, 87.7%) as a yellow gel.

### [Step 2] Synthesis of N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate

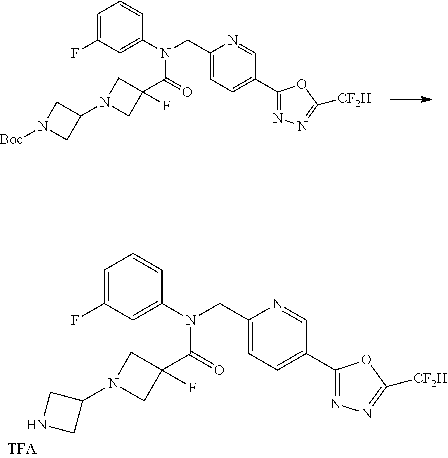 [SMILES: CC(C)(C)OC(=O)N1CC(N2CC(F)(C(=O)N(Cc3ccc(-c4nnc(C(F)F)o4)cn3)c3cccc(F)c3)C2)C1 ; O=C(N(Cc1ccc(-c2nnc(C(F)F)o2)cn1)c1cccc(F)c1)C1(F)CN(C2CNC2)C1]

Tert-butyl 3-(((5-(5-(difluoromethyl)-1,3,4-oxadiazol-2-yl)pyridin-2-yl)methyl)(3-fluorophenyl)carbamoyl)-3-fluoro-[1,3′-biazetidine]-1′-carboxylate (0.380 g, 0.659 mmol) prepared in step 1 and trifluoroacetic acid (1.009 mL, 13.182 mmol) were dissolved in dichloromethane (10 mL) at room temperature, and the resulting solution was stirred at the same temperature for 2 hours. After removing the solvent from the reaction mixture under reduced pressure, the title compound (0.314 g, 100.0%) was obtained as a brown gel.

### [Step 3] Synthesis of Compound 3219

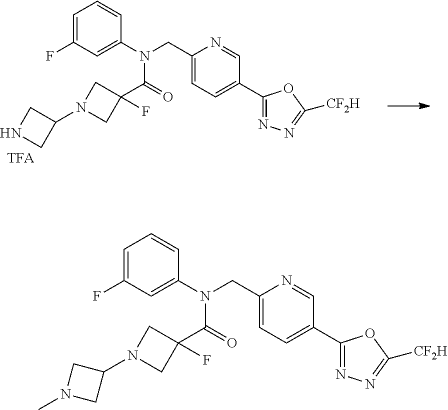 [SMILES: O=C(N(Cc1ccc(-c2nnc(C(F)F)o2)cn1)c1cccc(F)c1)C1(F)CN(C2CNC2)C1 ; CN1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2, formaldehyde (0.006 g, 0.210 mmol), acetic acid (0.006 mL, 0.105 mmol), and sodium triacetoxyborohydride (0.067 g, 0.315 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.023 g, 44.7%) as a yellow gel.

¹H NMR (400 MHz, CDCl₃) δ 9.28 (d, J=2.1 Hz, 1H), 8.40 (dd, J=8.2, 2.2 Hz, 1H), 7.56 (d, J=7.8 Hz, 1H), 7.36 (dd, J=14.5, 8.1 Hz, 1H), 7.13-6.80 (m, 4H), 5.10 (s, 2H), 3.82-3.67 (m, 2H), 3.61 (s, 2H), 3.44 (s, 1H), 3.32-3.19 (m, 2H), 3.13 (s, 2H), 2.48 (s, 3H);

LRMS (ES) m/z 492.3 (M++1).

### Example 85: Synthesis of Compound 3220, N-((5-(5-(difluoromethyl)-1,3,4-oxadiazol-2-yl)pyridin-2-yl)methyl)-1′-ethyl-3-fluoro-N-(3-fluorophenyl)-[1,3′-biazetidine]-3-carboxamide

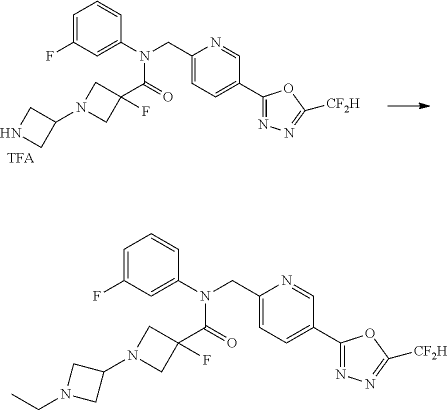 [SMILES: O=C(N(Cc1ccc(-c2nnc(C(F)F)o2)cn1)c1cccc(F)c1)C1(F)CN(C2CNC2)C1 ; CCN1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, acetaldehyde (0.009 g, 0.210 mmol), acetic acid (0.006 mL, 0.105 mmol), and sodium triacetoxyborohydride (0.067 g, 0.315 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.025 g, 47.2%) as a yellow gel.

¹H NMR (400 MHz, CDCl₃) δ 9.19 (d, J=1.7 Hz, 1H), 8.31 (dd, J=8.2, 2.2 Hz, 1H), 7.47 (d, J=8.2 Hz, 1H), 7.31-7.21 (m, 1H), 7.04-6.70 (m, 4H), 5.00 (s, 2H), 3.74-3.56 (m, 2H), 3.39 (d, J=27.2 Hz, 3H), 3.23-3.06 (m, 2H), 2.91 (s, 2H), 2.50 (s, 2H), 0.93 (t, J=7.1 Hz, 3H);

LRMS (ES) m/z 506.3 (M++1).

### Example 86: Synthesis of Compound 3221, N-((5-(5-(difluoromethyl)-1,3,4-oxadiazol-2-yl)pyridin-2-yl)methyl)-3-fluoro-N-(3-fluorophenyl)-1′-propyl-[1,3′-biazetidine]-3-carboxamide

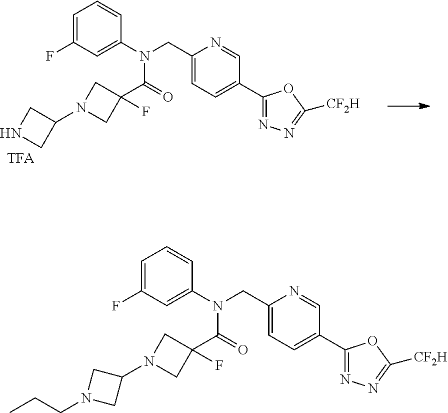 [SMILES: O=C(N(Cc1ccc(-c2nnc(C(F)F)o2)cn1)c1cccc(F)c1)C1(F)CN(C2CNC2)C1 ; CCCN1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, propioaldehyde (0.012 g, 0.210 mmol), acetic acid (0.006 mL, 0.105 mmol), and sodium triacetoxyborohydride (0.067 g, 0.315 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.025 g, 45.9%) as a yellow gel.

¹H NMR (400 MHz, CDCl₃) δ 9.28 (d, J=1.6 Hz, 1H), 8.40 (dd, J=8.2, 2.2 Hz, 1H), 7.56 (d, J=8.2 Hz, 1H), 7.40-7.31 (m, 1H), 7.02 (ddd, J=87.4, 42.7, 28.5 Hz, 4H), 5.10 (s, 2H), 3.81-3.65 (m, 2H), 3.44 (s, 3H), 3.25 (dd, J=21.9, 8.9 Hz, 2H), 3.00 (s, 2H), 2.49 (s, 2H), 1.42 (dd, J=14.5, 7.4 Hz, 2H), 0.91 (t, J=7.4 Hz, 3H);

LRMS (ES) m/z 520.4 (M++1).

### Example 87: Synthesis of Compound 3222, 1′-butyl-N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide

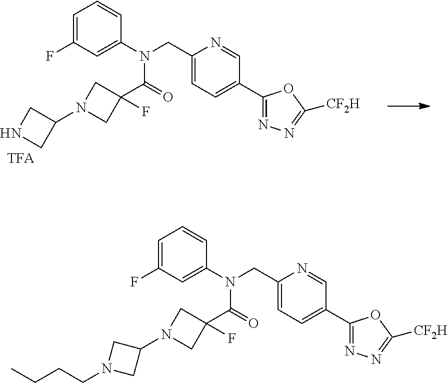 [SMILES: O=C(N(Cc1ccc(-c2nnc(C(F)F)o2)cn1)c1cccc(F)c1)C1(F)CN(C2CNC2)C1 ; CCCCN1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, butyraldehyde (0.015 g, 0.210 mmol), acetic acid (0.006 mL, 0.105 mmol), and sodium triacetoxyborohydride (0.067 g, 0.315 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.022 g, 39.4%) as a yellow gel.

¹H NMR (400 MHz, CDCl₃) δ 9.28 (d, J=2.0 Hz, 1H), 8.40 (dd, J=8.2, 2.2 Hz, 1H), 7.56 (d, J=8.1 Hz, 1H), 7.40-7.31 (m, 1H), 7.13-6.80 (m, 4H), 5.09 (s, 2H), 3.72 (d, J=22.8 Hz, 2H), 3.50 (d, J=34.3 Hz, 3H), 3.33-3.16 (m, 2H), 3.03 (s, 2H), 2.56 (s, 2H), 1.44-1.30 (m, 4H), 0.91 (t, J=7.1 Hz, 3H);

LRMS (ES) m/z 534.3 (M++1).

### Example 88: Synthesis of Compound 3223, N-((5-(5-(difluoromethyl)-1,3,4-oxadiazol-2-yl)pyridin-2-yl)methyl)-3-fluoro-N-(3-fluorophenyl)-1′-isobutyl-[1,3′-biazetidine]-3-carboxamide

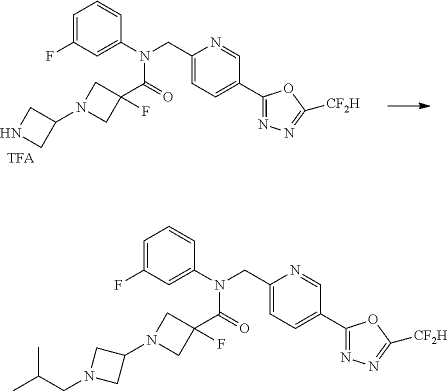 [SMILES: O=C(N(Cc1ccc(-c2nnc(C(F)F)o2)cn1)c1cccc(F)c1)C1(F)CN(C2CNC2)C1 ; CC(C)CN1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, isobutyraldehyde (0.015 g, 0.210 mmol), acetic acid (0.006 mL, 0.105 mmol), and sodium triacetoxyborohydride (0.067 g, 0.315 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.021 g, 37.6%) as a yellow gel.

¹H NMR (400 MHz, CDCl₃) δ 9.28 (d, J=1.9 Hz, 1H), 8.40 (dd, J=8.2, 2.2 Hz, 1H), 7.56 (d, J=8.1 Hz, 1H), 7.40-7.31 (m, 1H), 7.02 (ddd, J=87.3, 42.4, 27.7 Hz, 4H), 5.10 (s, 2H), 3.72 (dd, J=21.4, 9.1 Hz, 2H), 3.42 (s, 3H), 3.24 (dd, J=22.3, 9.7 Hz, 2H), 2.94 (s, 2H), 2.29 (d, J=6.3 Hz, 2H), 1.62 (dt, J=13.1, 6.7 Hz, 1H), 0.89 (d, J=6.7 Hz, 6H);

LRMS (ES) m/z 534.4 (M++1).

### Example 89: Synthesis of Compound 3224, N-((5-(5-(difluoromethyl)-1,3,4-oxadiazol-2-yl)pyridin-2-yl)methyl)-3-fluoro-N-(3-fluorophenyl)-1′-isobutyl-[1,3′-biazetidine]-3-carboxamide

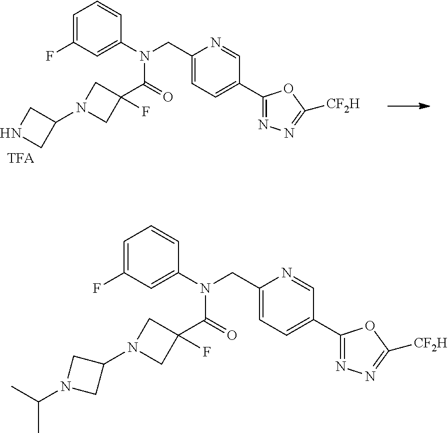 [SMILES: O=C(N(Cc1ccc(-c2nnc(C(F)F)o2)cn1)c1cccc(F)c1)C1(F)CN(C2CNC2)C1 ; CC(C)N1CC(N2CC(F)(C(=O)N(Cc3ccc(-c4nnc(C(F)F)o4)cn3)c3cccc(F)c3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, propan-2-one (0.012 g, 0.210 mmol), acetic acid (0.006 mL, 0.105 mmol), and sodium triacetoxyborohydride (0.067 g, 0.315 mmol) were dissolved in dichloromethane (5 mL) at room temperature, and the resulting solution was stirred at the same temperature for 18 hours. The solvent was removed from the reaction mixture under reduced pressure. A saturated aqueous sodium bicarbonate solution was poured into the obtained concentrate, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.6%) as a yellow gel.

¹H NMR (400 MHz, CDCl₃) δ 9.28 (d, J=1.7 Hz, 1H), 8.40 (dd, J=8.2, 2.2 Hz, 1H), 7.56 (d, J=8.1 Hz, 1H), 7.40-7.30 (m, 1H), 7.12-6.79 (m, 4H), 5.09 (s, 2H), 3.80-3.63 (m, 2H), 3.53 (s, 2H), 3.42 (s, 1H), 3.24 (dd, J=21.0, 9.7 Hz, 2H), 3.01 (s, 2H), 2.50 (s, 1H), 1.02 (d, J=5.6 Hz, 6H);

LRMS (ES) m/z 520.4 (M++1).

### Example 90: Synthesis of Compound 3389, N-((5-(5-(difluoromethyl)-1,3,4-oxadiazol-2-yl)pyridin-2-yl)methyl)-4-fluoro-N-(3-fluorophenyl)-1-(1-methylazetidine-3-yl)piperidine-4-carboxamide

### [Step 1] Synthesis of 3-(4-(((5-(5-(difluoromethyl)-1,3,4-oxadiazol-2-yl)pyridin-2-yl)methyl)(3-fluorophenyl)carbamoyl)-4-fluoropiperidin-1-yl)azetidine-1-carboxylate

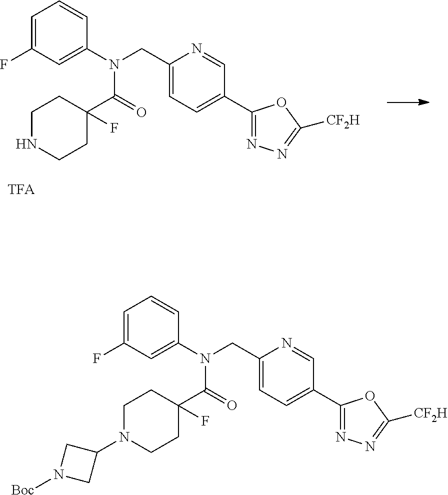 [SMILES: O=C(N(Cc1ccc(-c2nnc(C(F)F)o2)cn1)c1cccc(F)c1)C1(F)CCNCC1 ; CC(C)(C)OC(=O)N1CC(N2CCC(F)(C(=O)N(Cc3ccc(-c4nnc(C(F)F)o4)cn3)c3cccc(F)c3)CC2)C1]

N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate (0.458 g, 1.019 mmol) prepared in step 3 of Example 10, tert-butyl 3-oxoazetidine-1-carboxylate (0.349 g, 2.038 mmol), acetic acid (0.058 mL, 1.019 mmol), and sodium triacetoxyborohydride (0.648 g, 3.057 mmol) were dissolved in dichloromethane (10 mL) at room temperature, and the resulting solution was stirred at the same temperature for 18 hours. A saturated aqueous sodium bicarbonate solution was poured into the reaction mixture, followed by extraction with dichloromethane. The organic layer was washed with water, dried over anhydrous magnesium sulfate, filtered, and concentrated under reduced pressure. The concentrate was purified by column chromatography (SiO₂, 24 g cartridge; dichloromethane/methanol=0% to 10%) and concentrated to obtain the title compound (0.300 g, 48.7%) as a yellow gel.

### [Step 2] Synthesis of 1-(azetidine-3-yl)-N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate

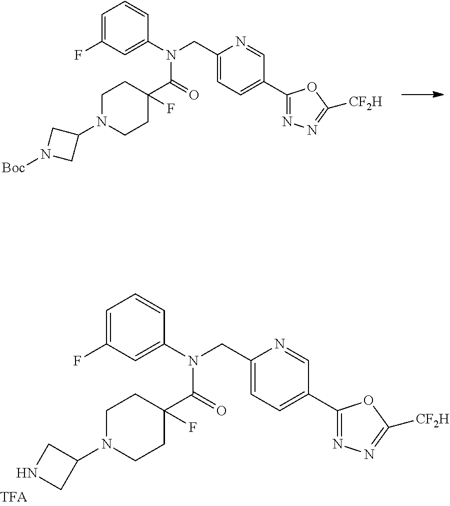 [SMILES: CC(C)(C)OC(=O)N1CC(N2CCC(F)(C(=O)N(Cc3ccc(-c4nnc(C(F)F)o4)cn3)c3cccc(F)c3)CC2)C1 ; O=C(N(Cc1ccc(-c2nnc(C(F)F)o2)cn1)c1cccc(F)c1)C1(F)CCN(C2CNC2)CC1]

Tert-butyl 3-(4-(((5-(5-(difluoromethyl)-1,3,4-oxadiazol-2-yl)pyridin-2-yl)methyl)(3-fluorophenyl)carbamoyl)-4-fluoropiperidin-1-yl)azetidine-1-carboxylate (0.300 g, 0.496 mmol) prepared in step 1 and trifluoroacetic acid (0.760 mL, 9.924 mmol) were dissolved in dichloromethane (10 mL) at room temperature, and the resulting solution was stirred at the same temperature for 2 hours. After removing the solvent from the reaction mixture under reduced pressure, the title compound (0.250 g, 99.9%) was obtained as a yellow gel.

### [Step 3] Synthesis of Compound 3389

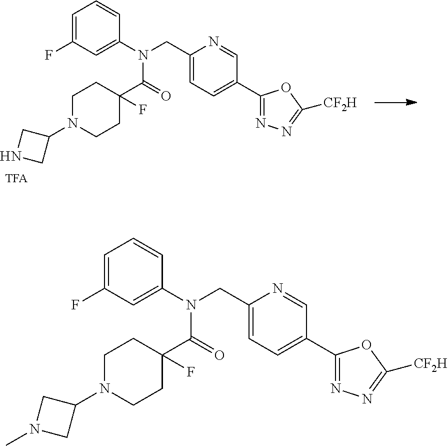 [SMILES: O=C(N(Cc1ccc(-c2nnc(C(F)F)o2)cn1)c1cccc(F)c1)C1(F)CCN(C2CNC2)CC1 ; CN1CC(N2CCC(F)(C(=O)N(Cc3ccc(-c4nnc(C(F)F)o4)cn3)c3cccc(F)c3)CC2)C1]

1-(Azetidine-3-yl)-N-((5-(5-(difluoromethyl)-1,3,4-oxadiazol-2-yl)pyridin-2-yl)meth yl)-4-fluoro-N-(3-fluorophenyl)piperidine-4-carboxamide 2,2,2-trifluoroacetate (0.050 g, 0.099 mmol) prepared in step 2, formaldehyde (0.006 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 29.2%) as a yellow gel.

¹H NMR (400 MHz, CDCl₃) δ 9.19 (d, J=2.0 Hz, 1H), 8.29 (dd, J=8.2, 2.2 Hz, 1H), 7.50-7.33 (m, 1H), 7.27-7.21 (m, 1H), 7.02-6.74 (m, 4H), 4.97 (s, 2H), 4.16 (s, 1H), 3.39 (d, J=21.9 Hz, 2H), 3.33-3.18 (m, 1H), 2.87-2.61 (m, 2H), 2.46 (d, J=9.9 Hz, 1H), 2.22 (ddd, J=30.1, 16.9, 8.7 Hz, 3H), 1.93 (dt, J=25.0, 11.9 Hz, 4H), 1.25-1.19 (m, 1H), 0.84-0.72 (m, 1H);

LRMS (ES) m/z 519.2 (M++1).

### Example 91: Synthesis of Compound 3390, N-((5-(5-(difluoromethyl)-1,3,4-oxadiazol-2-yl)pyridin-2-yl)methyl)-4-fluoro-N-(3-fluorophenyl)-1-(1-propylazetidine-3-yl)piperidin-4-carboxamide

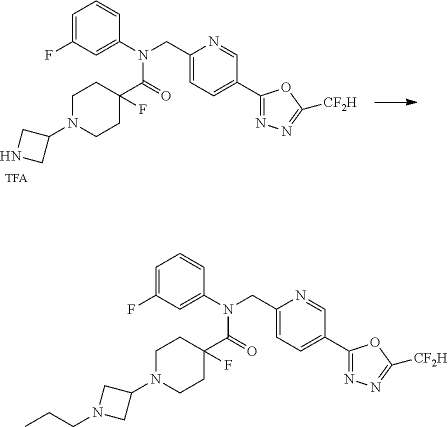 [SMILES: O=C(N(Cc1ccc(-c2nnc(C(F)F)o2)cn1)c1cccc(F)c1)C1(F)CCN(C2CNC2)CC1 ; CCCN1CC(N2CCC(F)(C(=O)N(Cc3ccc(-c4nnc(C(F)F)o4)cn3)c3cccc(F)c3)CC2)C1]

1-(Azetidine-3-v11-N-((5-(5-(difluoromethyl)-1,3,4-oxadiazol-2-yl)pyridin-2-yl)meth yl)-4-fluoro-N-(3-fluorophenyl)piperidine-4-carboxamide 2,2,2-trifluoroacetate (0.050 g, 0.099 mmol) prepared in step 2 of Example 90, propioaldehyde (0.012 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.7%) as a yellow gel.

¹H NMR (400 MHz, CDCl₃) δ 9.22-9.15 (m, 1H), 8.29 (dd, J=8.2, 2.2 Hz, 1H), 7.42 (d, J=8.2 Hz, 1H), 7.26-7.20 (m, 1H), 7.01-6.72 (m, 4H), 4.97 (s, 2H), 3.69 (d, J=25.8 Hz, 2H), 3.03 (dd, J=11.9, 5.6 Hz, 3H), 2.60-2.52 (m, 2H), 2.52-2.42 (m, 2H), 2.33-2.13 (m, 2H), 1.90 (dt, J=32.9, 11.9 Hz, 4H), 1.47-1.36 (m, 2H), 0.85 (t, J=7.4 Hz, 3H);

LRMS (ES) m/z 547.0 (M++1).

### Example 92: Synthesis of Compound 3391, 1-(1-butylazetidine-3-yl)-N-((5-(5-(difluoromethyl)-1,3,4-oxadiazol-2-yl)pyridin-2-yl)methyl)-4-fluoro-N-(3-fluorophenyl)piperidine-4-carboxamide

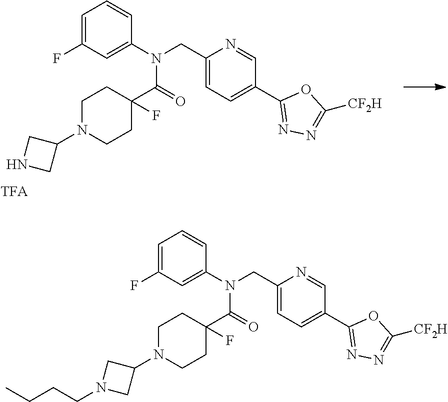 [SMILES: O=C(N(Cc1ccc(-c2nnc(C(F)F)o2)cn1)c1cccc(F)c1)C1(F)CCN(C2CNC2)CC1 ; CCCCN1CC(N2CCC(F)(C(=O)N(Cc3ccc(-c4nnc(C(F)F)o4)cn3)c3cccc(F)c3)CC2)C1]

1-(Azetidine-3-yl)-N-((5-(5-(difluoromethyl)-1,3,4-oxadiazol-2-yl)pyridin-2-yl)meth yl)-4-fluoro-N-(3-fluorophenyl)piperidine-4-carboxamide 2,2,2-trifluoroacetate (0.050 g, 0.099 mmol) prepared in step 2 of Example 90, butyraldehyde (0.014 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.0%) as a yellow gel.

¹H NMR (400 MHz, CDCl₃) δ 9.25-9.10 (m, 1H), 8.29 (dd, J=8.2, 2.2 Hz, 1H), 7.43 (t, J=9.3 Hz, 1H), 7.26-7.20 (m, 1H), 7.01-6.71 (m, 4H), 4.97 (s, 2H), 3.71 (s, 2H), 3.02 (dd, J=14.8, 6.1 Hz, 3H), 2.62-2.53 (m, 2H), 2.53-2.43 (m, 2H), 2.22 (dtd, J=25.2, 13.0, 8.2 Hz, 2H), 1.99-1.80 (m, 4H), 1.41-1.31 (m, 2H), 1.25 (td, J=14.4, 7.1 Hz, 2H), 0.83 (t, J=7.3 Hz, 3H);

LRMS (ES) m/z 561.1 (M++1).

### Example 93: Synthesis of Compound 3392, N-((5-(5-(difluoromethyl)-1,3,4-oxadiazol-2-yl)pyridin-2-yl)methyl)-4-fluoro-N-(3-fluorophenyl)-1-(1-isobutylazetidine-3-yl)piperidine-4-carboxamide

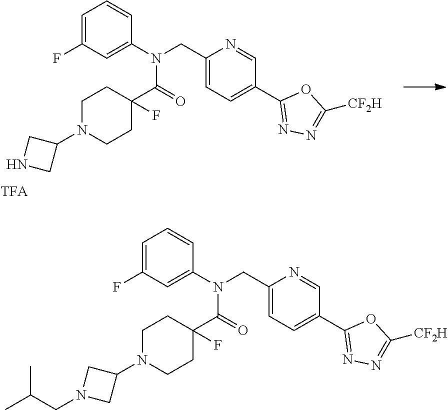 [SMILES: O=C(N(Cc1ccc(-c2nnc(C(F)F)o2)cn1)c1cccc(F)c1)C1(F)CCN(C2CNC2)CC1 ; CC(C)CN1CC(N2CCC(F)(C(=O)N(Cc3ccc(-c4nnc(C(F)F)o4)cn3)c3cccc(F)c3)CC2)C1]

1-(Azetidine-3-yl)-N-((5-(5-(difluoromethyl)-1,3,4-oxadiazol-2-yl)pyridin-2-yl)meth yl)-4-fluoro-N-(3-fluorophenyl)piperidine-4-carboxamide 2,2,2-trifluoroacetate (0.050 g, 0.099 mmol) prepared in step 2 of Example 90, isobutyraldehyde (0.014 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.0%) as a yellow gel.

¹H NMR (400 MHz, CDCl₃) δ 9.18 (d, J=1.6 Hz, 1H), 8.29 (dd, J=8.2, 2.2 Hz, 1H), 7.42 (d, J=8.2 Hz, 1H), 7.27-7.20 (m, 1H), 7.02-6.70 (m, 4H), 4.97 (s, 2H), 3.66 (s, 2H), 3.05-2.85 (m, 3H), 2.53-2.43 (m, 2H), 2.40-2.13 (m, 4H), 1.97-1.80 (m, 4H), 1.65 (dt, J=13.1, 6.7 Hz, 1H), 0.84 (d, J=6.7 Hz, 6H);

LRMS (ES) m/z 561.1 (M++1).

### Example 94: Synthesis of Compound 3393, N-((5-(5-(difluoromethyl)-1,3,4-oxadiazol-2-yl)pyridin-2-yl)methyl)-4-fluoro-N-(3-fluorophenyl)-1-(1-isopropylazetidine-3-yl)piperidin-4-carboxamide

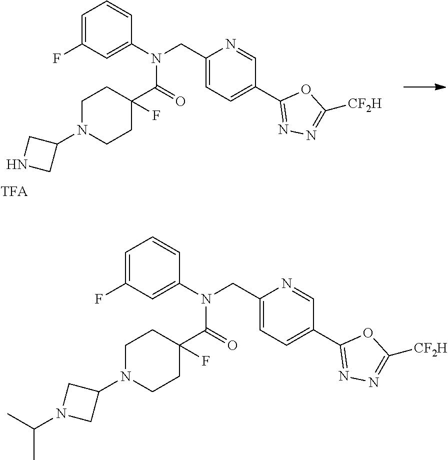 [SMILES: O=C(N(Cc1ccc(-c2nnc(C(F)F)o2)cn1)c1cccc(F)c1)C1(F)CCN(C2CNC2)CC1 ; CC(C)N1CC(N2CCC(F)(C(=O)N(Cc3ccc(-c4nnc(C(F)F)o4)cn3)c3cccc(F)c3)CC2)C1]

1-(Azetidine-3-yl)-N-((5-(5-(difluoromethyl)-1,3,4-oxadiazol-2-yl)pyridin-2-yl)meth yl)-4-fluoro-N-(3-fluorophenyl)piperidine-4-carboxamide 2,2,2-trifluoroacetate (0.050 g, 0.099 mmol) prepared in step 2 of Example 90, propan-2-one (0.012 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.7%) as a yellow gel.

¹H NMR (400 MHz, CDCl₃) δ 9.18 (d, J=1.6 Hz, 1H), 8.29 (dd, J=8.2, 2.2 Hz, 1H), 7.42 (d, J=8.2 Hz, 1H), 7.28-7.16 (m, 1H), 7.03-6.71 (m, 4H), 4.99 (d, J=13.6 Hz, 2H), 3.64 (s, 2H), 2.96 (s, 3H), 2.58-2.44 (m, 3H), 2.34-2.11 (m, 2H), 1.90 (dt, J=31.4, 11.9 Hz, 4H), 0.98 (d, J=6.3 Hz, 6H);

LRMS (ES) m/z 547.1 (M++1).

### Example 95: Synthesis of Compound 3394, 1′-cyclobutyl-N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide

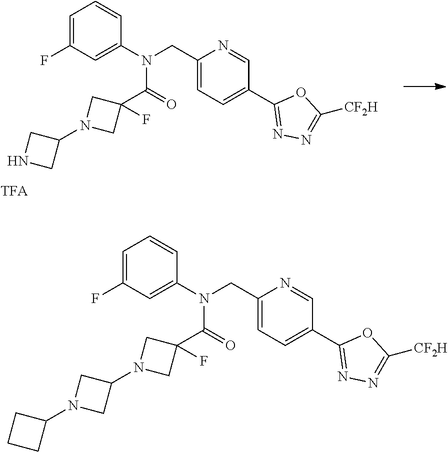 [SMILES: O=C(N(Cc1ccc(-c2nnc(C(F)F)o2)cn1)c1cccc(F)c1)C1(F)CN(C2CNC2)C1 ; O=C(N(Cc1ccc(-c2nnc(C(F)F)o2)cn1)c1cccc(F)c1)C1(F)CN(C2CN(C3CCC3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, cyclobutanone (0.015 g, 0.210 mmol), acetic acid (0.006 mL, 0.105 mmol), and sodium triacetoxyborohydride (0.067 g, 0.315 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 26.9%) as a yellow gel.

¹H NMR (400 MHz, CDCl₃) δ 9.18 (d, J=1.6 Hz, 1H), 8.30 (dd, J=8.2, 2.2 Hz, 1H), 7.47 (d, J=8.1 Hz, 1H), 7.26 (dt, J=8.0, 7.2 Hz, 1H), 6.92 (dt, J=85.9, 29.1 Hz, 4H), 5.00 (s, 2H), 3.63 (dd, J=21.5, 9.2 Hz, 2H), 3.40-3.27 (m, 3H), 3.15 (dd, J=17.6, 9.8 Hz, 3H), 2.93 (s, 2H), 1.96-1.84 (m, 2H), 1.80 (dd, J=19.3, 9.6 Hz, 2H), 1.75-1.66 (m, 1H), 1.60 (dt, J=10.3, 8.6 Hz, 1H);

LRMS (ES) m/z 531.0 (M++1).

### Example 96: Synthesis of Compound 3395, N-((5-(5-(difluoromethyl)-1,3,4-oxadiazol-2-yl)pyridin-2-yl)methyl)-3-fluoro-N-(3-fluorophenyl)-1′-(oxetan-3-yl)-[1,3′-biazetidine]-3-carboxamide

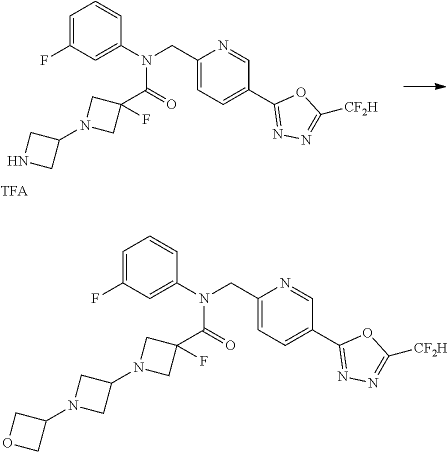 [SMILES: O=C(N(Cc1ccc(-c2nnc(C(F)F)o2)cn1)c1cccc(F)c1)C1(F)CN(C2CNC2)C1 ; O=C(N(Cc1ccc(-c2nnc(C(F)F)o2)cn1)c1cccc(F)c1)C1(F)CN(C2CN(C3COC3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, oxetan-3-one (0.015 g, 0.210 mmol), acetic acid (0.006 mL, 0.105 mmol), and sodium triacetoxyborohydride (0.067 g, 0.315 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 26.8%) as a yellow gel.

¹H NMR (400 MHz, CDCl₃) δ 9.19 (dd, J=4.2, 1.9 Hz, 1H), 8.31 (dd, J=8.2, 1.5 Hz, 1H), 7.49-7.42 (m, 1H), 7.32-7.22 (m, 1H), 7.04-6.69 (m, 4H), 5.00 (s, 2H), 4.60 (t, J=6.7 Hz, 1H), 4.48-4.37 (m, 1H), 4.06-3.91 (m, 1H), 3.88 (s, 1H), 3.66 (ddd, J=63.0, 30.5, 14.3 Hz, 4H), 3.34 (s, 1H), 3.25-3.13 (m, 2H), 3.00 (s, 1H), 1.30-1.20 (m, 1H), 0.83-0.72 (m, 1H);

LRMS (ES) m/z 533.4 (M++1).

### Example 97: Synthesis of Compound 3396, 1′-cyclopentyl-N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide

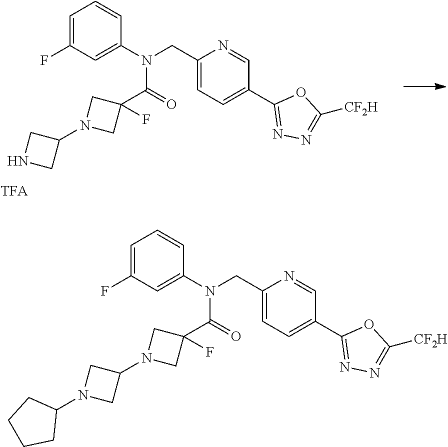 [SMILES: O=C(N(Cc1ccc(-c2nnc(C(F)F)o2)cn1)c1cccc(F)c1)C1(F)CN(C2CNC2)C1 ; O=C(N(Cc1ccc(-c2nnc(C(F)F)o2)cn1)c1cccc(F)c1)C1(F)CN(C2CN(C3CCCC3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, cyclopentanone (0.018 g, 0.210 mmol), acetic acid (0.006 mL, 0.105 mmol), and sodium triacetoxyborohydride (0.067 g, 0.315 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 26.2%) as a yellow gel.

¹H NMR (400 MHz, CDCl₃) δ 9.22-9.13 (m, 1H), 8.31 (dd, J=8.2, 2.2 Hz, 1H), 7.47 (d, J=8.1 Hz, 1H), 7.30-7.22 (m, 1H), 6.92 (dt, J=85.4, 29.4 Hz, 4H), 5.00 (s, 2H), 3.63 (dd, J=21.8, 9.3 Hz, 2H), 3.46 (t, J=7.3 Hz, 2H), 3.41-3.30 (m, 1H), 3.15 (dd, J=21.7, 9.2 Hz, 2H), 2.95 (d, J=7.0 Hz, 2H), 2.83 (dd, J=11.8, 6.0 Hz, 1H), 1.71-1.53 (m, 4H), 1.46 (ddd, J=9.2, 7.6, 2.5 Hz, 2H), 1.39-1.29 (m, 2H);

LRMS (ES) m/z 546.3 (M++1).

### Example 98: Synthesis of Compound 3397, N-((5-(5-(difluoromethyl)-1,3,4-oxadiazol-2-yl)pyridin-2-yl)methyl)-3-fluoro-N-(3-fluorophenyl)-1′-(tetrahydrofuran-3-yl)-[1,3′-biazetidine]-3-carboxamide

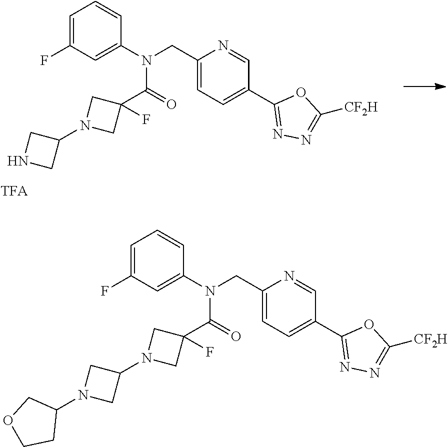 [SMILES: O=C(N(Cc1ccc(-c2nnc(C(F)F)o2)cn1)c1cccc(F)c1)C1(F)CN(C2CNC2)C1 ; O=C(N(Cc1ccc(-c2nnc(C(F)F)o2)cn1)c1cccc(F)c1)C1(F)CN(C2CN(C3CCOC3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, dihydrofuran-3(2H)-one (0.018 g, 0.210 mmol), acetic acid (0.006 mL, 0.105 mmol), and sodium triacetoxyborohydride (0.067 g, 0.315 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 26.2%) as a yellow gel.

¹H NMR (400 MHz, CDCl₃) δ 9.18 (d, J=1.6 Hz, 1H), 8.30 (dd, J=8.2, 2.2 Hz, 1H), 7.46 (d, J=8.1 Hz, 1H), 7.26 (td, J=8.1, 6.4 Hz, 1H), 7.02-6.70 (m, 4H), 5.00 (s, 2H), 3.80 (dd, J=15.6, 7.6 Hz, 1H), 3.59 (dddd, J=26.6, 12.0, 8.7, 3.9 Hz, 5H), 3.27 (s, 3H), 3.15 (dd, J=21.9, 9.2 Hz, 2H), 2.93 (td, J=8.0, 2.9 Hz, 1H), 2.82 (d, J=11.4 Hz, 2H), 1.78 (ddd, J=15.1, 12.8, 7.5 Hz, 1H), 1.60 (dddd, J=12.5, 7.5, 4.9, 2.9 Hz, 1H);

LRMS (ES) m/z 548.1 (M++1).

### Example 99: Synthesis of Compound 3398, 1′-cyclohexyl-N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide

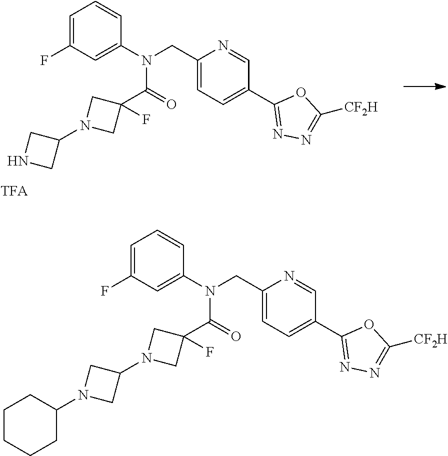 [SMILES: O=C(N(Cc1ccc(-c2nnc(C(F)F)o2)cn1)c1cccc(F)c1)C1(F)CN(C2CNC2)C1 ; O=C(N(Cc1ccc(-c2nnc(C(F)F)o2)cn1)c1cccc(F)c1)C1(F)CN(C2CN(C3CCCCC3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, cyclohexanone (0.021 g, 0.210 mmol), acetic acid (0.006 mL, 0.105 mmol), and sodium triacetoxyborohydride (0.067 g, 0.315 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 25.6%) as a yellow gel.

¹H NMR (400 MHz, CDCl₃) δ 9.19 (d, J=1.7 Hz, 1H), 8.31 (dd, J=8.2, 2.2 Hz, 1H), 7.47 (d, J=8.1 Hz, 1H), 7.26 (dt, J=8.0, 7.2 Hz, 1H), 6.92 (dt, J=85.6, 29.2 Hz, 4H), 5.00 (s, 2H), 3.64 (dd, J=21.3, 9.1 Hz, 2H), 3.54 (t, J=7.2 Hz, 2H), 3.46-3.35 (m, 1H), 3.15 (dd, J=21.5, 9.1 Hz, 2H), 3.00 (s, 2H), 2.15 (d, J=4.2 Hz, 1H), 1.68 (d, J=5.6 Hz, 4H), 1.54 (s, 1H), 1.09 (d, J=5.2 Hz, 5H);

LRMS (ES) m/z 559.1 (M++1).

### Example 100: Synthesis of Compound 3399, N-((5-(5-(difluoromethyl)-1,3,4-oxadiazol-2-yl)pyridin-2-yl)methyl)-3-fluoro-N-(3-fluorophenyl)-1′-(tetrahydro-2H-pyran-4-yl)-[1,3′-biazetidine]-3-carboxamide

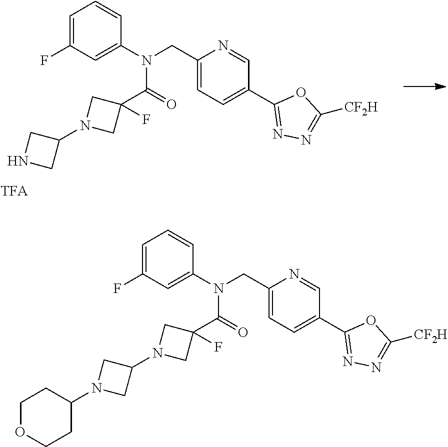 [SMILES: O=C(N(Cc1ccc(-c2nnc(C(F)F)o2)cn1)c1cccc(F)c1)C1(F)CN(C2CNC2)C1 ; O=C(N(Cc1ccc(-c2nnc(C(F)F)o2)cn1)c1cccc(F)c1)C1(F)CN(C2CN(C3CCOCC3)C2)C1]

N-((5-(5-(difluoromethyl)-1,3,4-oxadiazol-2-yl)pyridin-2-yl)methyl)-3-fluoro-N-(3-fluorophenyl)-[1,3′-biazetidine]-3-carboxamide 2,2,2-trifluoroacetate (0.050 g, 0.105 mmol) prepared in step 2 of Example 84, tetrahydro-4H-pyran-4-one (0.021 g, 0.210 mmol), acetic acid (0.006 mL, 0.105 mmol), and sodium triacetoxyborohydride (0.067 g, 0.315 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 25.5%) as a yellow gel.

¹H NMR (400 MHz, CDCl₃) δ 9.21-9.16 (m, 1H), 8.30 (dd, J=8.2, 2.2 Hz, 1H), 7.46 (d, J=8.1 Hz, 1H), 7.31-7.22 (m, 1H), 7.03-6.71 (m, 4H), 5.00 (s, 2H), 3.90-3.80 (m, 2H), 3.63 (dd, J=21.7, 9.2 Hz, 2H), 3.35-3.21 (m, 5H), 3.15 (dd, J=22.1, 9.2 Hz, 2H), 2.81 (d, J=6.6 Hz, 2H), 2.14 (ddd, J=14.0, 10.0, 3.9 Hz, 1H), 1.53 (d, J=11.9 Hz, 2H), 1.25 (ddd, J=13.9, 10.6, 3.9 Hz, 2H);

LRMS (ES) m/z 562.2 (M++1).

### Example 101: Synthesis of Compound 3400, N-((5-(5-(difluoromethyl)-1,3,4-oxadiazol-2-yl)pyridin-2-yl)methyl)-1-(1-ethylpiperidin-4-yl)-3-fluoro-N-(3-fluorophenyl)azetidine-3-carboxamide

### [Step 1] Synthesis of tert-butyl 4-(3-(((5-(5-(difluoromethyl)-1,3,4-oxadiazol-2-yl)pyridin-2-yl)methyl)(3-fluorophenyl)carbamoyl)-3-fluoroazetidine-1-yl)piperidine-1-carboxylate

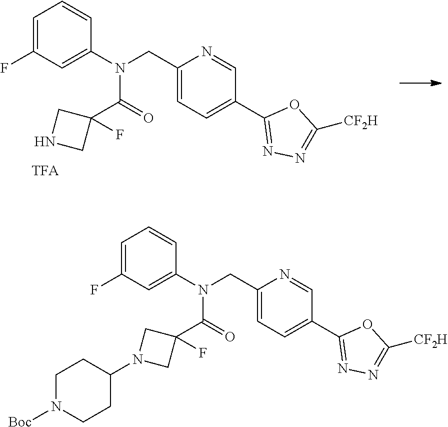 [SMILES: O=C(N(Cc1ccc(-c2nnc(C(F)F)o2)cn1)c1cccc(F)c1)C1(F)CNC1 ; CC(C)(C)OC(=O)N1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)azetidine-3-carboxamide 2,2,2-trifluoroacetate (0.513 g, 1.217 mmol) prepared in step 2 of Example 31, tert-butyl 4-oxopiperidine-1-carboxylate (0.485 g, 2.435 mmol), acetic acid (0.070 mL, 1.217 mmol), and sodium triacetoxyborohydride (0.774 g, 3.652 mmol) were dissolved in dichloromethane (10 mL) at room temperature, and the resulting solution was stirred at the same temperature for 18 hours. A saturated aqueous sodium bicarbonate solution was poured into the reaction mixture, followed by extraction with dichloromethane. The organic layer was washed with water, dried over anhydrous magnesium sulfate, filtered, and concentrated under reduced pressure. The concentrate was purified by column chromatography (SiO₂, 24 g cartridge; methanol/dichloromethane=0% to 10%) and concentrated to obtain the title compound (0.610 g, 82.9%) as white solid.

### [Step 2] Synthesis of N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate

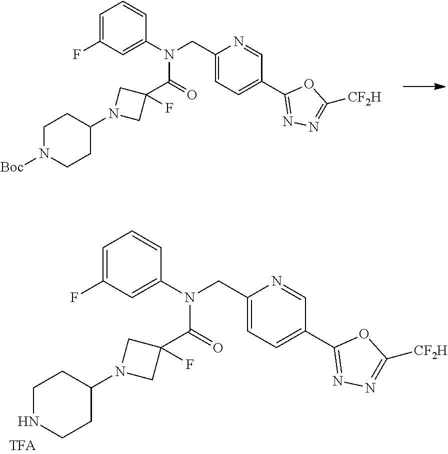 [SMILES: CC(C)(C)OC(=O)N1CCC(N2CC(F)(C(=O)N(Cc3ccc(-c4nnc(C(F)F)o4)cn3)c3cccc(F)c3)C2)CC1 ; O=C(N(Cc1ccc(-c2nnc(C(F)F)o2)cn1)c1cccc(F)c1)C1(F)CN(C2CCNCC2)C1]

Tert-butyl 4-(3-(((5-(5-(difluoromethyl)-1,3,4-oxadiazol-2-yl)pyridin-2-yl)methyl)(3-fluorophenyl)carbamoyl)-3-fluoroazetidine-1-yl)piperidin-1-carboxylate (0.610 g, 1.009 mmol) prepared in step 1 and trifluoroacetic acid (1.545 mL, 20.178 mmol) were dissolved in dichloromethane (10 mL) at room temperature, and the resulting solution was stirred at the same temperature for 2 hours. After removing the solvent from the reaction mixture under reduced pressure, the title compound (0.500 g, 98.2%) was obtained as a yellow gel.

### [Step 3] Synthesis of Compound 3400

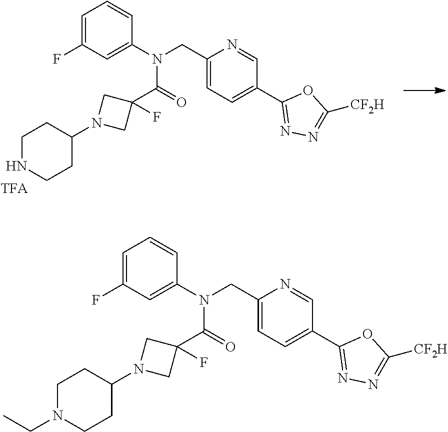 [SMILES: O=C(N(Cc1ccc(-c2nnc(C(F)F)o2)cn1)c1cccc(F)c1)C1(F)CN(C2CCNCC2)C1 ; CCN1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2, acetaldehyde (0.009 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 28.4%) as a yellow gel.

¹H NMR (400 MHz, CDCl₃) δ 9.19 (d, J=1.6 Hz, 1H), 8.30 (dd, J=8.2, 2.2 Hz, 1H), 7.46 (d, J=8.0 Hz, 1H), 7.32-7.22 (m, 1H), 6.90 (ddd, J=51.7, 26.5, 17.8 Hz, 4H), 5.75-5.43 (m, 2H), 5.00 (s, 2H), 3.60-3.43 (m, 2H), 3.08 (d, J=20.8 Hz, 4H), 2.82 (s, 2H), 1.84 (s, 2H), 1.65 (d, J=5.9 Hz, 3H), 1.51-1.33 (m, 3H);

LRMS (ES) m/z 533.4 (M++1).

### Example 102: Synthesis of Compound 3401, N-((5-(5-(difluoromethyl)-1,3,4-oxadiazol-2-yl)pyridin-2-yl)methyl)-3-fluoro-N-(3-fluorophenyl)-1-(1-propylpiperidin-4-yl)azetidine-3-carboxamide

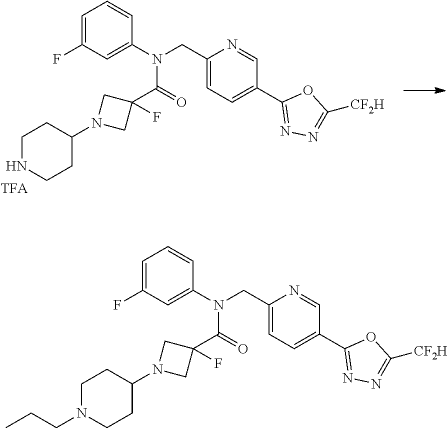 [SMILES: O=C(N(Cc1ccc(-c2nnc(C(F)F)o2)cn1)c1cccc(F)c1)C1(F)CN(C2CCNCC2)C1 ; CCCN1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, propioaldehyde (0.012 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.7%) as a yellow gel.

¹H NMR (400 MHz, CDCl₃) δ 9.19 (d, J=1.6 Hz, 1H), 8.30 (dd, J=8.2, 2.2 Hz, 1H), 7.46 (d, J=8.1 Hz, 1H), 7.30-7.22 (m, 1H), 7.04-6.72 (m, 4H), 5.00 (s, 2H), 3.61-3.44 (m, 2H), 3.09 (dd, J=21.3, 9.3 Hz, 2H), 2.86 (t, J=8.3 Hz, 2H), 2.50 (t, J=16.0 Hz, 4H), 2.20 (d, J=25.1 Hz, 1H), 1.89 (s, 2H), 1.61 (dd, J=15.6, 7.7 Hz, 2H), 1.47-1.37 (m, 2H), 0.85 (t, J=7.4 Hz, 3H);

LRMS (ES) m/z 547.3 (M++1).

### Example 103: Synthesis of Compound 3402, 1-(1-butylpiperidin-4-yl)-N-((5-(5-(difluoromethyl)-1,3,4-oxadiazol-2-yl)pyridin-2-yl)methyl)-3-fluoro-N-(3-fluorophenyl)azetidine-3-carboxamide

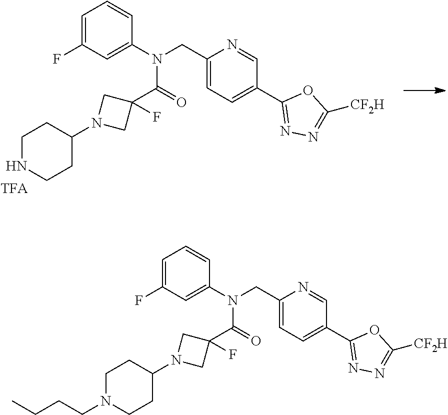 [SMILES: O=C(N(Cc1ccc(-c2nnc(C(F)F)o2)cn1)c1cccc(F)c1)C1(F)CN(C2CCNCC2)C1 ; CCCCN1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, butyraldehyde (0.014 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.0%) as a yellow gel.

¹H NMR (400 MHz, CDCl₃) δ 9.19 (d, J=1.6 Hz, 1H), 8.30 (dd, J=8.2, 2.2 Hz, 1H), 7.45 (d, J=8.1 Hz, 1H), 7.32-7.23 (m, 1H), 6.93 (dt, J=88.2, 28.3 Hz, 4H), 5.00 (s, 2H), 3.53 (dd, J=22.5, 9.0 Hz, 2H), 3.08 (dd, J=20.9, 9.0 Hz, 2H), 2.91 (t, J=9.2 Hz, 2H), 2.63 (dd, J=21.6, 13.8 Hz, 4H), 2.31 (s, 1H), 2.00 (d, J=5.0 Hz, 2H), 1.68-1.55 (m, 2H), 1.53-1.41 (m, 2H), 1.32-1.21 (m, 2H), 0.85 (t, J=7.4 Hz, 3H);

LRMS (ES) m/z 561.2 (M++1).

### Example 104: Synthesis of Compound 3403, N-((5-(5-(difluoromethyl)-1,3,4-oxadiazol-2-yl)pyridin-2-yl)methyl)-3-fluoro-N-(3-fluorophenyl)-1-(1-isobutylpiperidine-4-yl)azetidine-3-carboxamide

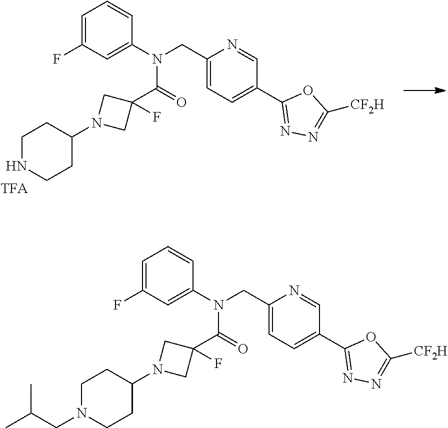 [SMILES: O=C(N(Cc1ccc(-c2nnc(C(F)F)o2)cn1)c1cccc(F)c1)C1(F)CN(C2CCNCC2)C1 ; CC(C)CN1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, isobutyraldehyde (0.014 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.015 g, 27.0%) as a yellow gel.

¹H NMR (400 MHz, CDCl₃) δ 9.18 (d, J=1.6 Hz, 1H), 8.30 (dd, J=8.2, 2.2 Hz, 1H), 7.47 (d, J=8.2 Hz, 1H), 7.29-7.21 (m, 1H), 7.02-6.72 (m, 4H), 5.00 (s, 2H), 3.61-3.42 (m, 2H), 3.09 (dd, J=21.4, 9.1 Hz, 2H), 2.77-2.68 (m, 2H), 2.13 (dd, J=21.2, 13.2 Hz, 5H), 1.70 (d, J=31.3 Hz, 3H), 1.28 (dd, J=9.0, 3.5 Hz, 2H), 0.83 (d, J=6.5 Hz, 6H);

LRMS (ES) m/z 561.1 (M++1).

### Example 105: Synthesis of Compound 3404, N-((5-(5-(difluoromethyl)-1,3,4-oxadiazol-2-yl)pyridin-2-yl)methyl)-3-fluoro-N-(3-fluorophenyl)-1-(1-isopropylpiperidin-4-yl)azetidine-3-carboxamide

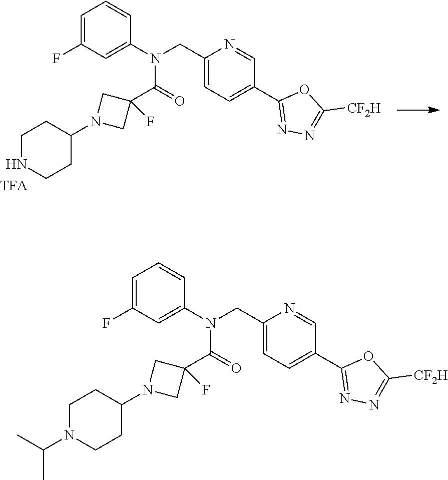 [SMILES: O=C(N(Cc1ccc(-c2nnc(C(F)F)o2)cn1)c1cccc(F)c1)C1(F)CN(C2CCNCC2)C1 ; CC(C)N1CCC(N2CC(F)(C(=O)N(Cc3ccc(-c4nnc(C(F)F)o4)cn3)c3cccc(F)c3)C2)CC1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, propan-2-one (0.012 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.7%) as a yellow gel.

¹H NMR (400 MHz, CDCl₃) δ 9.20 (d, J=1.6 Hz, 1H), 8.31 (dd, J=8.2, 2.2 Hz, 1H), 7.45 (d, J=8.1 Hz, 1H), 7.32-7.22 (m, 1H), 6.93 (dt, J=76.2, 29.2 Hz, 4H), 5.00 (s, 2H), 3.59-3.45 (m, 2H), 3.09 (dd, J=21.3, 9.8 Hz, 3H), 2.95 (t, J=10.1 Hz, 2H), 2.79 (s, 2H), 2.38 (s, 1H), 2.28-2.15 (m, 2H), 1.53 (d, J=13.5 Hz, 2H), 1.25 (d, J=6.4 Hz, 6H);

LRMS (ES) m/z 547.2 (M++1).

### Example 106: Synthesis of Compound 3405, 1-(1-cyclobutylpiperidin-4-yl)-N-((5-(5-(difluoromethyl)-1,3,4-oxadiazol-2-yl)pyridin-2-yl)methyl)-3-fluoro-N-(3-fluorophenyl)azetidine-3-carboxamide

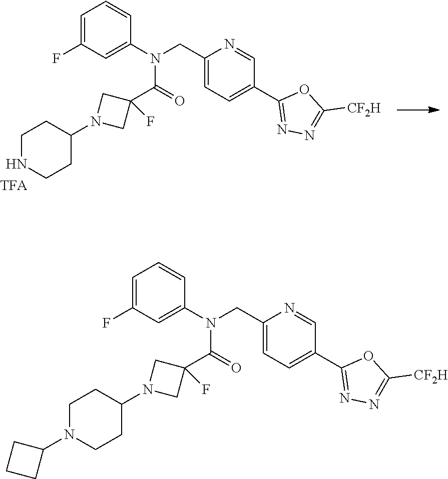 [SMILES: O=C(N(Cc1ccc(-c2nnc(C(F)F)o2)cn1)c1cccc(F)c1)C1(F)CN(C2CCNCC2)C1 ; O=C(N(Cc1ccc(-c2nnc(C(F)F)o2)cn1)c1cccc(F)c1)C1(F)CN(C2CCN(C3CCC3)CC2)C1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, cyclobutanone (0.014 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 27.1%) as a yellow gel.

¹H NMR (400 MHz, CDCl₃) δ 9.22-9.15 (m, 1H), 8.30 (dd, J=8.2, 2.2 Hz, 1H), 7.46 (d, J=8.1 Hz, 1H), 7.30-7.21 (m, 1H), 7.04-6.69 (m, 4H), 5.00 (s, 2H), 3.58-3.40 (m, 2H), 3.08 (dd, J=21.5, 9.3 Hz, 2H), 2.85 (s, 1H), 2.68 (d, J=7.6 Hz, 2H), 2.16 (dd, J=15.3, 7.8 Hz, 4H), 1.99 (dd, J=16.1, 8.2 Hz, 3H), 1.81 (d, J=27.2 Hz, 2H), 1.77-1.65 (m, 1H), 1.58 (dt, J=18.8, 9.5 Hz, 1H), 1.33 (d, J=23.7 Hz, 2H);

LRMS (ES) m/z 559.5 (M++1).

### Example 107: Synthesis of Compound 3406, N-((5-(5-(difluoromethyl)-1,3,4-oxadiazol-2-yl)pyridin-2-yl)methyl)-3-fluoro-N-(3-fluorophenyl)-1-(1-(oxetan-3-yl)piperidin-4-yl)azetidine-3-carboxamide

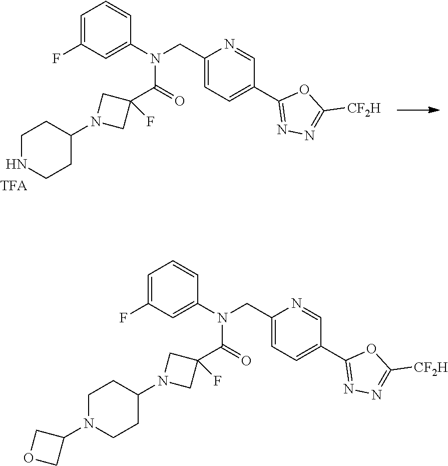 [SMILES: O=C(N(Cc1ccc(-c2nnc(C(F)F)o2)cn1)c1cccc(F)c1)C1(F)CN(C2CCNCC2)C1 ; O=C(N(Cc1ccc(-c2nnc(C(F)F)o2)cn1)c1cccc(F)c1)C1(F)CN(C2CCN(C3COC3)CC2)C1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, oxetan-3-one (0.014 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.015 g, 27.0%) as a yellow gel.

¹H NMR (400 MHz, CDCl₃) δ 9.19 (d, J=1.6 Hz, 1H), 8.30 (dd, J=8.2, 2.2 Hz, 1H), 7.47 (d, J=8.2 Hz, 1H), 7.31-7.21 (m, 1H), 7.03-6.70 (m, 4H), 5.00 (s, 2H), 4.55 (d, J=6.4 Hz, 4H), 3.52 (dd, J=22.3, 8.6 Hz, 2H), 3.41 (s, 1H), 3.11 (dd, J=21.4, 8.2 Hz, 2H), 2.55 (d, J=10.7 Hz, 2H), 2.05 (s, 1H), 1.85 (s, 2H), 1.58 (s, 2H), 1.34-1.21 (m, 2H);

LRMS (ES) m/z 561.1 (M++1).

### Example 108: Synthesis of Compound 3407, 1-(1-cyclopentylpiperidin-4-yl)-N-((5-(5-(difluoromethyl)-1,3,4-oxadiazol-2-yl)pyridin-2-yl)methyl)-3-fluoro-N-(3-fluorophenyl)azetidine-3-carboxamide

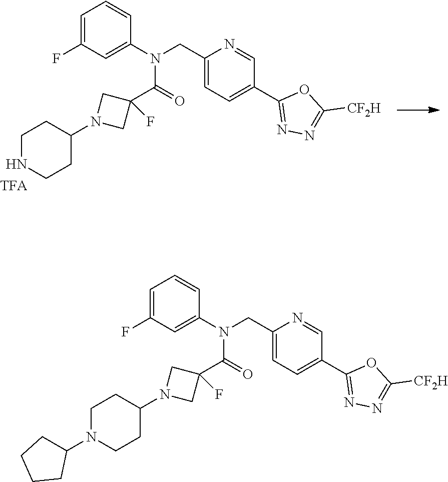 [SMILES: O=C(N(Cc1ccc(-c2nnc(C(F)F)o2)cn1)c1cccc(F)c1)C1(F)CN(C2CCNCC2)C1 ; O=C(N(Cc1ccc(-c2nnc(C(F)F)o2)cn1)c1cccc(F)c1)C1(F)CN(C2CCN(C3CCCC3)CC2)C1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, cyclopentanone (0.017 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.015 g, 26.4%) as a yellow gel.

¹H NMR (400 MHz, CDCl₃) δ 9.19 (d, J=1.5 Hz, 1H), 8.30 (dd, J=8.2, 2.2 Hz, 1H), 7.45 (d, J=8.1 Hz, 1H), 7.31-7.22 (m, 1H), 7.05-6.70 (m, 4H), 5.00 (s, 2H), 3.61-3.41 (m, 2H), 3.08 (dd, J=21.4, 9.1 Hz, 2H), 3.01-2.69 (m, 5H), 2.33 (s, 1H), 2.13 (dd, J=22.8, 15.0 Hz, 2H), 1.88 (d, J=10.2 Hz, 4H), 1.75 (s, 2H), 1.55-1.42 (m, 4H);

LRMS (ES) m/z 573.3 (M++1).

### Example 109: Synthesis of Compound 3408, N-((5-(5-(difluoromethyl)-1,3,4-oxadiazol-2-yl)pyridin-2-yl)methyl)-3-fluoro-N-(3-fluorophenyl)-1-(1-(tetrahydrofuran-3-yl)piperidin-4-yl)azetidine-3-carboxamide

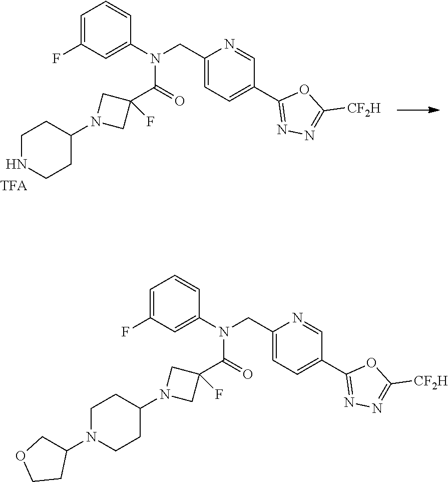 [SMILES: O=C(N(Cc1ccc(-c2nnc(C(F)F)o2)cn1)c1cccc(F)c1)C1(F)CN(C2CCNCC2)C1 ; O=C(N(Cc1ccc(-c2nnc(C(F)F)o2)cn1)c1cccc(F)c1)C1(F)CN(C2CCN(C3CCOC3)CC2)C1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, dihydrofuran-3(2H)-one (0.017 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.005 g, 8.8%) as a yellow gel.

¹H NMR (400 MHz, CDCl₃) δ 9.19 (d, J=1.5 Hz, 1H), 8.30 (dd, J=8.2, 2.2 Hz, 1H), 7.47 (d, J=8.2 Hz, 1H), 7.30-7.21 (m, 1H), 7.03-6.71 (m, 4H), 5.00 (s, 2H), 3.86 (td, J=8.6, 4.4 Hz, 1H), 3.77 (dd, J=8.7, 6.9 Hz, 1H), 3.69 (dd, J=16.2, 8.0 Hz, 1H), 3.63-3.41 (m, 3H), 3.10 (dd, J=21.6, 9.4 Hz, 2H), 2.92 (s, 1H), 2.77 (s, 1H), 2.61 (s, 1H), 2.15-1.93 (m, 4H), 1.80 (s, 1H), 1.59 (s, 2H), 1.29 (dd, J=27.5, 16.4 Hz, 2H);

LRMS (ES) m/z 575.4 (M++1).

### Example 110: Synthesis of Compound 3409, 1-(1-cyclohexylpiperidin-4-yl)-N-((5-(5-(difluoromethyl)-1,3,4-oxadiazol-2-yl)pyridin-2-yl)methyl)-3-fluoro-N-(3-fluorophenyl)azetidine-3-carboxamide

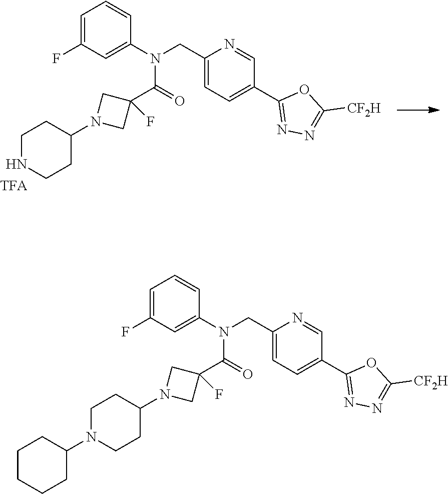 [SMILES: O=C(N(Cc1ccc(-c2nnc(C(F)F)o2)cn1)c1cccc(F)c1)C1(F)CN(C2CCNCC2)C1 ; O=C(N(Cc1ccc(-c2nnc(C(F)F)o2)cn1)c1cccc(F)c1)C1(F)CN(C2CCN(C3CCCCC3)CC2)C1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, cyclohexanone (0.019 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.005 g, 8.6%) as a yellow gel.

¹H NMR (400 MHz, CDCl₃) δ 9.20 (d, J=1.7 Hz, 1H), 8.31 (dd, J=8.1, 2.2 Hz, 1H), 7.44 (d, J=7.0 Hz, 1H), 7.28 (dd, J=14.6, 8.1 Hz, 1H), 7.05-6.70 (m, 4H), 5.00 (s, 2H), 3.04 (s, 4H), 2.46 (s, 2H), 2.16 (dd, J=18.4, 10.9 Hz, 2H), 1.83 (d, J=12.8 Hz, 2H), 1.58 (dd, J=32.7, 12.5 Hz, 6H), 1.37 (dt, J=26.8, 13.2 Hz, 3H), 1.23 (dd, J=17.5, 8.8 Hz, 5H);

LRMS (ES) m/z 587.5 (M++1).

### Example 111: Synthesis of Compound 3410, N-((5-(5-(difluoromethyl)-1,3,4-oxadiazol-2-yl)pyridin-2-yl)methyl)-3-fluoro-N-(3-fluorophenyl)-1-(1-(tetrahydro-2H-pyran-4-yl)piperidin-4-yl)azetidine-3-carboxamide

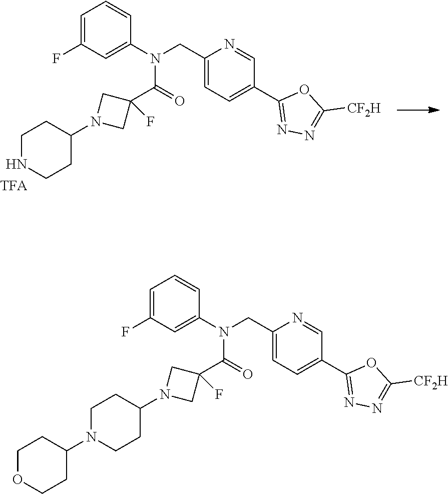 [SMILES: O=C(N(Cc1ccc(-c2nnc(C(F)F)o2)cn1)c1cccc(F)c1)C1(F)CN(C2CCNCC2)C1 ; O=C(N(Cc1ccc(-c2nnc(C(F)F)o2)cn1)c1cccc(F)c1)C1(F)CN(C2CCN(C3CCOCC3)CC2)C1]

N-((5-(5-(difluoromethyl)-1,3,4-oxadiazol-2-yl)pyridin-2-yl)methyl)-3-fluoro-N-(3-fluorophenyl)-1-(piperidin-4-yl)azetidine-3-carboxamide 2,2,2-trifluoroacetate (0.050 g, 0.099 mmol) prepared in step 2 of Example 101, tetrahydro-4H-pyran-4-one (0.020 g, 0.198 mmol), acetic acid (0.006 mL, 0.099 mmol), and sodium triacetoxyborohydride (0.063 g, 0.297 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.005 g, 8.6%) as a yellow gel.

¹H NMR (400 MHz, CDCl₃) δ 9.20 (d, J=1.6 Hz, 1H), 8.30 (dd, J=8.2, 2.2 Hz, 1H), 7.45 (d, J=8.1 Hz, 1H), 7.27 (dd, J=14.5, 8.2 Hz, 1H), 7.04-6.70 (m, 4H), 5.00 (s, 2H), 3.97 (d, J=10.7 Hz, 2H), 3.59-3.44 (m, 2H), 3.30 (t, J=11.2 Hz, 2H), 3.09 (dd, J=21.6, 9.4 Hz, 2H), 2.92 (s, 2H), 2.02-1.76 (m, 3H), 1.67 (s, 3H), 1.57 (s, 2H), 1.48 (s, 2H), 1.33-1.21 (m, 2H);

LRMS (ES) m/z 589.2 (M++1).

### Example 112: Synthesis of Compound 3429, N-(4-(5-(difluoromethyl)-1,3,4-oxadiazol-2-yl)-2-fluorobenzyl)-3-fluoro-1-methyl-N-phenylazetidine-3-carboxamide

### [Step 1] Synthesis of tert-butyl 3-(((4-(5-(difluoromethyl)-1,3,4-oxadiazol-2-yl)-2-fluorobenzyl)(phenyl)carbamoyl)-3-fluoroazetidine-1-carboxylate

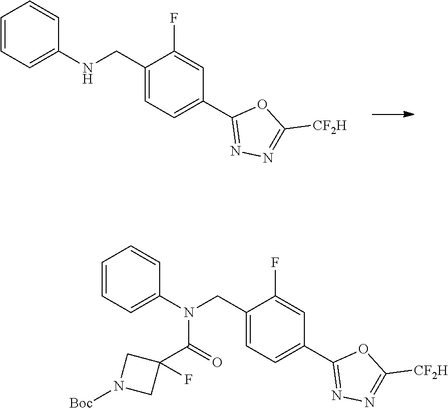 [SMILES: Fc1cc(-c2nnc(C(F)F)o2)ccc1CNc1ccccc1 ; CC(C)(C)OC(=O)N1CC(F)(C(=O)N(Cc2ccc(-c3nnc(C(F)F)o3)cc2F)c2ccccc2)C1]

To a solution in which N-(4-(5-(difluoromethyl)-1,3,4-oxadiazol-2-yl)-2-fluorobenzyl)aniline (1.000 g, 3.132 mmol) prepared in step 1 of Example 1 and triethylamine (1.436 mL, 9.396 mmol) were dissolved in dichloromethane (20 mL) at room temperature, tert-butyl 3-(chlorocarbonyl)-3-fluoroazetidine-1-carboxylate (0.968 g, 4.072 mmol) prepared in step 1 of Example 14 was added and stirred at the same temperature for 16 hours. A saturated aqueous ammonium chloride solution was poured into the reaction mixture, followed by extraction with dichloromethane. The organic layer was washed with water, dried over anhydrous magnesium sulfate, filtered, and concentrated under reduced pressure. The concentrate was purified by column chromatography (SiO 2, 12 g cartridge; ethyl acetate/hexane=0% to 30%) and concentrated to obtain the title compound (0.527 g, 32.3%) as a yellow solid.

### [Step 2] Synthesis of N-(4-(5-(difluoromethyl)-1,3,4-oxadiazol-2-yl)-2-fluorobenzyl)-3-fluoro-N-phenylazetidine-3-carboxamide 2,2,2-trifluoroacetate

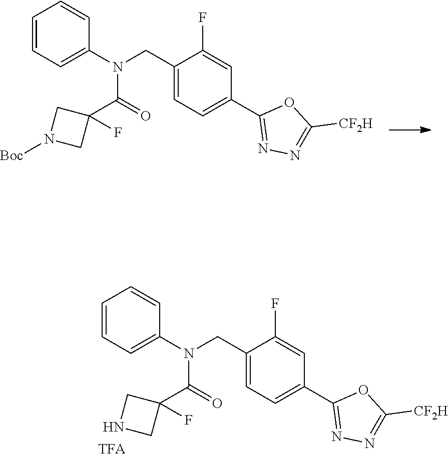 [SMILES: CC(C)(C)OC(=O)N1CC(F)(C(=O)N(Cc2ccc(-c3nnc(C(F)F)o3)cc2F)c2ccccc2)C1 ; O=C(N(Cc1ccc(-c2nnc(C(F)F)o2)cc1F)c1ccccc1)C1(F)CNC1]

Tert-butyl 3-((4-(5-(difluoromethyl)-1,3,4-oxadiazol-2-yl)-2-fluorobenzyl)(phenyl)carbamoyl)-3-fluoroazetidine-1-carboxylate (0.527 g, 1.013 mmol) prepared in step 1 and trifluoroacetic acid (1.551 mL, 20.251 mmol) were dissolved in dichloromethane (10 mL) at room temperature, and the resulting solution was stirred at the same temperature for 18 hours. After removing the solvent from the reaction mixture under reduced pressure, the title compound (0.425 g, 99.9%) was obtained as a brown gel.

### [Step 3] Synthesis of Compound 3429

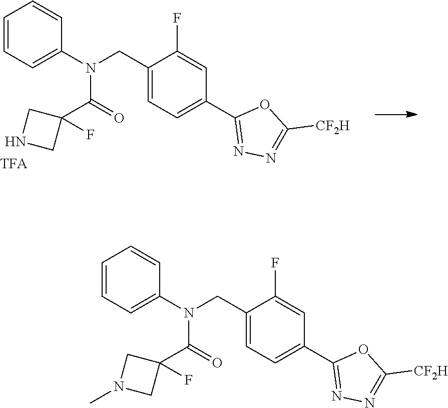 [SMILES: O=C(N(Cc1ccc(-c2nnc(C(F)F)o2)cc1F)c1ccccc1)C1(F)CNC1 ; CN1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2, formaldehyde (0.007 g, 0.238 mmol), acetic acid (0.007 mL, 0.119 mmol), and sodium triacetoxyborohydride (0.076 g, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 38.7%) as a yellow gel.

¹H NMR (400 MHz, CDCl₃) δ 7.80 (dd, J=8.0, 1.5 Hz, 1H), 7.65 (dd, J=9.8, 1.6 Hz, 1H), 7.52 (t, J=7.6 Hz, 1H), 7.26 (dd, J=6.8, 3.9 Hz, 3H), 7.04-6.97 (m, 2H), 6.84 (t, J=51.7 Hz, 1H), 4.98 (s, 2H), 3.54 (dd, J=22.2, 10.5 Hz, 2H), 3.15-2.99 (m, 2H), 2.27 (s, 3H);

LRMS (ES) m/z 436.1 (M++1).

### Example 113: Synthesis of Compound 3430, N-(4-(5-(difluoromethyl)-1,3,4-oxadiazol-2-yl)-2-fluorobenzyl)-1-ethyl-3-fluoro-N-phenylazetidine-3-carboxamide

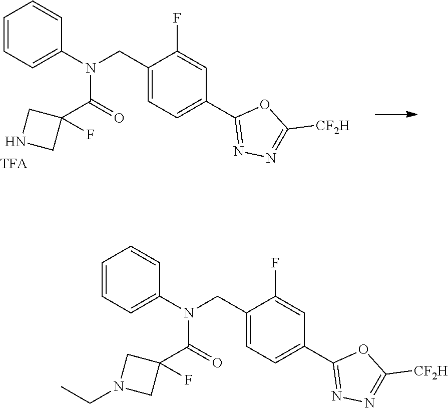 [SMILES: O=C(N(Cc1ccc(-c2nnc(C(F)F)o2)cc1F)c1ccccc1)C1(F)CNC1 ; CCN1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, acetaldehyde (0.010 g, 0.238 mmol), acetic acid (0.007 mL, 0.119 mmol), and sodium triacetoxyborohydride (0.076 g, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 37.5%) as a yellow gel.

¹H NMR (400 MHz, CDCl₃) δ 7.89 (dd, J=8.0, 1.5 Hz, 1H), 7.74 (dd, J=9.8, 1.6 Hz, 1H), 7.62 (t, J=7.6 Hz, 1H), 7.38-7.32 (m, 3H), 7.13-7.07 (m, 2H), 6.95 (dd, J=70.2, 33.2 Hz, 1H), 5.07 (s, 2H), 3.57 (dd, J=22.5, 10.3 Hz, 2H), 3.11 (dd, J=21.7, 10.4 Hz, 2H), 2.48 (q, J=7.1 Hz, 2H), 0.95 (t, J=7.2 Hz, 3H);

LRMS (ES) m/z 450.1 (M++1).

### Example 114: Synthesis of Compound 3431, N-(4-(5-(difluoromethyl)-1,3,4-oxadiazol-2-yl)-2-fluorobenzyl)-3-fluoro-1-isopropyl-N-phenylazetidine-3-carboxamide

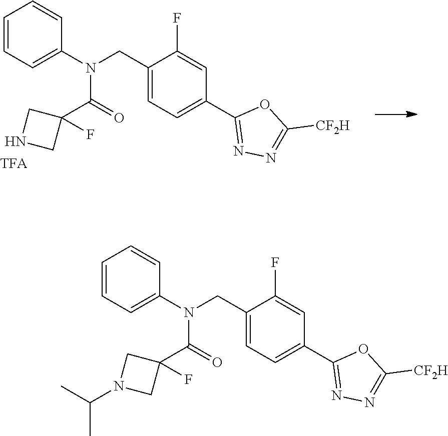 [SMILES: O=C(N(Cc1ccc(-c2nnc(C(F)F)o2)cc1F)c1ccccc1)C1(F)CNC1 ; CC(C)N1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, propan-2-one (0.014 g, 0.238 mmol), acetic acid (0.007 mL, 0.119 mmol), and sodium triacetoxyborohydride (0.076 g, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 36.4%) as a yellow gel.

¹H NMR (400 MHz, CDCl₃) δ 7.80 (dd, J=8.0, 1.5 Hz, 1H), 7.64 (dd, J=9.8, 1.6 Hz, 1H), 7.53 (t, J=7.6 Hz, 1H), 7.25 (dd, J=6.9, 3.7 Hz, 3H), 7.05-6.98 (m, 2H), 6.84 (t, J=51.7 Hz, 1H), 4.98 (s, 2H), 3.46 (dd, J=22.7, 10.3 Hz, 2H), 3.00 (dd, J=21.6, 10.4 Hz, 2H), 2.28-2.15 (m, 1H), 0.81 (d, J=6.2 Hz, 6H);

LRMS (ES) m/z 464.4 (M++1).

### Example 115: Synthesis of Compound 3432, 1-acetyl-N-(4-(5-(difluoromethyl)-1,3,4-oxadiazol-2-yl)-2-fluorobenzyl)-3-fluoro-N-phenylazetidine-3-carboxamide

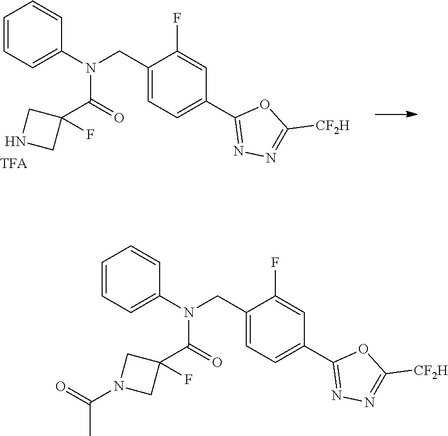 [SMILES: O=C(N(Cc1ccc(-c2nnc(C(F)F)o2)cc1F)c1ccccc1)C1(F)CNC1 ; CC(=O)N1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, acetyl chloride (0.013 mL, 0.178 mmol), and triethylamine (0.050 mL, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 36.4%) as a yellow gel.

¹H NMR (400 MHz, CDCl₃) δ 7.90 (dd, J=8.0, 1.4 Hz, 1H), 7.76 (dd, J=9.8, 1.5 Hz, 1H), 7.56 (t, J=7.6 Hz, 1H), 7.41-7.35 (m, 3H), 7.12-6.78 (m, 3H), 5.14 (d, J=14.6 Hz, 1H), 5.03 (d, J=14.8 Hz, 1H), 4.77 (dd, J=21.6, 10.9 Hz, 1H), 4.29 (dd, J=22.7, 12.4 Hz, 1H), 4.03 (dd, J=22.4, 10.2 Hz, 1H), 3.63 (dd, J=23.4, 11.9 Hz, 1H), 1.89 (s, 3H);

LRMS (ES) m/z 463.4 (M++1).

### Example 116: Synthesis of Compound 3433, N-(4-(5-(difluoromethyl)-1,3,4-oxadiazol-2-yl)-2-fluorobenzyl)-3-fluoro-N-phenyl-1-propionylazetidine-3-carboxamide

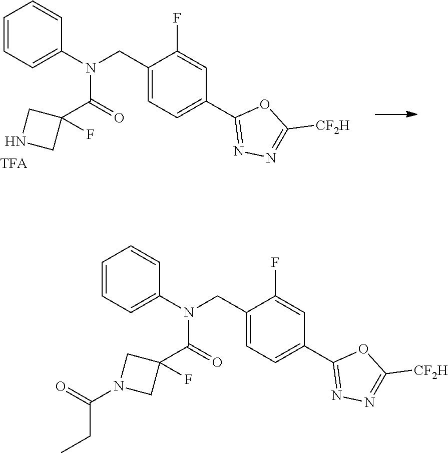 [SMILES: O=C(N(Cc1ccc(-c2nnc(C(F)F)o2)cc1F)c1ccccc1)C1(F)CNC1 ; CCC(=O)N1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, propionyl chloride (0.017 g, 0.178 mmol), and triethylamine (0.050 mL, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 35.3%) as a yellow gel.

¹H NMR (400 MHz, CDCl₃) δ 7.90 (dd, J=8.0, 1.5 Hz, 1H), 7.76 (dd, J=9.8, 1.6 Hz, 1H), 7.56 (t, J=7.6 Hz, 1H), 7.40-7.35 (m, 3H), 7.11-6.78 (m, 3H), 5.19-4.98 (m, 2H), 4.82-4.67 (m, 1H), 4.29 (dd, J=22.1, 11.3 Hz, 1H), 4.00 (dd, J=22.2, 8.8 Hz, 1H), 3.63 (dd, J=22.3, 10.6 Hz, 1H), 2.11 (q, J=7.5 Hz, 2H), 1.12 (t, J=7.5 Hz, 3H);

LRMS (ES) m/z 478.2 (M++1).

### Example 117: Synthesis of Compound 3434, N-(4-(5-(difluoromethyl)-1,3,4-oxadiazol-2-yl)-2-fluorobenzyl)-3-fluoro-1-isobutyryl N-phenylazetidine-3-carboxamide

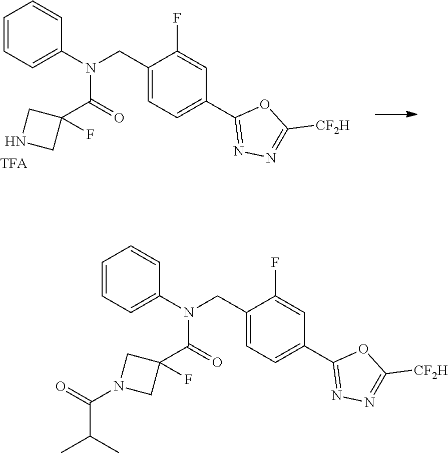 [SMILES: O=C(N(Cc1ccc(-c2nnc(C(F)F)o2)cc1F)c1ccccc1)C1(F)CNC1 ; CC(C)C(=O)N1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, isobutyryl chloride (0.019 g, 0.178 mmol), and triethylamine (0.050 mL, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 34.3%) as a yellow gel.

¹H NMR (400 MHz, CDCl₃) δ 7.90 (dd, J=8.0, 1.5 Hz, 1H), 7.76 (dd, J=9.8, 1.6 Hz, 1H), 7.56 (t, J=7.6 Hz, 1H), 7.41-7.35 (m, 3H), 7.13-6.79 (m, 3H), 5.20-4.98 (m, 2H), 4.80 (dd, J=21.6, 10.1 Hz, 1H), 4.28 (dd, J=22.6, 11.9 Hz, 1H), 4.04 (dd, J=22.6, 10.2 Hz, 1H), 3.62 (dd, J=23.4, 11.8 Hz, 1H), 2.41 (dt, J=13.6, 6.8 Hz, 1H), 1.09 (t, J=7.8 Hz, 6H);

LRMS (ES) m/z 492.3 (M++1).

### Example 118: Synthesis of Compound 3435, N-(4-(5-(difluoromethyl)-1,3,4-oxadiazol-2-yl)-2-fluorobenzyl)-3-fluoro-N-phenyl-1-(2,2,2-trifluoroacetyl)azetidine-3-carboxamide

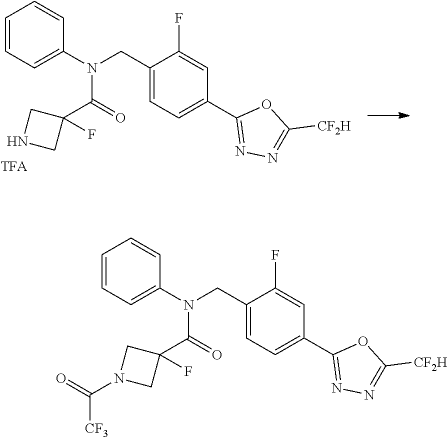 [SMILES: O=C(N(Cc1ccc(-c2nnc(C(F)F)o2)cc1F)c1ccccc1)C1(F)CNC1 ; O=C(N1CC(F)(C(=O)N(Cc2ccc(-c3nnc(C(F)F)o3)cc2F)c2ccccc2)C1)C(F)(F)F]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, 3,3,3-trifluoropropanoic anhydride (0.042 g, 0.178 mmol), and triethylamine (0.050 mL, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 32.6%) as a yellow gel.

¹H NMR (400 MHz, CDCl₃) δ 7.91 (dd, J=8.0, 1.6 Hz, 1H), 7.77 (dd, J=9.8, 1.6 Hz, 1H), 7.56 (t, J=7.6 Hz, 1H), 7.44-7.38 (m, 3H), 7.11-6.78 (m, 3H), 5.10 (dd, J=37.8, 14.6 Hz, 2H), 4.96 (dd, J=22.4, 11.8 Hz, 1H), 4.49 (dd, J=22.2, 12.5 Hz, 1H), 4.23 (dd, J=22.0, 11.9 Hz, 1H), 3.81 (dd, J=23.2, 13.5 Hz, 1H);

LRMS (ES) m/z 516.9 (M++1).

### Example 119: Synthesis of Compound 3436, N-(4-(5-(difluoromethyl)-1,3,4-oxadiazol-2-yl)-2-fluorobenzyl)-3-fluoro-1-(methylsulfonyl)-N-phenylazetidine-3-carboxamide

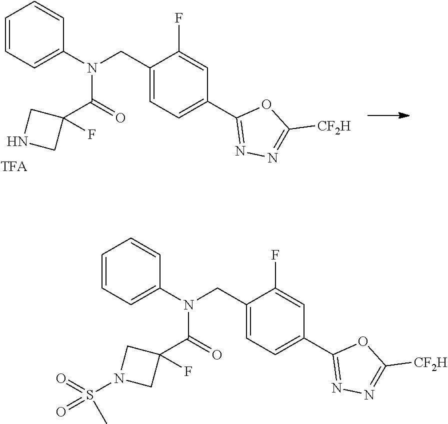 [SMILES: O=C(N(Cc1ccc(-c2nnc(C(F)F)o2)cc1F)c1ccccc1)C1(F)CNC1 ; CS(=O)(=O)N1CC(F)(C(=O)N(Cc2ccc(-c3nnc(C(F)F)o3)cc2F)c2ccccc2)C1]

N-(4-(5-(difluoromethyl)-1,3,4-oxadiazol-2-yl)-2-fluorobenzyl)-3-fluoro-N-phenylazetidine-3-carboxamide 2,2,2-trifluoroacetate (0.050 g, 0.119 mmol) prepared in step 2 of Example 112, methanesulfonyl chloride (0.014 mL, 0.178 mmol), and triethylamine (0.050 mL, 0.357 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 33.7%) as a yellow gel.

¹H NMR (400 MHz, CDCl₃) δ 7.90 (dd, J=8.0, 1.6 Hz, 1H), 7.76 (dd, J=9.8, 1.6 Hz, 1H), 7.57 (t, J=7.6 Hz, 1H), 7.42-7.35 (m, 3H), 7.12-6.78 (m, 3H), 5.09 (s, 2H), 4.40 (dd, J=23.1, 11.7 Hz, 2H), 3.68 (dd, J=22.2, 11.7 Hz, 2H), 2.89 (s, 3H);

LRMS (ES) m/z 499.0 (M++1).

### Example 120: Synthesis of Compound 3437, N-(4-(5-(difluoromethyl)-1,3,4-oxadiazol-2-yl)-2-fluorobenzyl)-4-fluoro-1-isopropyl-N-phenylpiperidine-4-carboxamide

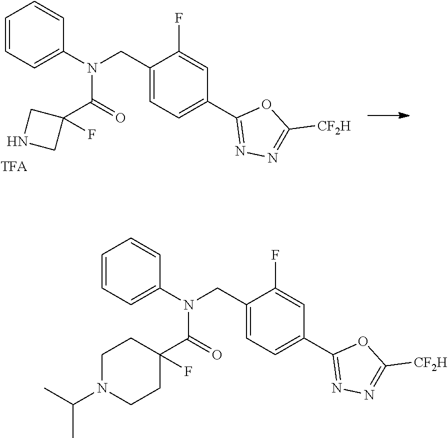 [SMILES: O=C(N(Cc1ccc(-c2nnc(C(F)F)o2)cc1F)c1ccccc1)C1(F)CNC1 ; CC(C)N1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.050 g, 0.112 mmol) prepared in step 4 of Example 1, propan-2-one (0.013 g, 0.223 mmol), acetic acid (0.007 mL, 0.112 mmol), and sodium triacetoxyborohydride (0.071 g, 0.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 36.6%) as a yellow gel.

¹H NMR (400 MHz, CDCl₃) δ 7.89 (dd, J=8.0, 1.6 Hz, 1H), 7.73 (dd, J=9.8, 1.6 Hz, 1H), 7.59 (t, J=7.6 Hz, 1H), 7.35-7.30 (m, 3H), 6.99 (ddd, J=92.0, 32.0, 26.7 Hz, 3H), 5.04 (s, 2H), 2.84 (d, J=29.5 Hz, 3H), 2.47 (d, J=46.4 Hz, 4H), 1.97 (s, 2H), 1.08 (d, J=6.3 Hz, 6H);

LRMS (ES) m/z 491.0 (M++1).

### Example 121: Synthesis of Compound 3438, 1-acetyl-N-(4-(5-(difluoromethyl)-1,3,4-oxadiazol-2-yl)-2-fluorobenzyl)-4-fluoro-N-phenylpiperidine-4-carboxamide

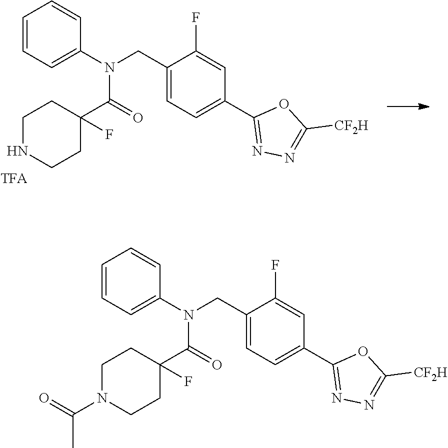 [SMILES: O=C(N(Cc1ccc(-c2nnc(C(F)F)o2)cc1F)c1ccccc1)C1(F)CCNCC1 ; CC(=O)N1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.050 g, 0.112 mmol) prepared in step 4 of Example 1, acetyl chloride (0.012 mL, 0.167 mmol), and triethylamine (0.047 mL, 0.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 36.6%) as a yellow gel.

¹H NMR (400 MHz, CDCl₃) δ 7.89 (dd, J=8.0, 1.6 Hz, 1H), 7.75 (dd, J=9.8, 1.6 Hz, 1H), 7.55 (t, J=7.6 Hz, 1H), 7.34 (dd, J=6.9, 3.7 Hz, 3H), 7.08-6.78 (m, 3H), 5.04 (d, J=7.9 Hz, 2H), 4.49 (d, J=10.3 Hz, 1H), 3.68 (s, 1H), 3.23 (s, 1H), 2.73 (s, 1H), 2.42-2.22 (m, 2H), 2.11 (s, 3H), 1.96 (d, J=8.9 Hz, 2H);

LRMS (ES) m/z 491.1 (M++1).

### Example 122: Synthesis of Compound 3439, N-(4-(5-(difluoromethyl)-1,3,4-oxadiazol-2-yl)-2-fluorobenzyl)-4-fluoro-N-phenyl-1-propionylpiperidine-4-carboxamide

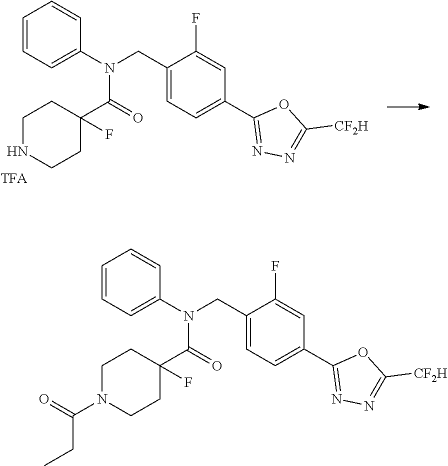 [SMILES: O=C(N(Cc1ccc(-c2nnc(C(F)F)o2)cc1F)c1ccccc1)C1(F)CCNCC1 ; CCC(=O)N1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.050 g, 0.112 mmol) prepared in step 4 of Example 1, propionyl chloride (0.015 mL, 0.167 mmol), and triethylamine (0.047 mL, 0.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 35.6%) as a yellow gel.

¹H NMR (400 MHz, CDCl₃) δ 7.89 (dd, J=8.0, 1.5 Hz, 1H), 7.75 (dd, J=9.8, 1.6 Hz, 1H), 7.56 (t, J=7.6 Hz, 1H), 7.37-7.31 (m, 3H), 6.99 (ddd, J=88.9, 33.1, 28.3 Hz, 3H), 5.04 (s, 2H), 4.49 (s, 1H), 3.74 (s, 1H), 3.16 (s, 1H), 2.73 (s, 1H), 2.41-2.12 (m, 4H), 1.95 (s, 2H), 1.16 (dd, J=8.9, 6.0 Hz, 3H);

LRMS (ES) m/z 505.3 (M++1).

### Example 123: Synthesis of Compound 3440, N-(4-(5-(difluoromethyl)-1,3,4-oxadiazol-2-yl)-2-fluorobenzyl)-4-fluoro-1-isobutyryl N-phenylpiperidine-4-carboxamide

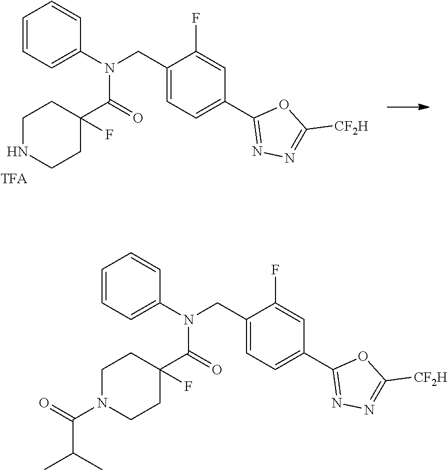 [SMILES: O=C(N(Cc1ccc(-c2nnc(C(F)F)o2)cc1F)c1ccccc1)C1(F)CCNCC1 ; CC(C)C(=O)N1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.050 g, 0.112 mmol) prepared in step 4 of Example 1, isobutyryl chloride (0.018 mL, 0.167 mmol), and triethylamine (0.047 mL, 0.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 34.6%) as a yellow gel.

¹H NMR (400 MHz, CDCl₃) δ 7.89 (dd, J=8.0, 1.6 Hz, 1H), 7.75 (dd, J=9.8, 1.6 Hz, 1H), 7.56 (t, J=7.6 Hz, 1H), 7.34 (dd, J=6.9, 3.7 Hz, 3H), 6.99 (ddd, J=90.2, 32.5, 27.7 Hz, 3H), 5.04 (d, J=9.1 Hz, 2H), 4.52 (d, J=11.0 Hz, 1H), 3.82 (d, J=9.3 Hz, 1H), 3.18 (d, J=14.5 Hz, 1H), 2.85-2.56 (m, 2H), 2.25 (ddd, J=52.7, 39.7, 12.9 Hz, 2H), 1.96 (d, J=10.4 Hz, 2H), 1.14 (dd, J=7.3, 4.9 Hz, 6H);

LRMS (ES) m/z 519.4 (M++1).

### Example 124: Synthesis of Compound 3441, N-(4-(5-(difluoromethyl)-1,3,4-oxadiazol-2-yl)-2-fluorobenzyl)-4-fluoro-N-phenyl-1-(2,2,2-trifluoroacetyl)piperidine-4-carboxamide

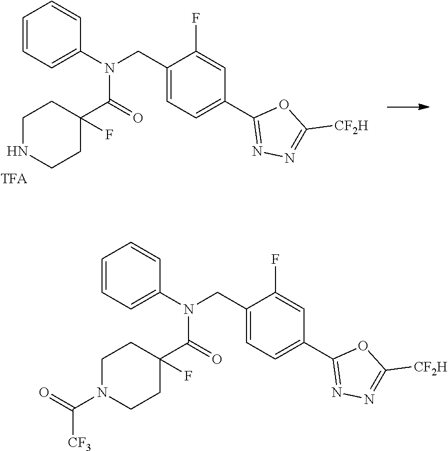 [SMILES: O=C(N(Cc1ccc(-c2nnc(C(F)F)o2)cc1F)c1ccccc1)C1(F)CCNCC1 ; O=C(N1CCC(F)(C(=O)N(Cc2ccc(-c3nnc(C(F)F)o3)cc2F)c2ccccc2)CC1)C(F)(F)F]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.050 g, 0.112 mmol) prepared in step 4 of Example 1, 3,3,3-trifluoropropanoic anhydride (0.040 g, 0.167 mmol), and triethylamine (0.047 mL, 0.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO 2 plate, 20×20×1 mm; dichloromethane/methanol=0% to 10%) and concentrated to obtain the title compound (0.020 g, 32.9%) as a yellow gel.

¹H NMR (400 MHz, CDCl₃) δ 7.89 (dd, J=8.0, 1.6 Hz, 1H), 7.76 (dd, J=9.8, 1.6 Hz, 1H), 7.55 (t, J=7.6 Hz, 1H), 7.38-7.33 (m, 3H), 7.08-6.79 (m, 3H), 5.04 (d, J=4.7 Hz, 2H), 4.42 (d, J=13.1 Hz, 1H), 3.91 (d, J=13.1 Hz, 1H), 3.29 (t, J=12.8 Hz, 1H), 2.94 (t, J=12.7 Hz, 1H), 2.46-2.22 (m, 2H), 2.08-1.98 (m, 2H);

LRMS (ES) m/z 546.2 (M++1).

### Example 125: Synthesis of Compound 3442, N-(4-(5-(difluoromethyl)-1,3,4-oxadiazol-2-yl)-2-fluorobenzyl)-4-fluoro-1-(methylsulfonyl)-N-phenylpiperidine-4-carboxamide

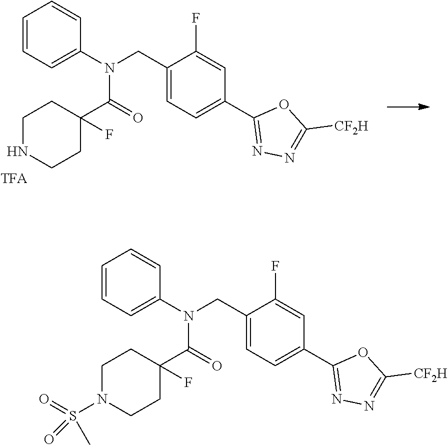 [SMILES: O=C(N(Cc1ccc(-c2nnc(C(F)F)o2)cc1F)c1ccccc1)C1(F)CCNCC1 ; CS(=O)(=O)N1CCC(F)(C(=O)N(Cc2ccc(-c3nnc(C(F)F)o3)cc2F)c2ccccc2)CC1]

N-(4-(5-(difluoromethyl)-1,3,4-oxadiazol-2-yl)-2-fluorobenzyl)-4-fluoro-N-phenylpiperidine-4-carboxamide 2,2,2-trifluoroacetate (0.050 g, 0.112 mmol) prepared in step 4 of Example 1, methanesulfonyl chloride (0.013 mL, 0.167 mmol), and triethylamine (0.047 mL, 0.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.020 g, 34.1%) as a yellow gel.

¹H NMR (400 MHz, CDCl₃) δ 7.90 (dd, J=8.0, 1.6 Hz, 1H), 7.75 (dd, J=9.9, 1.6 Hz, 1H), 7.56 (t, J=7.6 Hz, 1H), 7.38-7.31 (m, 3H), 7.10-6.79 (m, 3H), 5.05 (s, 2H), 3.69 (d, J=11.3 Hz, 2H), 2.89-2.78 (m, 2H), 2.75 (s, 3H), 2.57-2.32 (m, 2H), 2.06 (d, J=9.1 Hz, 2H);

LRMS (ES) m/z 527.1 (M++1).

### Example 126: Synthesis of Compound 3443, N-((5-(5-(difluoromethyl)-1,3,4-oxadiazol-2-yl)pyridin-2-yl)methyl)-1-ethyl-4-fluoro N-(3-fluorophenyl)piperidine-4-carboxamide

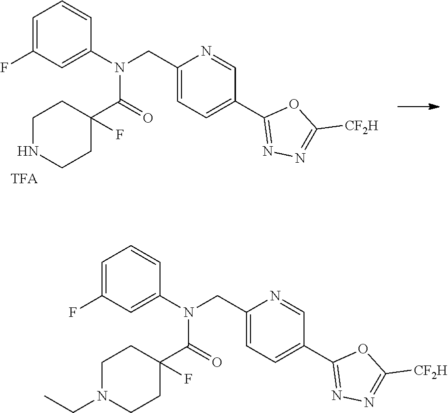 [SMILES: O=C(N(Cc1ccc(-c2nnc(C(F)F)o2)cn1)c1cccc(F)c1)C1(F)CCNCC1 ; CCN1CCC(F)(C(=O)N(Cc2ccc(-c3nnc(C(F)F)o3)cn2)c2cccc(F)c2)CC1]

N-((5-(5-(difluoromethyl)-1,3,4-oxadiazol-2-yl)pyridin-2-yl)methyl)-4-fluoro-N-(3-fluorophenyl)piperidine-4-carboxamide 2,2,2-trifluoroacetate (0.200 g, 0.445 mmol) prepared in step 3 of Example 10, acetaldehyde (0.039 g, 0.890 mmol), acetic acid (0.025 mL, 0.445 mmol), and sodium triacetoxyborohydride (0.283 g, 1.335 mmol) were dissolved in dichloromethane (4 mL) at room temperature, and the resulting solution was stirred at the same temperature for 18 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by chromatography (SiO₂ plate, 20×20×1 mm; dichloromethane/methanol=0% to 10%) and concentrated to obtain the title compound (0.080 g, 37.7%) as a white solid.

¹H NMR (400 MHz, CDCl₃) δ 9.27 (d, J=1.6 Hz, 1H), 8.39 (dd, J=8.2, 2.2 Hz, 1H), 7.53 (d, J=8.3 Hz, 1H), 7.37-7.29 (m, 1H), 7.11-6.78 (m, 4H), 5.07 (s, 2H), 2.86 (s, 2H), 2.43 (dd, J=39.0, 9.5 Hz, 4H), 2.24 (d, J=10.4 Hz, 2H), 2.02 (d, J=18.6 Hz, 2H), 1.12 (t, J=7.2 Hz, 3H);

LRMS (ES) m/z 478.3 (M++1).

### Example 127: Synthesis of Compound 6890, tert-butyl 3-fluoro-3-((3-fluorophenyl)((5-(5-(trifluoromethyl)-1,3,4-oxadiazol-2-yl)pyridin-2-yl)methyl)carbamoyl)azetidine-1-carboxylate

### [Step 1] Synthesis of 2-(6-methylpyridin-3-yl)-5-(trifluoromethyl)-1,3,4-oxadiazole

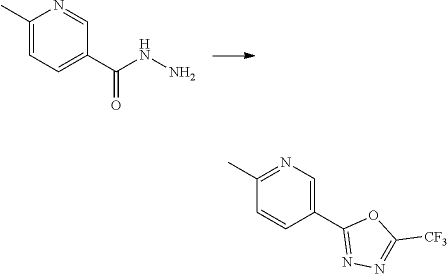 [SMILES: Cc1ccc(C(=O)NN)cn1 ; Cc1ccc(-c2nnc(C(F)(F)F)o2)cn1]

6-Methylnicotinohydrazide (2.000 g, 13.230 mmol) prepared in step 1 of Example 6 and imidazole (2.702 g, 39.690 mmol) were dissolved in dichloromethane (5 mL), and trifluoroacetic anhydride (5.606 mL, 39.690 mmol) was added at 0° C. and then heated to reflux for 16 hours. Next, the temperature was lowered to room temperature to terminate the reaction. Water was poured into the reaction mixture, followed by extraction with dichloromethane. The organic layer was washed with a saturated aqueous sodium chloride solution, and dried over anhydrous magnesium sulfate. The obtained product was filtered and concentrated under reduced pressure to obtain the title compound (2.650 g, 87.4%) as a yellow solid.

### [Step 2] Synthesis of 2-(6-(bromomethyl)pyridin-3-yl)-5-(trifluoromethyl)-1,3,4-oxadiazole

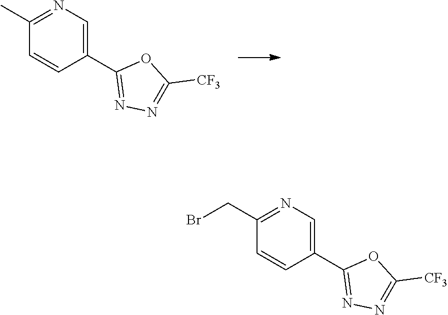 [SMILES: Cc1ccc(-c2nnc(C(F)(F)F)o2)cn1 ; FC(F)(F)c1nnc(-c2ccc(CBr)nc2)o1]

2-(6-Methylpyridin-3-yl)-5-(trifluoromethyl)-1,3,4-oxadiazole (2.650 g, 11.564 mmol) prepared in step 1 was dissolved in 1,2-dichloroethane (100 mL), and azobisisobutyronitrile (AIBN, 0.190 g, 1.156 mmol) and 1-bromopyrrolidine-2,5-one (NBS, 2.676 g, 15.033 mmol) were added at room temperature and heated to reflux for 16 hours. The reaction was terminated by lowering the temperature to room temperature. Water was poured into the reaction mixture, followed by extraction with dichloromethane. The organic layer was washed with a saturated aqueous sodium chloride solution, dried over anhydrous magnesium sulfate, filtered, and concentrated under reduced pressure. The concentrate was purified by column chromatography (SiO₂, 80 g cartridge; ethyl acetate/hexane=15%) and concentrated to obtain the title compound (0.750 g, 21.1%) as a red solid.

### [Step 3] Synthesis of 3-fluoro-N-((5-(5-(trifluoromethyl)-1,3,4-oxadiazol-2-yl)pyridin-2-yl)methyl)aniline

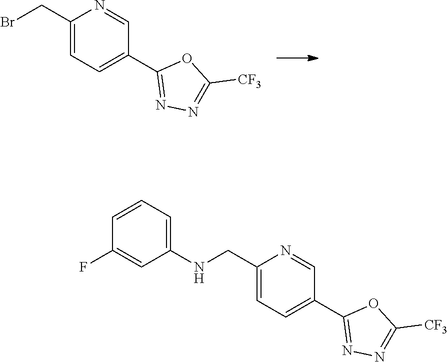 [SMILES: FC(F)(F)c1nnc(-c2ccc(CBr)nc2)o1 ; Fc1cccc(NCc2ccc(-c3nnc(C(F)(F)F)o3)cn2)c1]

2-(6-(Bromomethyl)pyridin-3-yl)-5-(trifluoromethyl)-1,3,4-oxadiazole (0.500 g, 1.623 mmol) prepared in step 2, 3-fluoroaniline (0.271 g, 2.435 mmol), potassium carbonate (0.336 g, 2.435 mmol), and potassium iodide (0.135 g, 0.812 mmol) were dissolved in N,N-dimethylformamide (50 mL) at room temperature, and the resulting solution was stirred at the same temperature for 18 hours. The solvent was removed from the reaction mixture under reduced pressure. A saturated aqueous ammonium chloride solution was poured into the obtained concentrate, followed by extraction with dichloromethane. The organic layer was washed with water, dried over anhydrous magnesium sulfate, filtered, and concentrated under reduced pressure. The concentrate was purified by column chromatography (SiO₂, 24 g cartridge; ethyl acetate/hexane=0% to 70%) and concentrated to obtain the title compound (0.280 g, 51.0%) as a yellow solid.

### [Step 4] Synthesis of Compound 6890

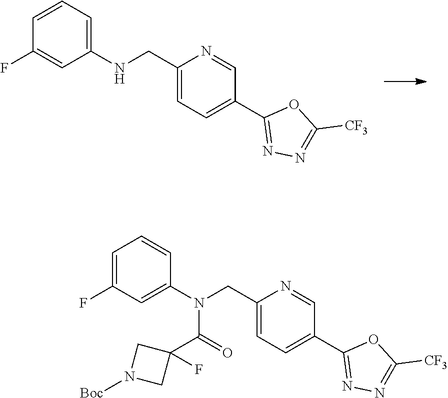 [SMILES: Fc1cccc(NCc2ccc(-c3nnc(C(F)(F)F)o3)cn2)c1 ; CC(C)(C)OC(=O)N1CC(F)(C(=O)N(Cc2ccc(-c3nnc(C(F)(F)F)o3)cn2)c2cccc(F)c2)C1]

To a solution in which tert-butyl 3-(chlorocarbonyl)-3-fluoroazetidine-1-carboxylate (0.137 g, 0.576 mmol) prepared in step 1 of Example 14 was dissolved in dichloromethane (20 mL) at room temperature, 3-fluoro-N-((5-(5-(trifluoromethyl)-1,3,4-oxadiazol-2-yl)pyridin-2-yl)methyl)aniline (0.150 g, 0.443 mmol) prepared in step 3 was added and stirred at the same temperature. Triethylamine (0.185 mL, 1.330 mmol) was added to the reaction mixture and further stirred at the same temperature for 16 hours. The solvent was removed from the reaction mixture under reduced pressure. A saturated aqueous ammonium chloride solution was poured into the obtained concentrate, followed by extraction with dichloromethane. The organic layer was washed with water, dried over anhydrous magnesium sulfate, filtered, and concentrated under reduced pressure. The concentrate was purified by column chromatography (SiO₂, 24 g cartridge; ethyl acetate/hexane=0% to 60%) and concentrated to obtain the title compound (0.150 g, 62.7%) as a yellow solid.

¹H NMR (400 MHz, CDCl₃) δ 9.30 (d, J=2.1 Hz, 1H), 8.41 (dt, J=7.8, 3.9 Hz, 1H), 7.56 (d, J=8.3 Hz, 1H), 7.37 (dd, J=14.7, 7.6 Hz, 1H), 7.09 (t, J=10.5 Hz, 3H), 5.10 (s, 2H), 4.63-4.33 (m, 2H), 3.83 (d, J=45.0 Hz, 2H), 1.44 (s, 9H).

### Example 128: Synthesis of Compound 6891, 3-fluoro-N-(3-fluorophenyl)-1-methyl-N-((5-(5-(trifluoromethyl)-1,3,4-oxadiazol-2-yl)pyridin-2-yl)methyl)azetidine-3-carboxamide

### [Step 1] Synthesis of 3-fluoro-N-(3-fluorophenyl)-N-((5-(5-(trifluoromethyl)-1,3,4-oxadiazol-2-yl)pyridin-2-yl)methyl)azetidine-3-carboxamide 2,2,2-trifluoroacetate

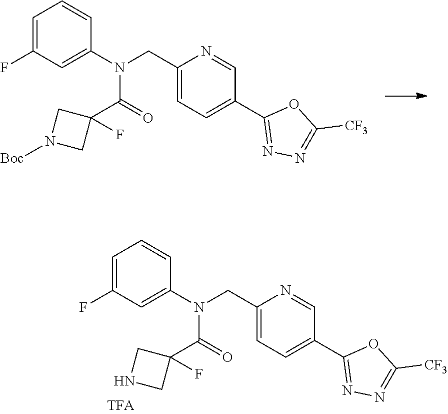 [SMILES: CC(C)(C)OC(=O)N1CC(F)(C(=O)N(Cc2ccc(-c3nnc(C(F)(F)F)o3)cn2)c2cccc(F)c2)C1 ; O=C(N(Cc1ccc(-c2nnc(C(F)(F)F)o2)cn1)c1cccc(F)c1)C1(F)CNC1]

Tert-butyl 3-fluoro-3-((3-fluorophenyl)((5-(5-(trifluoromethyl)-1,3,4-oxadiazol-2-yl)pyridin-2-yl)methyl)carbamoyl)azetidine-1-carboxylate (0.080 g, 0.148 mmol) prepared in step 4 of Example 127 and trifluoroacetic acid (0.227 mL, 2.966 mmol) were dissolved in dichloromethane (5 mL) at room temperature, and the resulting solution was stirred at the same temperature for 18 hours. After removing the solvent from the reaction mixture under reduced pressure, the title compound (0.065 g, 99.8%) was obtained as a yellow gel.

### [Step 2] Synthesis of Compound 6891

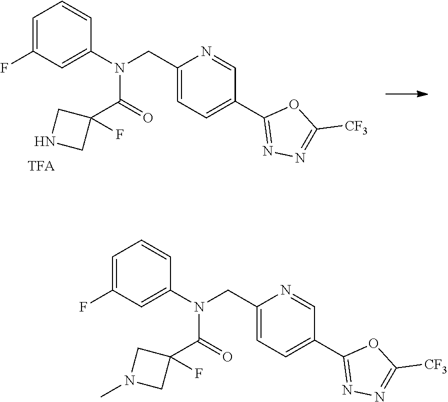 [SMILES: O=C(N(Cc1ccc(-c2nnc(C(F)(F)F)o2)cn1)c1cccc(F)c1)C1(F)CNC1 ; CN1CC(F)(C(=O)N(Cc2ccc(-c3nnc(C(F)(F)F)o3)cn2)c2cccc(F)c2)C1]

3-Fluoro-N-(3-fluorophenyl)-N-((5-(5-(trifluoromethyl)-1,3,4-oxadiazol-2-yl)pyridin-2-yl)methyl)azetidine-3-carboxamide 2,2,2-trifluoroacetate (0.070 g, 0.159 mmol) prepared in step 1, formaldehyde (0.010 g, 0.319 mmol), acetic acid (0.009 mL, 0.159 mmol), and sodium triacetoxyborohydride (0.101 g, 0.478 mmol) were dissolved in dichloromethane (5 mL) at room temperature, and the resulting solution was stirred at the same temperature for 3 hours. A saturated aqueous sodium bicarbonate solution was poured into the reaction mixture, followed by extraction with dichloromethane. The obtained product was filtered through a plastic filter to remove a solid residue and an aqueous layer, and then concentrated under reduced pressure. The concentrate was purified by column chromatography (SiO₂, 4 g cartridge; dichloromethane/methanol=0% to 10%) and concentrated to obtain the title compound (0.050 g, 69.2%) as a yellow gel.

¹H NMR (400 MHz, CDCl₃) δ 9.28 (d, J=2.0 Hz, 1H), 8.40 (dd, J=8.2, 2.2 Hz, 1H), 7.59 (d, J=8.2 Hz, 1H), 7.39-7.31 (m, 1H), 7.13-7.00 (m, 3H), 5.10 (s, 2H), 3.68 (dd, J=21.6, 9.4 Hz, 2H), 3.25 (dd, J=21.5, 8.8 Hz, 2H), 2.37 (s, 3H);

LRMS (ES) m/z 453.6 (M++1).

### Activity Measurement and Analysis Protocol of the Compounds of the Present Invention

### <Experimental Example 1> HDAC Enzyme Activity Inhibition Assay (In Vitro)

In order to confirm the selectivity of the compounds represented by Chemical Formula I of the present invention to HDAC6 through HDAC1 and HDAC6 enzyme activity inhibition experiments, a comparison experiment was performed using the compound that has already been developed as a control group.

HDAC enzyme activity was measured using the HDAC Fluorimetric Drug Discovery Kit (Enzo Life Sciences, Inc., BML-AK511, 516). For the HDAC1 enzyme activity test, human recombinant HDAC1 (BML-SE456) was used as an enzyme source and Fluor de Lys©-SIRT1 (BNL-K1177) was used as a substrate. After dispensing 5-fold diluted compounds into a 96-well plate, 0.3 μg of enzyme and 10 μM substrate were added to each well of the plate and allowed to react at 30° C. for 60 minutes. Next, Fluor de Lys© Developer II (BML-K1176) was added and reacted for 30 minutes to complete the reaction, and then the fluorescence values (Ex 360, Em 460) were measured using a multi-plate reader (Flexstation 3, Molecular Device). The HDAC6 enzymes were tested using human recombinant HDAC6 (382180) from Calbiochem Inc., according to the same protocol as the HDAC1 enzyme activity test method. With respect to the final result values, respective IC₅₀ values were calculated using GraphPad Prism 4.0 program (Table 2).

TABLE 2
Results of HDAC enzyme activity inhibition assay

| | | | | HDAC6 |
|---|---|---|---|---|
| | | | | selectivity |
| Ex. | Comp. | HDAC1 (nM) | HDAC6 (nM) | (fold) |
| 1 | 2865 | >30,000 | 1,000 | 30 |
| 2 | 2866 | >20,000 | 134.1 | 149 |
| 3 | 2867 | >20,000 | 204.2 | 97 |
| 4 | 2868 | >20,000 | 413.2 | 48 |
| 5 | 2869 | >50,000 | 107.7 | 464 |
| 6 | 2951 | 42,560 | 74.7 | 569 |
| 7 | 2952 | >50,000 | 99.1 | 504 |
| 8 | 2953 | 43,999 | 80.9 | 543 |
| 9 | 2954 | 39,564 | 63.4 | 624 |
| 10 | 2969 | >50,000 | 38.5 | 1,298 |
| 11 | 2970 | >50,000 | 47.8 | 1,046 |
| 12 | 2971 | >50,000 | 67.5 | 740 |
| 13 | 2972 | >50,000 | 42.0 | 1,190 |
| 14 | 2973 | >50,000 | 46.5 | 1,075 |
| 15 | 2974 | >50,000 | 47.4 | 1,054 |
| 16 | 2975 | >50,000 | 48.9 | 1,022 |
| 17 | 2976 | >50,000 | 51.6 | 968 |
| 18 | 2995 | >50,000 | 83.3 | 600 |
| 19 | 2996 | 24,893 | 56.1 | 443 |
| 20 | 2997 | 13,485 | 77.7 | 173 |
| 21 | 2998 | 17,269 | 39.0 | 442 |
| 22 | 2999 | >50,000 | 62.4 | 801 |
| 23 | 3000 | >50,000 | 43.2 | 1,157 |
| 24 | 3001 | >50,000 | 50.0 | 1,000 |
| 25 | 3002 | >50,000 | 53.0 | 943 |
| 26 | 3003 | 27,498 | 37.4 | 735 |
| 27 | 3004 | 26,065 | 28.4 | 917 |
| 28 | 3005 | 29,469 | 26.9 | 1,095 |
| 29 | 3006 | >50,000 | 24.6 | 2,032 |
| 30 | 3007 | >50,000 | 25.7 | 1,945 |
| 31 | 3047 | 20,781 | 85.7 | 242 |
| 32 | 3048 | 18,527 | 78.0 | 237 |
| 33 | 3049 | 21,590 | 55.0 | 392 |
| 34 | 3050 | >50,000 | 73.2 | 683 |
| 35 | 3051 | >50,000 | 53.7 | 931 |
| 36 | 3052 | >50,000 | 58.5 | 854 |
| 37 | 3053 | >50,000 | 74.8 | 668 |
| 38 | 3054 | >50,000 | 25.9 | 1,930 |
| 39 | 3055 | >50,000 | 46.2 | 1,082 |
| 40 | 3090 | >50,000 | 22.6 | 2,212 |
| 41 | 3091 | >50,000 | 34.8 | 1,436 |
| 42 | 3092 | >50,000 | 36.0 | 1,388 |
| 43 | 3093 | >50,000 | 32.4 | 1,543 |
| 44 | 3094 | >50,000 | 26.3 | 1,901 |
| 45 | 3095 | >50,000 | 21.1 | 2,369 |
| 46 | 3096 | >50,000 | 36.1 | 1,385 |
| 47 | 3097 | >50,000 | 31.0 | 1,612 |
| 48 | 3098 | >50,000 | 25.1 | 1,992 |
| 49 | 3105 | >50,000 | 64.8 | 771 |
| 50 | 3106 | >50,000 | 39.6 | 1,262 |
| 51 | 3107 | >50,000 | 47.2 | 1,059 |
| 52 | 3108 | >50,000 | 27.4 | 1,827 |
| 53 | 3109 | >50,000 | 29.4 | 1,700 |
| 54 | 3110 | >50,000 | 42.7 | 1,170 |
| 55 | 3111 | >50,000 | 53.7 | 931 |
| 56 | 3112 | >50,000 | 24.7 | 2,024 |
| 57 | 3113 | >50,000 | 32.4 | 1,543 |
| 58 | 3114 | >50,000 | 32.8 | 1,524 |
| 59 | 3115 | >50,000 | 24.5 | 2,040 |
| 60 | 3152 | >50,000 | 36.3 | 1,377 |
| 61 | 3153 | >50,000 | 37.1 | 1,347 |
| 62 | 3154 | >50,000 | 33.2 | 1,506 |
| 63 | 3155 | >50,000 | 37.7 | 1,326 |
| 64 | 3156 | >50,000 | 77.0 | 649 |
| 65 | 3157 | >50,000 | 53.1 | 941 |
| 66 | 3158 | >50,000 | 35.7 | 1,400 |
| 67 | 3159 | >50,000 | 30.3 | 1,650 |
| 68 | 3160 | >50,000 | 34.1 | 1,466 |
| 69 | 3161 | >50,000 | 42.4 | 1,179 |
| 70 | 3162 | >50,000 | 22.0 | 2,272 |
| 71 | 3163 | >50,000 | 23.2 | 2,155 |
| 72 | 3164 | >50,000 | 17.8 | 2,808 |
| 73 | 3165 | >50,000 | 24.2 | 2,066 |
| 74 | 3166 | >50,000 | 67.4 | 741 |
| 75 | 3167 | >50,000 | 23.3 | 2,145 |
| 76 | 3168 | >50,000 | 17.2 | 2,906 |
| 77 | 3169 | >50,000 | 17.8 | 2,808 |
| 78 | 3170 | >50,000 | 24.2 | 2,066 |
| 79 | 3171 | >50,000 | 67.4 | 741 |
| 80 | 3172 | >50,000 | 22.8 | 2,192 |
| 81 | 3216 | >50,000 | 57.1 | 875 |
| 82 | 3217 | >50,000 | 34.7 | 1,440 |
| 83 | 3218 | >50,000 | 42.1 | 1,187 |
| 84 | 3219 | >50,000 | 50.6 | 988 |
| 85 | 3220 | >50,000 | 39.3 | 1,272 |
| 86 | 3221 | >50,000 | 33.5 | 1,492 |
| 87 | 3222 | >50,000 | 35.2 | 1,420 |
| 88 | 3223 | >50,000 | 35.1 | 1,424 |
| 89 | 3224 | >50,000 | 37.7 | 1,326 |
| 90 | 3389 | 28,499 | 32.2 | 885 |
| 91 | 3390 | 24,171 | 23.8 | 1,015 |
| 92 | 3391 | 28,455 | 20.9 | 1,361 |
| 93 | 3392 | 44,624 | 19.0 | 2,348 |
| 94 | 3393 | >50,000 | 18.4 | 2,717 |
| 95 | 3394 | >50,000 | 26.5 | 1,886 |
| 96 | 3395 | >50,000 | 36.0 | 1,388 |
| 97 | 3396 | 31,556 | 19.5 | 1,618 |
| 98 | 3397 | 23,717 | 23.2 | 1,022 |
| 99 | 3398 | 27,494 | 20.0 | 1,374 |
| 100 | 3399 | 31,537 | 20.1 | 1,569 |
| 101 | 3400 | 36,613 | 21.6 | 1,695 |
| 102 | 3401 | 44,498 | 22.8 | 1,951 |
| 103 | 3402 | >50,000 | 20.8 | 2,403 |
| 104 | 3403 | 26,260 | 28.8 | 911 |
| 105 | 3404 | 25,435 | 32.9 | 773 |
| 106 | 3405 | 20,439 | 26.5 | 771 |
| 107 | 3406 | 26,583 | 36.2 | 734 |
| 108 | 3407 | 48,009 | 18.6 | 2,581 |
| 109 | 3408 | 37,009 | 32.6 | 1,135 |
| 110 | 3409 | >50,000 | 33.4 | 1,497 |
| 111 | 3410 | 34,654 | 25.8 | 1,343 |
| 112 | 3429 | 30,902 | 73.4 | 421 |
| 113 | 3430 | >50,000 | 80.1 | 624 |
| 114 | 3431 | >50,000 | 91.8 | 544 |
| 115 | 3432 | 29,097 | 79.5 | 366 |
| 116 | 3433 | 38,534 | 85.9 | 448 |
| 117 | 3434 | >50,000 | 113.2 | 441 |
| 118 | 3435 | >50,000 | 153.7 | 325 |
| 119 | 3436 | >50,000 | 132.1 | 378 |
| 120 | 3437 | >50,000 | 221.1 | 226 |
| 121 | 3438 | >50,000 | 165.5 | 302 |
| 122 | 3439 | >50,000 | 209.7 | 238 |
| 123 | 3440 | >50,000 | 280.1 | 178 |
| 124 | 3441 | >50,000 | 506.9 | 98 |
| 125 | 3442 | >50,000 | 324.2 | 154 |
| 126 | 3443 | >50,000 | 51.7 | 967 |
| 127 | 6890 | >50,000 | 877 | 57 |
| 128 | 6891 | >50,000 | 254.3 | 196 |

As shown in Table 2 above, it was found from the results of the activity inhibition assay for HDAC1 and HDAC6 that the 1,3,4-oxadiazole derivative compound of the present invention, the optical isomer thereof, or the pharmaceutically acceptable salt thereof exhibited about 30 to about 2906 times higher selective HDAC6 inhibitory activity.

### <Experimental Example 2> Analysis of Effect of HDAC6-Specific Inhibitor on Mitochondrial Axonal Transport (In Vitro)

The effect of the HDAC6-specific inhibitor on mitochondrial axonal transport was analyzed. Specifically, in order to confirm whether the compound represented by Chemical Formula I of the present invention selectively inhibited the HDAC6 activity to increase the acetylation of tubulin, which is a major substrate of HDAC6, thereby improving the mitochondrial axonal transport rates reduced by amyloid-beta treatment in neuronal axons, a comparison experiment was performed using the material that has already been developed as a control group.

Hippocampal neurons from Sprague-Dawley (SD) rat embryos at embryonic day 17-18 (E17-18) were cultured for 7 days in an extracellular matrix-coated culture dish for imaging, and then treated with 1M of amyloid-beta peptide fragments. After 24 hours, the compound was treated on the 8th day of in vitro culture, and 3 hours later, treated with MitoTracker Red CMXRos (Life Technologies, NY, USA) for the last 5 minutes to stain the mitochondria. With regard to the axonal transport of the stained neuron mitochondria, the transport rates of each mitochondrion were determined using the IMARIS analysis software (BITPLANE, Zurich, Switzerland) by taking images using a confocal microscope (Leica 5P8; Leica Microsystems, UK) at 1-second intervals for 1 minute.

As a result, it was confirmed that the 1,3,4-oxadiazole derivative compound of the present invention, the optical isomer thereof or the pharmaceutically acceptable salts thereof showed an improvement effect on the rates of mitochondrial axonal transport.

1. A 1,3,4-oxadiazole derivative compound represented by Chemical Formula I below, an optical isomer thereof, or a pharmaceutically acceptable salt thereof: 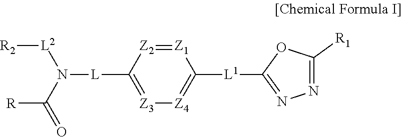
in the Chemical Formula I above,
Z₁ to Z₄ are each independently N, CH or CX, wherein Z₁ to Z₄ may not be three or more Ns at the same time;
L, L¹ or L² is each independently —(C₀-C₂alkylene)-;
R₁ is —CH₂X or —CX₃;
R₂ is aryl or heteroaryl, wherein at least one —H of the aryl or heteroaryl may each independently be substituted with —X, —OH, —(C₁-C₄alkyl) or —O(C₁-C₄alkyl);
R is 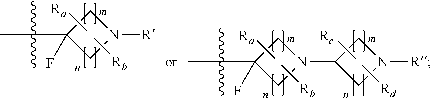
Rₐ to R_{d} are each independently —H or —(C₁-C₄alkyl);
R′ and R″ are each independently —H, —(C₁-C₄alkyl), —(C₃-C₇cycloalkyl), —(C₂-C₆heterocycloalkyl), —(C₁-C₄alkyl)-(C₃-C₇cycloalkyl), —(C₁-C₄alkyl)-(C₂-C₆heterocycloalkyl), —C(═O)—(C₁-C₄alkyl), —C(═O)—(C₃-C₇cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄alkyl) may be substituted with —X, —OH, —N(CH₃)₂ or —O(C₁-C₄alkyl), and at least one —H of —(C₃-C₇cycloalkyl), —(C₂-C₆heterocycloalkyl), —(C₁-C₄alkyl)-(C₃-C₇cycloalkyl), —(C₁-C₄alkyl)-(C₂-C₆heterocycloalkyl) or —C(═O)—(C₃-C₇cycloalkyl) ring may be substituted with —X, —OH or —(C₁-C₄alkyl);
m or n is each independently 1, 2 or 3; and
X is F, Cl, Br or I.

2. The 1,3,4-oxadiazole derivative compound represented by Chemical Formula I, the optical isomer thereof, or the pharmaceutically acceptable salt thereof according to claim 1, wherein in the Chemical Formula I above,
Z₁ to Z₄ are each independently N, CH or CX, wherein Z₁ to Z₄ may not be two or more Ns at the same time;
L, L¹ or L² is each independently —(C₀-C₂alkylene)-;
R₁ is —CH₂X or —CX₃;
R₂ is aryl, wherein at least one —H of the aryl may each independently be substituted with —X, —OH, —(C₁-C₄alkyl) or —O(C₁-C₄alkyl);
R is 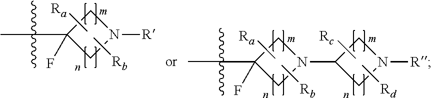
Rₐ to R_{d} are each independently —H or —(C₁-C₄alkyl);
R′ and R″ are each independently —H, —(C₁-C₄alkyl), —(C₃-C₇cycloalkyl), —(C₂-C₆heterocycloalkyl), —(C₁-C₄alkyl)-(C₃-C₇cycloalkyl), —(C₁-C₄alkyl)-(C₂-C₆heterocycloalkyl), —C(═O)—(C₁-C₄alkyl), —C(═O)—(C₃-C₇cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄alkyl) may be substituted with —X, —OH, —N(CH₃)₂ or —O(C₁-C₄alkyl), and at least one —H of —(C₃-C₇cycloalkyl), —(C₂-C₆heterocycloalkyl), —(C₁-C₄alkyl)-(C₃-C₇cycloalkyl), —(C₁-C₄alkyl)-(C₂-C₆heterocycloalkyl) or —C(═O)—(C₃-C₇cycloalkyl) ring may be substituted with —X, —OH or —(C₁-C₄alkyl);
m or n is each independently 1, 2 or 3; and
X is F, Cl or Br.

3. The 1,3,4-oxadiazole derivative compound represented by Chemical Formula I, the optical isomer thereof, or the pharmaceutically acceptable salt thereof according to claim 2, wherein in the Chemical Formula I above,
Z₁ to Z₄ are each independently N, CH or CX, wherein Z₁ to Z₄ may not be two or more Ns at the same time;
L is —(C₁alkylene)-;
L¹ or L² is each independently —(C₀alkylene)-;
R₁ is —CH₂X or —CX₃;
R₂ is aryl, wherein at least one —H of the aryl may each independently be substituted with —X;
R is 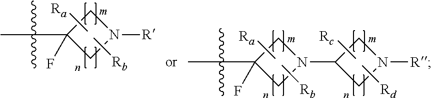
Rₐ to R_{d} are each independently —H;
R′ and R″ are each independently —H, —(C₁-C₄alkyl), —(C₃-C₇cycloalkyl), —(C₂-C₆heterocycloalkyl), —(C₁-C₄alkyl)-(C₃-C₇cycloalkyl), —(C₁-C₄alkyl)-(C₂-C₆heterocycloalkyl), —C(═O)—(C₁-C₄alkyl), —C(═O)—(C₃-C₇cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄alkyl) may be substituted with —X, —OH, —N(CH₃)₂ or —O(C₁-C₄alkyl), and at least one —H of —(C₃-C₇cycloalkyl) ring may be substituted with —X;
m or n is each independently 1 or 2; and
X is F or Cl.

4. The 1,3,4-oxadiazole derivative compound represented by Chemical Formula I, the optical isomer thereof, or the pharmaceutically acceptable salt thereof according to claim 3, wherein in the Chemical Formula I above,
Z₁ to Z₄ are each independently N, CH or CF, wherein Z₁ to Z₄ may not be two or more Ns at the same time;
L is —(C₁alkylene)-;
L¹ or L² is each independently —(C₀alkylene)-;
R₁ is —CF₂H or —CF₃;
R₂ is aryl, wherein at least one —H of the aryl may each independently be substituted with —F;
Rₐ is 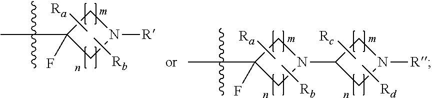
Rₐ to R_{d} are each independently —H;
R′ is —H, —(C₁-C₄alkyl), —(C₃-C₇cycloalkyl), —(C₂-C₆heterocycloalkyl), —(C₁-C₄alkyl)(C₃-C₇cycloalkyl), —(C₁-C₄alkyl)-(C₂-C₆heterocycloalkyl), —C(═O)—(C₁-C₄alkyl), —C(═O)—(C₃-C₇cycloalkyl), —C(═O)—O(C₁-C₄alkyl) or —S(═O)₂—(C₁-C₄alkyl), wherein at least one —H of —(C₁-C₄alkyl) or —C(═O)—(C₁-C₄alkyl) may be substituted with —X, —OH, —N(CH₃)₂ or —O(C₁-C₄alkyl), and at least one —H of —(C₃-C₇cycloalkyl) ring may be substituted with —X;
R″ is —(C₁-C₄alkyl), —(C₃-C₇cycloalkyl) or —(C₂-C₆heterocycloalkyl);
m or n is each independently 1 or 2; and
X is F or C₁.

5. The 1,3,4-oxadiazole derivative compound, the optical isomer thereof or the pharmaceutically acceptable salt thereof according to claim 1, wherein it is any one of compounds listed in the following table:

| Ex. | Comp. | Structure |
|---|---|---|
| 1 | 2865 | 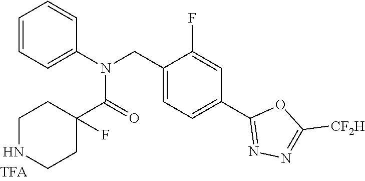 [SMILES: O=C(N(Cc1ccc(-c2nnc(C(F)F)o2)cc1F)c1ccccc1)C1(F)CCNCC1] |
| 2 | 2866 | 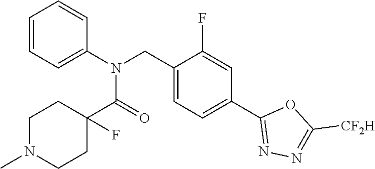 [SMILES: CN1CCC(F)(C(=O)N(Cc2ccc(-c3nnc(C(F)F)o3)cc2F)c2ccccc2)CC1] |
| 3 | 2867 | 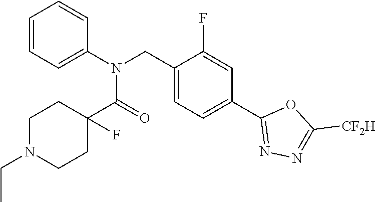 [SMILES: CCN1CCC(F)(C(=O)N(Cc2ccc(-c3nnc(C(F)F)o3)cc2F)c2ccccc2)CC1] |
| 4 | 2868 | 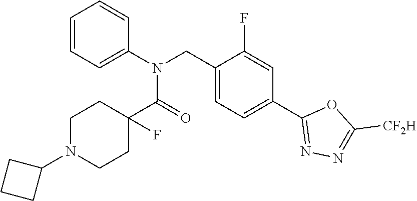 [SMILES: O=C(N(Cc1ccc(-c2nnc(C(F)F)o2)cc1F)c1ccccc1)C1(F)CCN(C2CCC2)CC1] |
| 5 | 2869 | 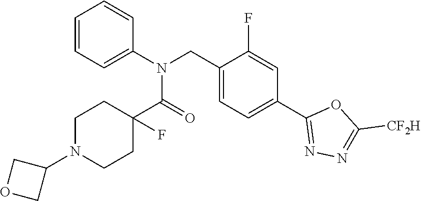 [SMILES: O=C(N(Cc1ccc(-c2nnc(C(F)F)o2)cc1F)c1ccccc1)C1(F)CCN(C2COC2)CC1] |
| 6 | 2951 | 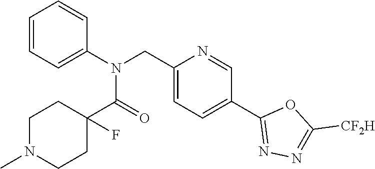 [SMILES: CN1CCC(F)(C(=O)N(Cc2ccc(-c3nnc(C(F)F)o3)cn2)c2ccccc2)CC1] |
| 7 | 2952 | 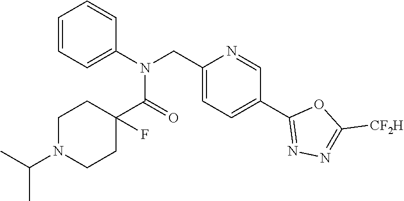 [SMILES: CC(C)N1CCC(F)(C(=O)N(Cc2ccc(-c3nnc(C(F)F)o3)cn2)c2ccccc2)CC1] |
| 8 | 2953 | 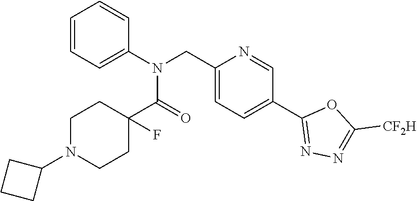 [SMILES: O=C(N(Cc1ccc(-c2nnc(C(F)F)o2)cn1)c1ccccc1)C1(F)CCN(C2CCC2)CC1] |
| 9 | 2954 | 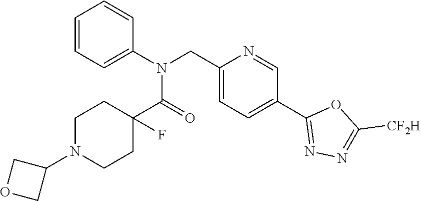 [SMILES: O=C(N(Cc1ccc(-c2nnc(C(F)F)o2)cn1)c1ccccc1)C1(F)CCN(C2COC2)CC1] |
| 10 | 2969 | 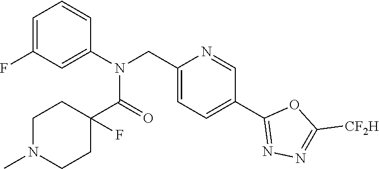 [SMILES: CN1CCC(F)(C(=O)N(Cc2ccc(-c3nnc(C(F)F)o3)cn2)c2cccc(F)c2)CC1] |
| 11 | 2970 | 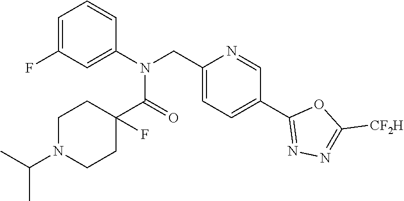 [SMILES: CC(C)N1CCC(F)(C(=O)N(Cc2ccc(-c3nnc(C(F)F)o3)cn2)c2cccc(F)c2)CC1] |
| 12 | 2971 | 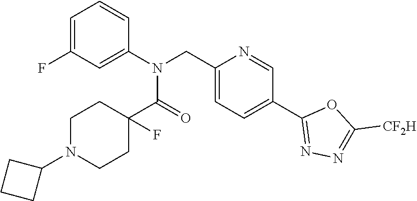 [SMILES: O=C(N(Cc1ccc(-c2nnc(C(F)F)o2)cn1)c1cccc(F)c1)C1(F)CCN(C2CCC2)CC1] |
| 13 | 2972 | 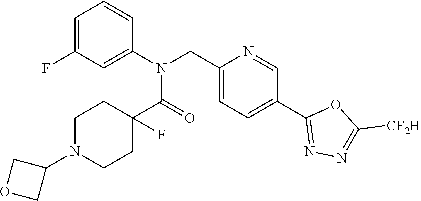 [SMILES: O=C(N(Cc1ccc(-c2nnc(C(F)F)o2)cn1)c1cccc(F)c1)C1(F)CCN(C2COC2)CC1] |
| 14 | 2973 | 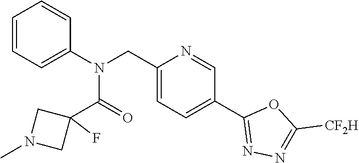 |
| 15 | 2974 | 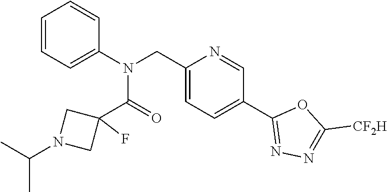 [SMILES: CC(C)N1CC(F)(C(=O)N(Cc2ccc(-c3nnc(C(F)F)o3)cn2)c2ccccc2)C1] |
| 16 | 2975 | 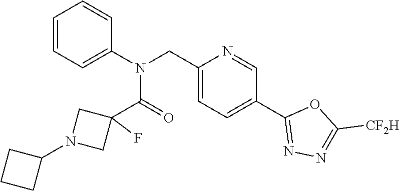 |
| 17 | 2976 | 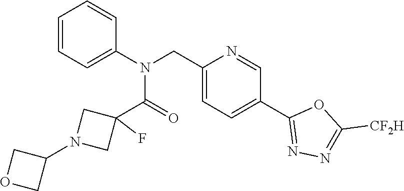 [SMILES: O=C(N(Cc1ccc(-c2nnc(C(F)F)o2)cn1)c1ccccc1)C1(F)CN(C2COC2)C1] |
| 18 | 2995 | 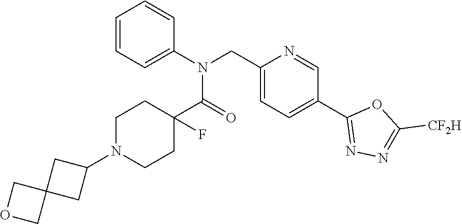 [SMILES: O=C(N(Cc1ccc(-c2nnc(C(F)F)o2)cn1)c1ccccc1)C1(F)CCN(C2CC3(COC3)C2)CC1] |
| 19 | 2996 | 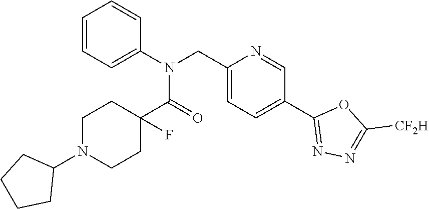 [SMILES: O=C(N(Cc1ccc(-c2nnc(C(F)F)o2)cn1)c1ccccc1)C1(F)CCN(C2CCCC2)CC1] |
| 20 | 2997 | 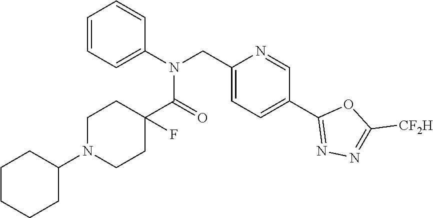 [SMILES: O=C(N(Cc1ccc(-c2nnc(C(F)F)o2)cn1)c1ccccc1)C1(F)CCN(C2CCCCC2)CC1] |
| 21 | 2998 | 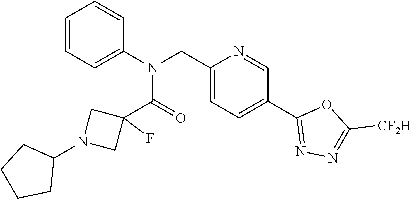 [SMILES: O=C(N(Cc1ccc(-c2nnc(C(F)F)o2)cn1)c1ccccc1)C1(F)CN(C2CCCC2)C1] |
| 22 | 2999 | 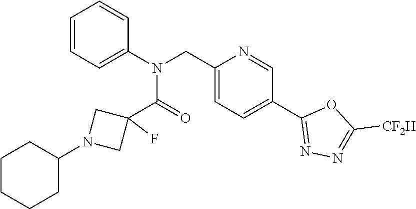 [SMILES: O=C(N(Cc1ccc(-c2nnc(C(F)F)o2)cn1)c1ccccc1)C1(F)CN(C2CCCCC2)C1] |
| 23 | 3000 | 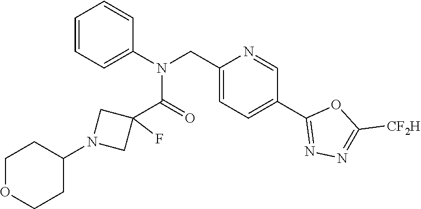 [SMILES: O=C(N(Cc1ccc(-c2nnc(C(F)F)o2)cn1)c1ccccc1)C1(F)CN(C2CCOCC2)C1] |
| 24 | 3001 | 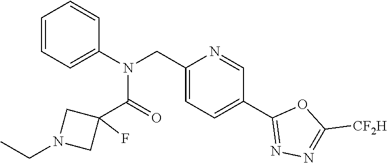 [SMILES: CCN1CC(F)(C(=O)N(Cc2ccc(-c3nnc(C(F)F)o3)cn2)c2ccccc2)C1] |
| 25 | 3002 | 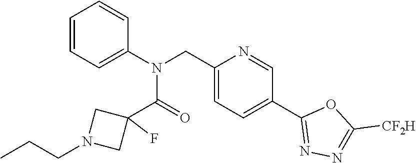 [SMILES: CCCN1CC(F)(C(=O)N(Cc2ccc(-c3nnc(C(F)F)o3)cn2)c2ccccc2)C1] |
| 26 | 3003 | 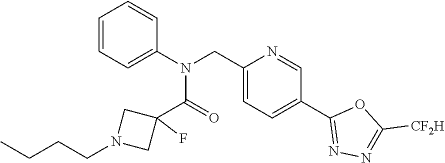 [SMILES: CCCCN1CC(F)(C(=O)N(Cc2ccc(-c3nnc(C(F)F)o3)cn2)c2ccccc2)C1] |
| 27 | 3004 | 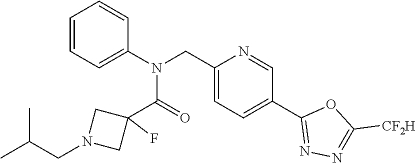 [SMILES: CC(C)CN1CC(F)(C(=O)N(Cc2ccc(-c3nnc(C(F)F)o3)cn2)c2ccccc2)C1] |
| 28 | 3005 | 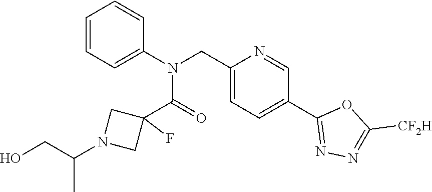 [SMILES: CC(CO)N1CC(F)(C(=O)N(Cc2ccc(-c3nnc(C(F)F)o3)cn2)c2ccccc2)C1] |
| 29 | 3006 | 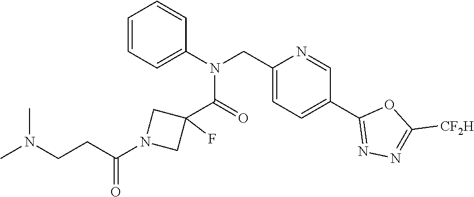 [SMILES: CN(C)CCC(=O)N1CC(F)(C(=O)N(Cc2ccc(-c3nnc(C(F)F)o3)cn2)c2ccccc2)C1] |
| 30 | 0730 | 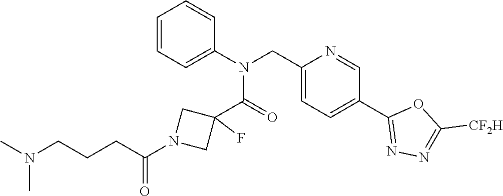 [SMILES: CN(C)CCCC(=O)N1CC(F)(C(=O)N(Cc2ccc(-c3nnc(C(F)F)o3)cn2)c2ccccc2)C1] |
| 31 | 3047 | 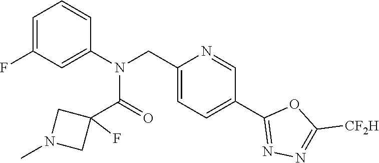 [SMILES: CN1CC(F)(C(=O)N(Cc2ccc(-c3nnc(C(F)F)o3)cn2)c2cccc(F)c2)C1] |
| 32 | 3048 | 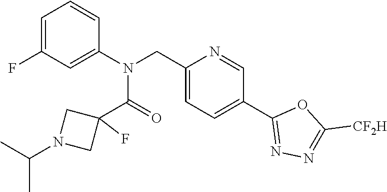 [SMILES: CC(C)N1CC(F)(C(=O)N(Cc2ccc(-c3nnc(C(F)F)o3)cn2)c2cccc(F)c2)C1] |
| 33 | 3049 | 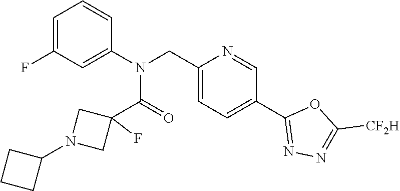 [SMILES: O=C(N(Cc1ccc(-c2nnc(C(F)F)o2)cn1)c1cccc(F)c1)C1(F)CN(C2CCC2)C1] |
| 34 | 3050 | 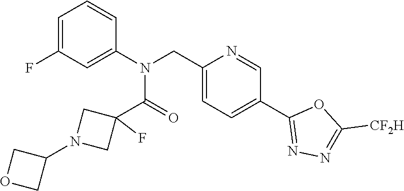 [SMILES: O=C(N(Cc1ccc(-c2nnc(C(F)F)o2)cn1)c1cccc(F)c1)C1(F)CN(C2COC2)C1] |
| 35 | 3051 | 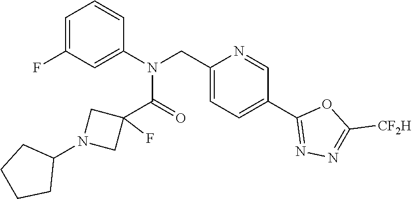 [SMILES: O=C(N(Cc1ccc(-c2nnc(C(F)F)o2)cn1)c1cccc(F)c1)C1(F)CN(C2CCCC2)C1] |
| 36 | 3052 | 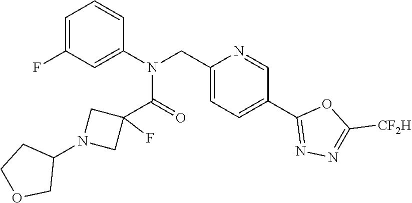 [SMILES: O=C(N(Cc1ccc(-c2nnc(C(F)F)o2)cn1)c1cccc(F)c1)C1(F)CN(C2CCOC2)C1] |
| 37 | 3053 | 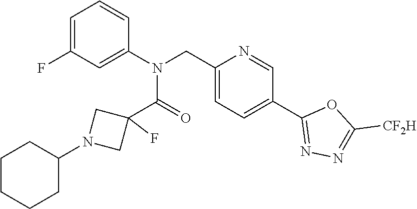 [SMILES: O=C(N(Cc1ccc(-c2nnc(C(F)F)o2)cn1)c1cccc(F)c1)C1(F)CN(C2CCCCC2)C1] |
| 38 | 3054 | 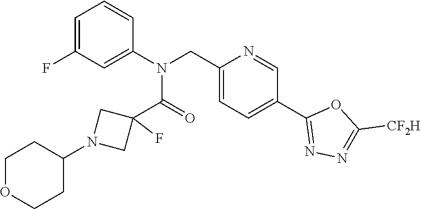 [SMILES: O=C(N(Cc1ccc(-c2nnc(C(F)F)o2)cn1)c1cccc(F)c1)C1(F)CN(C2CCOCC2)C1] |
| 39 | 3055 | 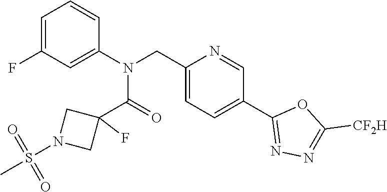 [SMILES: CS(=O)(=O)N1CC(F)(C(=O)N(Cc2ccc(-c3nnc(C(F)F)o3)cn2)c2cccc(F)c2)C1] |
| 40 | 3090 | 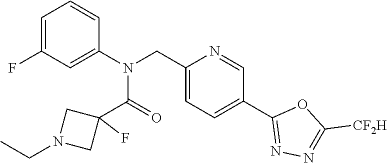 [SMILES: CCN1CC(F)(C(=O)N(Cc2ccc(-c3nnc(C(F)F)o3)cn2)c2cccc(F)c2)C1] |
| 41 | 3091 | 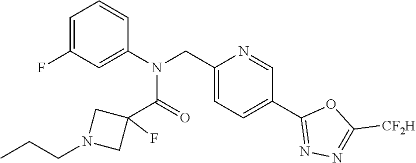 [SMILES: CCCN1CC(F)(C(=O)N(Cc2ccc(-c3nnc(C(F)F)o3)cn2)c2cccc(F)c2)C1] |
| 42 | 3092 | 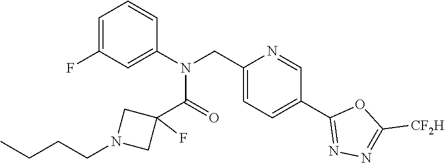 [SMILES: CCCCN1CC(F)(C(=O)N(Cc2ccc(-c3nnc(C(F)F)o3)cn2)c2cccc(F)c2)C1] |
| 43 | 3093 | 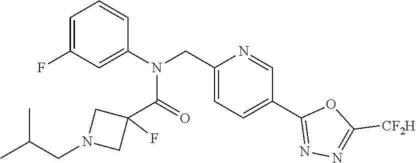 [SMILES: CC(C)CN1CC(F)(C(=O)N(Cc2ccc(-c3nnc(C(F)F)o3)cn2)c2cccc(F)c2)C1] |
| 44 | 3094 | 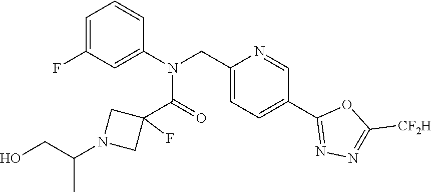 [SMILES: CC(CO)N1CC(F)(C(=O)N(Cc2ccc(-c3nnc(C(F)F)o3)cn2)c2cccc(F)c2)C1] |
| 45 | 3095 | 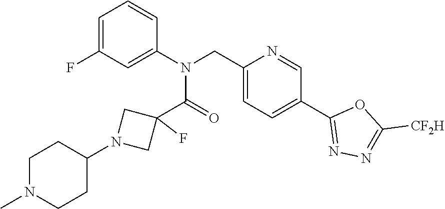 [SMILES: CN1CCC(N2CC(F)(C(=O)N(Cc3ccc(-c4nnc(C(F)F)o4)cn3)c3cccc(F)c3)C2)CC1] |
| 46 | 3096 | 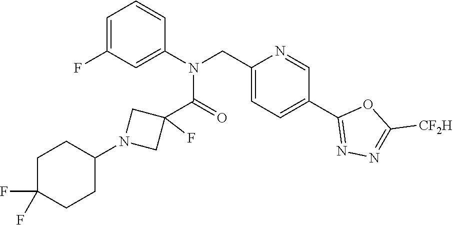 [SMILES: O=C(N(Cc1ccc(-c2nnc(C(F)F)o2)cn1)c1cccc(F)c1)C1(F)CN(C2CCC(F)(F)CC2)C1] |
| 47 | 3097 | 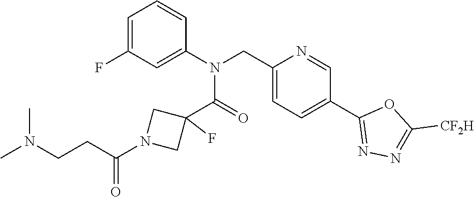 [SMILES: CN(C)CCC(=O)N1CC(F)(C(=O)N(Cc2ccc(-c3nnc(C(F)F)o3)cn2)c2cccc(F)c2)C1] |
| 48 | 3098 | 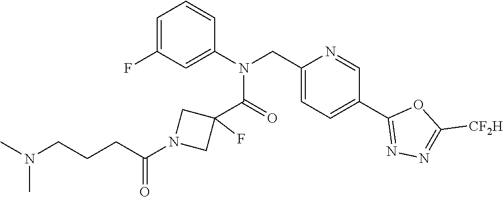 [SMILES: CN(C)CCCC(=O)N1CC(F)(C(=O)N(Cc2ccc(-c3nnc(C(F)F)o3)cn2)c2cccc(F)c2)C1] |
| 49 | 3105 | 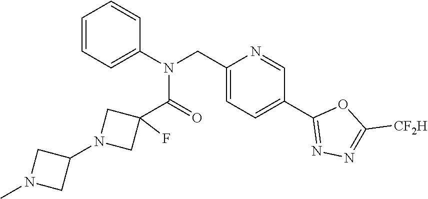 [SMILES: CN1CC(N2CC(F)(C(=O)N(Cc3ccc(-c4nnc(C(F)F)o4)cn3)c3ccccc3)C2)C1] |
| 50 | 3106 | 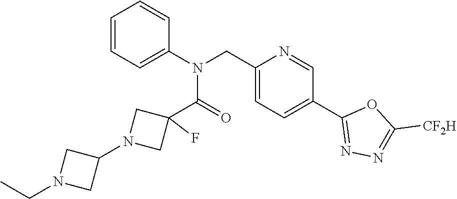 [SMILES: CCN1CC(N2CC(F)(C(=O)N(Cc3ccc(-c4nnc(C(F)F)o4)cn3)c3ccccc3)C2)C1] |
| 51 | 3107 | 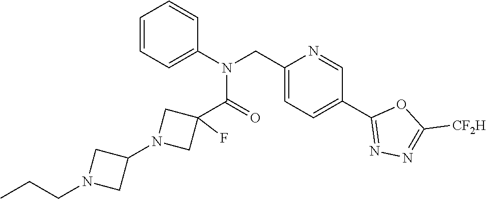 [SMILES: CCCN1CC(N2CC(F)(C(=O)N(Cc3ccc(-c4nnc(C(F)F)o4)cn3)c3ccccc3)C2)C1] |
| 52 | 3108 | 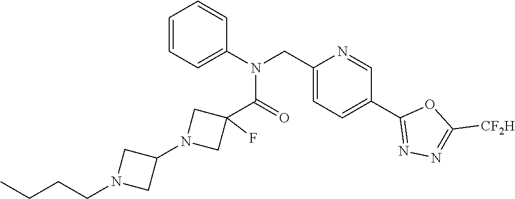 [SMILES: CCCCN1CC(N2CC(F)(C(=O)N(Cc3ccc(-c4nnc(C(F)F)o4)cn3)c3ccccc3)C2)C1] |
| 53 | 3109 | 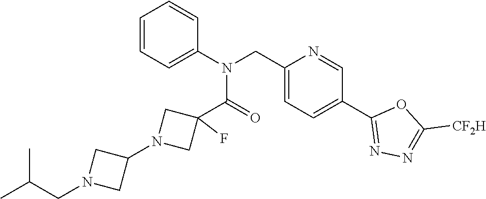 [SMILES: CC(C)CN1CC(N2CC(F)(C(=O)N(Cc3ccc(-c4nnc(C(F)F)o4)cn3)c3ccccc3)C2)C1] |
| 54 | 3110 | 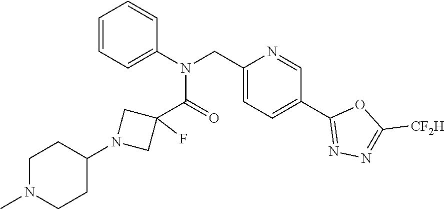 [SMILES: CN1CCC(N2CC(F)(C(=O)N(Cc3ccc(-c4nnc(C(F)F)o4)cn3)c3ccccc3)C2)CC1] |
| 55 | 3111 | 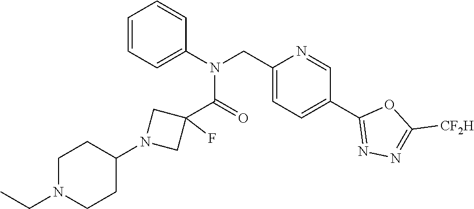 [SMILES: CCN1CCC(N2CC(F)(C(=O)N(Cc3ccc(-c4nnc(C(F)F)o4)cn3)c3ccccc3)C2)CC1] |
| 56 | 3112 | 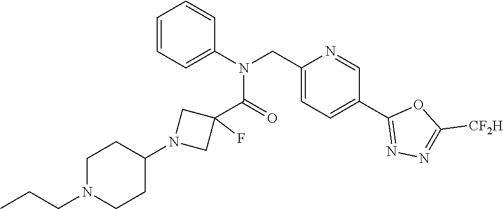 [SMILES: CCCN1CCC(N2CC(F)(C(=O)N(Cc3ccc(-c4nnc(C(F)F)o4)cn3)c3ccccc3)C2)CC1] |
| 57 | 3113 | 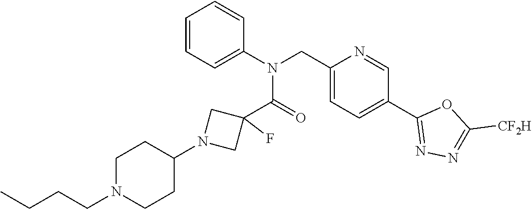 [SMILES: CCCCN1CCC(N2CC(F)(C(=O)N(Cc3ccc(-c4nnc(C(F)F)o4)cn3)c3ccccc3)C2)CC1] |
| 58 | 3114 | 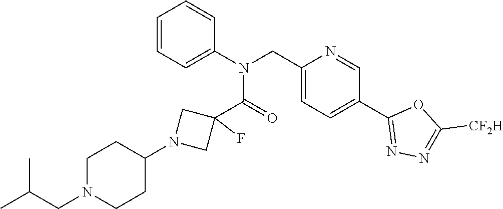 [SMILES: CC(C)CN1CCC(N2CC(F)(C(=O)N(Cc3ccc(-c4nnc(C(F)F)o4)cn3)c3ccccc3)C2)CC1] |
| 59 | 3115 | 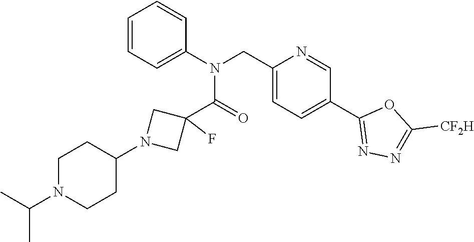 [SMILES: CC(C)N1CCC(N2CC(F)(C(=O)N(Cc3ccc(-c4nnc(C(F)F)o4)cn3)c3ccccc3)C2)CC1] |
| 60 | 3152 | 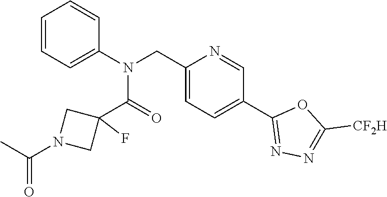 [SMILES: CC(=O)N1CC(F)(C(=O)N(Cc2ccc(-c3nnc(C(F)F)o3)cn2)c2ccccc2)C1] |
| 61 | 3153 | 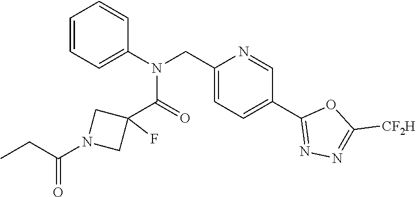 [SMILES: CCC(=O)N1CC(F)(C(=O)N(Cc2ccc(-c3nnc(C(F)F)o3)cn2)c2ccccc2)C1] |
| 62 | 3154 | 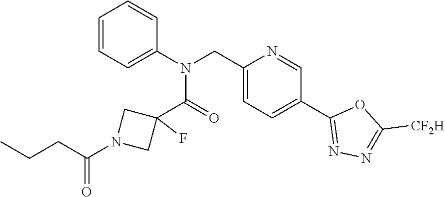 [SMILES: CCCC(=O)N1CC(F)(C(=O)N(Cc2ccc(-c3nnc(C(F)F)o3)cn2)c2ccccc2)C1] |
| 63 | 3155 | 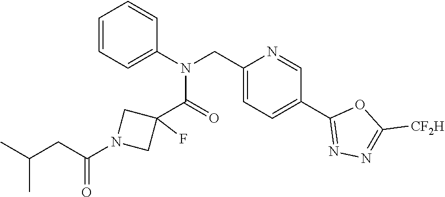 [SMILES: CC(C)CC(=O)N1CC(F)(C(=O)N(Cc2ccc(-c3nnc(C(F)F)o3)cn2)c2ccccc2)C1] |
| 64 | 3156 | 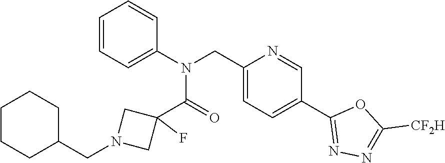 [SMILES: O=C(N(Cc1ccc(-c2nnc(C(F)F)o2)cn1)c1ccccc1)C1(F)CN(CC2CCCCC2)C1] |
| 65 | 3157 | 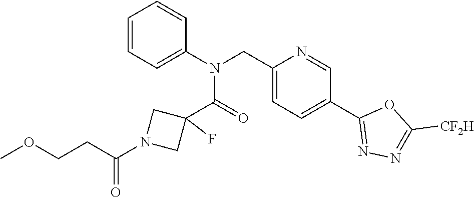 [SMILES: COCCC(=O)N1CC(F)(C(=O)N(Cc2ccc(-c3nnc(C(F)F)o3)cn2)c2ccccc2)C1] |
| 66 | 3158 | 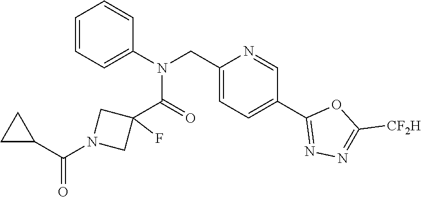 [SMILES: O=C(C1CC1)N1CC(F)(C(=O)N(Cc2ccc(-c3nnc(C(F)F)o3)cn2)c2ccccc2)C1] |
| 67 | 3159 | 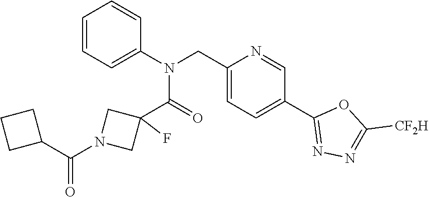 [SMILES: O=C(C1CCC1)N1CC(F)(C(=O)N(Cc2ccc(-c3nnc(C(F)F)o3)cn2)c2ccccc2)C1] |
| 68 | 3160 | 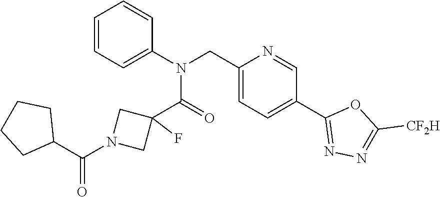 [SMILES: O=C(C1CCCC1)N1CC(F)(C(=O)N(Cc2ccc(-c3nnc(C(F)F)o3)cn2)c2ccccc2)C1] |
| 69 | 3161 | 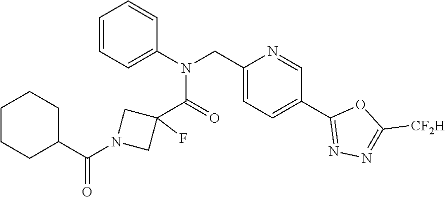 [SMILES: O=C(C1CCCCC1)N1CC(F)(C(=O)N(Cc2ccc(-c3nnc(C(F)F)o3)cn2)c2ccccc2)C1] |
| 70 | 3162 | 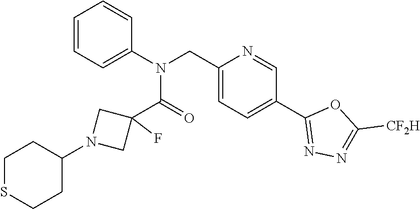 [SMILES: O=C(N(Cc1ccc(-c2nnc(C(F)F)o2)cn1)c1ccccc1)C1(F)CN(C2CCSCC2)C1] |
| 71 | 3163 | 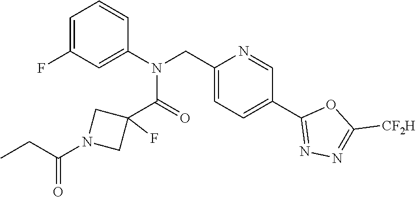 [SMILES: CCC(=O)N1CC(F)(C(=O)N(Cc2ccc(-c3nnc(C(F)F)o3)cn2)c2cccc(F)c2)C1] |
| 72 | 3164 | 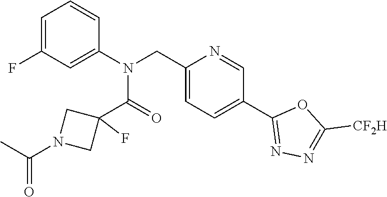 [SMILES: CC(=O)N1CC(F)(C(=O)N(Cc2ccc(-c3nnc(C(F)F)o3)cn2)c2cccc(F)c2)C1] |
| 73 | 3165 | 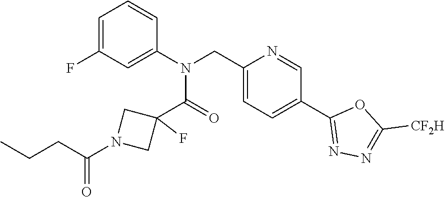 [SMILES: CCCC(=O)N1CC(F)(C(=O)N(Cc2ccc(-c3nnc(C(F)F)o3)cn2)c2cccc(F)c2)C1] |
| 74 | 3166 | 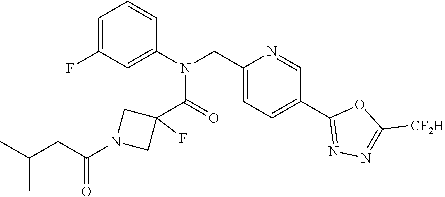 [SMILES: CC(C)CC(=O)N1CC(F)(C(=O)N(Cc2ccc(-c3nnc(C(F)F)o3)cn2)c2cccc(F)c2)C1] |
| 75 | 3167 | 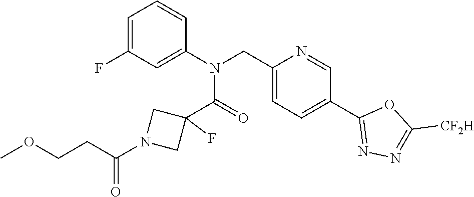 [SMILES: COCCC(=O)N1CC(F)(C(=O)N(Cc2ccc(-c3nnc(C(F)F)o3)cn2)c2cccc(F)c2)C1] |
| 76 | 3168 | 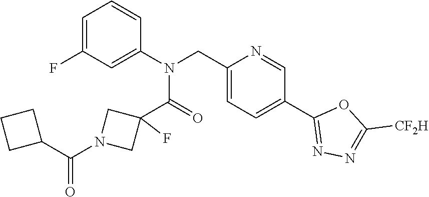 [SMILES: O=C(C1CCC1)N1CC(F)(C(=O)N(Cc2ccc(-c3nnc(C(F)F)o3)cn2)c2cccc(F)c2)C1] |
| 77 | 3169 | 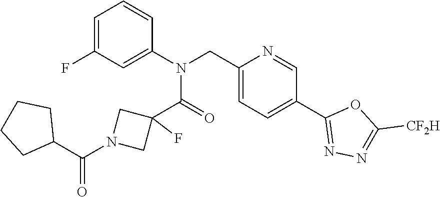 [SMILES: O=C(C1CCCC1)N1CC(F)(C(=O)N(Cc2ccc(-c3nnc(C(F)F)o3)cn2)c2cccc(F)c2)C1] |
| 78 | 3170 | 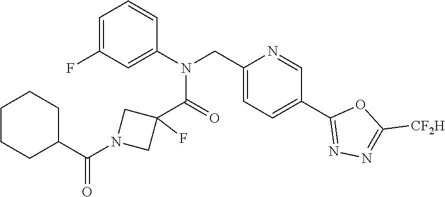 [SMILES: O=C(C1CCCCC1)N1CC(F)(C(=O)N(Cc2ccc(-c3nnc(C(F)F)o3)cn2)c2cccc(F)c2)C1] |
| 79 | 3171 | 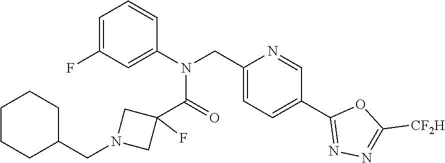 [SMILES: O=C(N(Cc1ccc(-c2nnc(C(F)F)o2)cn1)c1cccc(F)c1)C1(F)CN(CC2CCCCC2)C1] |
| 80 | 3172 | 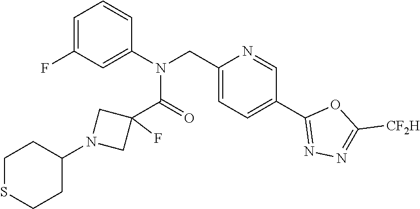 [SMILES: O=C(N(Cc1ccc(-c2nnc(C(F)F)o2)cn1)c1cccc(F)c1)C1(F)CN(C2CCSCC2)C1] |
| 81 | 3216 | 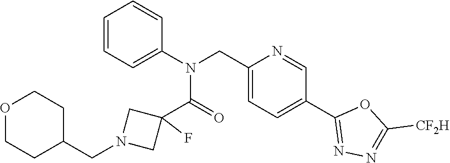 [SMILES: O=C(N(Cc1ccc(-c2nnc(C(F)F)o2)cn1)c1ccccc1)C1(F)CN(CC2CCOCC2)C1] |
| 82 | 3217 | 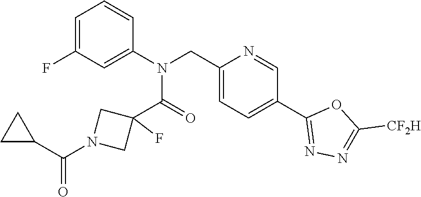 [SMILES: O=C(C1CC1)N1CC(F)(C(=O)N(Cc2ccc(-c3nnc(C(F)F)o3)cn2)c2cccc(F)c2)C1] |
| 83 | 3218 | 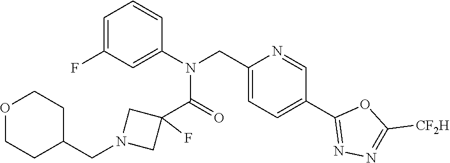 [SMILES: O=C(N(Cc1ccc(-c2nnc(C(F)F)o2)cn1)c1cccc(F)c1)C1(F)CN(CC2CCOCC2)C1] |
| 84 | 3219 | 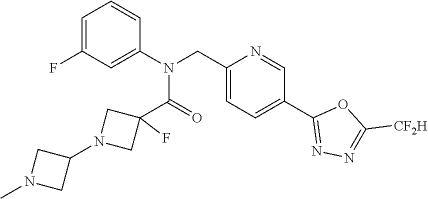 [SMILES: CN1CC(N2CC(F)(C(=O)N(Cc3ccc(-c4nnc(C(F)F)o4)cn3)c3cccc(F)c3)C2)C1] |
| 85 | 3220 | 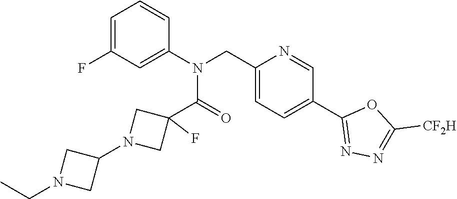 [SMILES: CCN1CC(N2CC(F)(C(=O)N(Cc3ccc(-c4nnc(C(F)F)o4)cn3)c3cccc(F)c3)C2)C1] |
| 86 | 3221 | 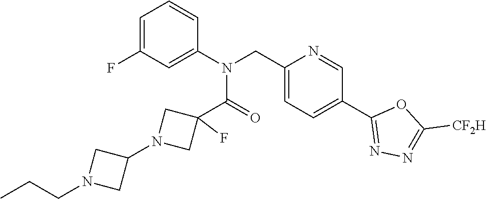 [SMILES: CCCN1CC(N2CC(F)(C(=O)N(Cc3ccc(-c4nnc(C(F)F)o4)cn3)c3cccc(F)c3)C2)C1] |
| 87 | 3222 | 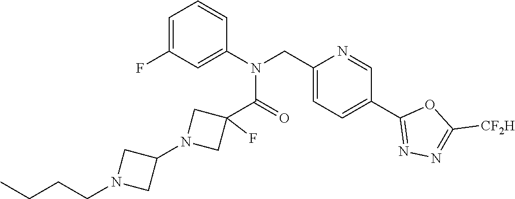 [SMILES: CCCCN1CC(N2CC(F)(C(=O)N(Cc3ccc(-c4nnc(C(F)F)o4)cn3)c3cccc(F)c3)C2)C1] |
| 88 | 3223 | 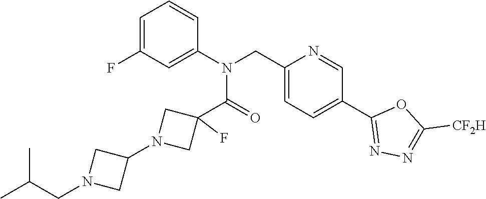 [SMILES: CC(C)CN1CC(N2CC(F)(C(=O)N(Cc3ccc(-c4nnc(C(F)F)o4)cn3)c3cccc(F)c3)C2)C1] |
| 89 | 3224 | 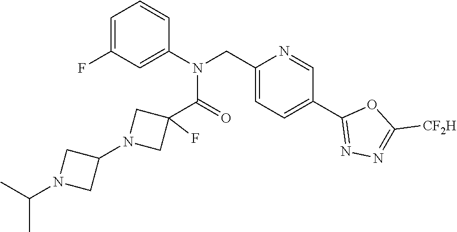 [SMILES: CC(C)N1CC(N2CC(F)(C(=O)N(Cc3ccc(-c4nnc(C(F)F)o4)cn3)c3cccc(F)c3)C2)C1] |
| 90 | 3389 | 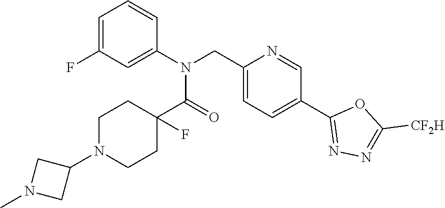 [SMILES: CN1CC(N2CCC(F)(C(=O)N(Cc3ccc(-c4nnc(C(F)F)o4)cn3)c3cccc(F)c3)CC2)C1] |
| 91 | 3390 | 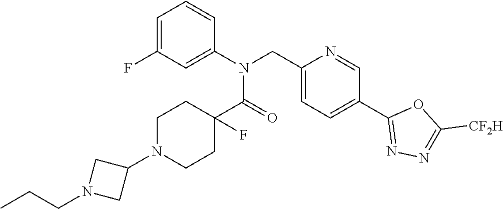 [SMILES: CCCN1CC(N2CCC(F)(C(=O)N(Cc3ccc(-c4nnc(C(F)F)o4)cn3)c3cccc(F)c3)CC2)C1] |
| 92 | 3391 | 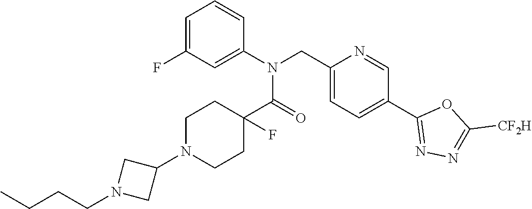 [SMILES: CCCCN1CC(N2CCC(F)(C(=O)N(Cc3ccc(-c4nnc(C(F)F)o4)cn3)c3cccc(F)c3)CC2)C1] |
| 93 | 3392 | 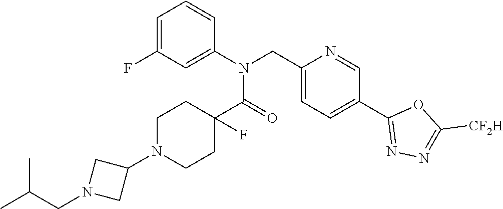 [SMILES: CC(C)CN1CC(N2CCC(F)(C(=O)N(Cc3ccc(-c4nnc(C(F)F)o4)cn3)c3cccc(F)c3)CC2)C1] |
| 94 | 3393 | 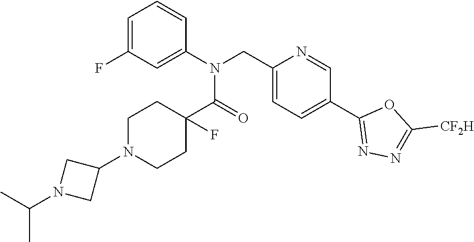 [SMILES: CC(C)N1CC(N2CCC(F)(C(=O)N(Cc3ccc(-c4nnc(C(F)F)o4)cn3)c3cccc(F)c3)CC2)C1] |
| 95 | 3394 | 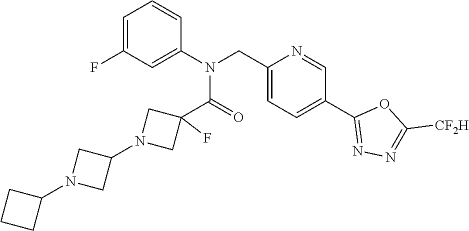 [SMILES: O=C(N(Cc1ccc(-c2nnc(C(F)F)o2)cn1)c1cccc(F)c1)C1(F)CN(C2CN(C3CCC3)C2)C1] |
| 96 | 3395 | 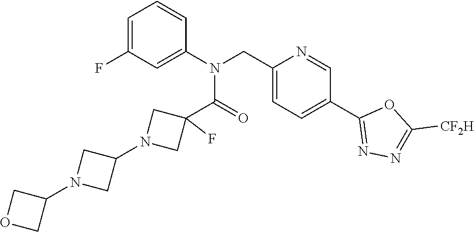 [SMILES: O=C(N(Cc1ccc(-c2nnc(C(F)F)o2)cn1)c1cccc(F)c1)C1(F)CN(C2CN(C3COC3)C2)C1] |
| 97 | 3396 | 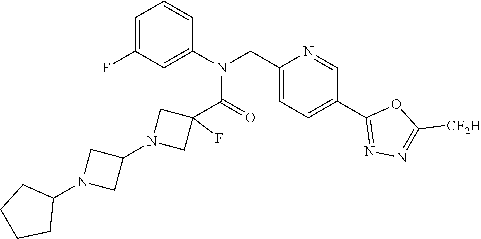 [SMILES: O=C(N(Cc1ccc(-c2nnc(C(F)F)o2)cn1)c1cccc(F)c1)C1(F)CN(C2CN(C3CCCC3)C2)C1] |
| 98 | 3397 | 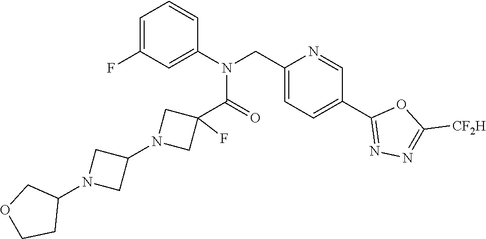 [SMILES: O=C(N(Cc1ccc(-c2nnc(C(F)F)o2)cn1)c1cccc(F)c1)C1(F)CN(C2CN(C3CCOC3)C2)C1] |
| 99 | 3398 | 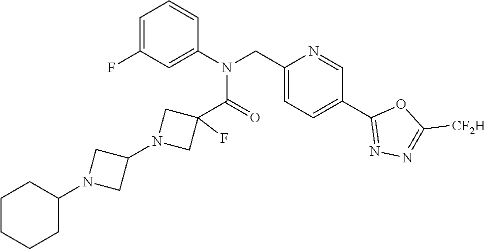 [SMILES: O=C(N(Cc1ccc(-c2nnc(C(F)F)o2)cn1)c1cccc(F)c1)C1(F)CN(C2CN(C3CCCCC3)C2)C1] |
| 100 | 3399 | 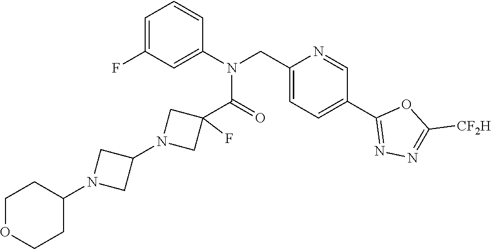 [SMILES: O=C(N(Cc1ccc(-c2nnc(C(F)F)o2)cn1)c1cccc(F)c1)C1(F)CN(C2CN(C3CCOCC3)C2)C1] |
| 101 | 3400 | 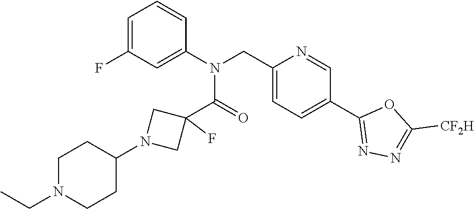 [SMILES: CCN1CCC(N2CC(F)(C(=O)N(Cc3ccc(-c4nnc(C(F)F)o4)cn3)c3cccc(F)c3)C2)CC1] |
| 102 | 3401 | 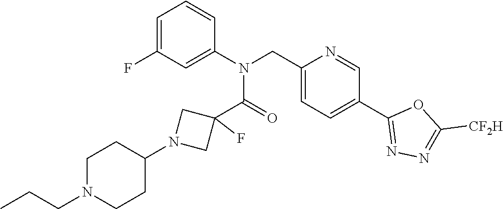 [SMILES: CCCN1CCC(N2CC(F)(C(=O)N(Cc3ccc(-c4nnc(C(F)F)o4)cn3)c3cccc(F)c3)C2)CC1] |
| 103 | 3402 | 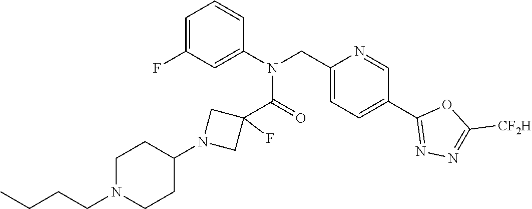 [SMILES: CCCCN1CCC(N2CC(F)(C(=O)N(Cc3ccc(-c4nnc(C(F)F)o4)cn3)c3cccc(F)c3)C2)CC1] |
| 104 | 3403 | 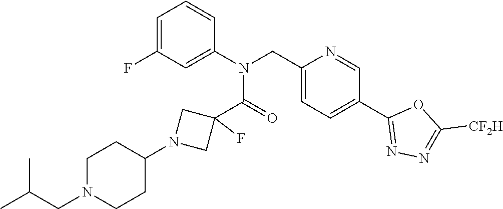 [SMILES: CC(C)CN1CCC(N2CC(F)(C(=O)N(Cc3ccc(-c4nnc(C(F)F)o4)cn3)c3cccc(F)c3)C2)CC1] |
| 105 | 3404 | 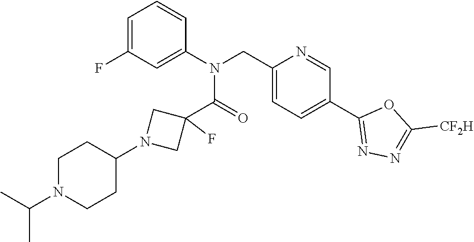 [SMILES: CC(C)N1CCC(N2CC(F)(C(=O)N(Cc3ccc(-c4nnc(C(F)F)o4)cn3)c3cccc(F)c3)C2)CC1] |
| 106 | 3405 | 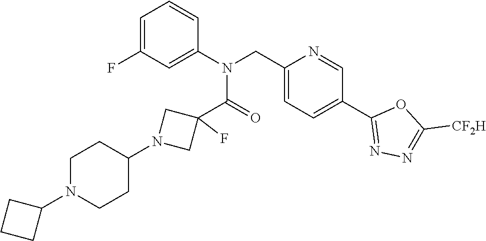 [SMILES: O=C(N(Cc1ccc(-c2nnc(C(F)F)o2)cn1)c1cccc(F)c1)C1(F)CN(C2CCN(C3CCC3)CC2)C1] |
| 107 | 3406 | 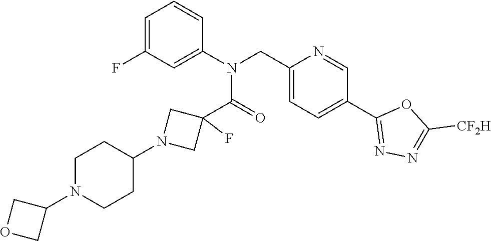 [SMILES: O=C(N(Cc1ccc(-c2nnc(C(F)F)o2)cn1)c1cccc(F)c1)C1(F)CN(C2CCN(C3COC3)CC2)C1] |
| 108 | 3407 | 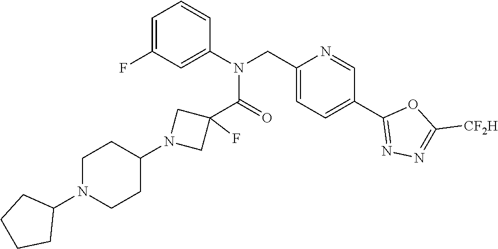 [SMILES: O=C(N(Cc1ccc(-c2nnc(C(F)F)o2)cn1)c1cccc(F)c1)C1(F)CN(C2CCN(C3CCCC3)CC2)C1] |
| 109 | 3408 | 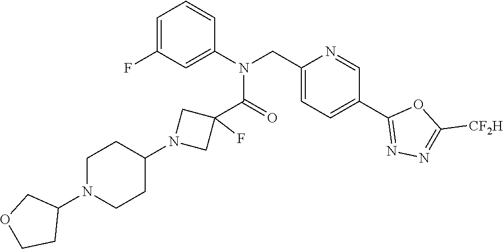 [SMILES: O=C(N(Cc1ccc(-c2nnc(C(F)F)o2)cn1)c1cccc(F)c1)C1(F)CN(C2CCN(C3CCOC3)CC2)C1] |
| 110 | 3409 | 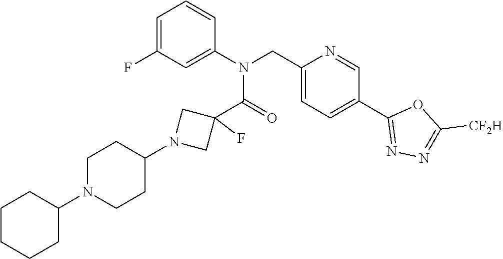 [SMILES: O=C(N(Cc1ccc(-c2nnc(C(F)F)o2)cn1)c1cccc(F)c1)C1(F)CN(C2CCN(C3CCCCC3)CC2)C1] |
| 111 | 3410 | 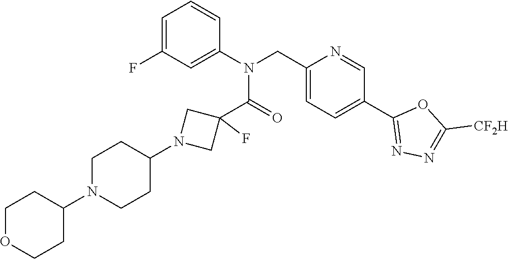 [SMILES: O=C(N(Cc1ccc(-c2nnc(C(F)F)o2)cn1)c1cccc(F)c1)C1(F)CN(C2CCN(C3CCOCC3)CC2)C1] |
| 112 | 3429 | 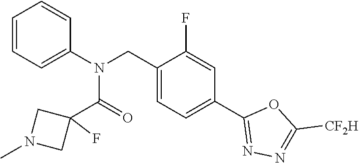 [SMILES: CN1CC(F)(C(=O)N(Cc2ccc(-c3nnc(C(F)F)o3)cc2F)c2ccccc2)C1] |
| 113 | 3430 | 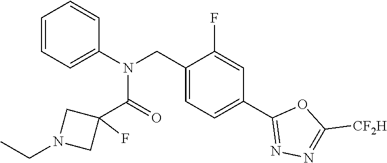 [SMILES: CCN1CC(F)(C(=O)N(Cc2ccc(-c3nnc(C(F)F)o3)cc2F)c2ccccc2)C1] |
| 114 | 3431 | 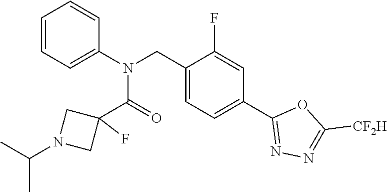 [SMILES: CC(C)N1CC(F)(C(=O)N(Cc2ccc(-c3nnc(C(F)F)o3)cc2F)c2ccccc2)C1] |
| 115 | 3432 | 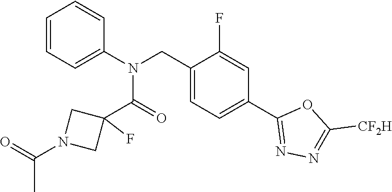 [SMILES: CC(=O)N1CC(F)(C(=O)N(Cc2ccc(-c3nnc(C(F)F)o3)cc2F)c2ccccc2)C1] |
| 116 | 3433 | 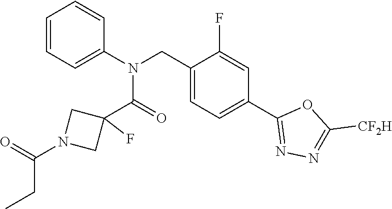 [SMILES: CCC(=O)N1CC(F)(C(=O)N(Cc2ccc(-c3nnc(C(F)F)o3)cc2F)c2ccccc2)C1] |
| 117 | 3434 | 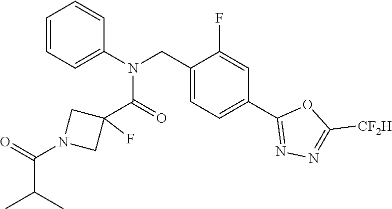 [SMILES: CC(C)C(=O)N1CC(F)(C(=O)N(Cc2ccc(-c3nnc(C(F)F)o3)cc2F)c2ccccc2)C1] |
| 118 | 3435 | 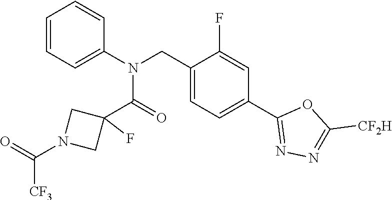 [SMILES: O=C(N1CC(F)(C(=O)N(Cc2ccc(-c3nnc(C(F)F)o3)cc2F)c2ccccc2)C1)C(F)(F)F] |
| 119 | 3436 | 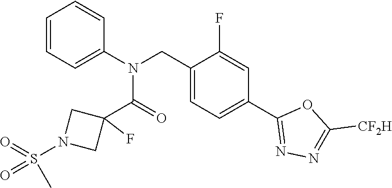 [SMILES: CS(=O)(=O)N1CC(F)(C(=O)N(Cc2ccc(-c3nnc(C(F)F)o3)cc2F)c2ccccc2)C1] |
| 120 | 3437 | 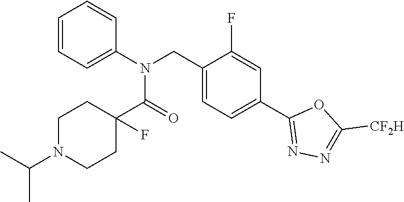 [SMILES: CC(C)N1CCC(F)(C(=O)N(Cc2ccc(-c3nnc(C(F)F)o3)cc2F)c2ccccc2)CC1] |
| 121 | 3438 | 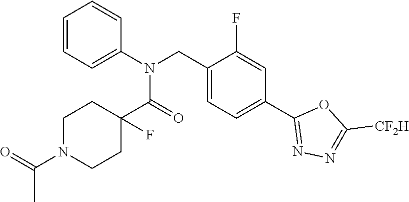 [SMILES: CC(=O)N1CCC(F)(C(=O)N(Cc2ccc(-c3nnc(C(F)F)o3)cc2F)c2ccccc2)CC1] |
| 122 | 3439 | 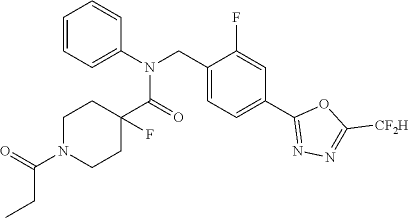 [SMILES: CCC(=O)N1CCC(F)(C(=O)N(Cc2ccc(-c3nnc(C(F)F)o3)cc2F)c2ccccc2)CC1] |
| 123 | 3440 | 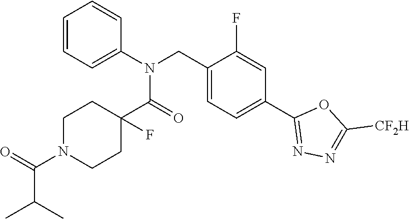 [SMILES: CC(C)C(=O)N1CCC(F)(C(=O)N(Cc2ccc(-c3nnc(C(F)F)o3)cc2F)c2ccccc2)CC1] |
| 124 | 3441 | 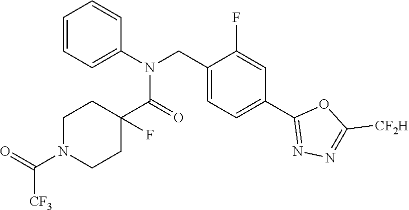 [SMILES: O=C(N1CCC(F)(C(=O)N(Cc2ccc(-c3nnc(C(F)F)o3)cc2F)c2ccccc2)CC1)C(F)(F)F] |
| 125 | 3442 | 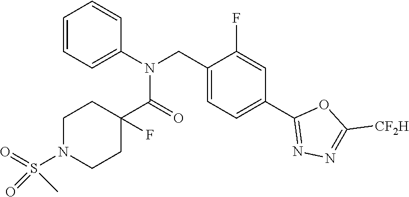 [SMILES: CS(=O)(=O)N1CCC(F)(C(=O)N(Cc2ccc(-c3nnc(C(F)F)o3)cc2F)c2ccccc2)CC1] |
| 126 | 3443 | 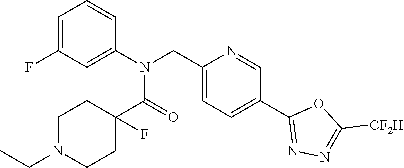 [SMILES: CCN1CCC(F)(C(=O)N(Cc2ccc(-c3nnc(C(F)F)o3)cn2)c2cccc(F)c2)CC1] |
| 127 | 6890 | 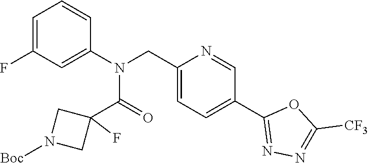 [SMILES: CC(C)(C)OC(=O)N1CC(F)(C(=O)N(Cc2ccc(-c3nnc(C(F)(F)F)o3)cn2)c2cccc(F)c2)C1] |
| 128 | 6891 | 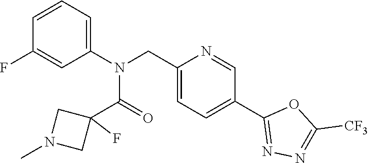 [SMILES: CN1CC(F)(C(=O)N(Cc2ccc(-c3nnc(C(F)(F)F)o3)cn2)c2cccc(F)c2)C1] |

6. A pharmaceutical composition for preventing or treating histone deacetylase 6-mediated diseases comprising the compound represented by Chemical Formula I, the optical isomer thereof, or the pharmaceutically acceptable salt thereof according to claim 1 as an active ingredient.

7. The pharmaceutical composition for preventing or treating the histone deacetylase 6-mediated diseases according to claim 6, wherein
the histone deacetylase 6-mediated diseases are infectious diseases; neoplasm; endocrine, nutritional and metabolic diseases; mental and behavioral disorders; neurological diseases; diseases of eyes and adnexa; circulatory diseases; respiratory diseases; digestive diseases; skin and subcutaneous tissue diseases; musculoskeletal and connective tissue diseases; or congenital malformations, alterations, or chromosomal abnormalities.

8. A method for preventing or treating the histone deacetylase 6-mediated diseases comprising administering a therapeutically effective amount of the compound represented by Chemical Formula L, the optical isomer thereof, or the pharmaceutically acceptable salt thereof according to claim 1 as an active ingredient.

9. The method for preventing or treating the histone deacetylase 6-mediated diseases according to claim 8, wherein the histone deacetylase 6-mediated diseases are infectious diseases; neoplasm; endocrine, nutritional and metabolic diseases; mental and behavioral disorders; neurological diseases; diseases of eyes and adnexa; circulatory diseases; respiratory diseases: digestive diseases; skin and subcutaneous tissue diseases; musculoskeletal and connective tissue diseases; or congenital malformations, alterations, or chromosomal abnormalities.
